SEQUENCE LISTING (194 sequences)

SEQ ID NO: 1 - protein, 5 aa, synthetic construct
GYWMH

SEQ ID NO: 2 - protein, 5 aa, synthetic construct
GYYMH

SEQ ID NO: 3 - protein, 5 aa, synthetic construct
GYFMH

SEQ ID NO: 4 - protein, 5 aa, synthetic construct
GYLMH

SEQ ID NO: 5 - protein, 17 aa, synthetic construct
EINPSNGRTNYNERFQG

SEQ ID NO: 6 - protein, 17 aa, synthetic construct
EINPSNGRTNYNEKFQG

SEQ ID NO: 7 - protein, 17 aa, synthetic construct
EINPSNGRTNYNERFKS

SEQ ID NO: 8 - protein, 17 aa, synthetic construct
EINPSNGRTNYNERLKS

SEQ ID NO: 9 - protein, 17 aa, synthetic construct
EINPSNARTNYNERFQG

SEQ ID NO: 10 - protein, 17 aa, synthetic construct
EINPSNARTNYNEKFQG

SEQ ID NO: 11 - protein, 17 aa, synthetic construct
EINPSNARTNYNERFKS

SEQ ID NO: 12 - protein, 17 aa, synthetic construct
EINPSNARTNYNERLKS

SEQ ID NO: 13 - protein, 11 aa, synthetic construct
DYYGTSYNFDY

SEQ ID NO: 14 - protein, 11 aa, synthetic construct
RANEDINNRLA

SEQ ID NO: 15 - protein, 11 aa, synthetic construct
KANEDINNRLA

SEQ ID NO: 16 - protein, 11 aa, synthetic construct
QANEDINNRLA

SEQ ID NO: 17 - protein, 11 aa, synthetic construct
RANEDINARLA

SEQ ID NO: 18 - protein, 11 aa, synthetic construct
KANEDINARLA

SEQ ID NO: 19 - protein, 11 aa, synthetic construct
QANEDINARLA

SEQ ID NO: 20 - protein, 11 aa, synthetic construct
RANEDINLRLA

SEQ ID NO: 21 - protein, 11 aa, synthetic construct
KANEDINLRLA

SEQ ID NO: 22 - protein, 11 aa, synthetic construct
QANEDINLRLA

SEQ ID NO: 23 - protein, 7 aa, synthetic construct
GATNLVT

SEQ ID NO: 24 - protein, 7 aa, synthetic construct
GASNLVS

SEQ ID NO: 25 - protein, 9 aa, synthetic construct
QQYWSTPFT

SEQ ID NO: 26 - protein, 9 aa, synthetic construct
QQYYSTPFT

SEQ ID NO: 27 - protein, 9 aa, synthetic construct
QQYFSTPFT

SEQ ID NO: 28 - protein, 120 aa, synthetic construct
QVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSS

SEQ ID NO: 29 - protein, 120 aa, synthetic construct
QVQLVQSGAEVKKPGASVKVSCKASGYTFTGYWMHWVRQAPGQGLEWIGEINPSNGRTNYNERFKSRVTLTVDKSISTAYMELSRLRSDDTAVYFCARDYYGTSYNFDYWGQGTLVTVSS

SEQ ID NO: 30 - protein, 120 aa, synthetic construct
QVQLQQWGAGLLKPSETLSLTCAAYGYTFTGYWMHWIRQPPGKGLEWIGEINPSNGRTNYNERLKSRVTLSVDKSKNQASLKLSSVTAADTAVYFCARDYYGTSYNFDYWGQGTLVTVSS

SEQ ID NO: 31 - protein, 120 aa, synthetic construct
QVQLVQSGAEVKKPGASVKVSCKASGYTFTGYWMHWVRQAPGQGLEWIGEINPSNGRTNYNEKFQGRVTLTVDKSISTAYMELSRLRSDDTAVYYCARDYYGTSYNFDYWGQGTLVTVSS

SEQ ID NO: 32 - protein, 120 aa, synthetic construct; identical to SEQ ID NO: 31

SEQ ID NO: 33 - protein, 107 aa, synthetic construct
DIQMTQSPSSLSASVGDRVTITCKANEDINNRLAWYQQKPGKAPKLLISGATNLVTGVPSRFSGSGSGKDYTLTISSLQPEDFATYYCQQYWSTPFTFGQGTKLEIK

SEQ ID NO: 34 - protein, 107 aa, synthetic construct
DIQMTQSPSSLSASVGDRVTITCKANEDINNRLAWYQQKPGKAPKLLISGATNLVTGVPSRFSGSGSGKDYTLTISSLQPEDIATYYCQQYWSTPFTFGQGTKLEIK

SEQ ID NO: 35 - protein, 107 aa, synthetic construct
EIVMTQSPATLSVSPGERATLSCRANEDINNRLAWYQQKPGQAPRLLISGATNLVTGIPARFSGSGSGKEFTLTISSLQSEDFAVYYCQQYWSTPFTFGQGTKLEIK

SEQ ID NO: 36 - protein, 107 aa, synthetic construct
DIQMTQSPSSLSASVGDRVTITCRANEDINNRLAWYQQKPGKAPKLLISGATNLVTGVPSRFSGSGSGKDYTLTISSLQPEDFATYYCQQYWSTPFTFGQGTKLEIK

SEQ ID NO: 37 - protein, 107 aa, synthetic construct
DIQMTQSPSSLSASVGDRVTITCRANEDINNRLAWYQQKPGKAPKLLISGASNLVSGVPSRFSGSGSGKDYTLTISSLQPEDFATYYCQQYWSTPFTFGQGTKLEIK

SEQ ID NO: 38 - protein, 30 aa, synthetic construct
QVQLVQSGAEVKKPGASVKVSCKASGYTFT

SEQ ID NO: 39 - protein, 30 aa, synthetic construct
QVQLQQWGAGLLKPSETLSLTCAAYGYTFT

SEQ ID NO: 40 - protein, 14 aa, synthetic construct
WVRQAPGQGLEWIG

SEQ ID NO: 41 - protein, 14 aa, synthetic construct
WIRQPPGKGLEWIG

SEQ ID NO: 42 - protein, 32 aa, synthetic construct
RVTLTVDKSISTAYMELSRLRSDDTAVYFCAR

SEQ ID NO: 43 - protein, 32 aa, synthetic construct
RVTLSVDKSKNQASLKLSSVTAADTAVYFCAR

SEQ ID NO: 44 - protein, 11 aa, synthetic construct
WGQGTLVTVSS

SEQ ID NO: 45 - protein, 23 aa, synthetic construct
DIQMTQSPSSLSASVGDRVTITC

SEQ ID NO: 46 - protein, 23 aa, synthetic construct
EIVMTQSPATLSVSPGERATLSC

SEQ ID NO: 47 - protein, 15 aa, synthetic construct
WYQQKPGKAPKLLIS

SEQ ID NO: 48 - protein, 15 aa, synthetic construct
WYQQKPGQAPRLLIS

SEQ ID NO: 49 - protein, 32 aa, synthetic construct
GVPSRFSGSGSGKDYTLTISSLQPEDIATYYC

SEQ ID NO: 50 - protein, 32 aa, synthetic construct
GIPARFSGSGSGKEFTLTISSLQSEDFAVYYC

SEQ ID NO: 51 - protein, 10 aa, synthetic construct
FGQGTKLEIK

SEQ ID NO: 52 - protein, 6 aa, synthetic construct
LCTPSR

SEQ ID NO: 53 - protein, 5 aa, synthetic construct
  VAR_SEQ 3: note = Any natural amino acid residue
LPXTG

SEQ ID NO: 54 - protein, 5 aa, synthetic construct
  MOD_RES 5: note = 6-azido-L-lysine
KAYAX

SEQ ID NO: 55 - protein, 120 aa, synthetic construct
QVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSS

SEQ ID NO: 56 - protein, 120 aa, synthetic construct
QVQLQQPGAELVKPGASVKLSCKASGYTFTGYYMHWVKQRPGHGLEWIGEINPSNGRTNYNERFKSKATLTVDKSSTTAFMQLSGLTSEDSAVYFCARDYYGTSYNFDYWGQGTTLTVSS

SEQ ID NO: 57 - protein, 120 aa, synthetic construct
QVQLQQPGAELVKPGASVKLSCKASGYTFTGYFMHWVKQRPGHGLEWIGEINPSNGRTNYNERFKSKATLTVDKSSTTAFMQLSGLTSEDSAVYFCARDYYGTSYNFDYWGQGTTLTVSS

SEQ ID NO: 58 - protein, 120 aa, synthetic construct
QVQLQQPGAELVKPGASVKLSCKASGYTFTGYLMHWVKQRPGHGLEWIGEINPSNGRTNYNERFKSKATLTVDKSSTTAFMQLSGLTSEDSAVYFCARDYYGTSYNFDYWGQGTTLTVSS

SEQ ID NO: 59 - protein, 120 aa, synthetic construct
QVQLQQPGAELVKPGASVKLSCKASGYTFTGYWMHWVKQRPGHGLEWIGEINPSNARTNYNERFKSKATLTVDKSSTTAFMQLSGLTSEDSAVYFCARDYYGTSYNFDYWGQGTTLTVSS

SEQ ID NO: 60 - protein, 107 aa, synthetic construct
DIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIK

SEQ ID NO: 61 - protein, 107 aa, synthetic construct
DIQMTQSSSSFSVSLGDRVTITCKANEDINLRLAWYQQTPGNSPRLLISGATNLVTGVPSRFSGSGSGKDYTLTITSLQAEDFATYYCQQYWSTPFTFGSGTELEIK

SEQ ID NO: 62 - protein, 107 aa, synthetic construct
DIQMTQSSSSFSVSLGDRVTITCKANEDINARLAWYQQTPGNSPRLLISGATNLVTGVPSRFSGSGSGKDYTLTITSLQAEDFATYYCQQYWSTPFTFGSGTELEIK

SEQ ID NO: 63 - protein, 107 aa, synthetic construct
DIQMTQSSSSFSVSLGDRVTITCKANEDINNRLAWYQQTPGNSPRLLISGATNLVTGVPSRFSGSGSGKDYTLTITSLQAEDFATYYCQQYYSTPFTFGSGTELEIK

SEQ ID NO: 64 - protein, 107 aa, synthetic construct
DIQMTQSSSSFSVSLGDRVTITCKANEDINNRLAWYQQTPGNSPRLLISGATNLVTGVPSRFSGSGSGKDYTLTITSLQAEDFATYYCQQYFSTPFTFGSGTELEIK

SEQ ID NO: 65 - protein, 98 aa, synthetic construct
QVQLVQSGAEVKKPGASVKVSCKASGYTFTGYWMHWVRQAPGQGLEWIGEINPSNGRTNYNERFKSRVTLTVDKSISTAYMELSRLRSDDTAVYFCAR

SEQ ID NO: 66 - protein, 98 aa, synthetic construct
QVQLVQSGAEVKKPGASVKVSCKASGYTFTGYWMHWVRQAPGQGLEWIGEINPSNGRTNYNERFQGRVTLTVDKSISTAYMELSRLRSDDTAVYYCAR

SEQ ID NO: 67 - protein, 98 aa, synthetic construct
QVQLVQSGAEVKKPGASVKVSCKASGYTFTGYWMHWVRQAPGQGLEWIGEINPSNGRTNYNEKFQGRVTLTVDKSISTAYMELSRLRSDDTAVYYCAR

SEQ ID NO: 68 - protein, 98 aa, synthetic construct
QVQLVQSGAEVKKPGASVKVSCKASGYTFTGYWMHWVRQAPGQGLEWMGEINPSNGRTNYNEKFQGRVTLTVDKSISTAYMELSRLRSDDTAVYYCAR

SEQ ID NO: 69 - protein, 98 aa, synthetic construct
QVQLQQWGAGLLKPSETLSLTCAAYGYTFTGYWMHWIRQPPGKGLEWIGEINPSNGRTNYNERLKSRVTLSVDKSKNQASLKLSSVTAADTAVYFCAR

SEQ ID NO: 70 - protein, 98 aa, synthetic construct
QVQLQQWGAGLLKPSETLSLTCAAYGYSFSGYWMHWIRQPPGKGLEWIGEINPSNGRTNYNERLKSRVTLSVDKSKNQASLKLSSVTAADTAVYYCAR

SEQ ID NO: 71 - protein, 98 aa, synthetic construct
QVQLQQWGAGLLKPSETLSLTCAAYGYSFSGYWMHWIRQPPGKGLEWIGEINPSNGRTNYNPSLKSRVTLSVDKSKNQASLKLSSVTAADTAVYYCAR

SEQ ID NO: 72 - protein, 107 aa, synthetic construct; identical to SEQ ID NO: 60

SEQ ID NO: 73 - protein, 95 aa, synthetic construct
DIQMTQSPSSLSASVGDRVTITCKANEDINNRLAWYQQKPGKAPKLLISGATNLVTGVPSRFSGSGSGKDYTLTISSLQPEDFATYYCQQYWSTP

SEQ ID NO: 74 - protein, 95 aa, synthetic construct
DIQMTQSPSSLSASVGDRVTITCRANEDINNRLAWYQQKPGKAPKLLISGATNLVTGVPSRFSGSGSGKDYTLTISSLQPEDFATYYCQQYWSTP

SEQ ID NO: 75 - protein, 95 aa, synthetic construct
DIQMTQSPSSLSASVGDRVTITCRANEDINNRLAWYQQKPGKAPKLLISGASNLVSGVPSRFSGSGSGKDYTLTISSLQPEDFATYYCQQYWSTP

SEQ ID NO: 76 - protein, 95 aa, synthetic construct
DIQMTQSPSSLSASVGDRVTITCRASQGINNRLAWYQQKPGKAPKLLISGASNLVSGVPSRFSGSGSGKDYTLTISSLQPEDFATYYCQQYWSTP

SEQ ID NO: 77 - protein, 95 aa, synthetic construct
DIQMTQSPSSLSASVGDRVTITCKANEDINNRLAWYQQKPGKAPKLLISGATNLVTGVPSRFSGSGSGKDYTLTISSLQPEDIATYYCQQYWSTP

SEQ ID NO: 78 - protein, 95 aa, synthetic construct
DIQMTQSPSSLSASVGDRVTITCQANEDINNRLAWYQQKPGKAPKLLISGATNLVTGVPSRFSGSGSGKDYTFTISSLQPEDIATYYCQQYWSTP

SEQ ID NO: 79 - protein, 95 aa, synthetic construct
DIQMTQSPSSLSASVGDRVTITCQANEDINNRLAWYQQKPGKAPKLLISGASNLVTGVPSRFSGSGSGKDFTFTISSLQPEDIATYYCQQYWSTP

SEQ ID NO: 80 - protein, 95 aa, synthetic construct
DIQMTQSPSSLSASVGDRVTITCQASQDINNRLAWYQQKPGKAPKLLISGASNLVTGVPSRFSGSGSGKDFTFTISSLQPEDIATYYCQQYWSTP

SEQ ID NO: 81 - protein, 95 aa, synthetic construct
EIVMTQSPATLSVSPGERATLSCKANEDINNRLAWYQQKPGQAPRLLISGATNLVTGIPARFSGSGSGKEYTLTISSLQSEDFAVYYCQQYWSTP

SEQ ID NO: 82 - protein, 95 aa, synthetic construct
EIVMTQSPATLSVSPGERATLSCRANEDINNRLAWYQQKPGQAPRLLISGATNLVTGIPARFSGSGSGKEFTLTISSLQSEDFAVYYCQQYWSTP

SEQ ID NO: 83 - protein, 95 aa, synthetic construct
EIVMTQSPATLSVSPGERATLSCRASEDINNRLAWYQQKPGQAPRLLISGASNLVTGIPARFSGSGSGKEFTLTISSLQSEDFAVYYCQQYWSTP

SEQ ID NO: 84 - protein, 95 aa, synthetic construct
EIVMTQSPATLSVSPGERATLSCRASQSINNRLAWYQQKPGQAPRLLISGASNLVTGIPARFSGSGSGKEFTLTISSLQSEDFAVYYCQQYWSTP

SEQ ID NO: 85 - DNA, 1433 bp, synthetic construct
  source 1..1433: mol_type = other DNA
gaattcgccgccaccatgaagcacctgtggttcttcctgctgctggtggctgccccccggtgggttctgagccaggtgcagctggtgcagagcggcgccgaggtgaagaagcctggcgcctccgtgaaggtgtcctgtaaggcctccggctataccttcaccggctactggatgcactgggtgaggcaggctcccggccagggactggagtggatcggcgagatcaatccttccaatggcaggaccaactataacgagaggtttaagtcccgggtgaccctgaccgtggataagagcatcagcaccgcttacatggagctgagcaggctgcggtccgacgataccgccgtgtacttttgtgctagggactattacggcaccagctacaattttgactattggggccagggcaccctggtgaccgtgagctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 86 - DNA, 1433 bp, synthetic construct
  source 1..1433: mol_type = other DNA
gaattcgccgccaccatgaagcacctgtggtttttcctgctgctggtggccgctcctcggtgggtgctgagccaggtgcagctggtgcagtccggcgccgaggtgaagaagcctggcgccagcgtgaaggtgagctgcaaggcttccggctacacctttaccggctactggatgcactgggtgcggcaggctcctggccagggactggagtggatcggcgagatcaacccttccaatggccggaccaactataatgagaggttccagggcagggtgaccctgaccgtggataagtccatctccaccgcctatatggagctgtccaggctgcggtccgacgacaccgccgtgtactattgcgcccgggactactacggcacctcctacaactttgactactggggccagggcaccctggtgaccgtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 87 - DNA, 1433 bp, synthetic construct
  source 1..1433: mol_type = other DNA
gaattcgccgccaccatgaagcacctgtggtttttcctgctgctggtggctgcccctaggtgggtgctgagccaggtgcagctgcagcagtggggcgccggactgctgaagcccagcgagaccctgtccctgacctgcgctgcctacggctacaccttcaccggctattggatgcactggatccggcagccccctggcaagggcctggagtggatcggcgagatcaatcctagcaacggccggaccaattataatgagaggctgaagagccgggtgaccctgtccgtggacaagagcaagaatcaggcttccctgaagctgtcctccgtgaccgctgccgacaccgccgtgtacttttgcgctagggactactatggcacctcctacaattttgattactggggccagggcaccctggtgaccgtgtccagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 88 - DNA, 728 bp, synthetic construct
  source 1..728: mol_type = other DNA
gaattcgccgccaccatggtgctgcagacccaggtgttcatctccctgctgctgtggatctccggcgcctacggcgacatccagatgacccagtcccctagctccctgagcgcctccgtgggcgacagagtgaccatcacctgcaaggccaatgaggatatcaacaaccggctggcttggtatcagcagaagcccggcaaggctcccaagctgctgatctccggcgctaccaatctggtgaccggcgtgccctccaggtttagcggcagcggctccggcaaggattacaccctgaccatcagcagcctgcagcccgaggacttcgctacctactattgccagcagtactggagcaccccttttacctttggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 89 - DNA, 728 bp, synthetic construct
  source 1..728: mol_type = other DNA
gaattcgccgccaccatggtgctgcagacccaggtgttcatctccctgctgctgtggatctccggcgcctacggcgatatccagatgacccagagccctagcagcctgtccgccagcgtgggcgatagggtgaccatcacctgcagggctaacgaggacatcaataacaggctggcctggtaccagcagaagcccggcaaggcccctaagctgctgatcagcggcgccaccaatctggtgaccggcgtgcccagcaggttcagcggcagcggaagcggcaaggactacaccctgaccatctcctccctgcagcctgaggactttgccacctattattgccagcagtactggagcacccctttcacctttggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 90 - DNA, 728 bp, synthetic construct
  source 1..728: mol_type = other DNA
gaattcgccgccaccatggtgctgcagacccaggtgtttatctccctgctgctgtggatcagcggcgcttatggcgacatccagatgacccagtccccctccagcctgtccgccagcgtgggagatcgggtgaccatcacctgtaaggctaatgaggacatcaataaccggctggcttggtaccagcagaagcccggcaaggctcccaagctgctgatctccggcgctaccaacctggtgaccggcgtgcctagcaggttttccggctccggcagcggcaaggattataccctgaccatctcctccctgcagcccgaggatatcgctacctactactgccagcagtattggtccaccccttttaccttcggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 91 - DNA, 728 bp, synthetic construct
  source 1..728: mol_type = other DNA
gaattcgccgccaccatggtgctgcagacccaggtgttcatcagcctgctgctgtggatctccggcgcctacggcgacatccagatgacccagagcccctccagcctgtccgcttccgtgggcgatagggtgaccatcacctgtcaggctaatgaggacatcaacaacaggctggcttggtaccagcagaagcccggcaaggcccccaagctgctgatcagcggcgccaccaacctggtgaccggcgtgccaagcaggttcagcggcagcggcagcggaaaggactataccttcaccatctcctccctgcagcctgaggacatcgctacctattactgtcagcagtactggtccacccccttcacctttggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 92 - DNA, 728 bp, synthetic construct
  source 1..728: mol_type = other DNA
gaattcgccgccaccatggtgctgcagacccaggtgtttatcagcctgctgctgtggatcagcggcgcctacggcgagatcgtgatgacccagtcccctgccaccctgtccgtgtcccctggcgagagggccaccctgtcttgcaaggctaatgaggacatcaacaataggctggcctggtatcagcagaagcctggccaggctcctcggctgctgatcagcggcgctaccaacctggtgaccggcatccctgcccggttctccggctccggaagcggaaaggagtataccctgaccatcagcagcctgcagagcgaggattttgccgtgtactattgccagcagtactggtccaccccctttacctttggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 93 - DNA, 728 bp, synthetic construct
  source 1..728: mol_type = other DNA
gaattcgccgccaccatggtgctgcagacccaggtgttcatctccctgctgctgtggatcagcggcgcctacggcgagatcgtgatgacccagagccctgccaccctgagcgtgtcccctggcgagagagccaccctgtcctgccgggctaacgaggacatcaacaaccggctggcttggtatcagcagaagcctggccaggctcctaggctgctgatctccggcgctaccaacctggtgaccggcatccctgccaggtttagcggcagcggctccggcaaggagttcaccctgaccatcagcagcctgcagtccgaggatttcgccgtgtattattgtcagcagtactggtccacccccttcaccttcggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 94 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNERFKSRVTLTVDKSISTAYMELSRLRSDDTAVYFCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 469 aa, synthetic construct
MKHLWFFLLLVAAPRWVLSQVQLQQWGAGLLKPSETLSLTCAAYGYTFTGYWMHWIRQPPGKGLEWIGEINPSNGRTNYNERLKSRVTLSVDKSKNQASLKLSSVTAADTAVYFCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 234 aa, synthetic construct
MVLQTQVFISLLLWISGAYGDIQMTQSPSSLSASVGDRVTITCKANEDINNRLAWYQQKPGKAPKLLISGATNLVTGVPSRFSGSGSGKDYTLTISSLQPEDFATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 234 aa, synthetic construct
MVLQTQVFISLLLWISGAYGDIQMTQSPSSLSASVGDRVTITCRANEDINNRLAWYQQKPGKAPKLLISGATNLVTGVPSRFSGSGSGKDYTLTISSLQPEDFATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 234 aa, synthetic construct
MVLQTQVFISLLLWISGAYGDIQMTQSPSSLSASVGDRVTITCKANEDINNRLAWYQQKPGKAPKLLISGATNLVTGVPSRFSGSGSGKDYTLTISSLQPEDIATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 234 aa, synthetic construct
MVLQTQVFISLLLWISGAYGDIQMTQSPSSLSASVGDRVTITCQANEDINNRLAWYQQKPGKAPKLLISGATNLVTGVPSRFSGSGSGKDYTFTISSLQPEDIATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 234 aa, synthetic construct
MVLQTQVFISLLLWISGAYGEIVMTQSPATLSVSPGERATLSCKANEDINNRLAWYQQKPGQAPRLLISGATNLVTGIPARFSGSGSGKEYTLTISSLQSEDFAVYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 234 aa, synthetic construct
MVLQTQVFISLLLWISGAYGEIVMTQSPATLSVSPGERATLSCRANEDINNRLAWYQQKPGQAPRLLISGATNLVTGIPARFSGSGSGKEFTLTISSLQSEDFAVYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - DNA, 1431 bp, synthetic construct
  source 1..1431: mol_type = other DNA
gaattcgccgccaccatgaagcacctgtggttcttcctgctgctggtggccgcccccagatgggtgctgtcccaggtgcagctggtgcagagcggcgccgaggtgaagaagcctggcgcctccgtgaaggtgagctgcaaggcctccggctacacattcactggctactggatgcactgggtgagacaggcccctggccagggcctggagtggatcggagagatcaaccccagcaatggcagaaccaattacaatgagagattccagggcagagtgaccctgaccgtggataagtccatcagcacagcctacatggagctgtccagactgagaagcgatgacaccgccgtgtactactgcgccagagactactacggcacctcctacaactttgattactggggccagggcacactggtgacagtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 104 - DNA, 1431 bp, synthetic construct
  source 1..1431: mol_type = other DNA
gaattcgccgccaccatgaagcacctgtggtttttcctgctgctggtggccgcccccaggtgggttctgtcccaggtgcagctggtgcagagcggcgccgaggtgaagaagcctggcgcctccgtgaaggtgtcctgcaaggccagcggctacaccttcacaggctactggatgcactgggtgaggcaggcccccggccagggactggaatggatcggcgagatcaaccctagcaacggcaggacaaattacaacgagaagttccagggcagagtgacactgacagtggacaagtccatcagcaccgcctacatggagctgtccagactgagatccgacgatacagccgtgtactactgcgccagggactactacggcaccagctacaattttgactactggggccagggcaccctggtgacagtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 105 - DNA, 1431 bp, synthetic construct
  source 1..1431: mol_type = other DNA
gaattcgccgccaccatgaagcacctgtggtttttcctgctgctggtggccgcccccaggtgggttctgagccaggtgcagctggtgcagagcggcgccgaggtgaagaagcccggcgcttccgtgaaggtgtcctgtaaggccagcggctacacattcaccggctactggatgcactgggtgagacaggcccccggccagggactggagtggatgggagagatcaatcctagcaacggcagaacaaattacaatgagaagttccagggcagagtgacactgacagtggacaagagcatctccacagcctacatggagctgtccagactgagatccgacgacaccgccgtgtactactgtgccagagattactacggcacatcctacaacttcgactactggggccagggcacactggtgacagtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 106 - DNA, 728 bp, synthetic construct
  source 1..728: mol_type = other DNA
gaattcgccgccaccatggtgctgcagacccaggtgttcatctccctgctgctgtggatcagcggcgcttatggcgacatccagatgacccagagcccttcctccctgtccgccagcgtgggcgacagggtgaccatcacctgtaaggccaatgaggatatcaataatcggctggcctggtatcagcagaagcccggcaaggcccccaagctgctgatctccggcgctaccaatctggtgaccggcgtgccttcccggttttccggctccggcagcggcaaggattataccctgaccatcagcagcctgcagcccgaggattttgctacctattattgccagcagtattggtccacccccttcacctttggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 107 - DNA, 728 bp, synthetic construct
  source 1..728: mol_type = other DNA
gaattcgccgccaccatggtgctgcagacccaggtgtttatctccctgctgctgtggatctccggcgcctacggcgacatccagatgacccagtcccccagctccctgtccgctagcgtgggcgacagggtgaccatcacctgtagggccaatgaggacatcaacaacaggctggcctggtatcagcagaagcccggcaaggctcctaagctgctgatctccggcgctaccaatctggtgaccggcgtgccttccaggttctccggctccggctccggaaaggattacaccctgaccatcagctccctgcagcctgaggactttgctacctattactgccagcagtactggtccaccccctttaccttcggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 108 - DNA, 728 bp, synthetic construct
  source 1..728: mol_type = other DNA
gaattcgccgccaccatggtgctgcagacccaggtgtttatctccctgctgctgtggatctccggcgcctacggcgatatccagatgacccagagcccctccagcctgagcgcctccgtgggagacagggtgaccatcacctgtagggccaacgaggatatcaacaacaggctggcctggtatcagcagaagcctggcaaggctcctaagctgctgatcagcggcgcttccaacctggtgagcggcgtgccctccaggtttagcggcagcggcagcggaaaggattacaccctgaccatcagctccctgcagcccgaggacttcgctacctactattgccagcagtactggagcacccctttcaccttcggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 109 - DNA, 728 bp, synthetic construct
  source 1..728: mol_type = other DNA
gaattcgccgccaccatggtgctgcagacccaggtgttcatctccctgctgctgtggatcagcggcgcttatggcgacatccagatgacccagtcccctagctccctgagcgcctccgtgggcgacagggtgaccatcacctgccgggccagccagggcatcaataatcggctggcttggtaccagcagaagcccggcaaggcccctaagctgctgatcagcggcgcctccaacctggtgagcggcgtgccttccaggttctccggcagcggcagcggcaaggactataccctgaccatcagcagcctgcagcctgaggactttgccacctactattgccagcagtattggtccaccccctttacctttggccagggcaccaagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 110 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNEKFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 469 aa, synthetic construct
MKHLWFFLLLVAAPRWVLSQVQLVQSGAEVKKPGASVKVSCKASGYTFTGYWMHWVRQAPGQGLEWMGEINPSNGRTNYNEKFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 234 aa, synthetic construct
MVLQTQVFISLLLWISGAYGDIQMTQSPSSLSASVGDRVTITCRANEDINNRLAWYQQKPGKAPKLLISGASNLVSGVPSRFSGSGSGKDYTLTISSLQPEDFATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 234 aa, synthetic construct
MVLQTQVFISLLLWISGAYGDIQMTQSPSSLSASVGDRVTITCRASQGINNRLAWYQQKPGKAPKLLISGASNLVSGVPSRFSGSGSGKDYTLTISSLQPEDFATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 1431 bp, synthetic construct
  source 1..1431: mol_type = other DNA
gaattcgccgccaccatgaagcacctgtggttcttcctgctgctggtggctgccccccggtgggttctgagccaggtgcagctggtgcagagcggcgccgaggtgaagaagcctggcgcctccgtgaaggtgtcctgtaaggcctccggctataccttcaccggctactggatgcactgggtgaggcaggctcccggccagggactggagtggatcggcgagatcaatccttccaatggcaggaccaactataacgagaggtttaagtcccgggtgaccctgaccgtggataagagcatcagcaccgcttacatggagctgagcaggctgcggtccgacgataccgccgtgtacttttgtgctagggactattacggcaccagctacaattttgactattggggccagggcaccctggtgaccgtgagctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 116 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNERFKSRVTLTVDKSISTAYMELSRLRSDDTAVYFCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 1431 bp, synthetic construct
  source 1..1431: mol_type = other DNA
gaattcgccgccaccatgaagcacctgtggtttttcctgctgctggtggccgcccctagatgggtgctgtcccaggtgcagctgcagcagcctggcgccgagctggtgaagcctggcgcttccgtgaagctgtcctgtaaggcctccggctacaccttcaccggctactacatgcactgggtgaagcagagacctggccacggcctggagtggatcggcgagatcaatcccagcaatggcaggacaaattacaatgagaggttcaagtccaaggccaccctgaccgtggataagagcagcacaacagccttcatgcagctgagcggcctgaccagcgaggattccgccgtgtacttttgtgccagagattactacggcaccagctacaacttcgactactggggccagggcacaacactgacagtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 118 - DNA, 1431 bp, synthetic construct
  source 1..1431: mol_type = other DNA
gaattcgccgccaccatgaagcacctgtggttcttcctgctgctggtggccgcccccaggtgggttctgagccaggtgcagctgcagcagcccggcgctgagctggtgaagcccggagcttccgtgaagctgtcctgcaaggccagcggctacacctttaccggctacttcatgcactgggtgaagcagagacccggccacggcctggagtggatcggcgaaatcaaccccagcaacggcagaaccaactacaatgagagattcaagtccaaggccaccctgaccgtggataagagcagcacaacagccttcatgcagctgtccggcctgacaagcgaggacagcgccgtgtacttctgtgccagagattactacggcaccagctacaatttcgattactggggccagggcacaaccctgacagtgtcctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 119 - DNA, 1431 bp, synthetic construct
  source 1..1431: mol_type = other DNA
gaattcgccgccaccatgaagcacctgtggttcttcctgctgctggtggccgcccctagatgggtgctgtcccaggtgcagctgcagcagcccggcgctgagctggtgaagcctggcgcttccgtgaagctgtcctgtaaggccagcggctacaccttcaccggctacctgatgcactgggtgaagcagagacctggccacggcctggagtggatcggcgagatcaatcccagcaatggcagaacaaattacaacgagaggttcaagtccaaggccaccctgaccgtggacaagtcctccaccaccgccttcatgcagctgtccggcctgaccagcgaggacagcgccgtgtacttctgtgccagggactactacggcaccagctacaatttcgactactggggccagggcaccacactgaccgtgtccagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 120 - DNA, 1431 bp, synthetic construct
  source 1..1431: mol_type = other DNA
gaattcgccgccaccatgaagcacctgtggttctttctgctgctggtggccgcccctaggtgggtgctgtcccaggtgcagctgcagcagcctggcgccgagctggtgaagcccggagcttccgtgaagctgtcctgcaaggccagcggctacaccttcacaggctactggatgcactgggtgaagcagaggcccggccacggcctggagtggatcggagagatcaacccctccaacgccaggaccaattacaacgagagattcaaatccaaggccaccctgaccgtggacaagagcagcacaacagccttcatgcagctgagcggcctgacaagcgaggattccgccgtgtacttttgcgccagggattactacggcacaagctacaattttgattactggggccagggcaccaccctgaccgtcagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 121 - DNA, 1431 bp, synthetic construct
  source 1..1431: mol_type = other DNA
gaattcgccgccaccatgaagcacctgtggttctttctgctgctggtggccgcccccaggtgggttctgagccaggtgcagctgcagcagcctggcgccgagctggtgaagcctggcgcttccgtgaagctgagctgcaaggcctccggctacacattcactggctactggatgcactgggtgaagcagagacctggccacggcctggagtggatcggcgagatcaatcccagcaatggcagaaccaactacaacgagaggttcaagtccaaggccaccctgaccgtggataagagctccaccaccgccttcatgcagctgtccggcctgaccagcgaggacagcgccgtgtacttttgtgccagagattactacggcaccagctacaactttgattactggggccagggcacaaccctgacagtgagctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 122 - DNA, 728 bp, synthetic construct
  source 1..728: mol_type = other DNA
gaattcgccgccaccatggtgctgcagacccaggtgtttatctccctgctgctgtggatcagcggcgcctacggcgatatccagatgacccagagctccagctcctttagcgtgagcctgggcgaccgggtgaccatcacctgtaaggctaatgaggacatcaataacaggctggcttggtaccagcagacccccggcaatagccctaggctgctgatcagcggcgctaccaatctggtgaccggcgtgcctagcaggtttagcggctccggctccggcaaggattacaccctgaccatcacctccctgcaggccgaggattttgctacctactattgtcagcagtactggagcaccccttttacctttggctccggcaccgagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 123 - DNA, 728 bp, synthetic construct
  source 1..728: mol_type = other DNA
gaattcgccgccaccatggtgctgcagacccaggtgttcatcagcctgctgctgtggatcagcggcgcttatggcgacatccagatgacccagagctcctcctccttttccgtgtccctgggcgatcgggtgaccatcacctgtaaggccaatgaggatatcaacctgaggctggcctggtaccagcagacccctggcaacagccctaggctgctgatctccggcgctaccaatctggtgaccggcgtgcctagccggtttagcggctccggcagcggcaaggactataccctgaccatcaccagcctgcaggccgaggactttgctacctattattgtcagcagtactggagcaccccctttacctttggctccggcaccgagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 124 - DNA, 728 bp, synthetic construct
  source 1..728: mol_type = other DNA
gaattcgccgccaccatggtgctgcagacccaggtgttcatctccctgctgctgtggatctccggcgcttatggcgatatccagatgacccagagctccagcagcttctccgtgagcctgggcgatagggtgaccatcacctgtaaggccaacgaggatatcaatgctaggctggcttggtatcagcagacccctggcaatagcccccggctgctgatctccggcgccacaaacctggtgaccggcgtgccttccaggttctccggcagcggctccggcaaggattataccctgaccatcaccagcctgcaggctgaggattttgctacctactactgtcagcagtattggagcaccccctttacctttggctccggcaccgagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 125 - DNA, 728 bp, synthetic construct
  source 1..728: mol_type = other DNA
gaattcgccgccaccatggtgctgcagacccaggtgtttatcagcctgctgctgtggatctccggcgcttatggcgatatccagatgacccagagctcctcctcctttagcgtgagcctgggcgatcgggtgaccatcacctgtaaggccaacgaggacatcaataacaggctggcttggtaccagcagacccctggcaacagccctcggctgctgatcagcggcgccaccaatctggtgaccggcgtgcccagcaggttcagcggcagcggatccggcaaggactacaccctgaccatcaccagcctgcaggctgaggactttgccacctattactgtcagcagtactattccacccctttcaccttcggctccggcaccgagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 126 - DNA, 728 bp, synthetic construct
  source 1..728: mol_type = other DNA
gaattcgccgccaccatggtgctgcagacccaggtgtttatctccctgctgctgtggatcagcggcgcttatggcgacatccagatgacccagagcagctccagcttctccgtgtccctgggcgaccgggtgaccatcacctgtaaggccaatgaggatatcaacaaccggctggcctggtatcagcagacccctggcaacagccccaggctgctgatctccggcgccaccaatctggtgaccggcgtgccctcccggttttccggcagcggaagcggcaaggattacaccctgaccatcacctccctgcaggctgaggatttcgctacctactattgccagcagtacttttccaccccctttacctttggctccggcaccgagctggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 127 - protein, 469 aa, synthetic construct
MKHLWFFLLLVAAPRWVLSQVQLQQPGAELVKPGASVKLSCKASGYTFTGYYMHWVKQRPGHGLEWIGEINPSNGRTNYNERFKSKATLTVDKSSTTAFMQLSGLTSEDSAVYFCARDYYGTSYNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 469 aa, synthetic construct
MKHLWFFLLLVAAPRWVLSQVQLQQPGAELVKPGASVKLSCKASGYTFTGYFMHWVKQRPGHGLEWIGEINPSNGRTNYNERFKSKATLTVDKSSTTAFMQLSGLTSEDSAVYFCARDYYGTSYNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 469 aa, synthetic construct
MKHLWFFLLLVAAPRWVLSQVQLQQPGAELVKPGASVKLSCKASGYTFTGYLMHWVKQRPGHGLEWIGEINPSNGRTNYNERFKSKATLTVDKSSTTAFMQLSGLTSEDSAVYFCARDYYGTSYNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 469 aa, synthetic construct
MKHLWFFLLLVAAPRWVLSQVQLQQPGAELVKPGASVKLSCKASGYTFTGYWMHWVKQRPGHGLEWIGEINPSNARTNYNERFKSKATLTVDKSSTTAFMQLSGLTSEDSAVYFCARDYYGTSYNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 469 aa, synthetic construct
MKHLWFFLLLVAAPRWVLSQVQLQQPGAELVKPGASVKLSCKASGYTFTGYWMHWVKQRPGHGLEWIGEINPSNGRTNYNERFKSKATLTVDKSSTTAFMQLSGLTSEDSAVYFCARDYYGTSYNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 234 aa, synthetic construct
MVLQTQVFISLLLWISGAYGDIQMTQSSSSFSVSLGDRVTITCKANEDINNRLAWYQQTPGNSPRLLISGATNLVTGVPSRFSGSGSGKDYTLTITSLQAEDFATYYCQQYWSTPFTFGSGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 234 aa, synthetic construct
MVLQTQVFISLLLWISGAYGDIQMTQSSSSFSVSLGDRVTITCKANEDINLRLAWYQQTPGNSPRLLISGATNLVTGVPSRFSGSGSGKDYTLTITSLQAEDFATYYCQQYWSTPFTFGSGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 234 aa, synthetic construct
MVLQTQVFISLLLWISGAYGDIQMTQSSSSFSVSLGDRVTITCKANEDINARLAWYQQTPGNSPRLLISGATNLVTGVPSRFSGSGSGKDYTLTITSLQAEDFATYYCQQYWSTPFTFGSGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 234 aa, synthetic construct
MVLQTQVFISLLLWISGAYGDIQMTQSSSSFSVSLGDRVTITCKANEDINNRLAWYQQTPGNSPRLLISGATNLVTGVPSRFSGSGSGKDYTLTITSLQAEDFATYYCQQYYSTPFTFGSGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 234 aa, synthetic construct
MVLQTQVFISLLLWISGAYGDIQMTQSSSSFSVSLGDRVTITCKANEDINNRLAWYQQTPGNSPRLLISGATNLVTGVPSRFSGSGSGKDYTLTITSLQAEDFATYYCQQYFSTPFTFGSGTELEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - DNA, 1431 bp, synthetic construct
  source 1..1431: mol_type = other DNA
gaattcgccgccaccatgaagcacctgtggttcttcctgctgctggtggccgctcctaggtgggtgctgagccaggtgcaactggtgcagtccggagctgaggtgaagaagcccggagcttccgtgaaggtgagctgtaaggcctccggctacacattcaccggctactacatgcactgggtgagacaggcccccggacaaggactggaatggatcggagagatcaatcctagcaacgccaggacaaactacaacgagaggttccagggcagggtgacactgacagtggacaagagcatcagcaccgcctacatggagctgagcagactgaggtccgatgacaccgccgtgtactactgtgccagagattactacggcacctcctacaatttcgactactggggccagggcaccctggttacagtgtcttccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 138 - DNA, 1431 bp, synthetic construct
  source 1..1431: mol_type = other DNA
gaattcgccgccaccatgaagcacctgtggttcttcctgctgctggtggccgctcctaggtgggtgctgagccaggtgcaactggtgcagtccggagctgaggtgaagaagcccggagcttccgtgaaggtgagctgtaaggcctccggctacacattcaccggctacttcatgcactgggtgagacaggcccccggacaaggactggaatggatcggagagatcaatcctagcaacgccaggacaaactacaacgagaggttccagggcagggtgacactgacagtggacaagagcatcagcaccgcctacatggagctgagcagactgaggtccgatgacaccgccgtgtactactgtgccagagattactacggcacctcctacaatttcgactactggggccagggcaccctggttacagtgtcttccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaagccgccggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacgcttccacctacagggtggtctccgtcctgacagtcctgcatcaggattggctgaacggcaaggagtacaagtgcaaggtctccaataaagccctgcctgccagcatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgaaagctt

SEQ ID NO: 139 - DNA, 728 bp, synthetic construct
  source 1..728: mol_type = other DNA
gaattcgccgccaccatggtgctgcagacccaggtgtttatcagcctgctgctgtggatctccggcgcttatggcgatatccagatgacccagtccccctcctccctgtctgcttctgtgggagatagggtgaccatcacctgtaaggctaatgaggatatcaacgctcggctggcctggtaccagcagaagcctggaaaggctcctaagctgctgatctccggcgccacaaacctggtgaccggagttccttcccggttctccggaagcggatccggaaaggactataccctgaccatctccagcctgcagcccgaagatttcgctacctattactgccagcagtattatagcacccccttcaccttcggccagggcaccaaactggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 140 - DNA, 728 bp, synthetic construct
  source 1..728: mol_type = other DNA
gaattcgccgccaccatggtgctgcagacccaggtgtttatcagcctgctgctgtggatctccggcgcttatggcgatatccagatgacccagtccccctcctccctgtctgcttctgtgggagatagggtgaccatcacctgtaaggctaatgaggatatcaacgctcggctggcctggtaccagcagaagcctggaaaggctcctaagctgctgatctccggcgccacaaacctggtgaccggagttccttcccggttctccggaagcggatccggaaaggactataccctgaccatctccagcctgcagcccgaagatttcgctacctattactgccagcagtatttcagcacccccttcaccttcggccagggcaccaaactggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 141 - protein, 469 aa, synthetic construct
MKHLWFFLLLVAAPRWVLSQVQLVQSGAEVKKPGASVKVSCKASGYTFTGYYMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 234 aa, synthetic construct
MVLQTQVFISLLLWISGAYGDIQMTQSPSSLSASVGDRVTITCKANEDINARLAWYQQKPGKAPKLLISGATNLVTGVPSRFSGSGSGKDYTLTISSLQPEDFATYYCQQYYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 234 aa, synthetic construct
MVLQTQVFISLLLWISGAYGDIQMTQSPSSLSASVGDRVTITCKANEDINARLAWYQQKPGKAPKLLISGATNLVTGVPSRFSGSGSGKDYTLTISSLQPEDFATYYCQQYFSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 330 aa, synthetic construct [sequence omitted]

SEQ ID NO: 146 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVKLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVDHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLRVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 447 aa, synthetic construct
LVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVKLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 447 aa, synthetic construct
LVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVDHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 447 aa, synthetic construct
LVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLRVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 447 aa, synthetic construct
LVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 469 aa, synthetic construct
MKHLWFFLLLVAAPRWVLSQVQLVQSGAEVKKPGASVKVSCKASGYTFPGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 469 aa, synthetic construct
MKHLWFFLLLVAAPRWVLSQVQLVQSGAEVKKPGASVKVSCKASGYTFTGYFMHWVRQRPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRATLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVKLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVDHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLRVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 469 aa, synthetic construct
MKHLWFFLLLVAAPRWVLSQVQLVQSGAEVKKPGASVKVSCKASGYTFPGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 469 aa, synthetic construct
MKHLWFFLLLVAAPRWVLSQVQLVQSGAEVKKPGASVKVSCKASGYTFTGYFMHWVRQRPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRATLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVKLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLRVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 469 aa, synthetic construct
MKHLWFFLLLVAAPRWVLSQVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 469 aa, synthetic construct
MKHLWFFLLLVAAPRWVLSQVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 450 aa, synthetic construct
QVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 450 aa, synthetic construct
QVQLVQSGAEVKKPGASVKVSCKASGYTFPGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 450 aa, synthetic construct
QVQLVQSGAEVKKPGASVKVSCKASGYTFTGYFMHWVRQRPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 450 aa, synthetic construct
QVQLVQSGAEVKKPGASVKVSCKASGYTFTGYFMHWVRQAPGQGLEWIGEINPSNARTNYNERFQGRATLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVKLFPPKPKDTLMISRTPEVTCVVVDVSHEDPEVKFNWYVDGVEVHNAKTKPREEQYASTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVDHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLRVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 450 aa, synthetic construct
QVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 450 aa, synthetic construct
QVQLVQSGAEVKKPGASVKVSCKASGYTFPGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 450 aa, synthetic construct
QVQLVQSGAEVKKPGASVKVSCKASGYTFTGYFMHWVRQRPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 450 aa, synthetic construct
QVQLVQSGAEVKKPGASVKVSCKASGYTFTGYFMHWVRQAPGQGLEWIGEINPSNARTNYNERFQGRATLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVKLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLRVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 450 aa, synthetic construct
QVQLVQSGAEVKKPGASVKVSCKASGYTFTGYFMHWVRQAPGQGLEWIGEINPSNARTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 450 aa, synthetic construct
QVQLVQSGAEVKKPGASVKVSCKASGYTFTGYWMHWVRQAPGQGLEWIGEINPSNGRTNYNERFQGRVTLTVDKSISTAYMELSRLRSDDTAVYYCARDYYGTSYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLPVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 214 aa, synthetic construct
DIQMTQSPSSLSASVGDRVTITCKANEDINNRLAWYQQKPGKAPKLLISGATNLVTGVPSRFSGSGSGKDYTLTISSLQPEDFATYYCQQYWSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 214 aa, synthetic construct
DIQMTQSPSSLSASVGDRVTITCKANEDINARLAWYQQKPGKAPKLLISGATNLVTGVPSRFSGSGSGKDYTLTISSLQPEDFATYYCQQYYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0055182 A1
(43) Pub. Date: Feb. 26, 2026

(54) ANTI-L1-CAM ANTIBODIES AND THEIR USES FOR DIAGNOSTIC AND THERAPEUTIC APPLICATIONS

(71) Applicant: CIS BIOPHARMA AG, Bubendorf (CH)

(72) Inventors: Christian GERATHS, Rheinfelden (DE); Daniela WINKLER, Basel (CH); Michael HACKEBEIL, Gorwihl (DE); Christoph SCHÄFER, Engelberg (CH)

(21) Appl. No.: 19/128,917

(22) PCT Filed: Nov. 9, 2023
(86) PCT No.: PCT/EP2023/081319

§ 371 Date: May 9, 2025

(30) Foreign Application Priority Data
Nov. 9, 2022 (EP) 22206311.7

(51) Int. Cl. C07K 16/28; A61K 39/00; A61K 47/68; A61P 35/00; G01N 33/574

(52) U.S. Cl. CPC C07K 16/2803; A61K 47/68031; A61K 47/6807; A61K 47/6849; A61P 35/00; G01N 33/57407; A61K 2039/505; C07K 2317/565; C07K 2317/92; G01N 2333/70596

(57) ABSTRACT

The present invention relates to an antibody or an antigen binding fragment thereof that specifically binds to L1-CAM (CD171), to a polynucleotide encoding at least one variable heavy chain sequence and/or at least one variable light chain sequence as in the antibody or an antigen binding fragment thereof of the present invention, to a host cell comprising the polynucleotide of the present invention, to an immunoconjugate comprising an antibody or an antigen-fragment binding thereof of the present invention and an active agent, to a pharmaceutical composition comprising the antibody or the antigen-binding fragment thereof of the present invention, or the immunoconjugate of the present invention, and their use in treatment and/or diagnosis. The antibodies, antigen-binding fragments thereof, the immunoconjugates and the pharmaceutical compositions described herein are particularly useful in treatment or diagnosis of an L1-CAM (CD171) associated cancer.

### FIELD OF THE INVENTION

The present invention relates to an antibody or an antigen binding fragment thereof that specifically binds to L1-CAM (CD171), to a polynucleotide encoding at least one variable heavy chain sequence and/or at least one variable light chain sequence as in the antibody or an antigen binding fragment thereof of the present invention, to a host cell comprising the polynucleotide of the present invention, to an immunoconjugate comprising an antibody or an antigen-fragment binding thereof of the present invention and an active agent, to a pharmaceutical composition comprising the antibody or the antigen-binding fragment thereof of the present invention, or the immunoconjugate of the present invention, and their use in treatment and/or diagnosis. The antibodies, antigen-binding fragments thereof, the immunoconjugates and the pharmaceutical compositions described herein are particularly useful in treatment or diagnosis of an L1-CAM (CD171) associated cancer.

### BACKGROUND OF THE INVENTION

The advent of chimeric antigen receptor (CAR) technology (Sadelain M et al., Cancer Discov 3:388-98 (2013)) is rapidly expanding the therapeutic investigations of anti-L1-CAM redirected gene-modified T cells, with several clinical trials using L1-CAM-CAR modified T cells: NCT00889954 (all L1-CAM(+) cancers), NCT01935843 (L1-CAM(+) solid tumors). See Hong H et al., J Immunother 37:93-104 (2014). While T cells can effectively target tumors having low levels of L1-CAM, there are concerns regarding potential bystander toxicity in normal tissues that have low levels of LI-CAM expression.

Apart from toxicity, cell harvesting, processing, storage, transport and product release regulations for lymphocyte therapy can be challenging, especially when the cells have to be genetically modified. T cell exhaustion, survival and homing are suboptimal despite the infusion of billions of these cells. Further, CAR-modified T cells are no exception to the immunosuppressive tumor microenvironment, where Tregs, tumor associated macrophages and myeloid suppressor cells work in concert to circumvent the anti-tumor properties of CAR modified T-cells. Furthermore, CAR modified T-cells are subject to the same immunosuppressive constraints confronted by classic T-cells, including anergy following engagement of CTLA4 by B7 or PD-1 by PD-L1 (B7-H1) on tumor cells. Clinically effective alternatives to CAR modified T-cell therapy for the treatment of cancer are thus needed.

L1-CAM has been shown to be expressed in many human cancers and be often associated with bad prognosis, mostly due to motility and invasion promoting function of L1-CAM (Altevogt et al., International Journal of Cancer, 138, 1565-1576 (2016)). Kiefel and coworkers (Cell Adhesion & Migration 6:4, 374-384) have described L1-CAM as a major drive for tumour cell invasion and motility.

Doberstein et al have demonstrated that L1 CAM is expressed in triple-negative breast cancers and is inversely correlated with androgen receptor (Dobberstein et al., BMC Cancer 2014, 14:958). Eliane Fischer et al have described L1-CAM-targeted antibody therapy and ¹⁷⁷Lu-radioimmunotherapy of disseminated ovarian cancer (E Fischer et al., Int. J. Cancer: 130, 2715-2721 (2012)). According to Wachowiak and coworkers, increased L1-CAM levels are associated with glioblastoma and metastatic brain tumors (Wachowiak et al.; Medicine (2018) 97:38). Rached et al have shown that L1-CAM knock-out radiosensitizes neuroblastoma IMR-32 cells by simultaneously decreasing MycN, but increasing PTEN protein expression (Rached et al., International; Journal of Oncology, 49: 1722-1730, 2016). L1-CAM has also been shown to increase adhesion-mediated proliferation and chemoresistance of retinoblastoma (Jo et al., Oncotarget, 2017, Vol 8, pp 15441-15452). Terraneo et al have shown that L1-CAM confers radioresistance to ovarian cancer and defines a new cancer stem cell population (Terraneo et al., Cancers 2020, 12, 217). Expression profile analysis in multiple human tumors identified L1-CAM as a molecular marker for differential diagnosis and targeted therapy (Huszar et al., Human Pathology (2006), 37, 1000-1008).

Therapeutic antibodies targeting L1-CAM have been previously described. Hoefnagel et al (European Journal of Nuclear Medicine, 2001, 28:359-368) disclose targeting of neuroblastoma with anti L1-CAM antibody mAb chCE7. Therapeutic efficacy in a neuroblastoma xenograft model and imaging of neuroblastoma patients are disclosed. The same antibody chCE7 has been shown to bind to an isoform of L1-CAM present in renal carcinoma cells (Meli et al., International Journal of Cancer, 83, 401-408, 1999). Novak-Hofer et al (J. Nucl. Med, 1992, 33:231-236) described radioimmunolocalization of neuroblastoma xenografts with chimeric antibody chCE7. L1-CAM has also been shown to define the regenerative origin of metastasis-initiating cells in colorectal cancer (Ganesh et al., 2020, 28-45).

Since L1-CAM is known to be expressed in the peripheral nerve tissue, the side effect of treatment targeting L1-CAM are common. In particular, said side effects include ADCC, CDC and other immune reactions. It is accordingly crucial to provide an antibody or a fragment thereof wherein said toxic effects would be diminished. It is particularly desirable in the case of targeted radionuclide therapy to provide an antibody with a low K_{D}, which is related to a low k-off once bound to the tumour, which would allow avoiding its diffusion to the healthy tissue.

Document WO 2018/232188 discloses certain anti-L1-CAM antibodies and uses thereof.

Amstutz et al. (Amstutz, Int. J. Cancer: 53, 147-152 (1993)) discloses production and characterization of a mouse/human chimeric antibody directed against human neuroblastoma.

Umana et al (Nature Biotechnology, 1999, 176-180) disclose engineered glycoforms of an anti-neuroblastoma IgG1 with optimized antibody-dependent cellular cytotoxic activity.

Document “Improvement pf Biophysical Properties and Affinity of a Human Anti-L1-CAM Therapeutic Antibody through Antibody Engineering Based on Computational Methods” in International Journal of Molecular Sciences, vol 22, no. 13, page 6696, describes certain variants of antibody Ab417 (which is a humanized antibody binding L1-CAM) possessing good properties in terms of production stability and anti-tumoral activity.

### SUMMARY OF THE INVENTION

The present inventors have found that, surprisingly, the antibodies or the antigen-binding fragments of the present invention show improved affinity in binding to L1-CAM in comparison to closely related antibodies, in particular to CE7 antibody (Amstutz et al.) that they are derived from. In particular, at least in part the antibodies of the present invention are humanised antibodies, and the skilled person would expect the humanization of the antibody to negatively impact the binding affinity of the antibody to its antigen. Accordingly, the present invention is based, at least in part, on an unexpected discovery that antibodies of the present invention, obtained upon humanization of CE7 antibody, exhibit surprisingly improved binding affinity to L1-CAM in comparison to CE7 antibody, as demonstrated, among others, in the SPR study performed by the present inventors (see Examples 6 and 7 hereinbelow).

It is further noted that, according to the present inventors, the antibodies or the antigen-binding fragments thereof, of the present invention show at least comparable (and not worse) affinity in comparison to CE7 antibody that they are derived from through a process of humanization.

The present inventors have further demonstrated that the antibodies or the antigen-binding fragments thereof of the present invention have surprisingly shown improved stability in comparison to closely related antibodies, in particular to CE7 antibody that they are derived from. Said improved stability is manifested, at least, in a higher aggregation onset temperature as demonstrated in the PANTA studies (see Example 23).

The invention will be summarized in the following embodiments.

In a first embodiment, the present invention relates to an antibody or an antigen binding fragment thereof that specifically binds to L1-CAM (CD171), the antibody or the antigen binding fragment thereof comprising:
a variable heavy chain region comprising:
   CDR-H1 characterized by a sequence selected from
   a sequence according to SEQ ID NO.:1 (GYWMH),
   a sequence according to SEQ ID NO.: 2 (GYYMH),
   a sequence according to SEQ ID NO.: 3 (GYFMH), and
   a sequence according to SEQ ID NO.: 4 (GYLMH); and
   CDR-H2 characterized by a sequence selected from
   a sequence according to SEQ ID NO.: 5 (EINPSNGRTNYNERFQG),
   a sequence according to SEQ ID NO.: 6 (EINPSNGRTNYNEKFQG),
   a sequence according to SEQ ID NO.: 7 (EINPSNGRTNYNERFKS),
   a sequence according to SEQ ID NO.: 8 (EINPSNGRTNYNERLKS),
   a sequence according to SEQ ID NO.: 9 (EINPSNARTNYNERFQG),
   a sequence according to SEQ ID NO.: 10 (EINPSNARTNYNEKFQG)
   a sequence according to SEQ ID NO.: 11 (EINPSNARTNYNERFKS) and
   a sequence according to SEQ ID NO.: 12 (EINPSNARTNYNERLKS); and
   CDR-H3 characterized by a sequence according to SEQ ID NO.: 13 (DYYGTSYNFDY); and/or
a variable light chain region comprising:
   CDR-L1 characterized by a sequence selected from
   a sequence according to SEQ ID NO.: 14 (RANEDINNRLA),
   a sequence according to SEQ ID NO.: 15 (KANEDINNRLA),
   a sequence according to SEQ ID NO.: 16 (QANEDINNRLA),
   a sequence according to SEQ ID NO.: 17 (RANEDINARLA),
   a sequence according to SEQ ID NO.: 18 (KANEDINARLA),
   a sequence according to SEQ ID NO.: 19 (QANEDINARLA),
   a sequence according to SEQ ID NO.: 20 (RANEDINLRLA),
   a sequence according to SEQ ID NO.: 21 (KANEDINLRLA), and
   a sequence according to SEQ ID NO.: 22 (QANEDINLRLA); and
   CDR-L2 characterized by a sequence selected from
   a sequence according to SEQ ID NO.: 23 (GATNLVT) and
   a sequence according to SEQ ID NO.: 24 (GASNLVS); and
   CDR-L3 characterized by a sequence selected from
   a sequence according to SEQ ID NO.: 25 (QQYWSTPFT),
   a sequence according to SEQ ID NO.: 26 (QQYYSTPFT) and
   a sequence according to SEQ ID NO.: 27 (QQYFSTPFT).

In a second embodiment, the present invention relates to a polynucleotide encoding at least one variable heavy chain sequence and/or at least one variable light chain sequence as described in the first embodiment.

In a third embodiment, the present invention relates to a host cell comprising the polynucleotide of the second embodiment.

In a fourth embodiment, the present invention relates to an immunoconjugate comprising an antibody or an antigen-fragment binding thereof of the first embodiment and an active agent, preferably wherein the active agent is a cytotoxic agent or a prodrug thereof.

In a fifth embodiment, the present invention relates to a pharmaceutical composition comprising the antibody or the antigen-binding fragment thereof of the first embodiment, or the immunoconjugate of the fourth embodiment, and a pharmaceutically acceptable carrier.

In a sixth embodiment, the present invention relates to the antibody or the antigen-binding fragment thereof of the first embodiment or the immunoconjugate of the fourth embodiment, for use as a medicament.

In a seventh embodiment, the present invention relates to the antibody or the antigen-binding fragment thereof of the first embodiment or the immunoconjugate of the fourth embodiment, for use in the treatment of an L1-CAM (CD171) associated cancer.

In an eighth embodiment, the present invention relates to the antibody or the antigen-binding fragment thereof of the first embodiment or the immunoconjugate of the fourth embodiment, for use in diagnosis.

In a ninth embodiment, the present invention relates to the antibody or the antigen-binding fragment thereof of the first embodiment or the immunoconjugate of the fourth embodiment, for use in the diagnosis of an L1-CAM (CD171) associated cancer.

### BRIEF DESCRIPTION OF FIGURES

The invention is further described in the following drawings/figures, which are not to be construed as limiting.

FIG. 1 presents purification profiles of the rAbs, variants 1 to 18, as in Example 2. Non-reduced PAGE with Coomassie blue staining. MW. Molecular weight marker. IN. Input. FT. Flow through. W. Washes. E. Eluted fractions.

FIG. 2 presents Final sample QC in Example 2. Coomassie blue staining. Reduced and Non-reduced PAGE analysis. MW. Molecular weight marker. 2 μg loaded per lane.

FIG. 3 presents purification profiles of the rAbs, variants 19 to 28, as in Example 3. Non-reduced PAGE with Coomassie blue staining. MW. Molecular weight marker. IN. Input. FT. Flow through. W. Washes. E. Eluted fractions.

FIG. 4 presents Final sample QC in Example 3. Coomassie blue staining. Reduced and Non-reduced PAGE analysis. MW. Molecular weight marker. 2 μg loaded per lane.

FIG. 5 presents Purification profiles of the rAbs as in Example 4. Non-reduced PAGE with Coomassie blue staining. MW. Molecular weight marker. IN. Input. FT. Flow through. W. Washes. E. Eluted fractions.

FIG. 6 presents Final sample QC in Example 4. Coomassie blue staining. Reduced and Non-reduced PAGE analysis. MW. Molecular weight marker. 2 μg loaded per lane.

FIG. 7 presents SEC-HPLC profiles obtained for CE7-variant4_AG (part 1), CE7-variant7_AG (part 2), CE7-variant18_AG (part 3), applied standard (part 4) and a blank sample (part 5).

FIG. 8 presents blank-subtracted sensorgrams for binding of CE7-variant4-AG (part 1), CE7-variant7-AG (part 2), CE7-variant18-AG (part 3), chimeric HCE7 (part 4) and deglycosylated cHCE7 (part 5), respectively, to L1-CAM, as described in Example 6.

FIG. 9 presents blank-subtracted sensorgrams for binding of CE7-variant7 (part 1), CE7-variant26 (part 2), CE7-variant27 (part 3), and CE7-variant28 (part 4), respectively, to L1-CAM, as described in Example 7.

FIG. 10 presents Purification profiles of the rAbs as in Example 8. Non-reduced PAGE with Coomassie blue staining. MW. Molecular weight marker. IN. Input. FT. Flow through. W. Washes. E. Eluted fractions.

FIG. 11 presents Final sample QC in Example 8. Coomassie blue staining. Reduced and Non-reduced PAGE analysis. MW. Molecular weight marker. 2 μg loaded per lane.

FIG. 12 presents SEC-HPLC profiles obtained for LV1-8. LV 1-8 are based on the chimeric CE7 antibody comprising (L234A, L235A, P331 S and N297A) mutation in the FC part. Aim of this study was to identify influence of sequence liability mutation on parent mAb.

FIG. 13 shows the double referenced, solvent corrected sensorgram of the interaction between A) cHCE7 degly and L1-CAM, and B) cHCE7 deglycosylated and L1-CAM, with measured responses illustrated in on the graph.

FIG. 14 presents Purification profiles of the rAbs as in Example 21. Non-reduced PAGE with Coomassie blue staining. MW. Molecular weight marker. IN. Input. FT. Flow through. W. Washes. E. Eluted fractions.

FIG. 15 presents Final sample QC in Example 21. Coomassie blue staining. Reduced and Non-reduced PAGE analysis. MW. Molecular weight marker. 2 μg loaded per lane.

FIG. 16 presents SEC-HPLC profiles obtained for LV29-32.

FIG. 17 presents survival of embryos after 3 days of intravenous injection of naked huCE7-V7AG. X-axis was shown as a log[mg/mL].

FIG. 18 presents biodistribution studies at 24, 48, 72 and 96 hours for chCE7 deglycosylated and Variant 7AG according to the present invention.

FIG. 19 shows a biodistribution of ¹⁷⁷Lu labeled huCE7-LV40-(PEG₄-DOTA)₂ & huCE7-LV40-(PEG₄-Polymer-α-DOTA₄)₂ in a mouse tumor xenograft model. Mouse model: CD1 nu/nu sc SKOVip3 (ovarian cancer, L1-CAM positive).

FIG. 20 presents efficacy of huCE7-V7AG-(PEG₄-DOTA)₂ labeled with 4 Mbq ¹⁷⁷Lu in a mouse tumor xenograft model. Mouse model: CD1 nu/nu sc SKOVip3 (ovarian cancer, L1-CAM positive). Mice were treated with a single injection of labeled mAb (therapy group, 10 mice) or PBS (negative control group, 10 mice) at day 12 after tumor cell inoculation. Tumor size, bodyweight and survival were monitored throughout the study. Mice were euthanized once tumor size reached threshold.

FIG. 21 presents efficacy evaluation of huCE7-V7AG and HUCE7-V7AG-(MMAE)₄ antibodies on MAXFTN-401 breast cancer cells in a zebrafish xenograft model. Antibodies were administered via i.v. right after tumor implantation. Data is presented as Mean±SEM, followed by Student's t-test, *p<0.05.

FIG. 22 shows A. FACS analysis of H2171 SCLC cells and B. FACS analysis of negative control cell line MDA-MB-468 (not expressing L1-CAM).

FIG. 23 presents normalized primary tumor size and metastatic dissemination of H2171 cells in a zebrafish xenograft model after 48 hours of treatment with HuCE7-V7AG and HuCE7-V7AG-(MMAE)₄ antibodies administered i.v. Normalized data with respect to the negative control group is shown. Data are presented as mean±SEM; One-way ANOVA was performed (p<0.0001) followed by two-tailed Student's t-test, *p<0.05 and ****p<0.0001.

### DETAILED DESCRIPTION OF THE INVENTION

The invention will be described in detail in the following. It is to be understood that, unless explicitly indicated to the contrary, all the disclosed features can be combined with each other.

In one embodiment, the present invention relates to an antibody or an antigen binding fragment thereof that specifically binds to L1-CAM (CD171).

L1-CAM refers to a transmembrane protein member of the L1 protein family encoded by the L1-CAM gene, as described in UniProt database under the reference P32004. The L1-CAM protein is a neuronal cell adhesion molecule with a strong implication in cell migration, adhesion, neurite outgrowth, myelination and neuronal differentiation. The L1-CAM protein has also been found to play a role in treatment-resistant cancers. The term “L1-CAM” may be used interchangeably with the term “CD171”, both of which are known to the skilled person.

Accordingly, an antibody or an antigen binding fragment thereof that specifically binds to L1-CAM may also be referred to as an antibody (or an antigen binding fragment thereof, respectively) that binds to an epitope within L1-CAM, preferably specifically binds to an epitope within L1-CAM. An “antibody that binds to an epitope” within a defined region of a protein is an antibody that requires the presence of one or more of the amino acids within that region for binding to the protein.

As it is to be understood herein, the antibodies and the antigen-binding fragments thereof of the present invention preferably bind to Ig6-like domain of L1-CAM and prevent the binding of L1-CAM to integrins. Accordingly, the antibodies or the antigen-binding fragments thereof preferably bind to the same epitope as chCE7 antibody, referred to herein.

As preferably referred to herein, specific binding to L1-CAM refers to the situation, wherein, the antibody or the fragment thereof is capable of binding L1-CAM with sufficient affinity that said antibody is useful as a diagnostic and/or therapeutic agent in targeting L1-CAM. Accordingly, and preferably, the extent of binding of such antibody to an unrelated, non-L1-CAM protein is less than about 10% of the binding of the antibody to L1-CAM, as measured e.g., in an immunoassay (for example a radioimmunoassay) or in an SPR assay (surface-plasmon resonance). More preferably, the extent of binding of such antibody to an unrelated, non-L1-CAM protein is less than about 5% of the binding of the antibody to L1-CAM, as measured e.g. in an immunoassay (for example a radioimmunoassay) or in an SPR assay (surface-plasmon resonance, e.g. performed used Biacore).

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with a specific antigen or a specific group of antigens of L1-CAM.

Cross-reactivity of antibodies or antigen-binding fragments thereof under investigation may be tested, for example, by assessing binding of said panel of antibodies or antigen-binding fragments thereof under conventional conditions (see, e.g., Harlow and Lane, Antibodies: A Laboratory Manual, Cold Spring Harbor Laboratory Press, (1988) and Using Antibodies: A Laboratory Manual, Cold Spring Harbor Laboratory Press, (1999)) to the (poly)peptide of interest as well as to a number of more or less (structurally and/or functionally) closely related (poly)peptides. Only those constructs (i.e. antibodies, antigen-binding fragments thereof and the like) that bind to the certain structure of L1-CAM as defined herein, e.g., a specific epitope or (poly) peptide/protein of L1-CAM as defined herein but do not or do not essentially bind to any of the other epitope or (poly) peptides of the same L1-CAM, are considered specific for the epitope or (poly) peptide/protein of interest and selected for further studies in accordance with the method provided herein. These methods may comprise, inter alia, binding studies, blocking and competition studies with structurally and/or functionally closely related molecules. These binding studies also comprise FACS analysis, surface plasmon resonance (SPR, e.g. with Biacore), analytical ultracentrifugation, isothermal titration calorimetry, fluorescence anisotropy, fluorescence spectroscopy or by radiolabeled ligand binding assays. Accordingly, specificity can be determined experimentally by methods known in the art and methods as described herein. Such methods comprise, but are not limited to Western Blots, ELISA-, RIA-, ECL-, IRMA-tests and peptide scans.

As preferably referred to herein, an antibody or an antigen-binding fragment is defined in the following.

In general, the term “antibody” is used herein in the broadest sense and encompasses various antibody structures, including but not limited to monoclonal antibodies, polyclonal antibodies, multispecific antibodies (e.g., bispecific antibodies), fully-human antibodies and antibody fragments so long as they exhibit the desired antigen-binding activity. Unless explicitly indicated to the contrary, when discussing the antibody or the properties thereof, whenever a reference is made to an antibody, an antibody of an antigen-binding fragment thereof is meant.

As preferably referred to herein, an “antigen-binding fragment” of an antibody refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

Preferably, the antibody or the antigen binding fragment thereof is a monoclonal antibody, a chimeric antibody, a recombinant antibody, an antigen-binding fragment of a recombinant antibody, a single chain antibody, a humanized antibody, a bispecific antibody, a multi-specific antibody, or an antibody displayed upon the surface of a phage or displayed upon the surface of a chimeric antigen receptor (CAR) T cell.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, Nature 256 (1975), 495.

The term “chimeric antibodies”, refers to an antibody which comprises a variable region of the present invention fused or chimerized with an antibody region (e.g., constant region) from another, human or non-human species (e.g., mouse, horse, rabbit, dog, cow, chicken).

The term “recombinant antibody” includes all antibodies that are prepared, expressed, created or isolated by recombinant means, such as antibodies isolated from an animal (e.g. a mouse) that is transgenic for human immunoglobulin genes, antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Accordingly, the term antibody also relates to recombinant human antibodies, heterologous antibodies and heterohybrid antibodies. Such recombinant human antibodies have variable and constant regions (if present) derived from human germline immunoglobulin sequences. Such antibodies can, however, be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The term “heterohybrid antibody” refers to an antibody having light and heavy chains of different organismal origins. For example, an antibody having a human heavy chain associated with a murine light chain is a heterohybrid antibody. Examples of heterohybrid antibodies include chimeric and humanized antibodies.

The term antibody also relates to humanized antibodies. “Humanized” forms of non-human (e.g. murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Often, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, humanized antibody may comprise residues, which are found neither in the recipient antibody nor in the imported CDR or framework sequences. These modifications are made to further refine and optimize antibody performance. In general, the humanized antibody will comprise substantially all of at least one, and typically two variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin. For further details, see: Jones Nature 321 (1986), 522-525; Reichmann Nature 332 (1998), 323-327 and Presta Curr Op Struct Biol 2 (1992), 593-596.

Accordingly, in context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully-human antibodies, chimeric antibodies, humanized antibodies, CDR-grafted antibodies and antibody constructs, like single chain Fvs (scFv) or antibody-fusion proteins.

A single chain antibody, i.e., “single-chain Fv” or “scFv” antibody fragments have, in the context of the invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315.

A “Fab fragment” as used herein is comprised of one light chain and the CH1 and variable regions of one heavy chain. The heavy chain of a Fab molecule cannot form a disulfide bond with another heavy chain molecule.

An “Fc” region contains two heavy chain fragments comprising the C_{H}2 and C_{H}3 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A bispecific antibody, as referred to herein, is an antibody that can simultaneously bind to two different types of antigen, or to two different epitopes of the same antigen. Upon development, bispecific antibodies can be manufactured in several structural formats, which are known to the skilled person. Within the invention, at least one of antigens relates to L1-CAM, as defined herein.

A multi-specific antibody, as referred to herein, is an antibody that can simultaneously bind to more than two different types of antigen, or to more than two different epitopes of the same antigen.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit. The term “Ig-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., Methods for General and Molecular Bacteriology ASM Press (1994); Lefkovits, Immunology Methods Manual: The Comprehensive Sourcebook of Techniques; Academic Press (1997); Golemis, Protein-Protein Interactions: A Molecular Cloning Manual Cold Spring Harbor Laboratory Press (2002)).

More preferably, the antibody or the antigen binding fragment thereof is a monoclonal antibody.

The antibody as referred to herein may be an IgG1, IgG2a or IgG2b, IgG3, IgG4, IgM, IgA1, IgA2, IgAsec, IgD, IgE. As used herein, “isotype” refers to the antibody class (e.g., IgM or IgG1) that is encoded by heavy chain constant region genes. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

Preferably, the monoclonal antibody as described herein is an IgG1 antibody.

Accordingly, the antibodies of the present invention can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)2 fragment and a Fv fragment.

In the present invention, the antibody or the antigen binding fragment thereof comprise a variable heavy chain region, as described hereinbelow, and/or a variable light chain region, as described hereinbelow.

As encompassed by the invention, a variable heavy chain region comprises:
CDR-H1 characterized by a sequence selected from
a sequence according to SEQ ID NO.:1 (GYWMH),
a sequence according to SEQ ID NO.: 2 (GYYMH),
a sequence according to SEQ ID NO.: 3 (GYFMH), and
a sequence according to SEQ ID NO.: 4 (GYLMH); and
CDR-H2 characterized by a sequence selected from
a sequence according to SEQ ID NO.: 5 (EINPSNGRTNYNERFQG),
a sequence according to SEQ ID NO.: 6 (EINPSNGRTNYNEKFQG),
a sequence according to SEQ ID NO.: 7 (EINPSNGRTNYNERFKS),
a sequence according to SEQ ID NO.: 8 (EINPSNGRTNYNERLKS),
a sequence according to SEQ ID NO.: 9 (EINPSNARTNYNERFQG),
a sequence according to SEQ ID NO.: 10 (EINPSNARTNYNEKFQG)
a sequence according to SEQ ID NO.: 11 (EINPSNARTNYNERFKS) and
a sequence according to SEQ ID NO.: 12 (EINPSNARTNYNERLKS); and
CDR-H3 characterized by a sequence according to SEQ ID NO.: 13 (DYYGTSYNFDY).

As encompassed by the invention, a variable light chain region comprises:
CDR-L1 characterized by a sequence selected from
a sequence according to SEQ ID NO.: 14 (RANEDINNRLA),
a sequence according to SEQ ID NO.: 15 (KANEDINNRLA),
a sequence according to SEQ ID NO.: 16 (QANEDINNRLA),
a sequence according to SEQ ID NO.: 17 (RANEDINARLA),
a sequence according to SEQ ID NO.: 18 (KANEDINARLA),
a sequence according to SEQ ID NO.: 19 (QANEDINARLA),
a sequence according to SEQ ID NO.: 20 (RANEDINLRLA),
a sequence according to SEQ ID NO.: 21 (KANEDINLRLA), and
a sequence according to SEQ ID NO.: 22 (QANEDINLRLA); and
CDR-L2 characterized by a sequence selected from
a sequence according to SEQ ID NO.: 23 (GATNLVT) and
a sequence according to SEQ ID NO.: 24 (GASNLVS); and
CDR-L3 characterized by a sequence selected from
a sequence according to SEQ ID NO.: 25 (QQYWSTPFT),
a sequence according to SEQ ID NO.: 26 (QQYYSTPFT) and
a sequence according to SEQ ID NO.: 27 (QQYFSTPFT).

As understood herein, the present invention is defined through a number of amino acid sequences, which are preferably provided using a single letter code, which is known to the skilled person. As defined herein, the sequences shall be given directly in the text of the present application, or by reference to sequence listing appended to the present application. In case of discrepancy between the sequence given directly in the text in the present application and the sequence referred to in the sequence listing through a particular sequence identification number (SEQ ID NO.), the present invention is understood to relate to both sequences, i.e., the sequence as given in the description or a sequence as given in the sequence listing, preferably the present invention is understood to relate to the sequence as given in the sequence listing.

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J. Mol. Biol. 196 (1987), 901-917 or Chothia Nature 342 (1989), 877-883.

Accordingly, the present invention is based, at least in part, on a surprising discovery that the antibodies or the fragments thereof of the present invention show an improved affinity for the L1-CAM in comparison to the antibodies of the prior art, in particular in comparison to the CE7 antibody that they are derived from.

Preferably, in the antibody or the antigen-binding fragment thereof of the present invention, the variable heavy chain region of said antibody or the antigen-binding fragment thereof comprises CDR-H1 characterized by a sequence selected from sequences according to SEQ ID NO.: 1, SEQ ID NO.: 2 and SEQ ID NO. 3. More preferably, in the antibody or the antigen-binding fragment thereof of the present invention, the variable heavy chain region of said antibody or the antigen-binding fragment thereof comprises CDR-H1 characterized by a sequence selected from sequences according to SEQ ID NO.: 1, and SEQ ID NO.: 2. Even more preferably, in the antibody or the antigen-binding fragment thereof of the present invention, the variable heavy chain region of said antibody or the antigen-binding fragment thereof comprises CDR-H1 characterized by a sequence according to SEQ ID NO.: 1.

Preferably, in the antibody or the antigen-binding or the antigen-binding fragment thereof of the present invention, the variable heavy chain region of said antibody or the antigen-binding fragment thereof comprises CDR-H2 characterized by a sequence selected from sequences according to SEQ ID NO.: 5, 6, 9 or 10. More preferably, in the antibody or the antigen-binding or the antigen-binding fragment thereof of the present invention, the variable heavy chain region of said antibody or the antigen-binding fragment thereof comprises CDR-H2 characterized by a sequence according to SEQ ID NO.: 5 or 6. In one specific embodiment, in the antibody or the antigen-binding or the antigen-binding fragment thereof of the present invention, the variable heavy chain region of said antibody or the antigen-binding fragment thereof comprises CDR-H2 characterized by a sequence selected from sequences according to SEQ ID NO.: 8 or 12.

As it is to be understood herein, in the antibody or the antigen-binding or the antigen-binding fragment thereof of the present invention, the variable heavy chain region of said antibody or the antigen-binding fragment thereof comprises CDR-H3 characterized by a sequence according to SEQ ID NO.: 13.

In a preferred embodiment, in the antibody or the antigen-binding or the antigen-binding fragment thereof of the present invention, the variable heavy chain region of said antibody or the antigen-binding fragment thereof comprises CDR-H1 as described hereinabove, CDR-H2 as described hereinabove, and CDR-H3 as described hereinabove.

Preferably, in the antibody or the antigen-binding or the antigen-binding fragment thereof of the present invention, the variable light chain region of said antibody or the antigen-binding fragment thereof comprises CDR-L1 characterized by a sequence according to SEQ ID NO.: 14, 15, 17, 18, or 21. More preferably, in the antibody or the antigen-binding or the antigen-binding fragment thereof of the present invention, the variable light chain region of said antibody or the antigen-binding fragment thereof comprises CDR-L1 characterized by a sequence according to SEQ ID NO.: 14 or 15.

Preferably, in the antibody or the antigen-binding or the antigen-binding fragment thereof of the present invention, the variable light chain region of said antibody or the antigen-binding fragment thereof comprises CDR-L2 characterized by a sequence according to SEQ ID NO.: 23.

Preferably, in the antibody or the antigen-binding or the antigen-binding fragment thereof of the present invention, the variable light chain region of said antibody or the antigen-binding fragment thereof comprises CDR-L3 characterized by a sequence according to SEQ ID NO.: 25 or 26. More preferably, in the antibody or the antigen-binding or the antigen-binding fragment thereof of the present invention, the variable light chain region of said antibody or the antigen-binding fragment thereof comprises CDR-L3 characterized by a sequence according to SEQ ID NO.: 25.

Preferably, in the antibody or the antigen-binding or the antigen-binding fragment thereof of the present invention, the variable light chain region of said antibody or the antigen-binding fragment thereof comprises CDR-L1 as described hereinabove, CDR-L2 as described hereinabove, and CDR-L3 as described hereinabove.

According to the present invention, the antibody or the antigen-binding fragment thereof of the present invention comprises the variable heavy chain region of said antibody or the antigen-binding fragment thereof comprises CDR-H1 as described hereinabove, CDR-H2 as described hereinabove, and CDR-H3 as described hereinabove, and/or the variable light chain region of said antibody or the antigen-binding fragment thereof comprises CDR-L1 as described hereinabove, CDR-L2 as described hereinabove, and CDR-L3 as described hereinabove.

Preferably, the antibody or the antigen-binding fragment thereof of the present invention comprises the variable heavy chain region of said antibody or the antigen-binding fragment thereof comprises CDR-H1 as described hereinabove, CDR-H2 as described hereinabove, and CDR-H3 as described hereinabove, and the variable light chain region of said antibody or the antigen-binding fragment thereof comprises CDR-L1 as described hereinabove, CDR-L2 as described hereinabove, and CDR-L3 as described hereinabove.

In one embodiment, the antibody or the antigen-binding fragment thereof of the present invention comprises the variable heavy chain region of said antibody or the antigen-binding fragment thereof comprises CDR-H1 as described hereinabove, CDR-H2 as described hereinabove, and CDR-H3 as described hereinabove, or the variable light chain region of said antibody or the antigen-binding fragment thereof comprises CDR-L1 as described hereinabove, CDR-L2 as described hereinabove, and CDR-L3 as described hereinabove.

In one embodiment of the antibody or the antigen-binding fragment thereof of the present invention, the variable heavy chain region of said antibody or the antigen-binding fragment thereof comprises CDR-H2 characterized by a sequence according to SEQ ID NO.: 9, 10, 11 or 12 and/or the variable light chain region of said antibody or the antigen-binding fragment thereof comprises CDR-L1 characterized by a sequence according to SEQ Id No.: 17, 18, or 19. Preferably, in said embodiment, the variable heavy chain region of said antibody or the antigen-binding fragment thereof comprises CDR-H2 characterized by a sequence according to SEQ ID NO.: 9, 10, 11 or 12 and the variable light chain region of said antibody or the antigen-binding fragment thereof comprises CDR-L1 characterized by a sequence according to SEQ Id No.: 17, 18, or 19. Preferably, in said specific embodiment, the variable heavy chain region of said antibody or the antigen-binding fragment thereof further comprises CDR-H1 characterized by a sequence according to SEQ ID NO.: 2 or 3, and/or the variable light chain region of said antibody or the antigen-binding fragment thereof further comprises CDR-L3 characterized by a sequence according to SEQ ID NO.: 26 or 27. More preferably, in said specific embodiment, the variable heavy chain region of said antibody or the antigen-binding fragment thereof further comprises CDR-H1 characterized by a sequence according to SEQ ID NO.: 2 or 3, and the variable light chain region of said antibody or the antigen-binding fragment thereof further comprises CDR-L3 characterized by a sequence according to SEQ ID NO.: 26 or 27.

Preferably, the antibody or the antigen-binding fragment thereof of the present invention is a humanised antibody.

Humanization approaches are well known in the art and in particular described for antibody molecules, e.g. Ig-derived molecules. The term “humanized” refers to humanized forms of non-human (e.g., murine) antibodies or fragments thereof (such as Fv, Fab, Fab′, F(ab′), scFvs, or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR (framework) regions are those of a human immunoglobulin consensus sequence. The humanized antibody optimally also will comprise at least a portion of an immunoglobulin constant region (Fc), typically that of a human immunoglobulin; see, inter alia, Jones et al., Nature 321 (1986), 522-525, Presta, Curr. Op. Struct. Biol. 2 (1992), 593-596. Methods for humanizing non-human antibodies are well known in the art. Generally, a humanized antibody has one or more amino acids introduced into it from a source which is non-human still retain the original binding activity of the antibody. Methods for humanization of antibodies/antibody molecules are further detailed in Jones et al., Nature 321 (1986), 522-525; Reichmann et al., Nature 332 (1988), 323-327; and Verhoeyen et al., Science 239 (1988), 1534-1536. Specific examples of humanized antibodies, e.g. antibodies directed against EpCAM, are known in the art, see e.g. (LoBuglio, Proceedings of the American Society of Clinical Oncology Abstract (1997), 1562 and Khor, Proceedings of the American Society of Clinical Oncology Abstract (1997), 847).

Preferably as referred to herein, “framework regions” also referred to as FR regions are the part of the variable domain of the antibody which is not CDR. Accordingly, in each variable domain sequence of the antibody four framework regions are present, which are separated from each other by hypervariable regions—CDRs. The framework regions typically may make up to 85% of the sequence of the variable domain and act as a scaffold for exposing CDRs so that they can interact with the antigen. As known to the skilled person, variations in the framework region may also affect the binding affinity of the antibody to the antigen specific for CDRs.

Accordingly, in the context of this invention, antibody molecules or antigen-binding fragments thereof are provided, which are humanized and can successfully be employed in pharmaceutical compositions.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

Preferably, in the antibody or the antigen-binding fragment thereof of the present invention, the variable heavy chain region is characterized by a sequence at least 80% identical to, preferably at least 85% identical to, more preferably at least 90% identical to, even more preferably at least 95% identical to, even more preferably identical to a sequence selected from
a sequence according to SEQ ID NO.: 28 (QVQLVQSGAEVKKPGASVKVSCKASGYTFTGYWMHWVRQAPGQGLEWIGEINPS NGRTNYNERFQGRVTLTVDKSISTAYMELSRLRSDDTAVYYCARDYYGTSYNFDY WGQGTLVTVSS),
a sequence according to SEQ ID NO.: 29 (QVQLVQSGAEVKKPGASVKVSCKASGYTFTGYWMHWVRQAPGQGLEWIGEINPS NGRTNYNERFKSRVTLTVDKSISTAYMELSRLRSDDTAVYFCARDYYGTSYNFDYW GQGTLVTVSS),
a sequence according to SEQ ID NO.: 30 (QVQLQQWGAGLLKPSETLSLTCAAYGYTFTGYWMHWIRQPPGKGLEWIGEINPSN GRTNYNERLKSRVTLSVDKSKNQASLKLSSVTAADTAVYFCARDYYGTSYNFDYW GQGTLVTVSS),
a sequence according to SEQ ID NO.: 31 (QVQLVQSGAEVKKPGASVKVSCKASGYTFTGYWMHWVRQAPGQGLEWIGEINPS NGRTNYNEkFQGRVTLTVDKSISTAYMELSRLRSDDTAVYYCARDYYGTSYNFDYW GQGTLVTVSS), and
a sequence according to SEQ ID NO.: 32 (QVQLVQSGAEVKKPGASVKVSCKASGYTFTGYWMHWVRQAPGQGLEWmGEINP SNGRTNYNEkFQGRVTLTVDKSISTAYMELSRLRSDDTAVYYCARDYYGTSYNFDY WGQGTLVTVSS).

More preferably, in the antibody or the antigen-binding fragment thereof of the present invention, the variable heavy chain region is characterized by a sequence at least 80% identical to, preferably at least 85% identical to, more preferably at least 90% identical to, even more preferably at least 95% identical to, even more preferably identical to a sequence selected from a sequence according to SEQ ID NO.: 28 or a sequence according to SEQ ID NO.: 32.

Even more preferably, in the antibody or the antigen-binding fragment thereof of the present invention, the variable heavy chain region is characterized by a sequence identical to a sequence selected from a sequence according to SEQ ID NO.: 28 or a sequence according to SEQ ID NO.: 32.

Even more preferably, in the antibody or the antigen-binding fragment thereof of the present invention, the variable heavy chain region is characterized by a sequence identical to a sequence according to SEQ ID NO.: 28. In one embodiment, in the antibody or the antigen-binding fragment thereof of the present invention, the variable heavy chain region is characterized by a sequence identical to a sequence according to SEQ ID NO.: 32.

Preferably, in the antibody or the antigen-binding fragment thereof of the present invention, the variable light chain region is characterized by a sequence at least 80% identical to, preferably at least 85% identical to, more preferably at least 90% identical to, even more preferably at least 95% identical to, even more preferably identical to a sequence selected from
a sequence according to SEQ ID NO.: 33 (DIQMTQSPSSLSASVGDRVTITCKANEDINNRLAWYQQKPGKAPKLLISGATNLVT GVPSRFSGSGSGKDYTLTISSLQPEDFATYYCQQYWSTPFTFGQGTKLEIK),
a sequence according to SEQ ID NO.: 34 (DIQMTQSPSSLSASVGDRVTITCKANEDINNRLAWYQQKPGKAPKLLISGATNLVT GVPSRFSGSGSGKDYTLTISSLQPEDIATYYCQQYWSTPFTFGQGTKLEIK),
a sequence according to SEQ ID NO.: 35 (EIVMTQSPATLSVSPGERATLSCRANEDINNRLAWYQQKPGQAPRLLISGATNLVT GIPARFSGSGSGKEFTLTISSLQSEDFAVYYCQQYWSTPFTFGQGTKLEIK),
a sequence according to SEQ ID NO.: 36 (DIQMTQSPSSLSASVGDRVTITCRANEDINNRLAWYQQKPGKAPKLLISGATNLVT GVPSRFSGSGSGKDYTLTISSLQPEDFATYYCQQYWSTPFTFGQGTKLEIK), and
a sequence according to SEQ ID NO.: 37 (DIQMTQSPSSLSASVGDRVTITCRANEDINNRLAWYQQKPGKAPKLLISGAsNLVsG VPSRFSGSGSGKDYTLTISSLQPEDFATYYCQQYWSTPFTFGQGTKLEIK).

More preferably, in the antibody or the antigen-binding fragment thereof of the present invention, the variable light chain region is characterized by a sequence at least 80% identical to, preferably at least 85% identical to, more preferably at least 90% identical to, even more preferably at least 95% identical to, even more preferably identical to a sequence selected from a sequence according to SEQ ID NO.: 33, a sequence according to SEQ ID NO.: 36 and a sequence according to SEQ ID NO. 37, preferably from a sequence according to SEQ ID NO.: 33 and a sequence according to SEQ ID NO.: 36.

Even more preferably, in the antibody or the antigen-binding fragment thereof of the present invention, the variable light chain region is characterized by a sequence identical to a sequence selected from a sequence according to SEQ ID NO.: 33, a sequence according to SEQ ID NO.: 36 and a sequence according to SEQ ID NO. 37, preferably from a sequence according to SEQ ID NO.: 33 and a sequence according to SEQ ID NO.: 36.

Even more preferably, in the antibody or the antigen-binding fragment thereof of the present invention, the variable light chain region is characterized by a sequence identical to a sequence according to SEQ ID NO.: 33. In one embodiment, in the antibody or the antigen-binding fragment thereof of the present invention, the variable light chain region is characterized by a sequence identical to a sequence according to SEQ ID NO.: 36. In one embodiment, in the antibody or the antigen-binding fragment thereof of the present invention, the variable light chain region is characterized by a sequence identical to a sequence according to SEQ ID NO.: 37.

As understood to the skilled person, the sequence of the variable heavy chain (or the sequence of the variable light chain) may also be defined through its frameworks and its CDR sequences. Accordingly, further sequences of the variable heavy chain, or the variable light chain, as encompassed by the present invention, are disclosed in the following.

In one embodiment, the present invention relates to an antibody or an antigen binding fragment thereof, wherein the variable heavy chain region is characterized by a sequence at least 80% identical to, preferably by a sequence at least 85% identical to, more preferably by a sequence at least 90% identical to, even more preferably by a sequence at least 95% identical to, more preferably identical to a sequence:

FH0-CDR-H1-FH1-CDR-H2-FH2-CDR-H3-FH3.

CDR-H1, CDR-H2 and CDR-H3 are as defined hereinabove.

FH0 is characterized by a sequence according to SEQ ID NO: 38 (QVQLVQSGAEVKKPGASVKVSCKASGYTFT) or a sequence according to SEQ ID NO: 39 (QVQLQQWGAGLLKPSETLSLTCAAYGYTFT).

FH1 is characterized by a sequence according to SEQ ID NO: 40 (WVRQAPGQGLEWIG) or a sequence according to SEQ ID NO.: 41 (WIRQPPGKGLEWIG).

FH2 is characterized by a sequence according to SEQ ID NO: 42 (RVTLTVDKSISTAYMELSRLRSDDTAVYFCAR) or a sequence according to SEQ ID NO.: 43 (RVTLSVDKSKNQASLKLSSVTAADTAVYFCAR).

FH3 is characterized by a sequence according to SEQ ID NO:44 (WGQGTLVTVSS).

In one embodiment, the present invention relates to an antibody or an antigen binding fragment thereof, wherein the variable light chain region is characterized by a sequence at least 80% identical to, preferably by a sequence at least 85% identical to, more preferably by a sequence at least 90% identical to, even more preferably by a sequence at least 95% identical to, more preferably identical to a sequence:

FL0 - CDR-L1 - FL1 - CDR-L2 - FL2 - CDR-L3 - FL3.

CDR-L1, CDR-L2 and CDR-L3 are as defined hereinabove.

FL0 is characterized by a sequence according to SEQ ID NO: 45 (DIQMTQSPSSLSASVGDRVTITC) or a sequence according to SEQ ID NO: 46 (EIVMTQSPATLSVSPGERATLSC).

FL1 is characterized by a sequence according to SEQ ID NO: 47 (WYQQKPGKAPKLLIS) or a sequence according to SEQ ID NO.: 48 (WYQQKPGQAPRLLIS).

FL2 is characterized by a sequence according to SEQ ID NO: 49 (GVPSRFSGSGSGKDYTLTISSLQPEDIATYYC) or a sequence according to SEQ ID NO.: 50 (GIPARFSGSGSGKEFTLTISSLQSEDFAVYYC).

FL3 is characterized by a sequence according to SEQ ID NO:51 (FGQGTKLEIK).

In one embodiment the present invention relates to an antibody or an antigen binding fragment thereof, wherein the variable heavy chain region is characterized by a sequence at least 80% identical to, preferably by a sequence at least 85% identical to, more preferably by a sequence at least 90% identical to, even more preferably by a sequence at least 95% identical to, more preferably identical to a sequence:

FH0 - CDR-H1 - FH1 - CDR-H2 - FH2 - CDR-H3 - FH3

wherein FH0, CDR-H1, FH1, CDR-H2, FH2, CDR-H3 and FH3 are as defined hereinabove,
and/or (preferably and)
wherein the variable light chain region is characterized by a sequence at least 80% identical to, preferably by a sequence at least 85% identical to, more preferably by a sequence at least 90% identical to, even more preferably by a sequence at least 95% identical to, more preferably identical to a sequence:

FL0 - CDR-L1 - FL1 - CDR-L2 - FL2 - CDR-L3 - FL3

wherein FL0, CDR-L1, FL1, CDR-L2, FL2, CDR-L3 and FL3 are as defined hereinabove.

Encompassed by the present invention is an antibody or the antigen-binding fragment thereof, wherein any combination of the variable light chain, as defined hereinabove, and the variable heavy chain, as defined hereinabove, is encompassed.

For the design of CDR-grafted versions of the CE7 murine VH, two human germlines, IGHV1-2*06 and IGHV4-34*01 were selected. The two human germlines have a sequence identity across the whole V gene of 66.3% and 53.1%, respectively. For the design of CDR-grafted versions of the CE7 murine VL, three human germlines; IGKV1-NL1*01, IGKV1-33*01 and IGKV3-15*01 were selected. The three human germlines have a sequence identity across the whole V-gene with the CE7 murine VL of 73.7%, 71.6% and 60.0%, respectively.

According to in silico immunogenicity modeling study performed by the present inventors, a number of mutations can be introduced into the germlines as described above in order to reduce their potential immunogenicity.

Accordingly, in VH of IGHV4-34*01 with CE7-CDRs grafted thereon, the following mutations are predicted to be particularly beneficial for reducing immunogenicity:
M residue at position 34 may be replaced by a residue selected from C, D and E;
M residue at position 35 may be replaced by C;
I residue at position 37 may be replaced by a residue selected from C, D and E;
R residue at position 63 may be replaced by a residue selected from D, E and P;
L residue at position 64 may be replaced by a residue selected from A, C, D, E, G, H, K, N, P, Q, S and T;
K residue at position 65 may be replaced by a residue selected from C, D, E, N and P;
S residue at position 66 may be replaced by a residue selected from C, and D;
R residue at position 67 may be replaced by a residue selected from D and P;
V residue at position 68 may be replaced by a residue selected from C, D, E, G, N and Q;
T residue at position 69 may be replaced by a residue selected from D and E;
L residue at position 70 may be replaced by a residue selected from C, D, E, G, N, P, Q, S and T; and/or
V residue at position 72 may be replaced by D.

Further accordingly, in VL of IGKV1-NL1*01 with CE7-CDRs grafted thereon, the following mutations are predicted to be particularly beneficial for reducing immunogenicity:
L residue at position 46 may be replaced by a residue selected from A, D, E, G, H, N, P, Q, S, and T;
L residue at position 47 may be replaced by a residue selected from D, E, G, N, P, S, and T;
A residue at position 51 may be replaced by E;
T residue at position 52 may be replaced by D;
N residue at position 53 may be replaced by D;
L residue at position 54 may be replaced by a residue selected from C, D, E, G, N, P, Q, S, and T; and/or
V residue at position 55 may be replaced by a residue selected from C, D, E, and G.

Further accordingly, in VH of IGHV1-2*06 with CE7-CDRs grafted thereon, the following mutations are predicted to be particularly beneficial for reducing immunogenicity:
L residue at position 70 may be replaced with A, C, D, E, G, H, K, N, P, Q, S and T;
T residue at position 71 may be replaced with C, and D;
V residue at position 72 may be replaced with A, C, D, E, G, H, K, N, P, Q, S, T, and W;
K residue at position 74 may be replaced with C, D, E, G, P, Q, and T,
S residue at position 75 may be replaced with C, D, E, G and Q;
I residue at position 76 may be replaced with C, D, E, G, K, N, P, Q, S and T;
S residue at position 77 may be replaced with C and E;
T residue at position 78 may be replaced with D and E; and/or
A residue at position 79 may be replaced with D.

Preferably, as encompassed by the present invention, the antibody (or if applicable, the antigen-binding fragment thereof) according to any of the above embodiments comprises a heavy chain constant region sequence comprising the amino acid sequence selected from sequences according to SEQ ID NO: 145, 146, 147, 148, 149 and 150, preferably from sequences according to SEQ ID NO: 146, 147, 148, 149 and 150, more preferably from sequences according to SEQ ID NO.: 147, 148, 149 and 150. In one embodiment, the antibody of the invention comprises a heavy chain constant region sequence having at least 90%, 91%, 92%, 93%, 94%, 95%, 96%, 97%, 98%, 99%, or 100% sequence identity to the amino acid sequence selected from sequences according to SEQ ID NO: 145, 146, 147, 148, 149 and 150, preferably from sequences according to SEQ ID NO: 146, 147, 148, 149 and 150, more preferably from sequences according to SEQ ID NO.: 147, 148, 149 and 150.

In one embodiment, the present invention relates to an antibody or an antigen-binding fragment thereof, wherein the heavy chain comprises a sequence at least 90% identical to, preferably at least 95% identical to, more preferably at least 98% identical to, even more preferably at least 99% identical to, still more preferably identical to a sequence selected from sequences according to SEQ ID NO.: 151, 152, 153 and 154.

As it is shown in Example 26, the antibody variants of the present invention, in particular the antibody variants of the embodiments described in the following, are characterized by reduced uptake in lymph nodes. This represents an important aspect of the here presented inventions as it can be postulated that a reduced uptake of huCE7 variants in healthy lymph nodes will results in a better side effect profile. The effect maybe a result of the CE7 epitope and sequence changes during humanization so that the antibody does not bind the version of L1-CAM expressed in lymph nodes.

In one embodiment, it is particularly preferred that in the antibody or an antigen binding fragment thereof of the present invention, the heavy chain comprises a sequence at least 90% identical to, preferably at least 95% identical to, more preferably at least 98% identical to, even more preferably at least 99% identical to, still more preferably identical to a sequence selected from sequences according to SEQ ID NO.: 155 to 163. In this embodiment, it is particularly preferred that in the antibody or an antigen-binding fragment thereof of the invention, the light chain comprises a sequence at least 90% identical to, preferably at least 95% identical to, more preferably at least 98% identical to, even more preferably at least 99% identical to, still more preferably identical to a sequence according to SEQ ID NO.: 143.

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 155 and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 143.

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 155 and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 97.

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 162 and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 143.

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 162 and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 97.

Accordingly, it is preferred that the antibody or the antigen-binding fragment thereof of the present invention comprises a variable heavy chain region comprising CDR-H1 according to SEQ ID NO.: 3, CDR-H2 according to SEQ ID NO.: 9, and CDR-H3 according to SEQ ID NO.: 13, and a variable light chain region comprising CDR-L1 according to SEQ ID NO.: 18, CDR-L2 according to SEQ ID NO.: 23, and CDR-L3 according to SEQ ID NO.: 26.

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 110 (CE7-1-2-VHB and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 97 (CE7-1-NL1-VLA).

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 142 (CE7-1-2-VHB+WH33F, GH55A) and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 97 (CE7-1-NL1-VLA).

In one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence according to SEQ ID NO. 110 and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence according to SEQ ID NO.: 143.

In one specific embodiment, the heavy chain of the antibody does not bear a mutation leading do deglycosylation of the antibody. Accordingly, in one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence selected from sequences according to SEQ ID NO. 110, 142, 155 to 162, wherein the mutation N297A has been reversed, and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence selected from sequences according to SEQ ID NO.: 97 and 143.

Accordingly, in one specific embodiment, in the antibody or an antigen-binding fragment thereof of the present invention the heavy chain comprises a sequence at least 80% identical to, preferably at least 85% identical to, more preferably at least 90% identical to, even more preferably at least 95% identical to, even more preferably at least 98% identical to, even more preferably at least 99% identical to, still even more preferably identical to a sequence selected from sequences according to SEQ ID NO: 95 and 164 to 172, and the light chain comprises a sequence at least 80% identical to, preferably at least 85% identical to, more preferably at least 90% identical to, even more preferably at least 95% identical to, more preferably at least 98% identical to, even more preferably at least 99% identical to, still even more preferably identical to a sequence selected from sequences according to SEQ ID NO.: 97 and 143.

In one specific embodiment, in the antibody or an antigen-binding fragment thereof of the present invention the heavy chain comprises a sequence at least 90% identical to, preferably at least 95% identical to, more preferably at least 98% identical to, even more preferably at least 99% identical to, still more preferably identical to a sequence according to SEQ ID NO. 110, 142, 155, 160 or 162, and the light chain comprises a sequence at least 90% identical to, preferably at least 95% identical to, more preferably at least 98% identical to, even more preferably at least 99% identical to, still more preferably identical to a sequence according to SEQ ID NO.: 97 or 143.

In one specific embodiment, in the antibody or an antigen-binding fragment thereof of the present invention the heavy chain comprises a sequence at least 90% identical to, preferably at least 95% identical to, more preferably at least 98% identical to, even more preferably at least 99% identical to, still more preferably identical to a sequence selected from sequences according to SEQ ID NO. 173 to 183, and the light chain comprises a sequence at least 90% identical to, preferably at least 95% identical to, more preferably at least 98% identical to, even more preferably at least 99% identical to, still more preferably identical to a sequence according to SEQ ID NO.: 193.

In one specific embodiment, in the antibody or an antigen-binding fragment thereof of the present invention the heavy chain comprises a sequence at least 90% identical to, preferably at least 95% identical to, more preferably at least 98% identical to, even more preferably at least 99% identical to, still more preferably identical to a sequence selected from sequences according to SEQ ID NO. 173 to 183, and the light chain comprises a sequence at least 90% identical to, preferably at least 95% identical to, more preferably at least 98% identical to, even more preferably at least 99% identical to, still more preferably identical to a sequence according to SEQ ID NO.: 194.

In one specific embodiment, in the antibody or an antigen-binding fragment thereof of the present invention the heavy chain comprises a sequence at least 90% identical to, preferably at least 95% identical to, more preferably at least 98% identical to, even more preferably at least 99% identical to, still more preferably identical to a sequence selected from sequences according to SEQ ID NO. 184 to 192, and the light chain comprises a sequence at least 90% identical to, preferably at least 95% identical to, more preferably at least 98% identical to, even more preferably at least 99% identical to, still more preferably identical to a sequence according to SEQ ID NO.: 193.

In one specific embodiment, in the antibody or an antigen-binding fragment thereof of the present invention the heavy chain comprises a sequence at least 90% identical to, preferably at least 95% identical to, more preferably at least 98% identical to, even more preferably at least 99% identical to, still more preferably identical to a sequence selected from sequences according to SEQ ID NO. 184 to 192, and the light chain comprises a sequence at least 90% identical to, preferably at least 95% identical to, more preferably at least 98% identical to, even more preferably at least 99% identical to, still more preferably identical to a sequence according to SEQ ID NO.: 194.

It is to be understood that whenever a heavy or a light chain is described to comprise a particular sequence, a direct reference is also made to a heavy or a light chain, respectively, having said sequence, or consisting of said sequence.

As it is to be understood herein, preferably, the antibody or the antigen-binding fragment thereof of the present invention is characterized by a dissociation constant K_{D} to L1-CAM (CD171) not exceeding 10⁻¹¹ M, more preferably not exceeding 10⁻¹² M. It is to be understood that said K_{D} is preferably measured in a Biacore-based assay.

Amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and FRs. Conservative substitutions are shown in Table D1 under the heading of “preferred substitutions.” More substantial changes are provided in Table D1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE D1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe | Leu |
| | Norleucine | |
| Leu (L) | Norleucine; Ile; Val; Met; Ala | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fe region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 AI, Presta, L; and WO 2004/056312 AI, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and antibody-dependent cellular cytotoxicity) are unnecessary or deleterious. In vitro and/or in vivo cytotoxicity assays can be conducted to confirm the reduction/depletion of CDC (complement-dependent cytotoxicity) and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)).

Accordingly and preferably, in the antibody or the antigen-binding fragment thereof (as far as applicable) according to the present invention, the heavy chain comprises at least one point mutation in Fc part that influences antibody-dependent cell-mediated cytotoxicity (ADCC), complement-dependent cytotoxicity (CDC), serum half-life and/or glycosylation status of the antibody. It is to be understood to the skilled person that only antigen-binding fragments of said antibody that comprise the heavy chain, or the fragment thereof, may be encompassed herein. The at least one point mutation as referred to herein is preferably selected from L234A, L234F, L235A, L235E, L235Q, G236A, M252Y, S254T, T256E, S267E, H268F, N297A, K322A, K322Q, S324T, P331S, and I332E. More preferably, the at least one point mutation is selected from L234A, L235A, P331S and N297A. It is to be understood herein that the at least one point mutation may refer to more than one mutation. For example, in one preferred embodiment of the present invention, the heavy chain comprises L234A, L235A, P331S and N297A point mutations. As it is preferably to be understood herein, the amino acid positions as recited herein refer to the residue numbering as in IgG1 isotype. Should any other antibody class or isotype be used, the skilled person will be in position to translate the amino acid positions to any other antibody class or isotype known in the art.

The skilled person may also envisage including at least one mutation that increases antibody-dependent cell-mediated cytotoxicity (ADCC) and/or complement-dependent cytotoxicity (CDC). One possible combination is at least one mutation selected from S267E/H268F/S324T/G236A/I332E (EFTAE modification), preferably the mutations S267E/H268F/S324T/G236A/I332E (EFTAE modification).

The skilled person may further envisage including at least one mutation that counters the negative effect of N297A mutation (which allows for the production of deglycosylated antibodies). One possible combination is at least one mutation selected from F241K, L309D, T307R and T307P, preferably the mutations F241K, L309D, T307R and T307P. According to the present inventors, the at least one mutation selected from F241K, L309D, T307R and T307P, preferably the mutations F241K, L309D, T307R and T307P may lead to increased thermal stability of the antibody. Without being bound by the theory, said mutation(s) increase the stability of the Fc part of the heavy chain of the antibody.

Encompassed by the present invention are further the antibodies, or antigen-binding fragments thereof as described herein, further comprising in its heavy chain one or more following mutations: G31D, G31E, G31K, G31P, G31R, H35K, T28D, T28E, T28K, T28P, T28R, T30D, T30E, T30K, T30P, T30R, W33R, Y27D, Y27E, and Y27K, preferably comprising a mutation selected from G31D, G31E, G31K, G31P, G31R, H35K, T28D, T28E, T28K, T28P, T28R, T30D, T30E, T30K, T30P, T30R, W33R, Y27D, Y27E, and Y27K. Preferably, encompassed by the present invention are the antibodies or antigen-binding fragments thereof as described herein, further comprising in its heavy chain one or more following mutations: T30P, T28K and T30E. According to the present inventors, at least one mutation selected from G31D, G31E, G31K, G31P, G31R, H35K, T28D, T28E, T28K, T28P, T28R, T30D, T30E, T30K, T30P, T30R, W33R, Y27D, Y27E, and Y27K, preferably selected from T30P, T28K and T30E stabilize the flanking region of CDR1 and/or may lead to improved affinity of the antibody or its fragment to L1-CAM.

Encompassed by the present invention are further the antibodies, or antigen-binding fragments thereof as described herein, further comprising in its heavy chain one or more following mutations: A40R, V68A, and L115T. Without being bound to the theory, these mutations correspond to incorporation of the residues originally present in the murine antibody, which are close to the aggregation-prone regions. According to the present inventors, the at least one mutation selected from A40R, V68A, and L115T may lead to increased thermal stability of the antibody. Without being bound by the theory, said mutation(s) increase the stability of the Fc part of the heavy chain of the antibody.

The antibody or an antigen binding thereof of the present invention, provided herein may be further modified to contain additional nonproteinaceous moieties that are known in the art and readily available. The moieties suitable for derivatization of the antibody include but are not limited to water soluble polymers. Nonlimiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Further suitable and preferred examples of water-soluble polymers, such as polymers that include acrylic backbone, are described hereinbelow.

The antibody or an antigen binding thereof of the present invention, provided herein may also be further modified to contain additional moieties, thus yielding immunoconjugates comprising the antibody or the antigen-binding fragment thereof of the present invention. Said immunoconjugates are described herein.

### Production of Antibodies

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extra chromosomally or at a chromosomal location that is different from its natural chromosomal location.

“Isolated nucleic acid encoding an anti-L1-CAM antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

In a further embodiment, one or more vectors (e.g., expression vectors) comprising such nucleic acid are provided. Accordingly, the present invention relates to a polynucleotide encoding at least one variable heavy chain sequence and/or at least one variable light chain sequence as described herein. It is to be understood that, unless indicated to the contrary, the terms polynucleotide and (isolated) nucleic acids may be used interchangeably.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

Accordingly, the present invention further relates to a vector comprising the polynucleotide encoding at least one variable heavy chain sequence and/or at least one variable light chain sequence as described herein.

In a further embodiment, a host cell comprising such nucleic acid is provided.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20). In one embodiment, a method of making an antibody of the present invention is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody of the present invention, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Val. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CVI line transformed by SV40 (COS-7); human embryonic kidney line (293 or 293 cells as described, e.g., in Graham et al., J. Gen Viral. 36:59 (1977)); baby hamster kidney cells (BHK); mouse sertoli cells (TM4 cells as described, e.g., in Mather, Biol. Reprod. 23:243-251 (1980)); macaque kidney cells (CV I); African green macaque kidney cells (VERO-76); human cervical carcinoma cells (HELA); canine kidney cells (MDCK; buffalo rat liver cells (BRL 3A); human lung cells (WI38); human liver cells (Hep G2); mouse mammary tumor (MMT 060562); TRI cells, as described, e.g., in Mather et al., Annals N.Y Acad. Sei. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. cii. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Val. 248 (B. K. C. Lo, ed., Humana Press, Totowa, NJ), pp. 255-268 (2003).

The antibody or the antigen-binding fragment thereof may in one embodiment include a further domain or a further amino acid sequence. For example, the antibody or the antigen-binding fragment thereof may include a localization sequence. Further examples of suitable include aldehyde tag and sortase recognition motif. The aldehyde tag is an artificial peptide tag recognized by the formylglycine-generating enzyme (FGE). A suitable example of an aldehyde tag is a tag according to sequence LCTPSR (SEQ ID NO.: 52), wherein upon FGE acting on said sequence, the cysteine residue is converted to formylglycine. The sortase recognition motif is according to sequence LPXTG (SEQ ID NO.: 53), wherein X can be any natural amino acid residue. The sortase enzyme, for example Staphylococcus aureus sortase, is a transpeptidase that attaches surface proteins to the cell wall; it cleaves between the Gly and Thr of the LPXTG motif and catalyses the formation of an amide bond between the carboxyl-group of threonine and the amino-group of the cell-wall peptidoglycan. As known to the skilled person, sortase recognition motif allows for attachment of further peptidic moieties.

The antibody or the antigen-binding fragment thereof may in one embodiment include further a domain or an amino acid sequence used for co-targeting to tumor microenvironment (e.g., targeting Fibroblast activation protein-α (FAP)); to overcome blood brain barrier (BBB) (e.g., targeting transferrin receptors); or to overcome endothelial cells (EC) barrier, (e.g., caveolae targeting of aminopeptidase P2 (APP2). Said additional targeting domain may thereby be directly included into the sequence of the antibody or the antigen-binding fragment to the C or N-terminus using a spacer peptide (fusion construct) or later attached e.g., by a site-specific functionalization.

The antibody or the antigen-binding fragment thereof may also optionally include one or more non-canonical amino acids to be used for coupling of said antibody or the fragment thereof with another chemical entity. Said amino acids may include residues that would be reactive in addition reactions known to the skilled person as click chemistry. Suitable examples of such residues include residues comprising azide moiety, or cyclooctyne moiety or a moiety being capable to perform an inverse-demand Diels-Alder cycloaddition reaction e.g., a trans-cyclooctene/tetrazine reaction pair. However, the invention is not meant to be limited to any of these examples, and other such residues known to the skilled person may also be used. The methods of producing antibodies or fragment thereof comprising non-canonical amino acid residue(s) using recombinant methods are known to the skilled person.

In one embodiment, the present invention further relates to an immunoconjugate comprising an antibody or an antigen-fragment binding thereof of the present invention, and an active agent. It is preferred, however not required that the active agent is a cytotoxic agent or a prodrug thereof, preferably a cytotoxic agent. Accordingly, as understood herein, An “immunoconjugate” is an antibody (or the antigen binding fragment thereof) conjugated to one or more heterologous molecule(s), including but not limited to a cytotoxic agent. Said cytotoxic agent or prodrug may also be referred to as drug.

The immunoconjugate of the present invention may also be referred to as an antibody-drug conjugate. Antibody-drug conjugates (ADC) are targeted chemotherapeutic molecules which combine properties of both antibodies and cytotoxic drugs by targeting potent cytotoxic drugs to antigen expressing tumor cells (Teicher, B. A. (2009) Current Cancer Drug Targets 9:982-1004), thereby enhancing the therapeutic index by maximizing efficacy and minimizing off-target toxicity (Carter, and Senter P. D. (2008) The Cancer Jour. 14(3):154-169; Chari, R. V. (2008) Ace. Chem. Res. 41:98-107.

The ADC compounds of the invention include those with anticancer activity. In some embodiments, the ADC compounds include an antibody conjugated, i.e. covalently attached, to the drug moiety. In some embodiments, the antibody is covalently attached to the drug moiety through a linker. The antibody-drug conjugates (ADC) of the invention selectively deliver an effective dose of a drug to tumor tissue whereby greater selectivity, i.e. a lower efficacious dose, may be achieved while increasing the therapeutic index (“therapeutic window”).

As referred to herein, linkers are not to be particularly limited and any linker conceivable to the skilled person as usable in the immunoconjugate, or antibody-drug conjugate, can be used within the scope of the present invention.

In one embodiment of the present invention, the linker takes the form of a polymeric linker. Accordingly, said polymeric linker includes at least two types of repeating unit, preferably more than two types of a repeating unit. It is to be understood that at least one type of the repeating unit is covalently attached to an active agent, for example a cytotoxic agent. Accordingly, at least one type of the repeating unit is free of said active agent, thus accordingly, the polymeric carrier may the active agent incorporated into some of its repeating units. Further accordingly, multiple molecules of the active agent may thus so be attached to a single attachment point on the antibody or an antigen-binding fragment thereof, by using a polymeric carrier.

Any polymeric carrier attachable to an antibody or to a fragment thereof may be used in the immunoconjugates of the present invention. Examples of polymeric carriers particularly suitable for use in the immunoconjugates of the present invention are described in the patent applications PCT/EP2020/080545 and PCT/EP2019/061769, each of which is herewith incorporated by reference in its entirety. One exemplary polymeric carrier will be defined in the following.

Preferably, the polymeric carrier comprises a repeating unit of a formula (R1)

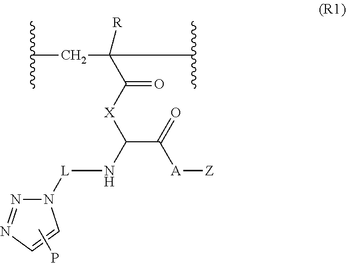

wherein R is —H, —CH₃, —CH₂—CH₃ or —(CH₂)₂—CH₃; X is —NH(CH₂)₄—, —NH(CH₂)₃—, —O—C₆H₄—CH₂—, —O—CH₂—, —O—CH(CH₃)—, —S—CH₂— or —NH—C₆H₄—CH₂—; Z is H (if A is —O—) or —CₙH₂ₙ₊₁ (with n=1-8); and A is —O— or —NH—; L is a spacer, and P comprises an active agent.

As preferably understood herein, if A is —O—, then Z is H or —CₙH₂ₙ₊₁ (with n=1-8), whereas if A is —NH—, Z is —CₙH₂ₙ₊₁ (with n=1-8). Alternatively, Z can preferably also be defined as Z is H or —CₙH₂ₙ₊₁ (with n=1-8). Preferably, if A is —O—, Z is preferably H. Preferably, the copolymer comprising the repeating unit of formula (R1a) or the copolymer comprising the repeating unit of formula (R1), further comprises a repeating unit of formula (R2):

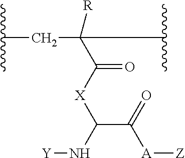

wherein R is —H, —CH3, —CH2-CH3 or —(CH2)2-CH3; X is —NH(CH2)4-, —NH(CH2)3-, —O—C6H4-CH2-, —O—CH2-, —O—CH(CH3)-, —S—CH2- or —NH—C6H4-CH2-; Y is H or —CO—CnH2n+1 (with n=1-8) or Y comprises a second payload molecule; Z is H (if A is —O—) or —CnH2n+1 (with n=1-8), or Z comprises a second payload molecule; and A is —O— or —NH—,
and/or a repeating unit of formula (R3):

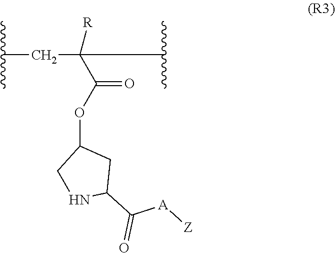

wherein: R is —H, —CH3, —CH2-CH3 or —(CH2)2-CH3; Z is H (if A is 0) or —CnH2n+1 (with n=1-8), or Z comprises a second payload molecule; and A is —O— or —NH—.

As preferably understood herein, if A is —O—, then Z is H or —CnH2n+1 (with n=1-8), whereas if A is —NH—, Z is —CnH2n+1 (with n=1-8). Alternatively, Z can preferably also be defined as Z is H or —CnH2n+1 (with n=1-8). Preferably, if A is —O—, Z is preferably H.

Preferably, Z can be H and/or Y can be H. Alternatively, Z and/or Y can comprise a second payload molecule. The payload molecule is as defined herein. Alternatively, in certain embodiments, Z is H or —CnH2n+1 (with n=1-8), or Z comprises a second active agent molecule.

Further preferably, the polymeric carrier as described herein comprises a further repeating unit obtainable by polymerization of N,N-dimethyl-acrylamide, N-isobutyl-acrylamide, N-tert. butyl-acrylamide, N-hydroxyethyl-acrylamide, N-(2-hydroxypropyl)-acrylamide, N-(3-hydroxypropyl)-acrylamide, N-(3-hydroxypropyl)-methacrylamide, N-(2-hydroxypropyl)-methacrylamide, N-(3-aminopropyl)-acrylamide hydrochloride, or N-(3-aminopropyl)-methacrylamide hydrochloride, or a repeating unit obtained through polymerization of methacrylic acid, 2-hydroxyethyl-acrylate, 2-hydroxypropyl-acrylate, 3-hydroxypropyl-acrylate, 2-hydroxy-1-methylethyl-acrylate, 2-aminoethyl acrylate hydrochloride, 3-hydroxypropyl-methacrylate, 2-hydroxy-1-methylethyl-methacrylate, 2-hydroxyethyl-methacrylate, 2-hydroxypropyl-methacrylate or 2-aminoethyl methacrylate hydrochloride.

Further preferably, in the polymeric carrier as described herein, wherein the repeating units of formulae (R2) and (R3) are absent, the average number of repeating units according to formula (R1) per molecule of copolymer is 2 to 12, preferably 2 to 8, more preferably 2 to 6.

Further preferably, in the polymeric carrier as described herein, wherein the repeating units of formulae (R2) or (R3) are not functionalized, as defined herein, the average number of repeating units according to formulae (R1), (R2) or (R3) per molecule of copolymer is 10 to 50, preferably 10 to 40, more preferably 10 to 30.

Further preferably, in the polymeric carrier as described herein, wherein the repeating units of formulae (R2) or (R3) are functionalized with a second payload molecule, the average number of repeating units according to formulae (R1), (R2) or (R3) per molecule of copolymer is 4 to 20, preferably 4 to 15, more preferably 4 to 10.

Particularly suitable examples of the polymeric carriers to be used in the immunoconjugates of the present invention include the carriers derived from the compounds selected from:

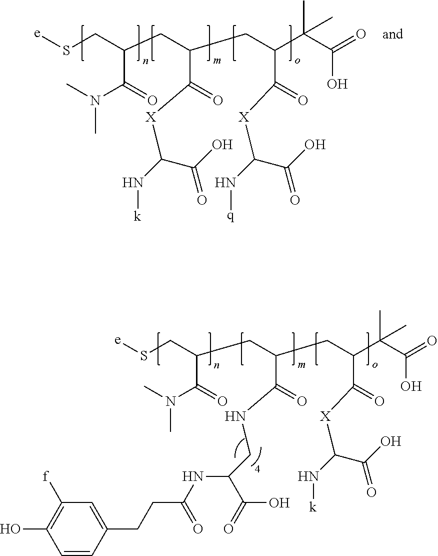

wherein each X is independently —NH(CH₂)₄—, or —NH—C₆H₄—CH₂—; n is 40-120; m is 4-16; o is 0-16; f is —H or —I (it is to be understood that I includes both cold isotopes of iodine as well as hot isotopes of iodine), k and q are designated as an active payload or could be H.

The end group, e, may be H or may be a modified end group, as discussed and exemplified in the following.

Concerning the end group modifications, the end group e may be introduced via a thiol-reactive group. Suitable reagents include but not limited to:
MC-DBCO
N-[3-(11,12-Didehydrodibenz[b,f]azocin-5(6H)-yl)-3-oxopropyl]-2,5-dihydro-2,5-dioxo-1H-pyrrole-1-propanamide
Methyltetrazine-PEG4-maleimide
3-(2,5-dioxopyrrol-1-yl)-N-[2-[2-[2-[2-[4-(6-methyl-1,2,4,5-tetrazin-3yl)phenoxy]ethoxy]ethoxy]-ethoxy]ethyl]propanamide
Cas 1802908-02-6

It is to be understood that the payload molecules k and q may be introduced via an amino-reactive group. Suitable reagents that could be used for introducing said payload molecules are selected from:
p-SCN-Bn-CHX-A″-DTPA: [(R)-2-Amino-3-(4-isothiocyanatophenyl)propyl]-trans-(S,S)-cyclohexane-1,2-diamine-pentaacetic acid;
p-SCN-Bn-DTPA: S-2-(4-Isothiocyanatobenzyl)-diethylenetriamine pentaacetic acid; DOTA-GA anhydride: 2,2′,2″-(10-(2,6-dioxotetrahydro-2H-pyran-3-yl)-1,4,7,10-tetraazacyclododecane-1,4,7-triyl)triacetic acid;
p-SCN-Bn-DOTA: S-2-(4-Isothiocyanatobenzyl)-1,4,7,10-tetraazacyclododecane tetraacetic acid;
NHS DOTA: 1,4,7,10-Tetraazacyclododecane-1,4,7,10-tetraacetic acid mono-N-hydroxysuccinimide ester;
p-SCN-Bn-NOTA: 2-S-(4-Isothiocyanatobenzyl)-1,4,7-triazacyclononane-1,4,7-triacetic acid;
NOTA-NHS ester: 2,2′-(7-(2-((2,5-dioxopyrrolidin-1-yl)oxy)-2-oxoethyl)-1,4,7-triazonane-1,4-diyl)diacetic acid;
NODA-GA-NHS ester: 2,2′-(7-(1-carboxy-4-((2,5-dioxopyrrolidin-1-yl)oxy)-4-oxobutyl)-1,4,7-triazonane-1,4-diyl)diacetic acid;
Paclitaxcel NHS: (1S,2R)-1-benzamido-3-(((2aR,4S,4aS,6R,9S,11 S,12S,12aR,12bS)-6,12b-diacetoxy-12-(benzoyloxy)-4,11-dihydroxy-4a,8,13,13-tetramethyl-5-oxo-2a,3,4,4a,5,6,9,10,11,12,12a,12b-dodecahydro-1H-7,11-methanocyclodeca[3,4]benzo[1,2-b]oxet-9-yl)oxy)-3-oxo-1-phenylpropan-2-yl (2,5-dioxopyrrolidin-1-yl) succinate;
MMAE-NHS: 4-(2-(2-((((2,5-dioxopyrrolidin-1-yl)oxy)carbonyl)amino)-3-methylbutanamido)-5-ureidopentanamido)benzyl (1-((1-((1-(2-(3-((1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate;
MMAF-NHS: N-[6-[(2,5-Dioxo-1-pyrrolidinyl)oxy]-6-oxohexyl]-N-methyl-L-valyl-L-valyl-(3R,4S,5S)-3-methoxy-5-methyl-4-(methylamino)heptanoyl-(αR,βR,2S)-β-methoxy-α-methyl-2-pyrrolidinepropanoyl-L-phenylalanine;
DM1-SMCC (Maytansinoid-NHS-derivative): N2′-deacetyl-N2′-[3-[[1-[[4-[[(2,5-dioxo-1-pyrrolidinyl)oxy]carbonyl]cyclohexyl]methyl]-2,5-dioxo-3-pyrrolidinyl]thio]-1-oxopropyl]-Maytansine;
SC-VC-PAB-DM1 (CAS: 2259318-47-1);
Doxorubicin-SMCC: 7,8,9,10-tetrahydro-6,8,11-trihydroxy-8-(2-hydroxyacetyl)-1-methoxy-10-[[2,3,6-trideoxy-3-[[[4-[(2,5-dihydro-2,5-dioxo-1H-pyrrol-1-yl)methyl]cyclohexyl]carbonyl]amino]-α-L-lyxo-hexopyranosyl]oxy]-, (8S,10S)-5,12-Naphthacenedione;
Reagents suitable for introducing a kinase inhibitor, preferably selected from Fasudil, Sirolimus, Imatinib, Gefitinib, Erlotinib, Soragenib, Sunitinib, Dasatinib, Lapatinib, Nilotinib, Temsirolimus, Everolimus, Pazopanib, Ruxolitinib, Vandetanib, Vemurafenib, Crizotinib, Icotinib, Axitinib, Tofacitinib, Bosutinib, Cabozantinib, Ponatinib, Regorafenib, Afatinib, Dabrafenib, Trametinib, Ibrutinib, Nintedanib, Idelasilib, Ceritinib, Apatinib, Ripasudil, Alectinib, Cobimetinib, Lenvatinib, Palbociclib, Radotinib, Osimertinib, Olmutunib, Neratinib, Ribociclib, Copanlisib, Abemaciclib, Acalabrunitib, Midostaurin, Brigatinib, Baricitinib, Netarsudil, Tivozanib, Simotinib, Fostamatinib, Encorafenib, Binimetinib, Catequentinib, Duvelisib, Dacomitinib, Lorlatinib, Larotrectinib, Gilteritinib, Pyrotinib, Fruquintinib, Erdafitinib, Alelisib, Umbralisib, Leniolisib, Pexidartinib, Entrectinib, Fedratinib, Zanubrutinib, Flumatinib, Peficitinib, Delgocitinib, Avapritinib, Selumetinib, Tucatinib, Pemigatinib, Capmatinib, Selpercatinib, Ripretinib, Tirabrutinib, Almonertinib, Pralsetinib, Filgotinib, Tirbanilubil, Orelabrutinib, Tepotinib, and Trilaciclib.

Reagents suitable for introducing doxorubicin or derivatives thereof. Preferably, derivatives of doxorubicin are understood as the compounds comprising the following moiety:

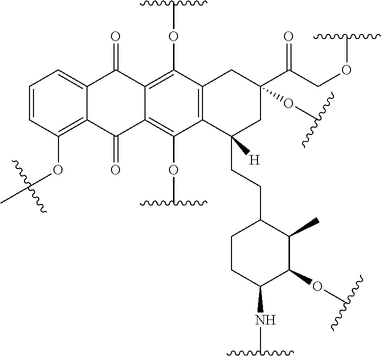 [SMILES: *N[C@H]1CCC(CC[C@H]2C[C@](O*)(C(=O)CO*)Cc3c(O*)c4c(c(O*)c32)C(=O)c2c(O*)cccc2C4=O)[C@@H](C)[C@H]1O*]

Preferably, said doxorubicin derivatives comprise DBCO moiety.

PNU-159682;

Alternatively, the payload molecules k an q may be introduced in a two-step process via click reaction. In this case, the amine-reactive reagent is an NHS ester of an azido-functionalized carboxylic acid and the payload molecules are but not limited to:
DBCO-DOTA
4,7,10-Tetraazacyclododecane-1,4,7-tris(acetic acid)-10-[3-oxo-3-(5-azadibenzocyclootyne)acetamide]
DBCO-PEG4-Val-Cit-PAB-MMAE
[4-[[(2S)-2-[[(2S)-2-[3-[2-[2-[2-[2-[[4-(2-azatricyclo[10.4.0.04,9]hexadeca-1(16),4,6,8,12,14-hexaen-10-yn-2-yl)-4-oxobutanoyl]amino]ethoxy]ethoxy]ethoxy]ethoxy]propanoylamino]-3-methylbutanoyl]amino]-5-(carbamoylamino)pentanoyl]amino]phenyl]methyl N-[(2S)-1-[[(2S)-1-[[(3R,4S,5S)-1-[(2S)-2-[(1R,2R)-3-[[(1S,2R)-1-hydroxy-1-phenylpropan-2-yl]amino]-1-methoxy-2-methyl-3-oxopropyl]pyrrolidin-1-yl]-3-methoxy-5-methyl-1-oxoheptan-4-yl]-methylamino]-3-methyl-1-oxobutan-2-yl]amino]-3-methyl-1-oxobutan-2-yl]-N-methylcarbamate
DBCO-Val-Cit-PAB-MMAE
DBCO-Val-Cit-PAB-MMAF
DBCO-PEG4-MMAF (CAS: 2360411-65-8)
DM1-PEG4-DBCO
DBCO-PEG4-Ahx-DM1

Reagents suitable for introducing a kinase inhibitor, preferably selected from Fasudil, Sirolimus, Imatinib, Gefitinib, Erlotinib, Soragenib, Sunitinib, Dasatinib, Lapatinib, Nilotinib, Temsirolimus, Everolimus, Pazopanib, Ruxolitinib, Vandetanib, Vemurafenib, Crizotinib, Icotinib, Axitinib, Tofacitinib, Bosutinib, Cabozantinib, Ponatinib, Regorafenib, Afatinib, Dabrafenib, Trametinib, Ibrutinib, Nintedanib, Idelasilib, Ceritinib, Apatinib, Ripasudil, Alectinib, Cobimetinib, Lenvatinib, Palbociclib, Radotinib, Osimertinib, Olmutunib, Neratinib, Ribociclib, Copanlisib, Abemaciclib, Acalabrunitib, Midostaurin, Brigatinib, Baricitinib, Netarsudil, Tivozanib, Simotinib, Fostamatinib, Encorafenib, Binimetinib, Catequentinib, Duvelisib, Dacomitinib, Lorlatinib, Larotrectinib, Gilteritinib, Pyrotinib, Fruquintinib, Erdafitinib, Alelisib, Umbralisib, Leniolisib, Pexidartinib, Entrectinib, Fedratinib, Zanubrutinib, Flumatinib, Peficitinib, Delgocitinib, Avapritinib, Selumetinib, Tucatinib, Pemigatinib, Capmatinib, Selpercatinib, Ripretinib, Tirabrutinib, Almonertinib, Pralsetinib, Filgotinib, Tirbanilubil, Orelabrutinib, Tepotinib, and Trilaciclib.

Reagents suitable for introducing doxorubicin or derivatives thereof. Preferably, derivatives of doxorubicin are understood as the compounds comprising the following moiety:

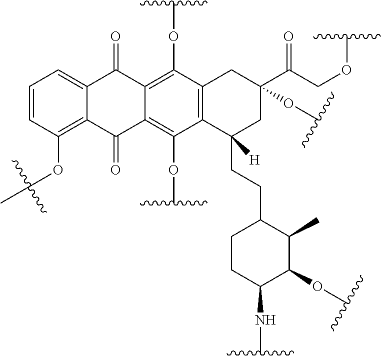 [SMILES: *N[C@H]1CCC(CC[C@H]2C[C@](O*)(C(=O)CO*)Cc3c(O*)c4c(c(O*)c32)C(=O)c2c(O*)cccc2C4=O)[C@@H](C)[C@H]1O*]

Preferably, said doxorubicin derivatives comprise DBCO moiety.

DBCO-PEG4-VC-PAB-DMEA-PNU-159682 (CAS: 2259318-56-2)

A reagent suitable for introducing AZD7648:

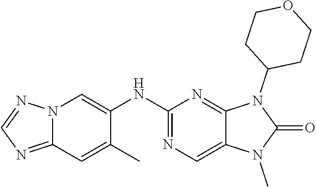 [SMILES: Cc1cc2ncnn2cc1Nc1ncc2c(n1)n(C1CCOCC1)c(=O)n2C]

The skilled person would understand the word derived as recited herein preferably as referring to attachment of any of the compounds to the antibody or the fragment thereof, e.g., through a condensation reaction involving the free carboxylic acid group. It is preferably to be understood that the formulas do not represent block copolymers, but statistical composition of the obtained polymer. It is however to be understood that the block copolymers or gradient copolymers may also be encompassed by the definitions provided herein, as their formation may also be feasible, depending on specific conditions in the reactor.

The drug moiety (D) of the antibody-drug conjugates (ADC) may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, calicheamicin, pyrrolobenzodiazepine (PBD), nemorubicin and its derivatives, PNU-159682, anthracycline, duocarmycin, vinca alkaloid, taxane, trichothecene, CC1065, camptothecin, Deruxtecan, Exatecan, elinafide, and stereoisomers, isosteres, analogs, and derivatives thereof that have cytotoxic activity. Nonlimiting examples of such immunoconjugates are discussed in further detail below. In one preferred embodiment, the drug moiety (D) is a topoisomerase inhibitor, i.e. an inhibitor of topoisomerase I and/or II, for example a derivative of Camptothecin, preferably selected from Exatecan, Deruxtecan, topotecan, irinotecan, SN38, and belotecan. In one embodiment, the active agent (i.e., the drug moiety (D)) is selected from maytansinoid, calicheamicin, pyrrolobenzodiazepine (PBD), nemorubicin and its derivatives, PNU-159682, anthracycline, duocarmycin, vinca alkaloid, taxane, trichothecene, CC1065, camptothecin, elinafide, Exatecan, Deruxtecan, topotecan, irinotecan, SN38, and belotecan.

The skilled person is capable of attaching the drug moiety to the antibody. For example, Exatecan can be attached as shown in the following schemes, wherein R represents, in general, the antibody or the antigen-binding fragment thereof, including the rest of the linkage between said antibody or said fragment thereof and the drug moiety (D):

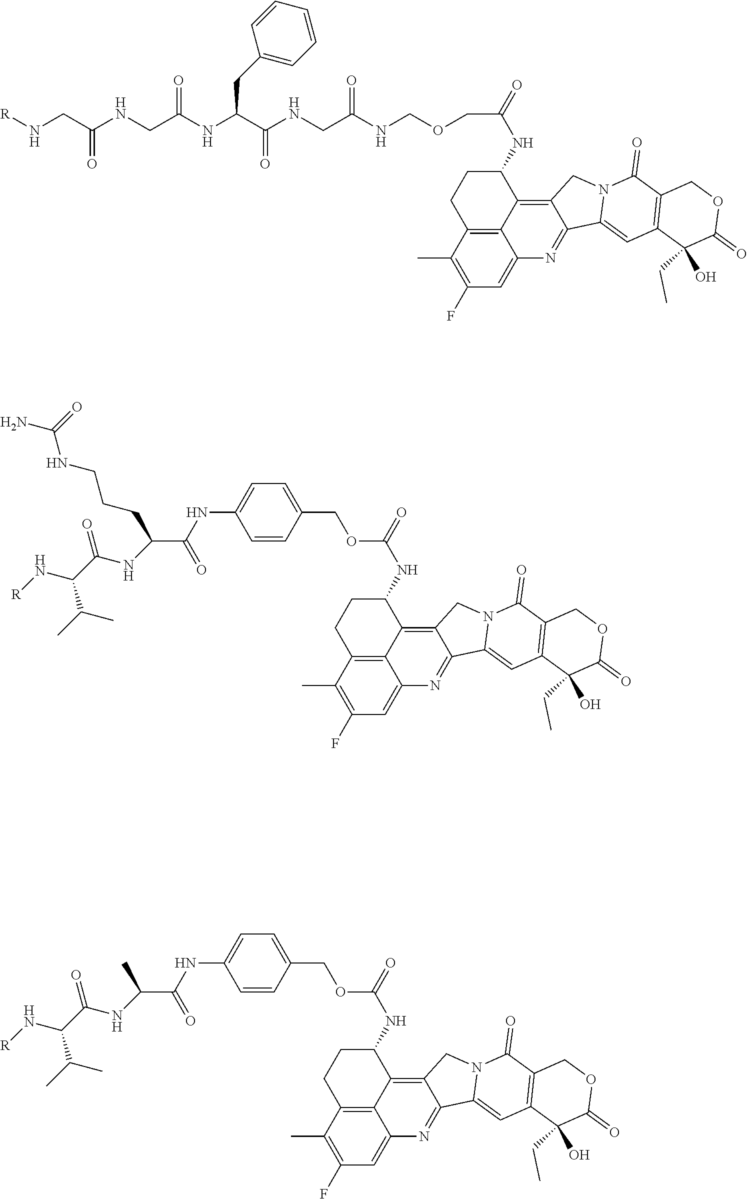

Another suitable example of moiety that could be integrated into antibody-drug conjugate of the present invention is a moiety derived from a compound according to formula:

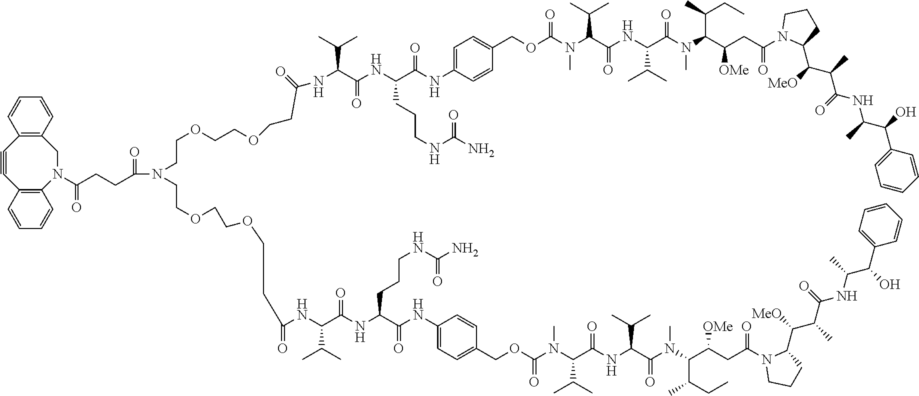

It is to be understood that an addition reaction, or a click chemistry reaction would be preferred for attachment of the compound as shown herein to the antibody or the antibody-binding fragment thereof.

Another suitable example of a drug moiety to be integrated into the antibody-drug conjugate of the invention is maitansine.

An exemplary embodiment of an antibody-drug conjugate (ADC) compound comprises an antibody (Ab) which targets a tumor cell, a drug moiety (D), and a linker moiety (L) that attaches Ab to D. In some embodiments, the antibody is attached to the linker moiety (L) through one or more amino acid residues, such as lysine and/or cysteine.

In one embodiment, the linker moiety is a polymeric carrier, as described hereinabove.

In one embodiment, more than one linker moiety is attached to the antibody or antigen-binding fragment thereof. Accordingly, in such embodiment, the immunoconjugate can be described according to the formula A-(L-D)ᵢ, wherein i is an integer number, wherein i>1.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Tb¹⁶¹, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed herein (e.g. topoisomerase inhibitors disclosed herein).

The terms “radioisotope/radionuclide” in the scope of this invention are preferably used synonymically and preferably represent an atom that has excess nuclear energy, making it unstable. This excess energy can be used in one of three ways: emitted from the nucleus as gamma radiation; transferred to one of its electrons to release it as a conversion electron; or used to create and emit a new particle (alpha particle or beta particle) from the nucleus. Radioisotope/radionuclide is herein preferably defined as an isotope which has a half-life of less than 10¹⁹ years.

Accordingly, in one embodiment the present invention relates to an immunoconjugate, wherein said immunoconjugate comprises a radioisotope as an active agent. Such conjugate may also be referred to as antibody-radionuclide conjugate.

The term “antibody radionuclide conjugate” (ARC) is preferably defined as a variant of an ADC where the “drug molecule or active molecule” represents a radionuclide/radioisotope either covalently bound to the antibody-polymer-conjugate e.g. in case of radioactive iodine or by a metal chelator complex e.g. with radioactive lutetium, actinium or terbium. The so formed ARC is capable to deliver a high amount of radiation to the tumor tissue thereby killing the tumor cells due to the damaging of DNA, essential enzymes etc. As apparent to the skilled person, the term ARC also encompasses antibody radionuclide conjugates not comprising polymer carrier, but instead comprising PEG or peptide linkers. One suitable example of such peptide linker is (Ac)-Lys-Ala-Tyr-Ala-Lys(Azide)-NH₂ [SEQ ID NO.: 54] useful for subsequent functionalization with DBCO-DOTA.

As recognizable to the skilled person, certain antibody-radionuclide conjugate may also be used for diagnostic purposes. Certain radionuclides as disclosed herein, can be monitored, for example terbium 161 due to its γ-emission can be visualized with gamma camera and hence used for detection of cancer tissue or cell-type, as targeted to by the antibody. Terbium-149 which can be used for targeted alpha therapy, has visibility in PET scans and thus can be monitored. According to the present disclosure, fluorine-18, scandium-43, scandium-44, copper-61, copper-64, gallium-68, zirconium-89, indium-111, iodine-123, terbium-152, terbium-155 are particularly useful in diagnostic application as described herein and may be referred to as radionuclides useful in diagnosis. As known to the skilled person, the radionuclides useful in diagnosis can be monitored by using a suitable method, for example Scintigraphy, Single Photon Emission Computed Tomography (SPE-CT); or Positron emission tomography Computed Tomography (PET-CT).

The skilled person will appreciate that an immunoconjugate wherein the active agent comprises a radionuclide useful for therapeutic application, for example selected from copper-67, strontium-89, yttrium-90, iodine-131, samarium-153, lutetium-177, radium-223 and actinium 225, (these radionuclides may be referred to as radionuclides useful in therapy) will have substantially the same biodistribution as the immunoconjugate wherein the active agent is a radionuclide useful for diagnostic application. Therefore, the immunoconjugates of the present invention can preferably be used for monitoring of biodistribution of therapeutic immunoconjugates during therapy. For example, an immunoconjugate comprising an active agent being a radionuclide useful in therapy can be supplemented for this purpose preferably with less than 10 weight % of an immunoconjugate wherein the active agent comprises a radionuclide useful in diagnosis, as defined herein. Further preferably, the immunoconjugate of the present invention for use in the combined therapeutic and diagnostic application may comprise two radionuclides, one radionuclide useful in therapy and one radionuclide useful in diagnosis, for example attached to a single polymeric carrier, to different repeating units thereof. Preferable are combinations wherein a radionuclide useful in therapy and a radionuclide useful in diagnosis are isotopes of the same element. Therefore, preferred combinations include scandium-43 and scandium-47, copper-61 and copper-67, copper-64 and copper-67, iodine-123 and iodine-131, terbium-152 and terbium-161, and terbium-155 and terbium-161. Further preferred combinations include isotopes of two different elements, for example indium-111 and lutetium-177, and indium-111 and terbium-161.

Pharmaceutical formulations of an antibody of the present invention or immunoconjugate of the present invention as described herein are prepared by mixing such antibody or immunoconjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA or GLDA; sugars such as sucrose, mannitol, trehalose or sorbitol; osmo-protectants like ectoin; salt-forming counter-ions such as sodium; metal complexes (e.g. Zn protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g. films, or microcapsules.

The formulations to be used for in vivo administration are generally sterile. Sterility may be readily accomplished, e.g., by filtration through sterile filtration membranes.

In one embodiment, the present invention relates to the antibody or antigen binding thereof of the present invention, the immunoconjugate of the present invention or pharmaceutical composition of the present invention for use as a medicament. In other words, the present invention relates to the antibody or antigen binding fragment thereof of the present invention, the immunoconjugate of the present invention or pharmaceutical composition of the present invention for use in therapy. It is to be understood that the antibodies, the immunoconjugate or the pharmaceutical compositions of the present invention can be used in the treatment of a disease or a disorder.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An antibody or immunoconjugate of the invention (and any additional therapeutic agent) can be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or immunoconjugates of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody or immunoconjugate need not be, but is optionally formulated with one or more agents currently used to prevent or treat the disorder in question. The effective amount of such other agents depends on the amount of antibody or immunoconjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or immunoconjugate of the invention (when used alone or in combination with one or more other additional therapeutic agents) will depend on the type of disease to be treated, the type of antibody or immunoconjugate, the severity and course of the disease, whether the antibody or immunoconjugate is administered for preventive or therapeutic purposes, previous therapy, the patient's clinical history and response to the antibody or immunoconjugate, and the discretion of the attending physician. The antibody or immunoconjugate is suitably administered to the patient at one time or over a series of treatments. Depending on the type and severity of the disease, about 1 μg/kg to 15 mg/kg (e.g. 0.1 mg/kg-10 mg/kg) of antibody or immunoconjugate can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or immunoconjugate would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

In one embodiment, the present invention relates to the antibody or antigen binding fragment thereof of the present invention, the immunoconjugate of the present invention or the pharmaceutical composition of the present invention for use in the treatment of an L1-CAM (CD171) associated cancer.

As preferably is to be understood herein, the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer and head and neck squamous cell carcinoma.

Antibodies or immunoconjugates of the invention can be used either alone or in combination with other agents in a therapy. For instance, an antibody or immunoconjugate of the invention may be co-administered with at least one additional therapeutic agent.

Such combination therapies noted above encompass combined administration (where two or more therapeutic agents are included in the same or separate formulations), and separate administration, in which case, administration of the antibody or immunoconjugate of the invention can occur prior to, simultaneously, and/or following, administration of the additional therapeutic agent and/or adjuvant. Antibodies or immunoconjugates of the invention can also be used in combination with radiation therapy.

Preferably, said antibody or the antigen-binding fragment thereof or the immunoconjugate is to be administered to a subject with an additional therapeutic agent, selected from alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, immune checkpoint inhibitors and bisphosphonate therapy agent.

Preferably, said antibody or the antigen-binding fragment thereof or the immunoconjugate of the invention is to be administered to a subject with an additional therapeutic agent, wherein said additional therapeutic agent may be an agent sensitizing the cells to radiotherapy, for example selected from protein kinase inhibitor and DNA intercalating agent. Preferably, protein kinase inhibitor is selected from Alisertib, MK1775, MK2206, Saracatinib, Temsirolimus, Crizotinib, Ceritinib, Alectinib, Brigatinib, Bosutinib, Dasatinib, Imatinib, Nilotinib, Ponatinib, Vemurafenib, Dabrafenib, Ibrutinib, Ibrutinib, Palbociclib, Sorafenib, Ribociclib, Crizotinib, Cabozantinib, Gefitinib, Erlotinib, Lapatinib, Vandetanib, Afatinib, Osimertinib, Ruxolitinib, Tofacitinib, Trametinib, Axitinib, Gefitinib, Imatinib, Lenvatinib, Nintedanib, Pazopanib, Regorafenib, Sorafenib, Sunitinib, Vandetanib, Bosutinib, Dasatinib, Ponatinib, Vandetanib, Axitinib, Lenvatinib, Nintedanib, Regorafenib, Pazopanib, Sorafenib, and Sunitinib, more preferably selected from Alisertib, MK1775, MK2206, Saracatinib, and Temsirolimus. Preferably, the DNA intercalating agent is selected from Doxorubicin and Nemorubicin.

An agent sensitizing the cells to radiotherapy may also be AZD7648:

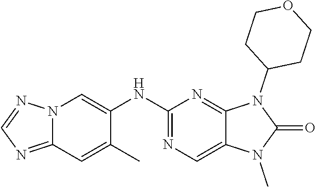 [SMILES: Cc1cc2ncnn2cc1Nc1ncc2c(n1)n(C1CCOCC1)c(=O)n2C]

As it will be clear to the skilled person in view of the disclosure hereinabove, the present invention further relates to the antibody or antigen binding fragment thereof of the present invention, the immunoconjugate of the present invention or the pharmaceutical composition of the present invention for use in the manufacture of a medicament for treating an L1-CAM associated cancer.

As it will further be clear to the skilled person in view of the disclosure hereinabove, the present invention further relates to the method of treatment of an L1-CAM associated cancer, the method comprising administering to an individual in need thereof of the antibody or antigen binding fragment thereof of the present invention, the immunoconjugate of the present invention or the pharmaceutical composition of the present invention. It is to be understood that the antibody or antigen binding fragment thereof of the invention, or the immunoconjugate of the invention, or the pharmaceutical composition of the invention, are to be administered in a therapeutically effective amount.

An “individual” or “subject” is a mammal. Mammals include, but are not limited to, domesticated animals (e.g., cows, sheep, cats, dogs, and horses), primates (e.g., humans and non human primates such as macaques), rabbits, and rodents (e.g., mice and rats). In certain embodiments, the individual or subject is a human.

An “effective amount” of an agent, e.g., a pharmaceutical formulation, refers to an amount effective, at dosages and for periods of time necessary, to achieve the desired therapeutic or prophylactic result.

The present invention further relates to the antibody or the antigen-binding fragment thereof of the present invention or the immunoconjugate of the present invention for use in diagnosis. The antibodies, antigen-binding fragments thereof and immunoconjugates of the present invention are particularly useful in diagnosis of L1-CAM (CD171) associated cancer, as described herein.

Preferably, the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer, and head and neck squamous cell carcinoma.

As recognized to the skilled person, the antibodies or the antigen-binding fragments thereof may be used in diagnostic methods, in particular in-vitro diagnostic methods, wherein said antibody or said fragment is used for detection of L1-CAM positive cells in tissue, wherein said antibody or said fragment thereof is then detected by a secondary antibody, preferably a monoclonal antibody.

As encompassed by the present invention, immunoconjugates useful in diagnosis comprise an active agent, wherein said active agent is a moiety that enables detection and localization of said immunoconjugate. Accordingly, said active agent, while being not particularly limited, as long as it is suitable for a diagnostic purpose, is preferably selected from radionuclide, MRI active compound, ultrasound contrast agent, fluorophore (preferably fluorophore in the far red/near-IR spectral region), marker for PET and SPECT, and Gd-based and Fe particle-based MRI contrast agent. The suitable imaging methods allowing the diagnostic applications are known to the skilled person. Preferably, said active agent is a radionuclide. Preferably, said radionuclide is a radionuclide useful in diagnosis, preferably selected from fluorine-18, scandium-43, scandium-44, copper-61, copper-64, gallium-68, zirconium-89, indium-111, iodine-123, terbium-152, and terbium-155.

Particularly useful in diagnosis are antibody radionuclide conjugate of the present invention, wherein the radionuclide is a radionuclide useful in diagnosis, preferably selected from fluorine-18, scandium-43, scandium-44, copper-61, copper-64, gallium-68, zirconium-89, indium-111, iodine-123, terbium-152, and terbium-155, as described hereinabove.

Further examples and/or embodiments of the present invention are disclosed in the following numbered items.
1. An antibody or an antigen binding fragment thereof that specifically binds to L1-CAM (CD171), the antibody or the antigen binding fragment thereof comprising:
   a variable heavy chain region comprising:
      CDR-H1 characterized by a sequence selected from
      a sequence according to SEQ ID NO.:1 (GYWMH),
      a sequence according to SEQ ID NO.: 2 (GYYMH),
      a sequence according to SEQ ID NO.: 3 (GYFMH), and
      a sequence according to SEQ ID NO.: 4 (GYLMH); and
      CDR-H2 characterized by a sequence selected from
      a sequence according to SEQ ID NO.: 5 (EINPSNGRTNYNERFQG),
      a sequence according to SEQ ID NO.: 6 (EINPSNGRTNYNEKFQG),
      a sequence according to SEQ ID NO.: 7 (EINPSNGRTNYNERFKS),
      a sequence according to SEQ ID NO.: 8 (EINPSNGRTNYNERLKS),
      a sequence according to SEQ ID NO.: 9 (EINPSNARTNYNERFQG),
      a sequence according to SEQ ID NO.: 10 (EINPSNARTNYNEKFQG)
      a sequence according to SEQ ID NO.: 11 (EINPSNARTNYNERFKS) and
      a sequence according to SEQ ID NO.: 12 (EINPSNARTNYNERLKS); and
      CDR-H3 characterized by a sequence according to SEQ ID NO.: 13 (DYYGTSYNFDY); and/or
      a variable light chain region comprising:
      CDR-L1 characterized by a sequence selected from
      a sequence according to SEQ ID NO.: 14 (RANEDINNRLA),
      a sequence according to SEQ ID NO.: 15 (KANEDINNRLA),
      a sequence according to SEQ ID NO.: 16 (QANEDINNRLA),
      a sequence according to SEQ ID NO.: 17 (RANEDINARLA),
      a sequence according to SEQ ID NO.: 18 (KANEDINARLA),
      a sequence according to SEQ ID NO.: 19 (QANEDINARLA),
      a sequence according to SEQ ID NO.: 20 (RANEDINLRLA),
      a sequence according to SEQ ID NO.: 21 (KANEDINLRLA), and
      a sequence according to SEQ ID NO.: 22 (QANEDINLRLA); and
      CDR-L2 characterized by a sequence selected from
      a sequence according to SEQ ID NO.: 23 (GATNLVT) and
      a sequence according to SEQ ID NO.: 24 (GASNLVS); and
      CDR-L3 characterized by a sequence selected from
      a sequence according to SEQ ID NO.: 25 (QQYWSTPFT),
      a sequence according to SEQ ID NO.: 26 (QQYYSTPFT) and
      a sequence according to SEQ ID NO.: 27 (QQYFSTPFT).
2. The antibody or the antigen-binding fragment thereof of item 1, wherein the antibody or the antigen binding fragment thereof is a monoclonal antibody, a chimeric antibody, a recombinant antibody, an antigen-binding fragment of a recombinant antibody, a single chain antibody, a humanized antibody, a bispecific antibody, a multi-specific antibody, or an antibody displayed upon the surface of a phage or displayed upon the surface of a chimeric antigen receptor (CAR) T cell, preferably wherein the antibody or the antigen binding fragment thereof is a monoclonal antibody, preferably an IgG1 antibody.
3. The antibody or the antigen-binding fragment thereof of item 1 or 2, wherein the variable heavy chain region comprises CDR-H1 characterized by a sequence according to SEQ ID NO.: 1.
4. The antibody or the antigen-binding fragment thereof of any one of items 1 to 3, wherein the variable heavy chain region comprises CDR-H2 characterized by a sequence according to SEQ ID NO.: 5, 6, 9 or 10, preferably characterized by a sequence according to SEQ ID NO.: 5 or 6.
5. The antibody or the antigen-binding fragment thereof of any one of items 1 to 4, wherein the variable light chain region comprises CDR-L1 characterized by a sequence according to SEQ ID NO.: 14, 15, 17, 18, or 21, preferably characterized by a sequence according to SEQ ID NO.: 14 or 15.
6. The antibody or the antigen-binding fragment thereof of any one of items 1 to 5, wherein the variable light chain comprises CDR-L2 characterized by a sequence according to SEQ ID NO.: 23.
7. The antibody or the antigen-binding fragment thereof of any one of items 1 to 6, wherein the variable light chain comprises CDR-L3 characterized by a sequence according to SEQ ID NO.: 25.
8. The antibody or the antigen-binding fragment thereof of item 1 or 2, wherein the variable heavy chain region is characterized by a sequence at least 90% identical to, preferably at least 95% identical to, more preferably identical to a sequence selected from
   a sequence according to SEQ ID NO.: 28 (QVQLVQSGAEVKKPGASVKVSCKASGYTFTGYWMHWVRQAPGQGLEWIGE INPSNGRTNYNERFQGRVTLTVDKSISTAYMELSRLRSDDTAVYYCARDYYGT SYNFDYWGQGTLVTVSS),
   a sequence according to SEQ ID NO.: 29 (QVQLVQSGAEVKKPGASVKVSCKASGYTFTGYWMHWVRQAPGQGLEWIGE INPSNGRTNYNERFKSRVTLTVDKSISTAYMELSRLRSDDTAVYFCARDYYGT SYNFDYWGQGTLVTVSS),
   a sequence according to SEQ ID NO.: 30 (QVQLQQWGAGLLKPSETLSLTCAAYGYTFTGYWMHWIRQPPGKGLEWIGEI NPSNGRTNYNERLKSRVTLSVDKSKNQASLKLSSVTAADTAVYFCARDYYGT SYNFDYWGQGTLVTVSS),
   a sequence according to SEQ ID NO.: 31 (QVQLVQSGAEVKKPGASVKVSCKASGYTFTGYWMHWVRQAPGQGLEWIGE INPSNGRTNYNEkFQGRVTLTVDKSISTAYMELSRLRSDDTAVYYCARDYYGT SYNFDYWGQGTLVTVSS), and
   a sequence according to SEQ ID NO.: 32 (QVQLVQSGAEVKKPGASVKVSCKASGYTFTGYWMHWVRQAPGQGLEWmG EINPSNGRTNYNEkFQGRVTLTVDKSISTAYMELSRLRSDDTAVYYCARDYYG TSYNFDYWGQGTLVTVSS), preferably characterized by a sequence according to SEQ ID NO.: 28 or a sequence according to SEQ ID NO.: 32.
9. The antibody or the antigen-binding fragment thereof of item 1 or 2, wherein the variable light chain region is characterized by a sequence at least 90% identical to, preferably at least 95% identical to, more preferably identical to a sequence selected from
   a sequence according to SEQ ID NO.: 33 (DIQMTQSPSSLSASVGDRVTITCKANEDINNRLAWYQQKPGKAPKLLISGATN LVTGVPSRFSGSGSGKDYTLTISSLQPEDFATYYCQQYWSTPFTFGQGTKLEI K),
   a sequence according to SEQ ID NO.: 34 (DIQMTQSPSSLSASVGDRVTITCKANEDINNRLAWYQQKPGKAPKLLISGATN LVTGVPSRFSGSGSGKDYTLTISSLQPEDIATYYCQQYWSTPFTFGQGTKLEI K),
   a sequence according to SEQ ID NO.: 35 (EIVMTQSPATLSVSPGERATLSCRANEDINNRLAWYQQKPGQAPRLLISGAT NLVTGIPARFSGSGSGKEFTLTISSLQSEDFAVYYCQQYWSTPFTFGQGTKLEI K),
   a sequence according to SEQ ID NO.: 36 (DIQMTQSPSSLSASVGDRVTITCRANEDINNRLAWYQQKPGKAPKLLISGATN LVTGVPSRFSGSGSGKDYTLTISSLQPEDFATYYCQQYWSTPFTFGQGTKLEI K), and
   a sequence according to SEQ ID NO.: 37 (DIQMTQSPSSLSASVGDRVTITCRANEDINNRLAWYQQKPGKAPKLLISGAsN LVsGVPSRFSGSGSGKDYTLTISSLQPEDFATYYCQQYWSTPFTFGQGTKLEI K), preferably characterized by a sequence selected from a sequence according to SEQ ID NO.: 33, a sequence according to SEQ ID NO.: 36 and a sequence according to SEQ ID NO. 37.
10. The antibody or the antigen-binding fragment thereof of any one of items 1 to 9,
   wherein the variable heavy chain region is characterized by a sequence according to SEQ ID NO.: 28 and the variable light chain region is characterized by a sequence according to SEQ ID NO.: 33; or
   wherein the variable heavy chain region is characterized by a sequence according to SEQ ID NO.: 32 and the variable light chain region is characterized by a sequence according to SEQ ID NO.: 36; or
   wherein the variable heavy chain region is characterized by a sequence according to SEQ ID NO.: 14 and the variable light chain region is characterized by a sequence according to SEQ ID NO.: 37.
11. The antibody or the antigen-binding fragment thereof of item 1 or 2,
   wherein the variable heavy chain region is characterized by a sequence at least 90% identical to, preferably at least 95% identical to, more preferably identical to a sequence:

FH0 - CDR-H1 - FH1 - CDR-H2 - FH2 - CDR-H3 - FH3

wherein CDR-H1, CDR-H2 and CDR-H3 are as defined in claim 1, wherein: FH0 is characterized by a sequence according to SEQ ID NO: 38 (QVQLVQSGAEVKKPGASVKVSCKASGYTFT) or a sequence according to SEQ ID NO: 39 (QVQLQQWGAGLLKPSETLSLTCAAYGYTFT),
FH1 is characterized by a sequence according to SEQ ID NO: 40 (WVRQAPGQGLEWIG) or a sequence according to SEQ ID NO.: 41 (WIRQPPGKGLEWIG)
FH2 is characterized by a sequence according to SEQ ID NO: 42 (RVTLTVDKSISTAYMELSRLRSDDTAVYFCAR) or a sequence according to SEQ ID NO.: 43 (RVTLSVDKSKNQASLKLSSVTAADTAVYFCAR), and
FH3 is characterized by a sequence according to SEQ ID NO:44 (WGQGTLVTVSS),
and/or
wherein the variable light chain region is characterized by a sequence at least 90% identical to, preferably at least 95% identical to, more preferably identical to a sequence:

FL0 - CDR-L1 - FL1 - CDR-L2 - FL2 - CDR-L3 - FL3

wherein CDR-L1, CDR-L2 and CDR-L3 are as defined in claim 1, wherein: FL0 is characterized by a sequence according to SEQ ID NO: 45 (DIQMTQSPSSLSASVGDRVTITC) or a sequence according to SEQ ID NO: 46 (EIVMTQSPATLSVSPGERATLSC),
FL1 is characterized by a sequence according to SEQ ID NO: 47 (WYQQKPGKAPKLLIS) or a sequence according to SEQ ID NO.: 48 (WYQQKPGQAPRLLIS),
FL2 is characterized by a sequence according to SEQ ID NO: 49 (GVPSRFSGSGSGKDYTLTISSLQPEDIATYYC) or a sequence according to SEQ ID NO.: 50 (GIPARFSGSGSGKEFTLTISSLQSEDFAVYYC), and
FL3 is characterized by a sequence according to SEQ ID NO:51 (FGQGTKLEIK).
12. The antibody or the antigen-binding fragment thereof of any one of items 1 to 11, wherein the heavy chain further comprises at least one point mutation in Fc part that influences antibody-dependent cell-mediated cytotoxicity (ADCC), complement-dependent cytotoxicity (CDC), serum half-life and/or glycosylation status of the antibody.
13. The antibody or the antigen-binding fragment thereof of item 11, wherein the at least one point mutation is selected from L234A, L234F, L235A, L235E, L235Q, G236A, M252Y, S254T, T256E, S267E, H268F, N297A, K322A, K322Q, S324T, P331 S, and I332E, preferably wherein the at least one point mutation is selected from L234A, L235A, P331S and N297A.
14. The antibody or the antigen-binding fragment thereof of any one of items 1 to 13, characterized by a dissociation constant K_{D} to L1-CAM (CD171) not exceeding 10⁻¹² M, as measured in a Biacore-based assay.
15. A polynucleotide encoding at least one variable heavy chain sequence and/or at least one variable light chain sequence as described in any one of items 1 to 11.
16. A host cell comprising the polynucleotide of item 15.
17. An immunoconjugate comprising an antibody or an antigen-fragment binding thereof of any one of items 1 to 14 and an active agent, preferably wherein the active agent is a cytotoxic agent or a prodrug thereof.
18. The immunoconjugate of item 17, wherein the antibody or the antigen-binding fragment thereof is linked to the active agent through a linker moiety, preferably wherein said linker moiety comprises a polymer carrier, to which at least one active agent is attached.
19. The immunoconjugate of item 17 or 18, wherein the active agent is a radionuclide, preferably a radionuclide useful for therapeutic application, preferably selected from copper-67, strontium-89, yttrium-90, iodine-131, samarium-153, terbium-161, lutetium-177, astatine-211, radium-223 and actinium 225, and/or a radionuclide useful in diagnosis, preferably selected from fluorine-18, scandium-43, scandium-44, copper-61, copper-64, gallium-68, zirconium-89, indium-111, iodine-123, terbium-152, and terbium-155.
20. A pharmaceutical composition comprising the antibody or the antigen-binding fragment thereof of any one of items 1 to 14 or the immunoconjugate of any one of items 17 to 19 and a pharmaceutically acceptable carrier.
21. The antibody or the antigen-binding fragment thereof of any one of items 1 to 14 or the immunoconjugate of any one of items 17 to 19, for use as a medicament.
22. The antibody or the antigen-binding fragment thereof of any one of items 1 to 14 or the immunoconjugate of any one of items 17 to 19 for use in the treatment of an L1-CAM (CD171) associated cancer.
23. The antibody or the antigen-binding fragment thereof for use or the immunoconjugate for use of item 22, wherein the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer and head and neck squamous cell carcinoma.
24. The antibody or the antigen-binding fragment thereof for use or the immunoconjugate for use of item 22 or 23, wherein said antibody or the antigen-binding fragment thereof or the immunoconjugate is to be administered to a subject with an additional therapeutic agent, selected from alkylating agents, platinum agents, taxanes, vinca agents, anti-estrogen drugs, aromatase inhibitors, ovarian suppression agents, VEGFNEGFR inhibitors, EGF/EGFR inhibitors, PARP inhibitors, cytostatic alkaloids, cytotoxic antibiotics, antimetabolites, endocrine/hormonal agents, immune checkpoint inhibitors and bisphosphonate therapy agent.
25. The antibody or the antigen-binding fragment thereof of any one of items 1 to 14 or the immunoconjugate of any one of items 17 to 19 for use in diagnosis.
26. The antibody or the antigen-binding fragment thereof of any one of items 1 to 14 or the immunoconjugate of any one of items 17 to 19 for use in diagnosis of L1-CAM (CD171) associated cancer.
27. The antibody or the antigen-binding fragment thereof for use or the immunoconjugate for use of item 26, wherein the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer, and head and neck squamous cell carcinoma.

Further examples or embodiments of the invention are disclosed in the following numbered clauses.
1. An antibody or an antigen binding fragment thereof that specifically binds to L1-CAM (CD171), the antibody or the antigen binding fragment thereof comprising:
   a variable heavy chain region comprising:
      CDR-H1 characterized by a sequence selected from
      a sequence according to SEQ ID NO.:1 (GYWMH),
      a sequence according to SEQ ID NO.: 2 (GYYMH),
      a sequence according to SEQ ID NO.: 3 (GYFMH), and
      a sequence according to SEQ ID NO.: 4 (GYLMH); and
      CDR-H2 characterized by a sequence selected from
      a sequence according to SEQ ID NO.: 5 (EINPSNGRTNYNERFQG),
      a sequence according to SEQ ID NO.: 6 (EINPSNGRTNYNEKFQG),
      a sequence according to SEQ ID NO.: 7 (EINPSNGRTNYNERFKS),
      a sequence according to SEQ ID NO.: 8 (EINPSNGRTNYNERLKS),
      a sequence according to SEQ ID NO.: 9 (EINPSNARTNYNERFQG),
      a sequence according to SEQ ID NO.: 10 (EINPSNARTNYNEKFQG)
      a sequence according to SEQ ID NO.: 11 (EINPSNARTNYNERFKS) and
      a sequence according to SEQ ID NO.: 12 (EINPSNARTNYNERLKS); and
      CDR-H3 characterized by a sequence according to SEQ ID NO.: 13 (DYYGTSYNFDY); and/or
   a variable light chain region comprising:
      CDR-L1 characterized by a sequence selected from
      a sequence according to SEQ ID NO.: 14 (RANEDINNRLA),
      a sequence according to SEQ ID NO.: 15 (KANEDINNRLA),
      a sequence according to SEQ ID NO.: 16 (QANEDINNRLA),
      a sequence according to SEQ ID NO.: 17 (RANEDINARLA),
      a sequence according to SEQ ID NO.: 18 (KANEDINARLA),
      a sequence according to SEQ ID NO.: 19 (QANEDINARLA),
      a sequence according to SEQ ID NO.: 20 (RANEDINLRLA),
      a sequence according to SEQ ID NO.: 21 (KANEDINLRLA), and
      a sequence according to SEQ ID NO.: 22 (QANEDINLRLA); and
      CDR-L2 characterized by a sequence selected from
      a sequence according to SEQ ID NO.: 23 (GATNLVT) and
      a sequence according to SEQ ID NO.: 24 (GASNLVS); and
      CDR-L3 characterized by a sequence selected from
      a sequence according to SEQ ID NO.: 25 (QQYWSTPFT),
      a sequence according to SEQ ID NO.: 26 (QQYYSTPFT) and
      a sequence according to SEQ ID NO.: 27 (QQYFSTPFT),
   preferably wherein the antibody or the antigen binding fragment thereof is a monoclonal antibody, a chimeric antibody, a recombinant antibody, an antigen-binding fragment of a recombinant antibody, a single chain antibody, a humanized antibody, a bispecific antibody, a multi-specific antibody, or an antibody displayed upon the surface of a phage or displayed upon the surface of a chimeric antigen receptor (CAR) T cell, more preferably wherein the antibody or the antigen binding fragment thereof is a monoclonal antibody, preferably an IgG1 antibody.
2. The antibody or the antigen-binding fragment thereof of clause 1, wherein the variable heavy chain region comprises CDR-H1 characterized by a sequence according to SEQ ID NO.: 1, and/or
   wherein the variable heavy chain region comprises CDR-H2 characterized by a sequence according to SEQ ID NO.: 5, 6, 9 or 10, preferably characterized by a sequence according to SEQ ID NO.: 5 or 6, and/or
   wherein the variable light chain region comprises CDR-L1 characterized by a sequence according to SEQ ID NO.: 14, 15, 17, 18, or 21, preferably characterized by a sequence according to SEQ ID NO.: 14 or 15, and/or
   wherein the variable light chain comprises CDR-L2 characterized by a sequence according to SEQ ID NO.: 23, and/or
   wherein the variable light chain comprises CDR-L3 characterized by a sequence according to SEQ ID NO.: 25.
3. The antibody or the antigen-binding fragment thereof of clause 1 or 2, wherein the variable heavy chain region is characterized by a sequence at least 90% identical to, preferably at least 95% identical to, more preferably identical to a sequence selected from
   a sequence according to SEQ ID NO.: 28 (QVQLVQSGAEVKKPGASVKVSCKASGYTFTGYWMHWVRQAPGQGLEWIGE INPSNGRTNYNERFQGRVTLTVDKSISTAYMELSRLRSDDTAVYYCARDYYGT SYNFDYWGQGTLVTVSS),
   a sequence according to SEQ ID NO.: 29 (QVQLVQSGAEVKKPGASVKVSCKASGYTFTGYWMHWVRQAPGQGLEWIGE INPSNGRTNYNERFKSRVTLTVDKSISTAYMELSRLRSDDTAVYFCARDYYGT SYNFDYWGQGTLVTVSS),
   a sequence according to SEQ ID NO.: 30 (QVQLQQWGAGLLKPSETLSLTCAAYGYTFTGYWMHWIRQPPGKGLEWIGEI NPSNGRTNYNERLKSRVTLSVDKSKNQASLKLSSVTAADTAVYFCARDYYGT SYNFDYWGQGTLVTVSS),
   a sequence according to SEQ ID NO.: 31 (QVQLVQSGAEVKKPGASVKVSCKASGYTFTGYWMHWVRQAPGQGLEWIGE INPSNGRTNYNEkFQGRVTLTVDKSISTAYMELSRLRSDDTAVYYCARDYYGT SYNFDYWGQGTLVTVSS), and
   a sequence according to SEQ ID NO.: 32 (QVQLVQSGAEVKKPGASVKVSCKASGYTFTGYWMHWVRQAPGQGLEWmG EINPSNGRTNYNEkFQGRVTLTVDKSISTAYMELSRLRSDDTAVYYCARDYYG TSYNFDYWGQGTLVTVSS), preferably characterized by a sequence according to SEQ ID NO.: 28 or a sequence according to SEQ ID NO.: 32,
   and/or
   wherein the variable light chain region is characterized by a sequence at least 90% identical to, preferably at least 95% identical to, more preferably identical to a sequence selected from
   a sequence according to SEQ ID NO.: 33 (DIQMTQSPSSLSASVGDRVTITCKANEDINNRLAWYQQKPGKAPKLLISGATN LVTGVPSRFSGSGSGKDYTLTISSLQPEDFATYYCQQYWSTPFTFGQGTKLEI K),
   a sequence according to SEQ ID NO.: 34 (DIQMTQSPSSLSASVGDRVTITCKANEDINNRLAWYQQKPGKAPKLLISGATN LVTGVPSRFSGSGSGKDYTLTISSLQPEDIATYYCQQYWSTPFTFGQGTKLEI K),
   a sequence according to SEQ ID NO.: 35 (EIVMTQSPATLSVSPGERATLSCRANEDINNRLAWYQQKPGQAPRLLISGAT NLVTGIPARFSGSGSGKEFTLTISSLQSEDFAVYYCQQYWSTPFTFGQGTKLEI K),
   a sequence according to SEQ ID NO.: 36 (DIQMTQSPSSLSASVGDRVTITCRANEDINNRLAWYQQKPGKAPKLLISGATN LVTGVPSRFSGSGSGKDYTLTISSLQPEDFATYYCQQYWSTPFTFGQGTKLEI K), and
   a sequence according to SEQ ID NO.: 37 (DIQMTQSPSSLSASVGDRVTITCRANEDINNRLAWYQQKPGKAPKLLISGAsN LVsGVPSRFSGSGSGKDYTLTISSLQPEDFATYYCQQYWSTPFTFGQGTKLEI K), preferably characterized by a sequence selected from a sequence according to SEQ ID NO.: 33, a sequence according to SEQ ID NO.: 36 and a sequence according to SEQ ID NO. 37.
4. The antibody or the antigen-binding fragment thereof of any one of clauses 1 to 3,
   wherein the variable heavy chain region is characterized by a sequence according to SEQ ID NO.: 28 and the variable light chain region is characterized by a sequence according to SEQ ID NO.: 33; or
   wherein the variable heavy chain region is characterized by a sequence according to SEQ ID NO.: 32 and the variable light chain region is characterized by a sequence according to SEQ ID NO.: 36; or
   wherein the variable heavy chain region is characterized by a sequence according to SEQ ID NO.: 14 and the variable light chain region is characterized by a sequence according to SEQ ID NO.: 37.
5. The antibody or the antigen-binding fragment thereof of clause 1,
   wherein the variable heavy chain region is characterized by a sequence at least 90% identical to, preferably at least 95% identical to, more preferably identical to a sequence:

FH0 - CDR-H1 - FH1 - CDR-H2 - FH2 - CDR-H3 - FH3

wherein CDR-H1, CDR-H2 and CDR-H3 are as defined in clause 1, wherein: FH0 is characterized by a sequence according to SEQ ID NO: 38 (QVQLVQSGAEVKKPGASVKVSCKASGYTFT) or a sequence according to SEQ ID NO: 39 (QVQLQQWGAGLLKPSETLSLTCAAYGYTFT),
FH1 is characterized by a sequence according to SEQ ID NO: 40 (WVRQAPGQGLEWIG) or a sequence according to SEQ ID NO.: 41 (WIRQPPGKGLEWIG)
FH2 is characterized by a sequence according to SEQ ID NO: 42 (RVTLTVDKSISTAYMELSRLRSDDTAVYFCAR) or a sequence according to SEQ ID NO.: 43 (RVTLSVDKSKNQASLKLSSVTAADTAVYFCAR), and
FH3 is characterized by a sequence according to SEQ ID NO:44 (WGQGTLVTVSS),
and/or
wherein the variable light chain region is characterized by a sequence at least 90% identical to, preferably at least 95% identical to, more preferably identical to a sequence:

FL0 - CDR-L1 - FL1 - CDR-L2 - FL2 - CDR-L3 - FL3

wherein CDR-L1, CDR-L2 and CDR-L3 are as defined in claim 1, wherein:
FL0 is characterized by a sequence according to SEQ ID NO: 45 (DIQMTQSPSSLSASVGDRVTITC) or a sequence according to SEQ ID NO: 46 (EIVMTQSPATLSVSPGERATLSC),
FL1 is characterized by a sequence according to SEQ ID NO: 47 (WYQQKPGKAPKLLIS) or a sequence according to SEQ ID NO.: 48 (WYQQKPGQAPRLLIS),
FL2 is characterized by a sequence according to SEQ ID NO: 49 (GVPSRFSGSGSGKDYTLTISSLQPEDIATYYC) or a sequence according to SEQ ID NO.: 50 (GIPARFSGSGSGKEFTLTISSLQSEDFAVYYC), and
FL3 is characterized by a sequence according to SEQ ID NO:51 (FGQGTKLEIK).
6. The antibody or the antigen-binding fragment thereof of any one of clauses 1 to 5, wherein the heavy chain further comprises at least one point mutation in Fc part that influences antibody-dependent cell-mediated cytotoxicity (ADCC), complement-dependent cytotoxicity (CDC), serum half-life and/or glycosylation status of the antibody, preferably wherein the at least one point mutation is selected from L234A, L234F, L235A, L235E, L235Q, G236A, M252Y, S254T, T256E, S267E, H268F, N297A, K322A, K322Q, S324T, P331 S, and I332E, more preferably wherein the at least one point mutation is selected from L234A, L235A, P331 S and N297A.
7. The antibody or the antigen-binding fragment thereof of any one of clauses 1 to 6, characterized by a dissociation constant K_{D} to L1-CAM (CD171) not exceeding 10⁻¹² M, as measured in a Biacore-based assay.
8. A polynucleotide encoding at least one variable heavy chain sequence and/or at least one variable light chain sequence as described in any one of clauses 1 to 6.
9. A host cell comprising the polynucleotide of clause 8.
10. An immunoconjugate comprising an antibody or an antigen-fragment binding thereof of any one of clauses 1 to 7 and an active agent, preferably wherein the active agent is a cytotoxic agent or a prodrug thereof,
   preferably wherein the antibody or the antigen-binding fragment thereof is linked to the active agent through a linker moiety, preferably wherein said linker moiety comprises a polymer carrier, to which at least one active agent is attached,
   and/or
   preferably wherein the active agent is a radionuclide, preferably a radionuclide useful for therapeutic application, preferably selected from copper-67, strontium-89, yttrium-90, iodine-131, samarium-153, terbium-161, lutetium-177, astatine-211, radium-223 and actinium 225, and/or a radionuclide useful in diagnosis, preferably selected from fluorine-18, scandium-43, scandium-44, copper-61, copper-64, gallium-68, zirconium-89, indium-111, iodine-123, terbium-152, and terbium-155.
11. A pharmaceutical composition comprising the antibody or the antigen-binding fragment thereof of any one of clauses 1 to 7 or the immunoconjugate of clause 10 and a pharmaceutically acceptable carrier.
12. The antibody or the antigen-binding fragment thereof of any one of clauses 1 to 7 or the immunoconjugate of clause 10, for use as a medicament.
13. The antibody or the antigen-binding fragment thereof of any one of clauses 1 to 7 or the immunoconjugate of clause 10 for use in the treatment of an L1-CAM (CD171) associated cancer,
   preferably wherein the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer and head and neck squamous cell carcinoma,
   and/or
   preferably wherein said antibody or the antigen-binding fragment thereof or the immunoconjugate is to be administered to a subject with an additional therapeutic agent, selected from alkylating agents, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, immune checkpoint inhibitors and bisphosphonate therapy agent.
14. The antibody or the antigen-binding fragment thereof of any one of clauses 1 to 7 or the immunoconjugate of clause 10 for use in diagnosis.
15. The antibody or the antigen-binding fragment thereof of any one of clauses 1 to 7 or the immunoconjugate of clause 10 for use in diagnosis of L1-CAM (CD171) associated cancer, preferably wherein the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer, and head and neck squamous cell carcinoma.

The invention will be illustrated in the following Examples which, however, are not meant to be construed as limiting. It is noted that, unless explicitly indicated to the contrary, the present invention may relate to each and every antibody or antigen-binding fragment thereof (in particular when defined through an amino acid sequence), and/or each and every polynucleotide as disclosed in the Examples.

### EXAMPLES

### Example 1: Humanization of CE7 Mouse Monoclonal Antibody

The CE7 murine antibody was humanized by grafting the three CDRs, as defined by the Kabat nomenclature, from the light chain variable region (VL) into a human germline VL that was as-homologous-as-possible to the murine antibody VL. Similarly, the three CDRs from the heavy chain variable region (VH) were grafted into a human germline VH that was as-homologous-as-possible to the murine antibody VH. In addition, a few amino acid residues in the framework regions of the selected human germline variable regions were changed to the amino acid residues that were present in the murine variable regions (so called back-mutations). Based upon information on the structure of immunoglobulin variable regions, and with the guidance of an homology molecular model of the Fv of the CE7murine monoclonal antibody, these few residues in the framework regions were identified as having key roles in either maintaining the CDRs in the right conformation or in VH/VL packing, and thus they were retained in the humanized version A or substituted with their human germline counterparts, if possible, in the subsequent humanized versions. Under guidance of the homology molecular model, in subsequent version B, when judged possible the CDR residues, as defined by Kabat, were also substituted for their human germline counterparts (so called germlining) in order to increase the degree of humanness (i.e. percentage sequence identity for both VH and VL between the humanized versions and the closest human germline used as acceptor sequence for the CDR-grafting).

The added-value of combining a structural model with pure sequence analysis is the potential to discriminate between paratope-facing and non-paratopic residues in the CDR regions. The purpose of the structural model is to permit expanding the limits of the humanization process, taking it beyond mere CDR-grafting. Also, the structural models permit making more intelligent choices regarding back-mutations in light of the particular germlines involved. Note that the Kabat CDR definitions are not as strictly structural as those of other systems; thus, for some germlines the Kabat definitions are too broad. For both chains, heavy and light, we can usually be fairly confident that the assignment of residues from CDR1 and 2 as paratopic and non-paratopic, based upon the structural model, is correct. Similarly, the light chain CDR3 is usually well-described with high probability. The difficult case is invariably CDR3 of the heavy chain.

The humanized versions A provided herein for both VH and VL are conservative versions that explicitly minimize/avoid alteration of CDR residues, these versions are expected to give a similar binding/potency activity to the chimeric antibody (CE7 mouse VH and VL fused to human constant regions) used as the reference molecule. The subsequent humanized version B for both VH and VL are designed to reach a higher percentage of sequence identity (to reach as close as possible to 85%) with the closest human germline. This can be achieved by germlining (substituting the mouse residue with the corresponding human germline residue) framework and/or Kabat CDR amino acid residues.

For the design of CDR-grafted versions of the CE7 murine VH, two human germlines, IGHV1-2*06 and IGHV4-34*01 were selected. The two human germlines have a sequence identity across the whole V gene of 66.3% and 53.1%, respectively.

For the design of CDR-grafted versions of the CE7 murine VL, three human germlines; IGKV1-NL1*01, IGKV1-33*01 and IGKV3-15*01 were selected. The three human germlines have a sequence identity across the whole V-gene with the CE7 murine VL of 73.7%, 71.6% and 60.0%, respectively.

The first step of the humanization process should be to select the best heavy and light chain combination between the humanized VH and humanized VL versions. For the VH we have humanized versions based on two different human germlines, IGHV1-2*06 with 4 versions (A to D) and IGHV4-34*01 with 3 versions (A to C). This makes a total of 7 different humanized VH. For the VL we have humanized versions based on three human germlines with 4 versions (A to D) for each of them. This makes a total of 12 different humanized VL. To have all the possible combinations between the humanized VH and VL it would be necessary to produce and purify a total of 7 (VH)×12 (VL)=84 humanized variants.

This number can be cut down to 18 by only testing versions A and B for VH humanized versions based on human germline IGHV1-2*06 and version A only for humanized versions based on human germline IGHV4-34*01 in combination with version A and B of VL humanized versions based on human germline IGKV1-NL1*01, IGKV1-33*01 and IGKV3-15*01; we would have 3 VH×6 VL=18 combinations.

From the results obtained with these 18 humanized mAbs, the best one or two VH/VL combinations will be selected and subsequent versions B, C and D could be then tested.

TABLE E1

| | Humanized variant | Heavy chain | Light chain |
|---|---|---|---|
| 1 | CE7-12-VHA-1NL1-VLA | CE7-1-2-VHA | CE7-1-NL1-VLA |
| 2 | CE7-12-VHA-1NL1-VLB | | CE7-1-NL1-VLB |
| 3 | CE7-12-VHA-133-VLA | | CE7-1-33-VLA |
| 4 | CE7-12-VHA-133-VLB | | CE7-1-33-VLB |
| 5 | CE7-12-VHA-315-VLA | | CE7-3-15-VLA |
| 6 | CE7-12-VHA-315-VLB | | CE7-3--15-VLB |
| 7 | CE7-12-VHB-1NL1-VLA | CE7-1-2-VHB | CE7-1-NL1-VLA |
| 8 | CE7-12-VHB-1NL1-VLB | | CE7-1-NL1-VLB |
| 9 | CE7-12-VHB-133-VLA | | CE7-1-33-VLA |
| 10 | CE7-12-VHB-133-VLB | | CE7-1-33-VLB |
| 11 | CE7-12-VHB-315-VLA | | CE7-3-15-VLA |
| 12 | CE7-12-VHB-315-VLB | | CE7-3--15-VLB |
| 13 | CE7-434-VHA-1NL1-VLA | CE7-4-34-VHA | CE7-1-NL1-VLA |
| 14 | CE7-434-VHA-1NL1-VLB | | CE7-1-NL1-VLB |
| 15 | CE7-434-VHA-133-VLA | | CE7-1-33-VLA |
| 16 | CE7-434-VHA-133-VLB | | CE7-1-33-VLB |
| 17 | CE7-434-VHA-315-VLA | | CE7-3-15-VLA |
| 18 | CE7-434-VHA-315-VLB | | CE7-3--15-VLB |

The selection of the best heavy and light chain combination between the 18 variants should be performed by assessing the following criteria:
The level of transient expression of the humanized versions produced in mammalian cells (HEK 293 or preferably CHO) as human IgG1/Kappa (as compared to the chimeric version). Using TO supernatant from transfected cells before harvest for purification using ELISA or Octet protein A measurement.
The binding capacity (EC50 by ELISA or FAGS; or preferably Kd by Biacore or Octet) as compared to the CE7chimeric human IgG1/Kappa version (chimeric meaning the combination of the parental murine VH and VL fused to human constant regions).
The biological activity of the humanized versions in a relevant in vitro cellular assay compared with that of the reference GE7 chimeric antibody.
The cross-reactivity with relevant orthologue species (in vitro binding activity) if relevant
A determination of the biophysical properties of the humanized versions as compared with GE7 chimeric:
   SEC-HPLC profile to determine the level of high molecular weight soluble aggregates,
   SDS-PAGE under non-reducing and reducing conditions,
   Analysis by differential scanning calorimetry (DSC) using Microcal VP-capillary DSC system to determine the Tm of Fab, CH2 and CH3.

Once the best 2 humanized VH and VL pairing have been identified, a second round of humanization could be initiated where the results of the sequence liability variants (see section “sequence liabilities”) will be taken into account. The positive CDR mutations identified amongst the sequence liability variants will be introduced in the CDRs of the best 2 humanized VH/VL combinations in order to obtain the final humanized candidates. The VH and VL humanized versions (C and D for IGHV1-2*01; B and C for IGHV4-34*01 and versions C and D for all the light chains could also be tested. The best final candidates will be selected following assessment using the same criteria as described above for the first-round candidates.

The humanization of murine CE7 monoclonal antibody was performed using standard CDR-grafting technology. The principle of this method is to reshape a human antibody containing only the complementarity determining regions (CDRs) from the murine monoclonal antibody with the aim of reducing immunogenicity when used as a therapeutic in humans. Humanization by CDR-grafting requires that the antigen-binding residues from the murine antibody be retained in the humanized antibody; thus, the identification of these residues obviously plays an important role in the protocol. To guide the humanization process and help in the decision to conserve parental murine residues or substitute them with their human germline counterparts, a homology molecular model of the Fv of the CE7 murine monoclonal antibody was built.

The CDR-grafting protocol used is a modernized version of the approach pioneered by Greg Winter and colleagues at the Medical Research Council, Cambridge, UK. The definition of the CDRs is based on the Kabat nomenclature. The selection of human framework acceptor regions into which CE7 murine CDR regions are grafted was accomplished by searching the IMGT murine and human V gene databases using IgBLAST, developed at NCBI to facilitate analysis of immunoglobulin V region sequences (http://www.ncbi.nlm.nih.gov/igblast/), with CE7 murine variable region sequences as input. The applied strategy is to use the human germline sequences that are natural human sequences not containing the idiosyncratic somatic mutations found in individual human antibody sequences.

### Sequence Liabilities

Over the past decade, drug developers have become increasingly proactive about early identification and risk assessment to combat attrition. One strategy commonly used to achieve drug-like properties with the aim to avoid late-stage failures is the so-called developability or molecular assessment approach which aims to optimize biophysical and chemical properties of molecules prior to product development. In addition to good biophysical properties (Jain et al., 2017 PNAS; 114: 944-949) mAbs also need to possess sufficient chemical stability to conform to stringent process development parameters. The predominant chemical modifications of interest for mAbs are oxidation (Met, M and Trp, W), deamidation (Asn, N) and isomerization (Asp, D). So identifying degradation sequence liabilities for re-engineering before committing resources to process and product development has the potential to reduce complications in formulation development and improve the potential for successful liquid formulations. Based on a recent study on deamidation and isomerization liability of 131 clinical-stage antibodies published in MABS (Lu et al., 2019 MABS, 11:45-57), we analyzed the CDRs of the CE7 VH and VL for deamidation and isomerization motifs which have been reported to be modified. The sequences of the CDRs as defined by the Kabat nomenclature are underscored. Within the CDRs, the potential sequence liabilities are in bold Only the potential sequence liabilities seen as real sequence liabilities will be discussed.

```
CE7 VH
[SEQ ID NO.: 55]
                                FR1 CDR1 FR2
QVQLQQPGAELVKPGASVKLSCKASGYTFTGYWMHWVKQRPGHGLEWIGE
  CDR2 FR3
INPSNGRTNYNERFKSKATLTVDKSSTTAFMQLSGLTSEDSAVYFCARDY
CDR3 FR4
YGTSYNFDYWGQGTTLTVSS
```

In CDR1 W at Kabat position Kabat H33. Inspection of the molecular model shows that it is buried, its sidechain flushes with level of paratopic surface, packed against VH-CDR3. Indole nitrogen may have access to antigen, sidechain contacts VH-CDR2 residues E(H55), I(H56) (mc), N(H57), R(H64), VH-CDR3 residue Y(H109); substitution W(H33)Y lowest energy rotamer induces excess strain (30.5) and alters contact structure; W(H33)F is similarly negative; W(H33)L induces moderate strain (17.6) but loses contacts at R(H64) (may be the route to amelioration of oxidation liability). So F, Y and L substitutions could be tried with the best chance for L to work. See below the sequences of the WH33F, WH33Y and WH33L variants that could be tested.

In CDR2 NG motif at Kabat positions H54 and H55. Inspection of the molecular model shows that N H54 is surface-exposed, at the edge of the paratopic surface, its sidechain may contact antigen. G H55 is surface-exposed, its sidechain situated on the non-antigen-facing surface of VH-CDR2, alpha-carbon makes no vernier or CDR contacts. Substitution G H55 to A looks like the best option for amelioration for the ‘NG’ liability. So A substitution at H55 should be tried. See below the sequences of the GH55A variant that were tested.

In CDR3 NF motif at Kabat positions H100B and H100C. Inspection of the molecular model shows that both residues are buried and therefore the risk of deamidation is considered as low.

```
CE7-VH-chimeric-WH33Y
[SEQ ID NO.: 56]
QVQLQQPGAELVKPGASVKLSCKASGYTFTGYYMHWVKQRPGHGLEWIGE
INPSNGRTNYNERFKSKATLTVDKSSTTAFMQLSGLTSEDSAVYFCARDY
YGTSYNFDYWGQGTTLTVSS
CE7-VH-chimeric-WH33F
[SEQ ID NO.: 57]
QVQLQQPGAELVKPGASVKLSCKASGYTFTGYFMHWVKQRPGHGLEWIGE
INPSNGRTNYNERFKSKATLTVDKSSTTAFMQLSGLTSEDSAVYFCARDY
YGTSYNFDYWGQGTTLTVSS
CE7-VH-chimeric-WH33L
[SEQ ID NO.: 58]
QVQLQQPGAELVKPGASVKLSCKASGYTFTGYLMHWVKQRPGHGLEWIGE
INPSNGRTNYNERFKSKATLTVDKSSTTAFMQLSGLTSEDSAVYFCARDY
YGTSYNFDYWGQGTTLTVSS
CE7-VH-chimeric-GH55A
[SEQ ID NO.: 59]
QVQLQQPGAELVKPGASVKLSCKASGYTFTGYWMHWVKQRPGHGLEWIGE
INPSNARTNYNERFKSKATLTVDKSSTTAFMQLSGLTSEDSAVYFCARDY
YGTSYNFDYWGQGTTLTVSS
CE7 VL:
[SEQ ID NO.: 60]
                FR1 CDR1 FR2
DIQMTQSSSSFSVSLGDRVTITCKANEDINNRLAWYQQTPGNSPRLLISG
  CDR2 FR3 CDR3
ATNLVTGVPSRFSGSGSGKDYTLTITSLQAEDFATYYCQQYWSTPFTFGS
FR4
GTELEIK
```

In CDR1 the motif NN at Kabat positions L30 and L31. Inspection of the molecular model shows that N L30 is surface-exposed, its sidechain is adjacent to VL CDR3 at the edge of paratopic surface and may contact antigen. N L31 is surface-exposed, its sidechain is at the edge of the paratopic surface, directed away from its center, sidechain contacts vernier residues G(L80), Y(L87). Substitutions of N L31 to L or A look the best option for the NN liability. See below the sequence of the NL31 L and NL31A variants that should be tested.

In CDR3 W at Kabat position L92. Inspection of the molecular model shows that W L92 is surface-exposed, sidechain near center of paratopic surface with high probability to contact antigen. Oxidation liability can be best addressed by substituting W with Y (Phenol ring overlaps with indol ring but the binding will it be maintained) or F. See below the sequences of the WL92Y and WL92F variants that were tested.

CE7-VL-chimeric-NL31L
[SEQ ID NO.: 61]
DIQMTQSSSSFSVSLGDRVTITCKANEDINLRLAWYQQTPGNSPRLLISG
ATNLVTGVPSRFSGSGSGKDYTLTITSLQAEDFATYYCQQYWSTPFTFGS
GTELEIK
CE7-VL-chimeric-NL31A
[SEQ ID NO.: 62]
DIQMTQSSSSFSVSLGDRVTITCKANEDINARLAWYQQTPGNSPRLLISG
ATNLVTGVPSRFSGSGSGKDYTLTITSLQAEDFATYYCQQYWSTPFTFGS
GTELEIK
CE7-VL-chimeric-WL92Y
[SEQ ID NO.: 63]
DIQMTQSSSSFSVSLGDRVTITCKANEDINNRLAWYQQTPGNSPRLLISG
ATNLVTGVPSRFSGSGSGKDYTLTITSLQAEDFATYYCQQYYSTPFTFGS
GTELEIK
CE7-VL-chimeric-WL92F
[SEQ ID NO.: 64]
DIQMTQSSSSFSVSLGDRVTITCKANEDINNRLAWYQQTPGNSPRLLISG
ATNLVTGVPSRFSGSGSGKDYTLTITSLQAEDFATYYCQQYFSTPFTFGS
GTELEIK

### Heavy Chain Design

Amino Acid Differences with Most Homologous Murine Germline, IGHV1S81*02

Amino acid sequence of the VH of the murine CE7 hybridoma

FR1 CDR1 FR2
QVQLQQPGAELVKPGASVKLSCKASGYTFTGYWMHWVKQRPGHGLEWIGE
CDR2 FR3
INPSNGRTNYNERFKSKATLTVDKSSTTAFMQLSGLTSEDSAVYFCARDY
CDR3 FR4
YGTSYNFDYWGQGTTLTVSS

[the sequence shown is a CE7 VH sequence according to SEQ ID NO.: 55]

91.8% (90 identical residues out of a total of 98 residues in the V gene) between the CE7 murine heavy-chain variable (VH) region and the murine germline immunoglobulin VH 1S81*02 (IGHV1 S81*02). Underscored in CE7 VH are the 8 residues that differ between the CE7 VH and the murine germline IGHV1S81*02.

| | |
|---|---|
| | <-------------FR1-----------><CDR><------FR2----> |
| CE7-VH | QVQLQQPGAELVKPGASVKLSCKASGYTFTGYWMHWVKQRPGHGLEWIG |
| IGHV1S81*02 | ..............................S...........Q...... |
| | <----------CDR2----><---------------FR3------------> |
| CE7-VH | EINPSNGRTNYNERFKSKATLTVDKSSTTAFMQLSGLTSEDSAVYFCAR |
| IGHV1S81*02 | .............K.............S..Y....SP........Y... |

Residues in bold are part of the classification of immunoglobulin VH domain frameworks as defined by Honegger and Pluckthun (J. Mol. Biol., 2001, 309: 687-699). The authors have reported that immunoglobulin VH frameworks could be grouped into four distinct types (I to IV), depending on the main-chain conformation of framework 1 (in particular that of Kabat residues 6, 7, 8 and 9). Mouse CE7 VH has Gln at Kabat position H6 and Pro at position H7 which is typical of Honneger type IV. Human germlines IGHV1-2*06 and IGHV4-34*01 which will be used as acceptor sequences for CDR-grafting are both of Honnerger type III (Gln at Kabat position H6 and not Pro at position H7). See sheets VH-IGHV1-2 columns N and O and VH-IGHV4-34 columns L and M.

### Selection of Human Framework Acceptor VH Regions

The selection of human framework acceptor VH regions into which the CE7 murine CDR regions are grafted was accomplished by searching the IMGT human VH gene database using IgBLAST (https://www.ncbi.nlm.nih.gov/igblast/igblast.cgi) with the murine CE7 VH amino acid sequence as input. Based on the sequence alignment of the Parental antibody to the human germlines, the closest matching entries were identified. The identification of the optimal human germline as acceptor was based on the following ordered criteria: sequence identity across the framework as defined by Kabat, and identity and/or compatibility of inter-chain interface residues and support loops with the canonical conformations of the Parental CDRs.

One of the important criteria in selecting a suitable human germline candidate is to be able to reach close to 85% sequence identity between this particular germline and the designed humanized version. We analyzed a large number of human germline sequences and selected two human germlines as best candidates; one belonging to the human family 1; IGHV1-2*06 and one belonging to the human family 4; IGHV4-34*01. Human germline IGHV1-2*06 has a high sequence identity of 66.3% (65 identical amino acid residues out of a total of 98) with mouse CE7 VH whereas human germline IGHV4-34*01 has a sequence identity of 53.1% (52 identical amino acid residues out of a total of 98).

In framework 4, the gene segment of the murine CE7 VH corresponding to the J gene was identified as most homologous to the murine germline J2 gene (see Table 1 VH, sheets VH-IGHV1-2 and VH-IGHV4-34 columns E, F and G). The murine J2-segment gene was compared to the human J-segment genes over CDR3 and FR4, and found to be most homologous to human J4*01-segment.

### Design Using IGHV1-2*06 Human Germlines as Framework Acceptor Regions

### Sequence Alignment Between Mouse CE7 VH and Human Germline IGHV1-2*06

66.3% identity (65 identical residues out of a total of 98 residues in the V gene) between the CE7 murine heavy-chain variable (VH) region and the human germlines immunoglobulin VH 1-2*06 (IGHV1-2*06).

CDR residues as defined by the Kabat nomenclature are highlighted in yellow. In the framework regions and in CDR, residues that differ between mouse CE7 VH and human 13 germlines IGHV1-2*06 are bolded and underscored, respectively.

| | |
|---|---|
| | <-------------FR1------------><CDR><-----FR2----> |
| CE7-VH | QVQLQQPGAELVKPGASVKLSCKASGYTFTGYWMHWVKQRPGHGLEWIG |
| IGHV1-2*06 | ....V.S...VK.......V............Y....R.A..Q....M. |
| | <-----CDR2-------><-------------FR3-------------> |
| CE7-VH | EINPSNGRTNYNERFKSKATLTVDKSSTTAFMQLSGLTSEDSAVYFCAR |
| IGHV1-2*06 | R...NS.G...AQK.QGRV.M.R.T.IS..Y.E..R.R.D.T...Y... |

### Humanized Version A

Murine CDRs as defined by the Kabat nomenclature were grafted into IGHV1-2*06 to obtain the hereunder detailed sequence. Residues highlighted in green are framework murine residues (non-CDR residues) conserved from the Parental murine CE7 VH sequence; they have been conserved because they might be structurally important for maintaining the full activity of the antibody.

Version A (VHA)
[SEQ ID NO.: 65]
FR1 CDR1 FR2
QVQLVQSGAEVKKPGASVKVSCKASGYTFTGYWMHWVRQAPGQGLEWIG
CDR2 FR3
EINPSNGRTNYNERFKSRVTLTVDKSISTAYMELSRLRSDDTAVYFCAR

84.7% identity (83 identical residues out of a total of 98 residues in the V gene) of humanized version A (CE7-1-2-VHA) with IGHV1-2*06 human germline. In the framework regions and in CDR, residues that differ between humanized version A and human germline IGHV1-2*06 are bolded and underscored, respectively

| | |
|---|---|
| | <-------------FR1------------><CDR><-----FR2----> |
| CE7-1-2-VHA | QVQLVQSGAEVKKPGASVKVSCKASGYTFTGYWMHWVRQAPGQGLEWIG |
| IGHV1-2*06 | ................................Y..............M. |
| | <-----CDR2-----><---------------FR3-------------> |
| CE7-1-2-VHA | EINPSNGRTNYNERFKSRVTLTVDKSISTAYMELSRLRSDDTAVYFCAR |
| IGHV1-2*06 | R...NS.G...AQK.QG...M.R.T....................Y... |

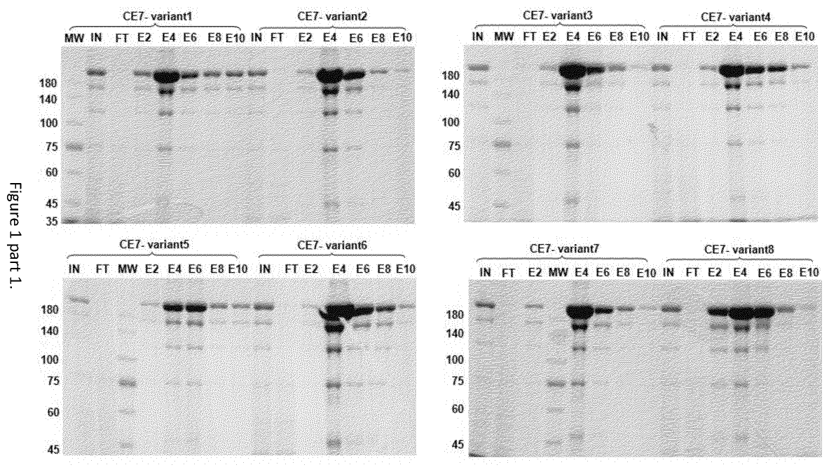

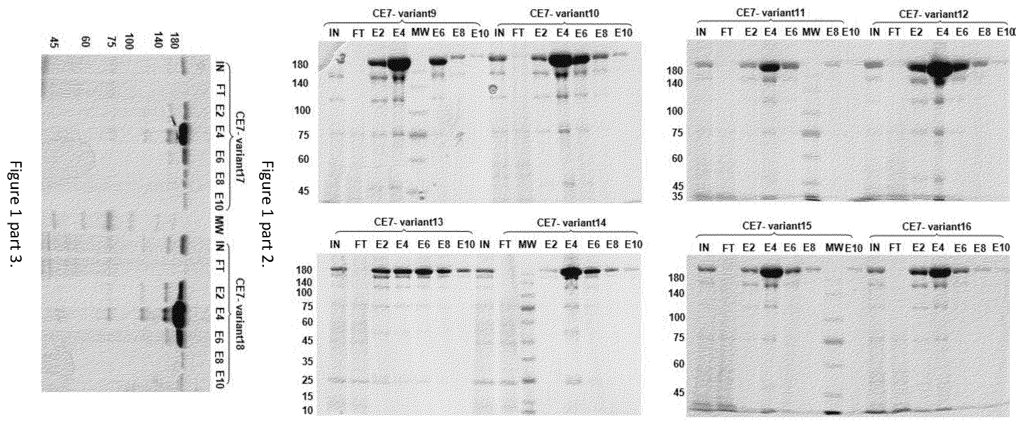

Refer to the full length amino acid sequence of version A in FIG. 1, to the comments for each relevant amino acid position upon inspection of the homology molecular model in Table 3 CE7 VH Design and to the sequence comparison in Table 1 VH (sheet VH-IGHV1-2 columns E, G and I, corresponding to murine CE7 VH, IGHV1-2*06 human germline and humanized version A, respectively) for detailed information.

### Humanized Version B

In version B, as compared to version A, 3 amino acid residues were germlined (i.e. the murine residue was substituted by the corresponding IGHV1-2*06 human germline residue). In CDR2 Kabat Lys (K) H64 and Kabat Ser (S) H65 were substituted with their corresponding human germline IGHV1-2*06 residues, Gln (Q) and Gly (G), respectively; and in FR3 Kabat Phe (F) H91 was substituted with the corresponding human germline IGHV1-2*06 residue, Tyr (Y).

The amino acid residues that differ between humanized version A and B are underscored.

Version B (VHB)
[SEQ ID NO.: 66]
FR1 CDR1 FR2
QVQLVQSGAEVKKPGASVKVSCKASGYTFTGYWMHWVRQAPGQGLEWIG
CDR2 FR3
EINPSNGRTNYNERFQGRVTLTVDKSISTAYMELSRLRSDDTAVYYCAR

87.8% identity (86 identical residues out of a total of 98 residues in the V gene) of humanized version B (CE7-1-2-VHB) with IGHV1-2*06 human germline. Residues that differ between humanized version A and B are underscored.

| | |
|---|---|
| | <------FR1------------------><CDR><------FR2----> |
| CE7-1-2-VHB | QVQLVQSGAEVKKPGASVKVSCKASGYTFTGYWMHWVRQAPGQGLEWIG |
| IGHV1-2*06 | ................................Y...............Y. |
| | <-----CDR2------><FR3----------FR3--------------> |
| CE7-1-2-VHB | EINPSNGRTNYNERFQGRVTLTVDKSISTAYMELSRLRSDDTAVYYCAR |
| IGHV1-2*06 | R...NS.G...AQK......M.R.T........................ |

### Humanized Version C

In version C as compared to version B, 1 amino acid residue was germlined (i.e. the murine residue was substituted by the corresponding IGHV1-2*06 human germline residue). In CDR2 Kabat Arg (R) H62) was substituted with its corresponding human germline IGHV1-2*06 residues, Lys (K).

The amino acid residue that differs between humanized version B and C is underscored.

```
Version C (VHC)
[SEQ ID NO.: 67]
              FR1 CDR1 FR2
QVQLVQSGAEVKKPGASVKVSCKASGYTFTGYWMHWVRQAPGQGLEWIG
    CDR2 FR3
EINPSNGRTNYNEKFQGRVTLTVDKSISTAYMELSRLRSDDTAVYYCAR
```

88.8% identity (87 identical residues out of a total of 98 residues in the V gene) of humanized version C (CE7-1-2-VHC) with IGHV1-2*06 human germline. The residue that differs between humanized version B and C is underscored.

| | |
|---|---|
| | <-------------FR1-----------><CDR><------FR2----> |
| CE7-1-2-VHC | QVQLVQSGAEVKKPGASVKVSCKASGYTFTGYWMHWVRQAPGQGLEWIG |
| IGHV1-2*06 | ................................Y..............M. |
| | <-----CDR2------><--------------FR3-------------> |
| CE7-1-2-VHC | EINPSNGRTNYNEKFQGRVTLTVDKSISTAYMELSRLRSDDTAVYYCAR |
| IGHV1-2*06 | R...NS.G...AQ..-----M.R.T........................ |

### Humanized Version D

In version D as compared to version C, 1 amino acid residue was germlined (i.e. the murine residue was substituted by the corresponding IGHV1-2*06 human germline residue). In FR2 Kabat Ile (I) H48 was substituted with its corresponding human germline IGHV1-2*06 residues Met (M).

The amino acid residue that differs between humanized version D and E is underscored.

```
Version D (VHD)
[SEQ ID NO.: 68]
              FR1 CDR1 FR2
QVQLVQSGAEVKKPGASVKVSCKASGYTFTGYWMHWVRQAPGQGLEWMG
      CDR2 FR3
EINPSNGRTNYNEKFQGRVTLTVDKSISTAYMELSRLRSDDTAVYYCAR
```

89.8% identity (88 identical residues out of a total of 98 residues in the V gene) of humanized version D (CE7-1-2-VHD) with IGHV1-2*06 human germline. The residue that differs between humanized version C and D is underscored.

| | |
|---|---|
| | <-------------FR1------------><CDR><-----FR2----> |
| CE7-1-2-VHD | QVQLVQSGAEVKKPGASVKVSCKASGYTFTGYWMHWVRQAPGQGLEWMG |
| IGHV1-2*06 | |
| | <-----CDR2------><--------------FR3-------------> |
| CE7-1-2-VHD | EINPSNGRTNYNEKFQGRVTLTVDKSISTAYMELSRLRSDDTAVYYCAR |
| IGHV1-2*06 | R...NS.G...AQ.......M.R.T........................ |

### Design Using IGHV4-34*01 Human Germline as Framework Acceptor Regions

### Sequence Alignment Between Mouse CE7 VH and Human Germline IGHV4-34*01

53.1% sequence identity (52 identical residues out of a total of 98 residues in the V gene) between the CE7 murine heavy-chain variable (VH) region and the human germline immunoglobulin VH 4-34*01 (IGHV4-34*01). Residues that differ between mouse CE7 VH and human germline IGHV4-34*01 in the frameworks and CDRs are bolded or underscored, respectively.

| | |
|---|---|
| | <-------------FR1------------><CDR><-----FR2----> |
| CE7-VH | QVQLQQPGAELVKPGASVKLSCKASGYTFTGYWMHWVKQRPGHGLEWIG |
| IGHV4-34*01 | ......W..G.L..SETLS.T.AVY.GS.S..YWS.IR.P..K...... |
| | <-----CDR2------><--------------FR3-------------> |
| CE7-VH | EINPSNGRTNYNERFKSKATLTVDKSSTTAFMQLSGLTSEDSAVYFCAR |
| IGHV4-34*01 | ...H.-.S....PSL..RV.IS..T.KNQFSLK..SV.AA.T...Y... |

### Humanized Version A

Murine CDRs (highlighted in yellow) as defined by the Kabat nomenclature were grafted into IGHV4-34*01 to obtain the hereunder detailed sequence. Framework murine residues (non-CDR residues) have been conserved from the Parental murine CE7 VH sequence; they have been conserved because they might be structurally important for maintaining the full activity of the antibody.

Version A
[SEQ ID NO.: 69]
FR1 CDR1 FR2
QVQLQQWGAGLLKPSETLSLTCAAYGYTFTGYWMHWIRQPPGKGLEWIG
CDR2 FR3
EINPSNGRTNYNERLKSRVTLSVDKSKNQASLKLSSVTAADTAVYFCAR

83.7% identity (82 identical residues out of a total of 98 residues in the V gene) of humanized version A (CE7-4-34-VHA) with IGHV4-34*01 human germline. Residues that differ between CE7-434-VHA and human germline IGHV4-34*01 in framework regions and in CDRs are bolded or underscored, respectively.

| | |
|---|---|
| | <-------------FR1------------><CDR><-----FR2----> |
| CE7-434-VHA | QVQLQQWGAGLLKPSETLSLTCAAYGYTFTGYWMHWIRQPPGKGLEWIG |
| IGHV4-34*01 | .......................V..GS.S..YWS.............. |
| | <-----CDR2------><--------------FR3-------------> |
| CE7-434-VHA | EINPSNGRTNYNERLKSRVTLSVDKSKNQASLKLSSVTAADTAVYFCAR |
| IGHV4-34*01 | ...H.-.S....PS......I...T....F...............Y... |
| .Y ... | |

### Humanized Version B

In version B, as compared to version A, three amino acid residues were germlined (i.e. the murine residue was substituted by the corresponding IGHV4-34*01 human germline residue); in framework 3 (FR3) Kabat Thr (T) H28 and Kabat Thr (T) H30 were both substituted with Ser (S) and in framework 3 (FR3) Kabat Phe (F) H91 was substituted with Tyr (Y).

```
Version B
[SEQ ID NO.: 70]
              FR1 CDR1 FR2
QVQLQQWGAGLLKPSETLSLTCAAYGYSFSGYWMHWIRQPPGKGLEWIG
    CDR2 FR3
EINPSNGRTNYNERLKSRVTLSVDKSKNQASLKLSSVTAADTAVYYCAR
```

86.7% identity (85 identical residues out of a total of 98 residues in the V gene) of humanized version B (CE7-434-VHB) with IGHV4-34*01 human germline. The amino acid residues that have been germlined in humanized version B as compared to humanized version A are underscored.

| | |
|---|---|
| | <-------------FR1------------><CDR><-----FR2----> |
| CE7-434-VHB | QVQLQQWGAGLLKPSETLSLTCAAYGYSFSGYWMHWIRQPPGKGLEWIG |
| IGHV4-34*01 | .......................V..G.....YWS.............. |
| | <-----CDR2------><--------------FR3-------------> |
| CE7-434-VHB | EINPSNGRTNYNERLKSRVTLSVDKSKNOASLKLSSVTAADTAVYYCAR |
| IGHV4-34*01 | ...H.-.S....PS......I...T....F................... |

### Humanized Version C

In version C, as compared to version B, two amino acid residues were germlined (i.e. the murine residues were substituted by the corresponding IGHV4-34*01 human germline residues); in CDR2 Kabat Glu (E) H61 and Kabat Arg (R) H62 were substituted with Pro (P) and Ser (S), respectively.

```
Version C
[SEQ ID NO.: 71]
              FR1 CDR1 FR2
QVQLQQWGAGLLKPSETLSLTCAAYGYSFSGYWMHWIRQPPGKGLEWIG
    CDR2 FR3
EINPSNGRTNYNPSLKSRVTLSVDKSKNQASLKLSSVTAADTAVYYCAR
```

88.8% identity (87 identical residues out of a total of 98 residues in the V gene) of humanized version C (CE7-434-VHC) with IGHV4-34*01 human germline. The amino acid residue that have been germlined in humanized version C as compared to humanized version B are underscored.

| | |
|---|---|
| | <-------------FR1------------><CDR><-----FR2----> |
| CE7-434-VHC | QVQLQQWGAGLLKPSETLSLTCAAYGYSFSGYWMHWIRQPPGKGLEWIG |
| IGHV4-34*01 | .......................V..G.....YWS.............. |
| | <-----CDR2------><--------------FR3-------------> |
| CE7-434-VHC | EINPSNGRTNYNPSLKSRVTLSVDKSKNQASLKLSSVTAADTAVYYCAR |
| IGHV4-34*01 | ...H.-.S............I...T....F................... |

### Light Chain Design

Amino Acid Differences with Most Homologous Murine Germline IGKV13-84*01

Amino acid sequence of the VL of the murine CE7 hybridoma (CDR regions as defined by the Kabat nomenclature are underscored)

```
[SEQ ID NO.: 72]
                FR1 CDR1 FR2
DIQMTQSSSSFSVSLGDRVTITCKANEDINNRLAWYQQTPGNSPRLLIS
  CDR2 FR3 CDR3
GATNLVTGVPSRFSGSGSGKDYTLTITSLQAEDFATYYCQQYWSTPFTF
    FR4
GSGTELEIK
```

90.5% identity (86 identical residues out of a total of 95 residues for the V gene) between the CE7 murine light-chain variable (VL) region and the murine germline immunoglobulin IGKV13-84*01. Underscored are the residues that differ between the CE7 VL and the murine germline IGKV13-84*01.

| | |
|---|---|
| | <---------FR1---------><--CDR---><------FR2-----> |
| CE7-VL | DIQMTQSSSSFSVSLGDRVTITCKANEDINNRLAWYQQTPGNSPRLLIS |
| IGKV13-84*01 | .........................S...Y........K...A...... |
| | <CDR2-><--------------FR3-------------><--CDR3 |
| CE7-VL | GATNLVTGVPSRFSGSGSGKDYTLTITSLQAEDFATYYCQQYWSTP |
| IGKV13-84*01 | ...S.E..................S.....T..V............ |

### Selection of Human Framework Acceptor VL Regions

The selection of human framework acceptor VL regions into which the CE7 murine CDR regions are grafted was accomplished by searching the IMGT human VL gene database using IgBLAST with the murine VL region amino acid sequence as input. Based on the sequence alignment of the Parental antibody to the human germlines, the closest matching entries were identified. The identification of the optimal human germline as acceptor was based on the following ordered criteria: sequence identity across the framework as defined by Kabat, and identity and/or compatibility of inter-chain interface residues and support loops with the canonical conformations of the Parental CDRs. One of the important criteria in selecting a suitable human germline candidate is to be able to reach close to 85% sequence identity between this particular germline and the designed humanized version. We analyzed a large number of human germlines and selected three human germlines; IGKV1-NL1*01, IGKV1-33*01 and IGKV3-15*01; for the design of CDR-grafted humanized versions.

The gene segment of the murine CE7 VL corresponding to the J gene was identified as the most homologous murine germline J4 gene (IGKJ4*01). The murine J4-gene segment was compared to the human J-segment genes over CDR3 and FR4, and the human J-segment IGKJ2 (IGKJ2*01) was found to have the highest overall homology.

### Design Using IGKV1-NL1*01 Human Germline as Framework Acceptor Regions

### Sequence Alignment Between Mouse CE7 VH and Human Germline IGKV1-NL1*01

73.7% identity (70 identical residues out of a total of 95 residues in the V gene) between the CE7 murine light chain variable (VL) region and the human germline immunoglobulin VL 1-NL1*01 (IGKV1-NL1*01). CDR residues as defined by the Kabat nomenclature are highlighted in yellow. Residues that differ between mouse CE7 VL and human germline IGKV1-NL1*01 in framework regions and CDRs are bolded and underscored, respectively.

| | |
|---|---|
| | <---------FR1---------><--CDR1---><-----FR2-----> |
| CE7-VL | DIQMTQSSSSFSVSLGDRVTITCKANEDINNRLAWYQQTPGNSPRLLIS |
| IGKV1-NL1*01 | .......P..L.A.V........R.SQG.S.S......K..KA.K..LY |
| | <CDR2-><--------------FR3-------------><--CDR3 |
| CE7-VL | GATNLVTGVPSRFSGSGSGKDYTLTITSLQAEDFATYYCQQYWSTP |
| IGKV1-NL1*01 | A.SR.ES............T......S...P...........Y... |

### Humanized Version A

Murine CDRs as defined by the Kabat numbering were grafted into IGKV1-NL1*01 to obtain the hereunder detailed sequence.

```
Version A
[SEQ ID NO.: 73]
                FR1 CDR1 FR2
DIQMTQSPSSLSASVGDRVTITCKANEDINNRLAWYQQKPGKAPKLLIS
  CDR2 FR3 CDR3
GATNLVTGVPSRFSGSGSGKDYTLTISSLQPEDFATYYCQQYWSTP
```

84.2% identity (80 identical residues out of a total of 95 residues in the V gene) of humanized version A (CE7-1-NL1-VLA) with IGKV1-NL1*01. Murine framework residues (non-CDR residues—bolded) were conserved from the parental murine CE7 VL sequence; they have been conserved because they might be structurally important for maintaining the full activity of the antibody. Residues that are underscored are positions that differ within CDRs between humanized version A and the human germline IGKV1-NL1*01.

| | |
|---|---|
| | <---------FR1---------><--CDR1---><-----FR2-----> |
| CE7-1-NL1-VLA | DIQMTQSPSSLSASVGDRVTITCKANEDINNRLAWYQQKPGKAPKLLIS |
| IGKV1-NL1*01 | .......................R.SQG.S.S...............LY |
| | <CDR2-><--------------FR3-------------><--CDR3 |
| CE7-1-NL1-VLA | GATNLVTGVPSRFSGSGSGKDYTLTISSLQPEDFATYYCQQYWSTP |
| IGKV1-NL1*01 | A.SR.ES............T......................Y... |

### Humanized Version B

In version B, compared to version A, one amino acid residue has been germlined (i.e. substituted by the corresponding human germline residue). In CDR1 Lys (K) Kabat L24 was germlined and mutated to Arg (R). Inspection of the molecular model shows that this residue is not likely to make direct contact with antigen and that it does not play a critical structural role; thus, it may be substituted for its corresponding human germline IGKV1-NL1*01 counterparts. The main reason for germlining this residue within the Kabat CDR L1 is that this change will increase the percentage identity of the humanized version B relative to the human germline IGKV1-NL1*01 to 85.3%.

Version B
[SEQ ID NO.: 74]
FR1 CDR1 FR2
DIQMTQSPSSLSASVGDRVTITCRANEDINNRLAWYQQKPGKAPKLLIS
CDR2 FR3 CDR3
GATNLVTGVPSRFSGSGSGKDYTLTISSLQPEDFATYYCQQYWSTP

85.3% identity (81 identical residues out of a total of 95 residues of the V gene) identity of humanized version B (CE7-1-NL1-VLB) with the IGKV1-NL1*01 human Kappa light chain germline. The amino acid residue that has been germlined in humanized version B as compared to humanized version A is underscored.

| | |
|---|---|
| | <---------FR1---------><--CDR1---><-----FR2-----> |
| CE7-1-NL1-VLB | DIQMTQSPSSLSASVGDRVTITCRANEDINNRLAWYQQKPGKAPKLLIS |
| IGKV1-NL1*01 | .........................SQG.S.S...............LY |
| | <CDR2-><--------------FR3-------------><--CDR3 |
| CE7-1-NL1-VLB | GATNLVTGVPSRFSGSGSGKDYTLTISSLQPEDFATYYCQQYWSTP |
| IGKV1-NL1*01 | A.SR.ES............T......................Y... |

### Humanized Version C

In version C, compared to version B, two amino acid residues have been germlined (i.e. substituted by the corresponding human germline residue). In CDR2 Thr (T) Kabat L52 and Thr (T) Kabat L56 were germlined and both mutated to Ser (S). Inspection of the molecular model show that these residues are not likely to make direct contact with antigen and that they do not play a critical structural role; thus, they may be substituted for their corresponding human germline IGKV1-NL1*01 counterparts. The main reason for germlining residues within the Kabat CDRs is that these changes will increase the percentage identity of the humanized version C relative to the human germline IGKV1-NL1*01 to 87.4%. 24

Version C
[SEQ ID NO.: 75]
FR1 CDR1 FR2
DIQMTQSPSSLSASVGDRVTITCRANEDINNRLAWYQQKPGKAPKLLIS
CDR2 FR3 CDR3
GASNLVSGVPSRFSGSGSGKDYTLTISSLQPEDFATYYCQQYWSTP

87.4% identity (83 identical residues out of a total of 95 residues of the V gene) identity of humanized version C (CE7-1-NL1-VLC) with the IGKV1-NL1*01 human Kappa light chain germline. The amino acid residues that have been germlined in humanized version C as compared to humanized version B are underscored.

| | |
|---|---|
| | <---------FR1---------><--CDR1---><-----FR2-----> |
| CE7-1-NL1-VLC | DIQMTQSPSSLSASVGDRVTITCRANEDINNRLAWYQQKPGKAPKLLIS |
| IGKV1-NL1*01 | .........................SQG.S.S...............LY |
| | <CDR2-><--------------FR3-------------><--CDR3 |
| CE7-1-NL1-VLC | GASNLVSGVPSRFSGSGSGKDYTLTISSLQPEDFATYYCQQYWSTP |
| IGKV1-NL1*01 | A..R.E.............T......................Y... |

### Humanized Version D

In version D, compared to version C, three amino acid residues have been germlined (i.e. substituted by the corresponding human germline residue). In CDR1 TAsn (N) Kabat L26, Glu (E) L27 and Asp (D) Kabat L28 were germlined and mutated to Ser (S), Gln (Q) and Gly (G), respectively. Inspection of the molecular model shows that these residues are not likely to make direct contact with antigen and that they do not play a critical structural role; thus, they may be substituted for their corresponding human germline IGKV1-NL1*01 counterparts. The main reason for germlining residues within the Kabat CDRs is that these changes will increase the percentage identity of the humanized version D relative to the human germline IGKV1-NL1*01 to 90.5%.

Version D
[SEQ ID NO.: 76]
FR1 CDR1 FR2
DIQMTQSPSSLSASVGDRVTITCRASQGINNRLAWYQQKPGKAPKLLIS
CDR2 FR3 CDR3
GASNLVSGVPSRFSGSGSGKDYTLTISSLQPEDFATYYCQQYWSTP

90.5% identity (86 identical residues out of a total of 95 residues of the V gene) identity of humanized version D (CE7-1-NL1-VLD) with the IGKV1-NL1*01 human Kappa light chain germline. The amino acid residues that have been germlined in humanized version D as compared to humanized version C are underscored.

| | |
|---|---|
| | <---------FR1---------><--CDR1---><-----FR2-----> |
| CE7-1-NL1-VLD | DIQMTQSPSSLSASVGDRVTITCRASQGINNRLAWYQQKPGKAPKLLIS |
| IGKV1-NL1*01 | .............................S.S...............LY |
| | <CDR2-><--------------FR3-------------><--CDR3 |
| CE7-1-NL1-VLD | GASNLVSGVPSRFSGSGSGKDYTLTISSLQPEDFATYYCQQYWSTP |
| IGKV1-NL1*01 | A..R.E.............T......................Y... |

### Design Using IGKV1-33*01 Human Germline as Framework Acceptor Regions

### Sequence Alignment Between Mouse CE7 VL and Human Germline IGKV1-33*01

71.6% identity (68 identical residues out of a total of 95 residues in the V gene) between the CE7 murine light chain variable (VL) region and the human germline immunoglobulin VL 1-33*01 (IGKV1-33*01). CDR residues as defined by the Kabat nomenclature are highlighted in yellow. Residues that differ between mouse CE7 VL and human germline IGKV1-33*01 in framework regions and CDRs are bolded and underscored, respectively.

| | |
|---|---|
| | <---------FR1---------><--CDR1---><-----FR2-----> |
| CE7-VL | DIQMTQSSSSFSVSLGDRVTITCKANEDINNRLAWYQQTPGNSPRLLIS |
| IGKV1-33*01 | .......P..L.A.V........Q.SQ..S.Y.N....K..KA.K...Y |
| | <CDR2-><--------------FR3-------------><--CDR3 |
| CE7-VL | GATNLVTGVPSRFSGSGSGKDYTLTITSLQAEDFATYYCQQYWSTP |
| IGKV1-33*01 | D.S..E.............T.F.F..S...P..I........DNL. |

### Humanized Version A

Murine CDRs as defined by the Kabat numbering were grafted into IGKV1-33*01 to obtain the hereunder detailed sequence.

Version A
[SEQ ID NO.: 77]
FR1 CDR1 FR2
DIQMTQSPSSLSASVGDRVTITCKANEDINNRLAWYQQKPGKAPKLLIS
CDR2 FR3 CDR3
GATNLVTGVPSRFSGSGSGKDYTLTISSLQPEDIATYYCQQYWSTP

83.2% identity (79 identical residues out of a total of 95 residues in the V gene) of humanized version A (CE7-1-33-VLA) with IGKV1-33*01. Residues that are bolded are murine framework residues (non-CDR residues) conserved from the parental murine CE7 VL sequence; they have been conserved because they might be structurally important for maintaining the full activity of the antibody. Residues that are underscored are positions that differ within CDRs between humanized version A and the human germline IGKV1-33*01.

| | |
|---|---|
| | <---------FR1---------><--CDR1---><-----FR2-----> |
| CE7-1-33-VLA | DIQMTQSPSSLSASVGDRVTITCKANEDINNRLAWYQQKPGKAPKLLIS |
| IGKV1-33*01 | .......................Q.SQ..S.Y.N..............Y |
| | <CDR2-><--------------FR3-------------><--CDR3 |
| CE7-1-33-VLA | GATNLVTGVPSRFSGSGSGKDYTLTISSLQPEDFATYYCQQYWSTP |
| IGKV1-33*01 | D.S..E.............T.F.F..................DNL. |

### Humanized Version B

In version B, compared to version A, two amino acid residues have been germlined (i.e substituted by the corresponding human germline residue). In CDR1 Lys (K) Kabat L24 was germlined and mutated to Gln (Q). In framework 3 (FR3) Leu (L) Kabat L73 was germlined and mutated to Phe (F). Inspection of the molecular model shows that these residues are not likely to make direct contact with antigen, and that they do not play a critical structural role; thus, they may be substituted for their corresponding human germline IGKV1-33*01 27 counterparts. The main reason for germlining residues within the Kabat CDRs is that these changes will increase the percentage identity of the humanized version B relative to the human germline IGKV1-33*01 to 85.3%.

Version B
[SEQ ID NO.: 78]
FR1 CDR1 FR2
DIQMTQSPSSLSASVGDRVTITCQANEDINNRLAWYQQKPGKAPKLLIS
CDR2 FR3 CDR3
GATNLVTGVPSRFSGSGSGKDYTFTISSLQPEDIATYYCQQYWSTP

85.3% identity (81 identical residues out of a total of 95 residues of the V gene) identity of humanized version B (CE7-1-33-VLB) with the IGKV1-33*01 human Kappa light chain germline. The amino acid residues that have been germlined in humanized version B as compared to humanized version A are underscored.

| | |
|---|---|
| | <---------FR1---------><--CDR1---><-----FR2-----> |
| CE7-1-33-VLB | DIQMTQSPSSLSASVGDRVTITCQANEDINNRLAWYQQKPGKAPKLLIS |
| IGKV1-33*01 | .........................SQ..S.Y.N..............Y |
| | <CDR2-><--------------FR3-------------><--CDR3 |
| CE7-1-33-VLB | GATNLVTGVPSRFSGSGSGKDYTFTISSLQPEDFATYYCQQYWSTP |
| IGKV1-33*01 | D.S..E.............T.F....................DNL. |

### Humanized Version C

In version C, compared to version B, two amino acid residues have been germlined (i.e substituted by the corresponding human germline residue). In CDR2 Thr (T) Kabat L52 was germlined and mutated to Ser (S). In framework 3 (FR3) Tyr (Y) Kabat L71 was germlined and mutated to Phe (F). Inspection of the molecular model shows that these residues are not likely to make direct contact with antigen, and that they do not play a critical structural role; thus, they may be substituted for their corresponding human germline IGKV1-33*01 counterparts. The main reason for germlining residues within the Kabat CDRs is that these changes will increase the percentage identity of the humanized version C relative to the human germline IGKV1-33*01 to 87.4%.

Version C
[SEQ ID NO.: 79]
FR1 CDR1 FR2
DIQMTQSPSSLSASVGDRVTITCQANEDINNRLAWYQQKPGKAPKLLIS
CDR2 FR3 CDR3
GASNLVTGVPSRFSGSGSGKDFTFTISSLQPEDIATYYCQQYWSTP

87.4% identity (83 identical residues out of a total of 95 residues of the V gene) identity of humanized version C (CE7-1-33-VLC) with the IGKV1-33*01 human Kappa light chain germline. The amino acid residues that have been germlined in humanized version C as compared to humanized version B are underscored.

| | |
|---|---|
| | <---------FR1---------><--CDR1---><-----FR2-----> |
| CE7-1-33-VLC | DIQMTQSPSSLSASVGDRVTITCQANEDINNRLAWYQQKPGKAPKLLIS |
| IGKV1-33*01 | .........................SQ..S.Y.N..............Y |
| | <CDR2-><--------------FR3-------------><--CDR3 |
| CE7-1-33-VLC | GASNLVTGVPSRFSGSGSGKDFTFTISSLQPEDFATYYCQQYWSTP |
| IGKV1-33*01 | D....E.............T......................DNL. |

### Humanized Version D

In version D, compared to version C, two amino acid residues have been germlined (i.e substituted by the corresponding human germline residue). In CDR1 Asn (N) Kabat L26 and Glu (E) Kabat L27 were germlined and mutated to Ser (S) and Gln (Q), respectively. Inspection of the molecular model shows that these residues are not likely to make direct contact with antigen, and that they do not play a critical structural role; thus, they may be substituted for their corresponding human germline IGKV1-33*01 counterparts. The main reason for germlining residues within the Kabat CDRs is that these changes will increase the percentage identity of the humanized version C relative to the human germline IGKV1-33*01 to 89.5%.

Version D
[SEQ ID NO.: 80]
FR1 CDR1 FR2
DIQMTQSPSSLSASVGDRVTITCQASQDINNRLAWYQQKPGKAPKLLIS
CDR2 FR3 CDR3
GASNLVTGVPSRFSGSGSGKDFTFTISSLQPEDIATYYCQQYWSTP

89.5% identity (85 identical residues out of a total of 95 residues of the V gene) identity of humanized version D (CE7-1-33-VLD) with the IGKV1-33*01 human Kappa light chain germline. The amino acid residues that have been germlined in humanized version D as compared to humanized version C are underscored.

| | |
|---|---|
| | <---------FR1---------><--CDR1---><-----FR2-----> |
| CE7-1-33-VLD | DIQMTQSPSSLSASVGDRVTITCQASQDINNRLAWYQQKPGKAPKLLIS |
| IGKV1-33*01 | .............................S.Y.N..............Y |
| | <CDR2-><--------------FR3-------------><--CDR3 |
| CE7-1-33-VLD | GASNLVTGVPSRFSGSGSGKDFTFTISSLQPEDFATYYCQQYWSTP |
| IGKV1-33*01 | D....E.............T......................DNL. |

### Design Using IGKV3-15*01 Human Germline as Framework Acceptor Regions

### Sequence Alignment Between Mouse CE7 VL and Human Germline IGKV3-15*01

60.0% identity (57 identical residues out of a total of 95 residues in the V gene) between the CE7 murine light chain variable (VL) region and the human germline immunoglobulin VL 3-15*01 (IGKV3-15*01). CDR residues as defined by the Kabat nomenclature are indicated. Residues that differ between mouse CE7 VL and human germline IGKV3-15*01 in framework regions and CDRs are bolded and underscored, respectively.

| | |
|---|---|
| | <---------FR1---------><--CDR1---><-----FR2-----> |
| CE7-VL | DIQMTQSSSSFSVSLGDRVTITCKANEDINNRLAWYQQTPGNSPRLLIS |
| IGKV3-15*01 | E.V....PATL...P.E.A.LS.R.SQSVSSN......K..QA.....Y |
| | <CDR2-><--------------FR3-------------><--CDR3 |
| CE7-VL | GATNLVTGVPSRFSGSGSGKDYTLTITSLQAEDFATYYCQQYWSTP |
| IGKV3-15*01 | ..STRA..I.A........TEF....S...S....V......NNW. |

### Humanized Version A

Murine CDRs as defined by the Kabat numbering were grafted into IGKV3-15*01 to obtain the hereunder detailed sequence.

Version A
[SEQ ID NO.: 81]
FR1 CDR1 FR2
EIVMTQSPATLSVSPGERATLSCKANEDINNRLAWYQQKPGQAPRLLIS
CDR2 FR3 CDR3
GATNLVTGIPARFSGSGSGKEYTLTISSLQSEDFAVYYCQQYWSTP

81.1% identity (77 identical residues out of a total of 95 residues in the V gene) of humanized version A (CE7-3-15-VLA) with IGKV3-15*01. Residues that are bolded are murine framework residues (non-CDR residues) conserved from the parental murine CE7 VL sequence; they have been conserved because they might be structurally important for maintaining the full activity of the antibody. Residues that are underscored are positions that differ within CDRs between humanized version A and the human germline IGKV3-15*01.

| | |
|---|---|
| | <---------FR1---------><--CDR1---><-----FR2-----> |
| CE7-3-15-VLA | EIVMTQSPATLSVSPGERATLSCKANEDINNRLAWYQQKPGQAPRLLIS |
| IGKV3-15*01 | .......................R.SQSVSSN................Y |
| | <CDR2-><--------------FR3-------------><--CDR3 |
| CE7-3-15-VLA | GATNLVTGIPARFSGSGSGKEYTLTISSLQSEDFAVYYCQQYWSTP |
| IGKV3-15*01 | ..STRA.............T.F....................NNW. |

### Humanized Version B

In version B, compared to version A, two amino acid residues have been germlined (i.e substituted by the corresponding human germline residue). In CDR1 Lys (K) Kabat L24 was germlined and mutated to Arg (R). In framework 3 (FR3) Tyr (Y) Kabat L71 was germlined and mutated to Phe (F). Inspection of the molecular model shows that these residues are not likely to make direct contact with antigen, and that they do not play a critical structural role; thus, they may be substituted for their corresponding human germline IGKV3-15*01 counterparts. The main reason for germlining residues within the Kabat CDRs is that these changes will increase the percentage identity of the humanized version B relative to the human germline IGKV3-15*01 to 83.2%.

Version B
[SEQ ID NO.: 82]
FR1 CDR1 FR2
EIVMTQSPATLSVSPGERATLSCRANEDINNRLAWYQQKPGQAPRLLIS
CDR2 FR3 CDR3
GATNLVTGIPARFSGSGSGKEFTLTISSLQSEDFAVYYCQQYWSTP

83.2% identity (79 identical residues out of a total of 95 residues in the V gene) of humanized version B (CE7-3-15-VLB) with IGKV3-15*01. Residues that are bolded are murine framework residues (non-CDR residues) conserved from the parental murine CE7 VL sequence; they have been conserved because they might be structurally important for maintaining the full activity of the antibody. The amino acid residues that have been germlined in humanized version B as compared to humanized version A are underscored.

| | |
|---|---|
| | <---------FR1---------><--CDR1---><-----FR2-----> |
| CE7-3-15-VLB | EIVMTQSPATLSVSPGERATLSCRANEDINNRLAWYQQKPGQAPRLLIS |
| IGKV3-15*01 | .........................SQSVSSN................Y |
| | <CDR2-><--------------FR3-------------><--CDR3 |
| CE7-3-15-VLB | GATNLVTGIPARFSGSGSGKEFTLTISSLQSEDFAVYYCQQYWSTP |
| IGKV3-15*01 | ..STRA.............T......................NNW. |

### Humanized Version C

In version C, compared to version B, two amino acid residues have been germlined (i.e substituted by the corresponding human germline residue). In CDR1 Asn (N) Kabat L24 was germlined and mutated to Ser (S). In CDR2 Thr (T) Kabat L52 was germlined and mutated to Ser (S). Inspection of the molecular model shows that these residues are not likely to make direct contact with antigen, and that they do not play a critical structural role; thus, they may be substituted for their corresponding human germline IGKV3-15*01 counterparts. The main reason for germlining residues within the Kabat CDRs is that these changes will increase the percentage identity of the humanized version C relative to the human germline IGKV3-15*01 to 85.3%.

Version C
[SEQ ID NO.: 83]
FR1 CDR1 FR2
EIVMTQSPATLSVSPGERATLSCRASEDINNRLAWYQQKPGQAPRLLIS
CDR2 FR3 CDR3
GASNLVTGIPARFSGSGSGKEFTLTISSLQSEDFAVYYCQQYWSTP

85.3% identity (81 identical residues out of a total of 95 residues in the V gene) of humanized version B (CE7-3-15-VLC) with IGKV3-15*01. The amino acid residues that have been germlined in humanized version C as compared to humanized version B are underscored.

| | |
|---|---|
| | <---------FR1---------><--CDR1---><-----FR2-----> |
| CE7-3-15-VLC | EIVMTQSPATLSVSPGERATLSCRASEDINNRLAWYQQKPGQAPRLLIS |
| IGKV3-15*01 | ..........................QSVSSN................Y |
| | <CDR2-><--------------FR3-------------><--CDR3 |
| CE7-3-15-VLC | GASNLVTGIPARFSGSGSGKEFTLTISSLQSEDFAVYYCQQYWSTP |
| IGKV3-15*01 | ...TRA.............T......................NNW. |

### Humanized Version D

In version D, compared to version C, two amino acid residues have been germlined (i.e substituted by the corresponding human germline residue). In CDR1 Glu (E) Kabat L27 and Asp (D) Kabat L28 were germlined and mutated to Gln (Q) and Ser (S), respectively. Inspection of the molecular model shows that these residues are not likely to make direct contact with antigen, and that they do not play a critical structural role; thus, they may be substituted for their corresponding human germline IGKV3-15*01 counterparts. The main reason for germlining residues within the Kabat CDRs is that these changes will increase the percentage identity of the humanized version C relative to the human germline IGKV3-15*01 to 87.4%.

Version D
[SEQ ID NO.: 84]
FR1 CDR1 FR2
EIVMTQSPATLSVSPGERATLSCRASQSINNRLAWYQQKPGQAPRLLIS
CDR2 FR3 CDR3
GASNLVTGIPARFSGSGSGKEFTLTISSLQSEDFAVYYCQQYWSTP

87.4% identity (83 identical residues out of a total of 95 residues in the V gene) of humanized version D (CE7-3-15-VLD) with IGKV3-15*01. The amino acid residues that have been germlined in humanized version D as compared to humanized version C are underscored.

| | |
|---|---|
| | <---------FR1---------><--CDR1---><-----FR2-----> |
| CE7-3-15-VLD | EIVMTQSPATLSVSPGERATLSCRASQSINNRLAWYQQKPGQAPRLLIS |
| IGKV3-15*01 | ............................VSSN................Y |
| | <CDR2-><--------------FR3-------------><--CDR3 |
| CE7-3-15-VLD | GASNLVTGIPARFSGSGSGKEFTLTISSLQSEDFAVYYCQQYWSTP |
| IGKV3-15*01 | ...TRA.............T......................NNW. |

### Example 2—Recombinant Antibody Production—Variants 1 to 18

### Genes and Expression Vector

Genes Coding for the rAbs

Starting from sequences of the Heavy chain (HC) and light chain (LC) variable regions of the 18 humanized variants antibodies resulted from the in-silico humanization step, full-length human IgG1 Kappa antibodies harboring mutations L234A, L235A and P331S were designed.

The cDNA coding for the variable regions of the HC and LC were chemically synthesized with optimization for expression in CHO cells and subcloned in ProteoGenix's proprietary mammalian cells expression vectors containing backbones for human IgG1 heavy chain constant region and human kappa light chain constant region [https://www.proteogenix.science/product/xtencho-starter-kit/]. Sequences coding for signal peptides were added in 5′/Nter position. The sequences are illustrated below.

> CE7-1-2-VHA (L234A L235A + P331S)
[SEQ ID NO.: 85]
gaattcgccgccaccATGAAGCACCTGTGGTTCTTCCTGCTGCTGGTGGCTGCCCCCCGGTGGGTTCT
GAGCCAGGTGCAGCTGGTGCAGAGCGGCGCCGAGGTGAAGAAGCCTGGCGCCTCCGTGAAGGTGTCCT
GTAAGGCCTCCGGCTATACCTTCACCGGCTACTGGATGCACTGGGTGAGGCAGGCTCCCGGCCAGGGA
CTGGAGTGGATCGGCGAGATCAATCCTTCCAATGGCAGGACCAACTATAACGAGAGGTTTAAGTCCCG
GGTGACCCTGACCGTGGATAAGAGCATCAGCACCGCTTACATGGAGCTGAGCAGGCTGCGGTCCGACG
ATACCGCCGTGTACTTTTGTGCTAGGGACTATTACGGCACCAGCTACAATTTTGACTATTGGGGCCAG
GGCACCCTGGTGACCGTGAGCTCCgctagcACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACAACTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATG
ACAAAAAATCAGGTCAGCCTGACATGCCTGGTCAAAGGCTTTTATCCCAGCGACATTGCCGTCGAGTG
GGAGTCCAATGGCCAGCCCGAGAATAATTATAAAACAACACCCCCCGTCCTGGACAGCGACGGCAGCT
TTTTTCTGTATAGCAAACTGACAGTCGATAAAAGCAGGTGGCAGCAGGGCAATGTCTTTTCCTGCAGC
GTCATGCACGAGGCCCTGCACAATCACTATACTCAGAAAAGCCTGAGCCTGTCCCCCGGGAAATGAGC
GGCCGC
> CE7-1-2-VHB (L234A L235A + P331S)
[SEQ ID NO.: 86]
gaattcgccgccaccATGAAGCACCTGTGGTTTTTCCTGCTGCTGGTGGCCGCTCCTCGGTGGGTGCTGA
GCCAGGTGCAGCTGGTGCAGTCCGGCGCCGAGGTGAAGAAGCCTGGCGCCAGCGTGAAGGTGAGCTGCAA
GGCTTCCGGCTACACCTTTACCGGCTACTGGATGCACTGGGTGCGGCAGGCTCCTGGCCAGGGACTGGAG
TGGATCGGCGAGATCAACCCTTCCAATGGCCGGACCAACTATAATGAGAGGTTCCAGGGCAGGGTGACCC
TGACCGTGGATAAGTCCATCTCCACCGCCTATATGGAGCTGTCCAGGCTGCGGTCCGACGACACCGCCGT
GTACTATTGCGCCCGGGACTACTACGGCACCTCCTACAACTTTGACTACTGGGGCCAGGGCACCCTGGTG
ACCGTGTCCTCCgctagcACCAAGGGACCTTCTGTGTTCCCTCTGGCTCCTTCTTCTAAGTCCACTTCCG
GTGGTACAGCAGCTCTGGGTTGTCTGGTGAAGGATTACTTCCCAGAACCAGTGACTGTGTCCTGGAACTC
CGGAGCTCTGACTTCTGGAGTGCATACTTTCCCAGCAGTGCTGCAATCTAGCGGACTGTACTCTCTGTCT
TCCGTGGTGACTGTGCCTTCTTCTTCCCTGGGGACTCAAACTTACATCTGCAACGTGAACCACAAGCCCT
CCAACACCAAGGTGGACAAGAAGGTGGAGCCAAAGAGCTGCGATAAGACCCACACCTGTCCACCTTGTCC
AGCTCCAGAAGCCGCCGGTGGGCCTTCTGTGTTTCTGTTCCCACCTAAGCCAAAGGATACCCTGATGATC
TCTAGGACCCCAGAAGTGACCTGTGTGGTCGTCGATGTGTCTCATGAAGACCCTGAAGTGAAGTTCAACT
GGTACGTGGACGGGGTGGAAGTGCATAACGCAAAGACCAAGCCCAGGGAAGAGCAATACAACTCCACCTA
CAGGGTGGTCTCCGTCCTGACAGTCCTGCATCAGGATTGGCTGAACGGCAAGGAGTACAAGTGCAAGGTC
TCCAATAAAGCCCTGCCTGCCAGCATCGAGAAAACCATTAGCAAAGCCAAAGGCCAGCCCAGGGAGCCCC
AGGTCTATACACTGCCCCCCAGCAGGGAGGAGATGACAAAAAATCAGGTCAGCCTGACATGCCTGGTCAA
AGGCTTTTATCCCAGCGACATTGCCGTCGAGTGGGAGTCCAATGGCCAGCCCGAGAATAATTATAAAACA
ACACCCCCCGTCCTGGACAGCGACGGCAGCTTTTTTCTGTATAGCAAACTGACAGTCGATAAAAGCAGGT
GGCAGCAGGGCAATGTCTTTTCCTGCAGCGTCATGCACGAGGCCCTGCACAATCACTATACTCAGAAAAG
CCTGAGCCTGTCCCCCGGGAAATGAGCGGCCGC
> CE7-4-34-VHA (L234A L235A + P331S)
[SEQ ID NO.: 87]
gaattcgccgccaccATGAAGCACCTGTGGTTTTTCCTGCTGCTGGTGGCTGCCCCTAGGTGGGTGCT
GAGCCAGGTGCAGCTGCAGCAGTGGGGCGCCGGACTGCTGAAGCCCAGCGAGACCCTGTCCCTGACCT
GCGCTGCCTACGGCTACACCTTCACCGGCTATTGGATGCACTGGATCCGGCAGCCCCCTGGCAAGGGC
CTGGAGTGGATCGGCGAGATCAATCCTAGCAACGGCCGGACCAATTATAATGAGAGGCTGAAGAGCCG
GGTGACCCTGTCCGTGGACAAGAGCAAGAATCAGGCTTCCCTGAAGCTGTCCTCCGTGACCGCTGCCG
ACACCGCCGTGTACTTTTGCGCTAGGGACTACTATGGCACCTCCTACAATTTTGATTACTGGGGCCAG
GGCACCCTGGTGACCGTGTCCAGCgctagcACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACAACTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAGCG
GCCGC
> CE7-1-NL1-VLA
[SEQ ID NO.: 88]
gaattcgccgccaccATGGTGCTGCAGACCCAGGTGTTCATCTCCCTGCTGCTGTGGATCTCCGGCGCCT
ACGGCGACATCCAGATGACCCAGTCCCCTAGCTCCCTGAGCGCCTCCGTGGGCGACAGAGTGACCATCAC
CTGCAAGGCCAATGAGGATATCAACAACCGGCTGGCTTGGTATCAGCAGAAGCCCGGCAAGGCTCCCAAG
CTGCTGATCTCCGGCGCTACCAATCTGGTGACCGGCGTGCCCTCCAGGTTTAGCGGCAGCGGCTCCGGCA
AGGATTACACCCTGACCATCAGCAGCCTGCAGCCCGAGGACTTCGCTACCTACTATTGCCAGCAGTACTG
GAGCACCCCTTTTACCTTTGGCCAGGGCACCAAGCTGGAGATCAAGcgtacqGTGGCTGCACCTTCTGTG
TTCATCTTCCCTCCATCTGATGAGCAGCTGAAGTCTGGAACCGCATCTGTCGTCTGTCTGCTGAACAACT
TTTACCCCAGGGAGGCTAAGGTCCAATGGAAGGTGGACAACGCCCTGCAGTCTGGTAATAGCCAGGAAAG
CGTGACCGAACAGGATTCCAAGGACTCCACCTACTCCCTGTCCTCCACACTGACACTGAGCAAAGCCGAC
TATGAAAAGCACAAAGTGTATGCCTGCGAGGTCACTCATCAGGGCCTGTCCAGCCCCGTGACTAAAAGCT
TTAATAGGGGGGAGTGCTGAGCGGCCGC
> CE7-1-NL1-VLB
[SEQ ID NO.: 89]
gaattcgccgccaccATGGTGCTGCAGACCCAGGTGTTCATCTCCCTGCTGCTGTGGATCTCCGGCGCCT
ACGGCGATATCCAGATGACCCAGAGCCCTAGCAGCCTGTCCGCCAGCGTGGGCGATAGGGTGACCATCAC
CTGCAGGGCTAACGAGGACATCAATAACAGGCTGGCCTGGTACCAGCAGAAGCCCGGCAAGGCCCCTAAG
CTGCTGATCAGCGGCGCCACCAATCTGGTGACCGGCGTGCCCAGCAGGTTCAGCGGCAGCGGAAGCGGCA
AGGACTACACCCTGACCATCTCCTCCCTGCAGCCTGAGGACTTTGCCACCTATTATTGCCAGCAGTACTG
GAGCACCCCTTTCACCTTTGGCCAGGGCACCAAGCTGGAGATCAAGcgtacgGTGGCTGCACCTTCTGTG
TTCATCTTCCCTCCATCTGATGAGCAGCTGAAGTCTGGAACCGCATCTGTCGTCTGTCTGCTGAACAACT
TTTACCCCAGGGAGGCTAAGGTCCAATGGAAGGTGGACAACGCCCTGCAGTCTGGTAATAGCCAGGAAAG
CGTGACCGAACAGGATTCCAAGGACTCCACCTACTCCCTGTCCTCCACACTGACACTGAGCAAAGCCGAC
TATGAAAAGCACAAAGTGTATGCCTGCGAGGTCACTCATCAGGGCCTGTCCAGCCCCGTGACTAAAAGCT
TTAATAGGGGGGAGTGCTGAGCGGCCGC
> CE7-1-33-VLA
[SEQ ID NO.: 90]
gaattcgccgccaccATGGTGCTGCAGACCCAGGTGTTTATCTCCCTGCTGCTGTGGATCAGCGGCGC
TTATGGCGACATCCAGATGACCCAGTCCCCCTCCAGCCTGTCCGCCAGCGTGGGAGATCGGGTGACCA
TCACCTGTAAGGCTAATGAGGACATCAATAACCGGCTGGCTTGGTACCAGCAGAAGCCCGGCAAGGCT
CCCAAGCTGCTGATCTCCGGCGCTACCAACCTGGTGACCGGCGTGCCTAGCAGGTTTTCCGGCTCCGG
CAGCGGCAAGGATTATACCCTGACCATCTCCTCCCTGCAGCCCGAGGATATCGCTACCTACTACTGCC
AGCAGTATTGGTCCACCCCTTTTACCTTCGGCCAGGGCACCAAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
> CE7-1-33-VLB
[SEQ ID NO.: 91]
gaattcgccgccaccATGGTGCTGCAGACCCAGGTGTTCATCAGCCTGCTGCTGTGGATCTCCGGCGC
CTACGGCGACATCCAGATGACCCAGAGCCCCTCCAGCCTGTCCGCTTCCGTGGGCGATAGGGTGACCA
TCACCTGTCAGGCTAATGAGGACATCAACAACAGGCTGGCTTGGTACCAGCAGAAGCCCGGCAAGGCC
CCCAAGCTGCTGATCAGCGGCGCCACCAACCTGGTGACCGGCGTGCCAAGCAGGTTCAGCGGCAGCGG
CAGCGGAAAGGACTATACCTTCACCATCTCCTCCCTGCAGCCTGAGGACATCGCTACCTATTACTGTC
AGCAGTACTGGTCCACCCCCTTCACCTTTGGCCAGGGCACCAAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
> CE7-3-15-VLA
[SEQ ID NO.: 92]
gaattcgccgccaccATGGTGCTGCAGACCCAGGTGTTTATCAGCCTGCTGCTGTGGATCAGCGGCGCCT
ACGGCGAGATCGTGATGACCCAGTCCCCTGCCACCCTGTCCGTGTCCCCTGGCGAGAGGGCCACCCTGTC
TTGCAAGGCTAATGAGGACATCAACAATAGGCTGGCCTGGTATCAGCAGAAGCCTGGCCAGGCTCCTCGG
CTGCTGATCAGCGGCGCTACCAACCTGGTGACCGGCATCCCTGCCCGGTTCTCCGGCTCCGGAAGCGGAA
AGGAGTATACCCTGACCATCAGCAGCCTGCAGAGCGAGGATTTTGCCGTGTACTATTGCCAGCAGTACTG
GTCCACCCCCTTTACCTTTGGCCAGGGCACCAAGCTGGAGATCAAGcgtacqGTGGCTGCACCTTCTGTG
TTCATCTTCCCTCCATCTGATGAGCAGCTGAAGTCTGGAACCGCATCTGTCGTCTGTCTGCTGAACAACT
TTTACCCCAGGGAGGCTAAGGTCCAATGGAAGGTGGACAACGCCCTGCAGTCTGGTAATAGCCAGGAAAG
CGTGACCGAACAGGATTCCAAGGACTCCACCTACTCCCTGTCCTCCACACTGACACTGAGCAAAGCCGAC
TATGAAAAGCACAAAGTGTATGCCTGCGAGGTCACTCATCAGGGCCTGTCCAGCCCCGTGACTAAAAGCT
TTAATAGGGGGGAGTGCTGAGCGGCCGC
> CE7-3-15-VLB
[SEQ ID NO.: 93]
gaattcgccgccaccATGGTGCTGCAGACCCAGGTGTTCATCTCCCTGCTGCTGTGGATCAGCGGCGCCT
ACGGCGAGATCGTGATGACCCAGAGCCCTGCCACCCTGAGCGTGTCCCCTGGCGAGAGAGCCACCCTGTC
CTGCCGGGCTAACGAGGACATCAACAACCGGCTGGCTTGGTATCAGCAGAAGCCTGGCCAGGCTCCTAGG
CTGCTGATCTCCGGCGCTACCAACCTGGTGACCGGCATCCCTGCCAGGTTTAGCGGCAGCGGCTCCGGCA
AGGAGTTCACCCTGACCATCAGCAGCCTGCAGTCCGAGGATTTCGCCGTGTATTATTGTCAGCAGTACTG
GTCCACCCCCTTCACCTTCGGCCAGGGCACCAAGCTGGAGATCAAGcgtacgGTGGCTGCACCTTCTGTG
TTCATCTTCCCTCCATCTGATGAGCAGCTGAAGTCTGGAACCGCATCTGTCGTCTGTCTGCTGAACAACT
TTTACCCCAGGGAGGCTAAGGTCCAATGGAAGGTGGACAACGCCCTGCAGTCTGGTAATAGCCAGGAAAG
CGTGACCGAACAGGATTCCAAGGACTCCACCTACTCCCTGTCCTCCACACTGACACTGAGCAAAGCCGAC
TATGAAAAGCACAAAGTGTATGCCTGCGAGGTCACTCATCAGGGCCTGTCCAGCCCCGTGACTAAAAGCT
TTAATAGGGGGGAGTGCTGAGCGGCCGC

### Sequences of Expressed Proteins

> CE7-1-2-VHA (L234A L235A + P331S)
[SEQ ID NO.: 94]
MKHLWFFLLLVAAPRWVLSQVQLVQSGAEVKKPGASVKVSCKASGYTFTGYWMHWVRQAPGQGLEWIG
EINPSNGRTNYNERFKSRVTLTVDKSISTAYMELSRLRSDDTAVYFCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPKDT
LMISRTPEVTCVVVDVSHEDPEVKENWYVDGVEVHNAKTKPREEQYNSTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
> CE7-1-2-VHB (L234A L235A + P331S)
[SEQ ID NO.: 95]
MKHLWFFLLLVAAPRWVLSQVQLVQSGAEVKKPGASVKVSCKASGYTFTGYWMHWVRQAPGQGLEWIG
EINPSNGRTNYNERFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYNSTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
> CE7-4-34-VHA (L234A L235A + P331S)
[SEQ ID NO.: 96]
MKHLWFFLLLVAAPRWVLSQVQLQQWGAGLLKPSETLSLTCAAYGYTFTGYWMHWIRQPPGKGLEWIG
EINPSNGRTNYNERLKSRVTLSVDKSKNQASLKLSSVTAADTAVYFCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPKDT
LMISRTPEVTCVVVDVSHEDPEVKENWYVDGVEVHNAKTKPREEQYNSTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
> CE7-1-NL1-VLA
[SEQ ID NO.: 97]
MVLQTQVFISLLLWISGAYGDIQMTQSPSSLSASVGDRVTITCKANEDINNRLAWYQQKPGKAPKLLI
SGATNLVTGVPSRFSGSGSGKDYTLTISSLQPEDFATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
> CE7-1-NL1-VLB
[SEQ ID NO.: 98]
MVLQTQVFISLLLWISGAYGDIQMTQSPSSLSASVGDRVTITCRANEDINNRLAWYQQKPGKAPKLLI
SGATNLVTGVPSRESGSGSGKDYTLTISSLQPEDFATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
> CE7-1-33-VLA
[SEQ ID NO.: 99]
MVLQTQVFISLLLWISGAYGDIQMTQSPSSLSASVGDRVTITCKANEDINNRLAWYQQKPGKAPKLLI
SGATNLVTGVPSRFSGSGSGKDYTLTISSLQPEDIATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
> CE7-1-33-VLB
[SEQ ID NO.: 100]
MVLQTQVFISLLLWISGAYGDIQMTQSPSSLSASVGDRVTITCQANEDINNRLAWYQQKPGKAPKLLI
SGATNLVTGVPSRESGSGSGKDYTFTISSLQPEDIATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
> CE7-3-15-VLA
[SEQ ID NO.: 101]
MVLQTQVFISLLLWISGAYGEIVMTQSPATLSVSPGERATLSCKANEDINNRLAWYQQKPGQAPRLLI
SGATNLVTGIPARFSGSGSGKEYTLTISSLQSEDFAVYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
> CE7-3-15-VLB
[SEQ ID NO.: 102]
MVLQTQVFISLLLWISGAYGEIVMTQSPATLSVSPGERATLSCRANEDINNRLAWYQQKPGQAPRLLI
SGATNLVTGIPARESGSGSGKEFTLTISSLQSEDFAVYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC

### Small-Scale Production and Purification Tests

### Short Protocol Description

An endotoxin-free DNA preparation was done for the constructions obtained. The eighteen antibodies were expressed by combining HC and LC as shown in the Table E1. Using the proprietary Xten transfection protocol, the plasmids were transiently co-transfected in our proprietary XtenCHO cells. Cell culture medium samples were collected when viability dropped under 50% (14 days after transfection) and the rAbs were then purified on a protein A resin by using a standard method:
Clarification by 0.22 μm filtration
Equilibration, binding, and wash with PBS pH7.5
Elution by pH shift with citric acid
Neutralization with 1 M Tris-HCl pH 9.0
Analysis by PAGE and pool of the fractions of interest.
Final QC by PAGE: qualitative and quantitative by SDS-PAGE

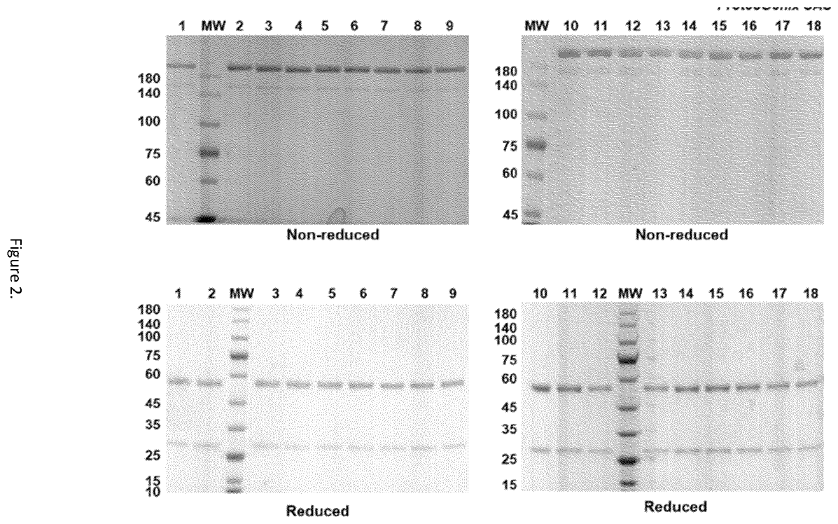

Elution fractions were pooled and buffer exchanged vs PBS, pH7.5 by dialysis method. Final samples were filtered by 0.22 um Millipore filter. Purification profiles and final QC are illustrated in FIG. 1 and FIG. 2. The yield and purity obtained are summarized in the Table E2.

TABLE E2
Yield and purity obtained for the rAb pilot productions.

| | | Yield* | | |
|---|---|---|---|---|
| Antibody | Concentration | (mg/30 ml) | | Purity** |
| CE7- | 1.00 mg/ml | 3.8 | mg | >90% |
| variant1 | | | | |
| CE7- | 1.00 mg/ml | 3.52 | mg | >90% |
| variant2 | | | | |
| CE7- | 1.00 mg/ml | 3.4 | mg | >90% |
| variant3 | | | | |
| CE7- | 1.00 mg/ml | 2.8 | mg | >90% |
| variant4 | | | | |
| CE7- | 1.00 mg/ml | 0.8 | mg | >90% |
| variant5 | | | | |
| CE7- | 1.00 mg/ml | 5.36 | mg | >90% |
| variant6 | | | | |
| CE7- | 1.00 mg/ml | 3.86 | mg | >90% |
| variant7 | | | | |
| CE7- | 1.00 mg/ml | 4.5 | mg | >90% |
| variant8 | | | | |
| CE7- | 1.00 mg/ml | 1.83 | mg | >90% |
| variant9 | | | | |
| CE7- | 1.00 mg/ml | 5.1 | mg | >90% |
| variant10 | | | | |
| CE7- | 1.00 mg/ml | 0.8 | mg | >90% |
| variant11 | | | | |
| CE7- | 1.00 mg/ml | 6.28 | mg | >90% |
| variant12 | | | | |
| CE7- | 1.00 mg/ml | 8.45 | mg | >90% |
| variant13 | | | | |
| CE7- | 1.00 mg/ml | 3.1 | mg | >90% |
| variant14 | | | | |
| CE7- | 1.00 mg/ml | 1.80 | mg | >90% |
| variant15 | | | | |
| CE7- | 1.00 mg/ml | 2.65 | mg | >90% |
| variant16 | | | | |
| CE7- | 1.00 mg/ml | 1.33 | mg | >90% |
| variant17 | | | | |
| CE7- | 1.00 mg/ml | 4.7 | mg | >90% |
| variant18 | | | | |

*Obtained after purification for the 30 ml-culture test.
**Based on full length antibody observed on non-reduced PAGE analyses in FIG. 2.

### Example 3—Recombinant Antibody Production—Variants 19 to 28

### Genes and Expression Vector

Genes Coding for the rAbs

Starting from sequences of the Heavy chain (HC) and light chain (LC) variable regions of the additional 12 humanized variants antibodies resulted from the in-silico humanization step, full-length human IgG1 Kappa antibodies harboring mutations L234A, L235A, P331S and N297A were designed.

The cDNA coding for the variable regions of the HC and LC were chemically synthesized with optimization for expression in CHO cells and subcloned in ProteoGenix's proprietary mammalian cells expression vectors containing backbones for human IgG1 heavy chain constant region and human kappa light chain constant region (https://www.proteogenix.science/product/xtencho-starter-kit/). Sequences coding for signal peptides were added in 5′/Nter position. The sequences are illustrated below.

> CE7-1-2-VHB (L234A L235A + P331S) (14047) [SEQ ID NO.: 86],
as above
gaattcgccgccaccATGAAGCACCTGTGGTTTTTCCTGCTGCTGGTGGCCGCTCCTCGGTGGGTGCT
GAGCCAGGTGCAGCTGGTGCAGTCCGGCGCCGAGGTGAAGAAGCCTGGCGCCAGCGTGAAGGTGAGCT
GCAAGGCTTCCGGCTACACCTTTACCGGCTACTGGATGCACTGGGTGCGGCAGGCTCCTGGCCAGGGA
CTGGAGTGGATCGGCGAGATCAACCCTTCCAATGGCCGGACCAACTATAATGAGAGGTTCCAGGGCAG
GGTGACCCTGACCGTGGATAAGTCCATCTCCACCGCCTATATGGAGCTGTCCAGGCTGCGGTCCGACG
ACACCGCCGTGTACTATTGCGCCCGGGACTACTACGGCACCTCCTACAACTTTGACTACTGGGGCCAG
GGCACCCTGGTGACCGTGTCCTCCgctagcACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACAACTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAGCG
GCCGC
>CE7-1-2-VHB (L234A L235A + P331S N297A)
[SEQ ID NO.: 103]
GAATTCgccgccaccATGAAGCACCTGTGGTTCTTCCTGCTGCTGGTGGCCGCCCCCAGATGGGTGCT
GTCCCAGGTGCAGCTGGTGCAGAGCGGCGCCGAGGTGAAGAAGCCTGGCGCCTCCGTGAAGGTGAGCT
GCAAGGCCTCCGGCTACACATTCACTGGCTACTGGATGCACTGGGTGAGACAGGCCCCTGGCCAGGGC
CTGGAGTGGATCGGAGAGATCAACCCCAGCAATGGCAGAACCAATTACAATGAGAGATTCCAGGGCAG
AGTGACCCTGACCGTGGATAAGTCCATCAGCACAGCCTACATGGAGCTGTCCAGACTGAGAAGCGATG
ACACCGCCGTGTACTACTGCGCCAGAGACTACTACGGCACCTCCTACAACTTTGATTACTGGGGCCAG
GGCACACTGGTGACAGTGAGCAGCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-1-2-VHC (L234A L235A + P331S N297A)
[SEQ ID NO.: 104]
GAATTCgccgccaccATGAAGCACCTGTGGTTTTTCCTGCTGCTGGTGGCCGCCCCCAGGTGGGTTCT
GTCCCAGGTGCAGCTGGTGCAGAGCGGCGCCGAGGTGAAGAAGCCTGGCGCCTCCGTGAAGGTGTCCT
GCAAGGCCAGCGGCTACACCTTCACAGGCTACTGGATGCACTGGGTGAGGCAGGCCCCCGGCCAGGGA
CTGGAATGGATCGGCGAGATCAACCCTAGCAACGGCAGGACAAATTACAACGAGAAGTTCCAGGGCAG
AGTGACACTGACAGTGGACAAGTCCATCAGCACCGCCTACATGGAGCTGTCCAGACTGAGATCCGACG
ATACAGCCGTGTACTACTGCGCCAGGGACTACTACGGCACCAGCTACAATTTTGACTACTGGGGCCAG
GGCACCCTGGTGACAGTGTCCTCCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-1-2-VHD (L234A L235A + P331S N297A)
[SEQ ID NO.: 105]
GAATTCgccgccaccATGAAGCACCTGTGGTTTTTCCTGCTGCTGGTGGCCGCCCCCAGGTGGGTTCT
GAGCCAGGTGCAGCTGGTGCAGAGCGGCGCCGAGGTGAAGAAGCCCGGCGCTTCCGTGAAGGTGTCCT
GTAAGGCCAGCGGCTACACATTCACCGGCTACTGGATGCACTGGGTGAGACAGGCCCCCGGCCAGGGA
CTGGAGTGGATGGGAGAGATCAATCCTAGCAACGGCAGAACAAATTACAATGAGAAGTTCCAGGGCAG
AGTGACACTGACAGTGGACAAGAGCATCTCCACAGCCTACATGGAGCTGTCCAGACTGAGATCCGACG
ACACCGCCGTGTACTACTGTGCCAGAGATTACTACGGCACATCCTACAACTTCGACTACTGGGGCCAG
GGCACACTGGTGACAGTGTCCTCCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-1-NL1-VLA
[SEQ ID NO.: 106]
GAATTCgccgccaccATGGTGCTGCAGACCCAGGTGTTCATCTCCCTGCTGCTGTGGATCAGCGGCGC
TTATGGCGACATCCAGATGACCCAGAGCCCTTCCTCCCTGTCCGCCAGCGTGGGCGACAGGGTGACCA
TCACCTGTAAGGCCAATGAGGATATCAATAATCGGCTGGCCTGGTATCAGCAGAAGCCCGGCAAGGCC
CCCAAGCTGCTGATCTCCGGCGCTACCAATCTGGTGACCGGCGTGCCTTCCCGGTTTTCCGGCTCCGG
CAGCGGCAAGGATTATACCCTGACCATCAGCAGCCTGCAGCCCGAGGATTTTGCTACCTATTATTGCC
AGCAGTATTGGTCCACCCCCTTCACCTTTGGCCAGGGCACCAAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-1-NL1-VLB
[SEQ ID NO.: 107]
GAATTCgccgccaccATGGTGCTGCAGACCCAGGTGTTTATCTCCCTGCTGCTGTGGATCTCCGGCGC
CTACGGCGACATCCAGATGACCCAGTCCCCCAGCTCCCTGTCCGCTAGCGTGGGCGACAGGGTGACCA
TCACCTGTAGGGCCAATGAGGACATCAACAACAGGCTGGCCTGGTATCAGCAGAAGCCCGGCAAGGCT
CCTAAGCTGCTGATCTCCGGCGCTACCAATCTGGTGACCGGCGTGCCTTCCAGGTTCTCCGGCTCCGG
CTCCGGAAAGGATTACACCCTGACCATCAGCTCCCTGCAGCCTGAGGACTTTGCTACCTATTACTGCC
AGCAGTACTGGTCCACCCCCTTTACCTTCGGCCAGGGCACCAAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-1-NL1-VLC
[SEQ ID NO.: 108]
GAATTCgccgccaccATGGTGCTGCAGACCCAGGTGTTTATCTCCCTGCTGCTGTGGATCTCCGGCGC
CTACGGCGATATCCAGATGACCCAGAGCCCCTCCAGCCTGAGCGCCTCCGTGGGAGACAGGGTGACCA
TCACCTGTAGGGCCAACGAGGATATCAACAACAGGCTGGCCTGGTATCAGCAGAAGCCTGGCAAGGCT
CCTAAGCTGCTGATCAGCGGCGCTTCCAACCTGGTGAGCGGCGTGCCCTCCAGGTTTAGCGGCAGCGG
CAGCGGAAAGGATTACACCCTGACCATCAGCTCCCTGCAGCCCGAGGACTTCGCTACCTACTATTGCC
AGCAGTACTGGAGCACCCCTTTCACCTTCGGCCAGGGCACCAAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-1-NL1-VLD
[SEQ ID NO.: 109]
GAATTCgccgccaccATGGTGCTGCAGACCCAGGTGTTCATCTCCCTGCTGCTGTGGATCAGCGGCGC
TTATGGCGACATCCAGATGACCCAGTCCCCTAGCTCCCTGAGCGCCTCCGTGGGCGACAGGGTGACCA
TCACCTGCCGGGCCAGCCAGGGCATCAATAATCGGCTGGCTTGGTACCAGCAGAAGCCCGGCAAGGCC
CCTAAGCTGCTGATCAGCGGCGCCTCCAACCTGGTGAGCGGCGTGCCTTCCAGGTTCTCCGGCAGCGG
CAGCGGCAAGGACTATACCCTGACCATCAGCAGCCTGCAGCCTGAGGACTTTGCCACCTACTATTGCC
AGCAGTATTGGTCCACCCCCTTTACCTTTGGCCAGGGCACCAAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC

The sequences for the expected proteins produced are illustrated below.

> CE7-1-2-VHB (L234A L235A + P331S) (14047) [SEQ ID NO.: 94] as
seen above
MKHLWFFLLLVAAPRWVLSQVQLVQSGAEVKKPGASVKVSCKASGYTFTGYWMHWVRQAPGQGLEWIG
EINPSNGRTNYNERFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTEPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYNSTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-1-2-VHB (L234A L235A + P331S N297A)
[SEQ ID NO.: 110]
MKHLWFFLLLVAAPRWVLSQVQLVQSGAEVKKPGASVKVSCKASGYTFTGYWMHWVRQAPGQGLEWIG
EINPSNGRTNYNERFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKFNWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-1-2-VHC (L234A L235A + P331S N297A)
[SEQ ID NO.: 111]
MKHLWFFLLLVAAPRWVLSQVQLVQSGAEVKKPGASVKVSCKASGYTFTGYWMHWVRQAPGQGLEWIG
EINPSNGRTNYNEKFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-1-2-VHD (L234A L235A + P331S N297A)
[SEQ ID NO.: 112]
MKHLWFFLLLVAAPRWVLSQVQLVQSGAEVKKPGASVKVSCKASGYTFTGYWMHWVRQAPGQGLEWMG
EINPSNGRTNYNEKFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-1-NL1-VLA
[SEQ ID NO.: 97]
MVLQTQVFISLLLWISGAYGDIQMTQSPSSLSASVGDRVTITCKANEDINNRLAWYQQKPGK
APKLLISGATNLVTGVPSRFSGSGSGKDYTLTISSLQPEDFATYYCQQYWSTPFTFGQGTKL
EIKRTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSENRGEC
>CE7-1-NL1-VLB
[SEQ ID NO.: 98]
MVLQTQVFISLLLWISGAYGDIQMTQSPSSLSASVGDRVTITCRANEDINNRLAWYQQKPGKAPKLLI
SGATNLVTGVPSRESGSGSGKDYTLTISSLQPEDFATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-1-NL1-VLC
[SEQ ID NO.: 113]
MVLQTQVFISLLLWISGAYGDIQMTQSPSSLSASVGDRVTITCRANEDINNRLAWYQQKPGKAPKLLI
SGASNLVSGVPSRFSGSGSGKDYTLTISSLQPEDFATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-1-NL1-VLD
[SEQ ID NO.: 114]
MVLQTQVFISLLLWISGAYGDIQMTQSPSSLSASVGDRVTITCRASQGINNRLAWYQQKPGKAPKLLI
SGASNLVSGVPSRESGSGSGKDYTLTISSLQPEDFATYYCQQYWSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC

### Small-Scale Production and Purification Tests

### Short Protocol Description

An endotoxin-free DNA preparation was done for the constructions obtained as described herein. The twelve antibodies were expressed by combining HC and LC as follows:

TABLE E3

| Antibody ID | Heavy Chain | Light Chain |
|---|---|---|
| CE7- variant7 | pTXs1-CE7-1-2-VHB | pTXs1-CE7-1-NL1-VLA |
| | (14047) | (14047) |
| CE7- variant19 | pTXs1-CE7-1-2-VHB | pTXs1-CE7-1-NL1-VLC |
| | (L234A L235A + | |
| | P331S + N297A) | |
| CE7- variant20 | pTXs1-CE7-1-2-VHB | pTXs1-CE7-1-NL1-VLD |
| | (L234A L235A + | |
| | P331S + N297A) | |
| CE7- variant21 | pTXs1-CE7-1-2-VHC | pTXs1-CE7-1-NL1-VLA |
| | (L234A L235A + | |
| | P331S + N297A) | |
| CE7- variant22 | pTXs1-CE7-1-2-VHC | pTXs1-CE7-1-NL1-VLB |
| | (L234A L235A + | |
| | P331S + N297A) | |
| CE7- variant23 | pTXs1-CE7-1-2-VHC | pTXs1-CE7-1-NL1-VLC |
| | (L234A L235A + | |
| | P331S + N297A) | |
| CE7- variant24 | pTXs1-CE7-1-2-VHC | pTXs1-CE7-1-NL1-VLD |
| | (L234A L235A + | |
| | P331S + N297A) | |
| CE7- variant25 | pTXs1-CE7-1-2-VHD | pTXs1-CE7-1-NL1-VLA |
| | (L234A L235A + | |
| | P331S + N297A) | |
| CE7- variant26 | pTXs1-CE7-1-2-VHD | pTXs1-CE7-1-NL1-VLB |
| | (L234A L235A + | |
| | P331S + N297A) | |
| CE7- variant27 | pTXs1-CE7-1-2-VHD | pTXs1-CE7-1-NL1-VLC |
| | (L234A L235A + | |
| | P331S + N297A) | |
| CE7- variant28 | pTXs1-CE7-1-2-VHD | pTXs1-CE7-1-NL1-VLD |
| | (L234A L235A + | |
| | P331S + N297A) | |
| CE7- variant7-AG | pTXs1-CE7-1-2-VHB | pTXs1-CE7-1-NL1-VLA |
| | (L234A L235A + | (14047) |
| | P331S + N297A) | |

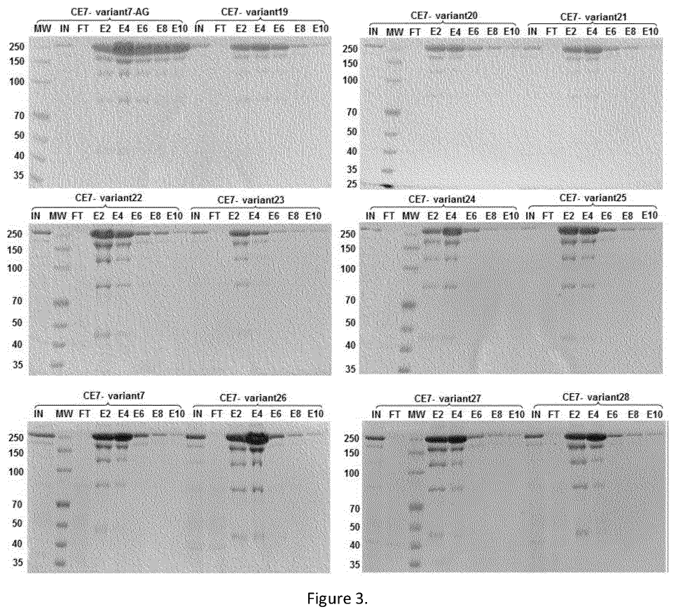

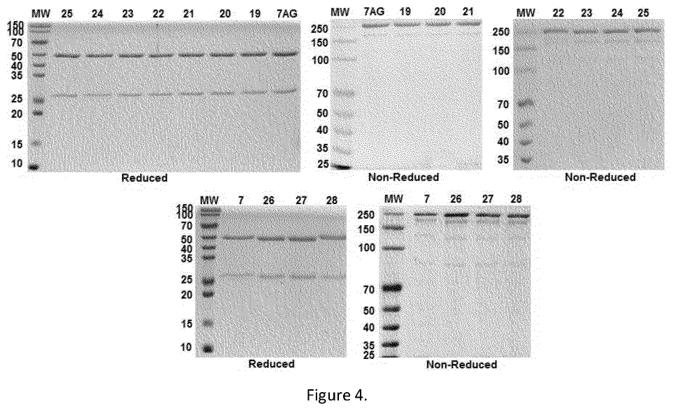

The small scale antibody production was performed as described in Example 2. The results are shown in FIGS. 3 and 4. The yield and purity obtained are summarized in Table E4.

TABLE E4
Yield and purity obtained for the rAb pilot productions.

| Antibody | Concentration | Specification | Quantity | Yield* | Purity** |
|---|---|---|---|---|---|
| CE7- | 0.90 mg/ml | 1.70 ml/vial | 2vials | 3.06 mg/ | >90% |
| variant7 | | | | 30 ml | |
| CE7- | 0.77 mg/ml | 1.83 ml/vial | 2vials | 2.82 mg/ | >90% |
| variant19 | | | | 30 ml | |
| CE7- | 0.72 mg/ml | 1.50 ml/vial | 2 vials | 2.16 mg/ | >90% |
| variant20 | | | | 30 ml | |
| CE7- | 0.65 mg/ml | 1.52 ml/vial | 2 vials | 1.98 mg/ | >90% |
| variant21 | | | | 30 ml | |
| CE7- | 1.17 mg/ml | 1.86 ml/vial | 1 vial | 2.18 mg/ | >90% |
| variant22 | | | | 30 ml | |
| CE7- | 0.70 mg/ml | 1.75 ml/vial | 1 vial | 1.23 mg/ | >90% |
| variant23 | | | | 30 ml | |
| CE7- | 0.67 mg/ml | 1.60 ml/vial | 2 vials | 2.14 mg/ | >90% |
| variant24 | | | | 30 ml | |
| CE7- | 0.82 mg/ml | 1.41 ml/vial | 2 vials | 2.31 mg/ | >90% |
| variant25 | | | | 30 ml | |
| CE7- | 0.67 mg/ml | 1.60 ml/vial | 2 vials | 2.14 mg/ | >90% |
| variant26 | | | | 30 ml | |
| CE7- | 0.94 mg/ml | 1.60 ml/vial | 1 vial | 1.50 mg/ | >90% |
| variant27 | | | | 30 ml | |
| CE7- | 0.97 mg/ml | 1.70 ml/vial | 1 vial | 1.65 mg/ | >90% |
| variant28 | | | | 30 ml | |
| CE7- | 1.66 mg/ml | 1.58 ml/vial | 6vials | 15.74 mg/ | >90% |
| variant7-AG | | | | 200 ml | |

*Obtained after purification for the 30 ml-culture test.
**Based on full length antibody observed on non-reduced PAGE analyses in FIG. 2.
Final buffer: PBS pH 7.5.

### Example 4—Expression and Purification Text Results for Variants 4, 7, 15, 17 and 18 in Aglycosylated Form

Starting from sequences of the Heavy chain (HC) and light chain (LC) variable regions of the 18 humanized variants antibodies resulted from the in-silico humanization step, full-length human IgG1Kappa antibodies harboring mutations L234A, L235A, P331 S and N297A were designed. The cDNA coding for the HC and LC were chemically synthesized with optimization for expression in CHO cells and subcloned in ProteoGenix's proprietary mammalian cells expression vectors containing backbones for human IgG1 heavy chain constant region and human kappa light chain constant region (https://www.proteogenix.science/product/xtencho-starter-kit/). Sequences coding for signal peptides were added in 5′/Nter position. The sequences are illustrated below.

> CE7-1-2-VHA (L234A L235A + P331S + N297A)
[SEQ ID NO.: 115]
gaattcgccgccaccATGAAGCACCTGTGGTTCTTCCTGCTGCTGGTGGCTGCCCCCCGGTGGGTTCT
GAGCCAGGTGCAGCTGGTGCAGAGCGGCGCCGAGGTGAAGAAGCCTGGCGCCTCCGTGAAGGTGTCCT
GTAAGGCCTCCGGCTATACCTTCACCGGCTACTGGATGCACTGGGTGAGGCAGGCTCCCGGCCAGGGA
CTGGAGTGGATCGGCGAGATCAATCCTTCCAATGGCAGGACCAACTATAACGAGAGGTTTAAGTCCCG
GGTGACCCTGACCGTGGATAAGAGCATCAGCACCGCTTACATGGAGCTGAGCAGGCTGCGGTCCGACG
ATACCGCCGTGTACTTTTGTGCTAGGGACTATTACGGCACCAGCTACAATTTTGACTATTGGGGCCAG
GGCACCCTGGTGACCGTGAGCTCCgctagcACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt

The following sequences are as referred to hereinabove:

> CE7-1-2-VHB (L234A L235A + P331S + N297A)
[SEQ ID NO.: 103]
> CE7-4-34-VHA (L234A L235A + P331S + N297A)
[SEQ ID NO.: 87]
> CE7-1-NL1-VLA
[SEQ ID NO.: 88]
> CE7-1-33-VLA
[SEQ ID NO.: 90]
> CE7-1-33-VLB
[SEQ ID NO.: 91]
> CE7-3-15-VLA
[SEQ ID NO.: 92]
> CE7-3-15-VLB
[SEQ ID NO.: 93]

Expected protein product is as follows or as defined hereinabove, depending on the sequence:

> CE7-1-2-VHA (L234A L235A + P331S + N297A)
[SEQ ID NO.: 116]
MKHLWFFLLLVAAPRWVLSQVQLVQSGAEVKKPGASVKVSCKASGYTFTG
YWMHWVRQAPGQGLEWIGEINPSNGRTNYNERFKSRVTLTVDKSISTAYM
ELSRLRSDDTAVYFCARDYYGTSYNFDYWGQGTLVTVSSASTKGPSVFPL
APSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPP
CPAPEAAGGPSVFLFPPKPKDTLMISRTPEVTCVVVDVSHEDPEVKENWY
VDGVEVHNAKTKPREEQYASTYRVVSVLIVLHQDWLNGKEYKCKVSNKAL
PASIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK

### Small-Scale Production and Purification Tests

### Short Protocol Description

An endotoxin-free DNA preparation was done for the constructions obtained as described herein. The five antibodies were expressed by combining HC and LC as follows:

TABLE E5

| Antibody ID | Heavy Chain | Light Chain |
|---|---|---|
| CE7- variant4_AG | pTXs1-CE7-1-2- | pTXs1-CE7-1-33- |
| | VHA (L234A L235A + | VLB |
| | P331S + N297A) | |
| CE7- variant7_AG | pTXs1-CE7-1-2- | pTXs1-CE7-1-NL1- |
| | VHB (L234A L235A + | VLA |
| | P331S + N297A) | |
| CE7- variant15_AG | pTXs1-CE7-4-34- | pTXs1-CE7-1-33- |
| | VHA (L234A L235A + | VLA |
| | P331S + N297A) | |
| CE7- variant17_AG | pTXs1-CE7-4-34- | pTXs1-CE7-3-15- |
| | VHA (L234A L235A + | VLA |
| | P331S + N297A) | |
| CE7- variant18_AG | pTXs1-CE7-4-34- | pTXs1-CE7-3-15- |
| | VHA (L234A L235A + | VLB |
| | P331S + N297A) | |

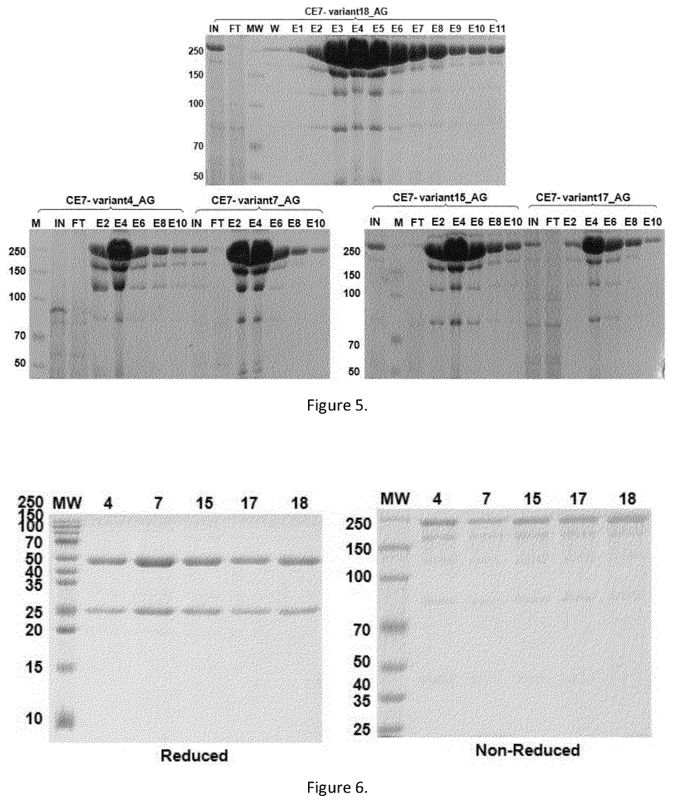

Results of the small-scale production are summarized in FIGS. 5 and 6. The yield and purity data is summarized in Table E6.

TABLE E6
Yield and purity obtained for the rAb pilot productions.

| | | Yield* | | |
|---|---|---|---|---|
| Antibody | Concentration | (mg/80 ml) | | Purity** |
| CE7- | 2.54 mg/ml | 11.43 | mg | >90% |
| variant4_AG | | | | |
| CE7- | 3.26 mg/ml | 11.41 | mg | >90% |
| variant7_AG | | | | |
| CE7- | 1.94 mg/ml | 10.36 | mg | >90% |
| variant15_AG | | | | |
| CE7- | 1.09 mg/ml | 3.73 | mg | >90% |
| variant17_AG | | | | |
| CE7- | 1.99 mg/ml | 9.67 | mg | >90% |
| variant18_AG | | | | |

*Obtained after purification for the 30 ml-culture test.
**Based on full length antibody observed on non-reduced PAGE analyses in FIG. 6.

### Example 5—SEC HPLC Analysis

Starting from samples of each protein obtained after final conditioning and aliquoting as described in Example 4, SEC-HPLC analysis was performed to evaluate the aggregation level of the top three candidates: GE7-variant4_AG, GE7-variant7_AG and GE7-variant17_AG. The analyses were performed with a HPLC Waters 2695 device equipped with a Photodiode Array Detector (2996).

### Buffer Preparation

Mobile phase: 100 mM Sodium sulfate, 100 mM Phosphate salt, pH 6.7 buffer (Na2SO4, 14.2 g/L, Na2HPO4·12H2O 15.6 g/L, NaH2PO4 6.8 g/L) Air bubbles are removed from all buffers.

### Sample Preparation

Protein preparation: Starting from one extra aliquot of each final sample store at −80° C. for one night, dilution of the protein samples to 1 mg/ml with DD water—Centrifugation at 12000 g during 10 min and collection of supernatants

### Analysis Conditions

Blank control: dilution (5×) of the sample with DD water
Column: G3000SWXL, TOSOH, 7.8×300 mm
HPLC: Waters 2695
Elution gradient: mobile phase isocratic elution
Flow rate: 0.8 ml/min
Temperature: 25° C.
Injection volume: 30 μl
Detection wavelength: 280 nm
Collect date time: 25 min
Equilibration with mobile phase 100% for 5 min
Run Samples: Injection of one blank control and then injection of samples
Data Analysis

The results are shown in Table E7.

TABLE E7
SEC HPLC Data

| | Retention time | Area | Height | Purity |
|---|---|---|---|---|
| Antibody | (min) | (μV/s) | (μV/s) | (%) |
| CE7- | 7.890 | 98671 | 2743 | |
| variant4_AG | 9.390 | 1793192 | 26239 | 95% |
| CE7- | 7.876 | 59047 | 1603 | |
| variant7_AG | 9.378 | 2228798 | 35475 | 97.5% |
| CE7- | 7.840 | 25690 | 711 | |
| variant18_AG | 9.453 | 1180986 | 17872 | 98% |
| SEC 300A | 6.805 | 64909 | 1413 | |
| Protein | 9.589 | 80486 | 1521 | |
| Standard | 11.082 | 41812 | 1115 | |
| | 12.453 | 60826 | 1430 | |
| | 15.074 | 10117 | 259 | |

*Purity of full antibody single molecules detected in the sample.

When several peaks are detected, data corresponding to the peak of full antibody single molecules is indicated with underscore. When a single peak is detected, an estimate of the concentration (purity) is given.

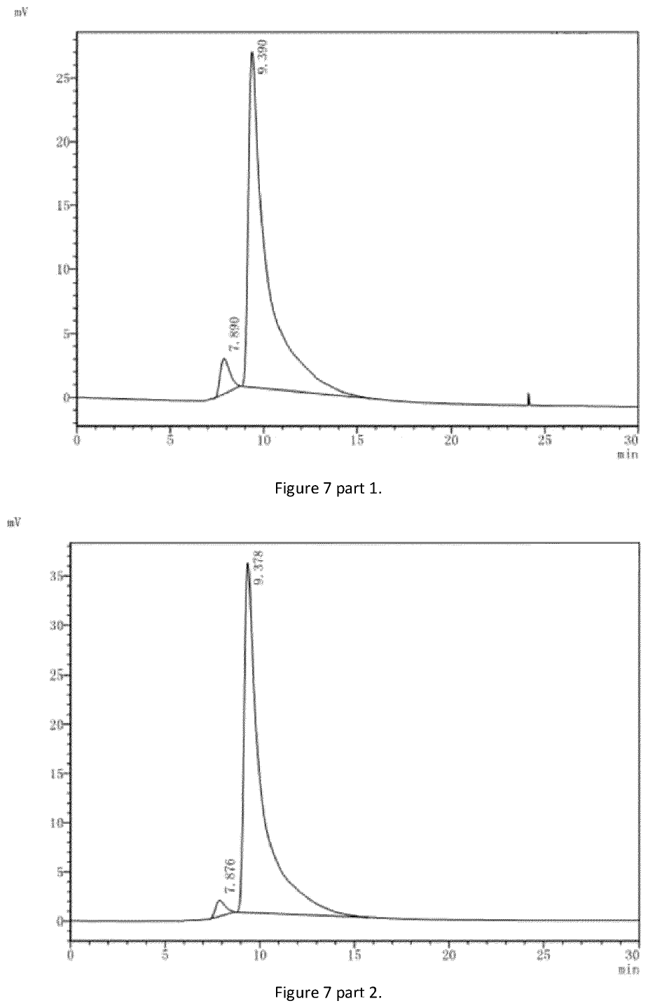

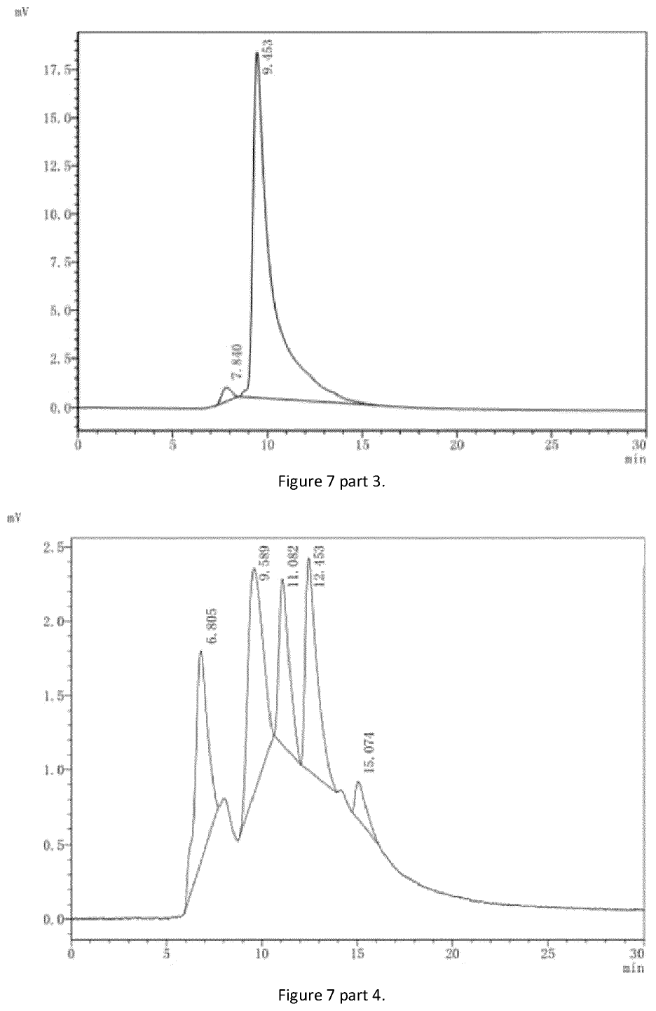

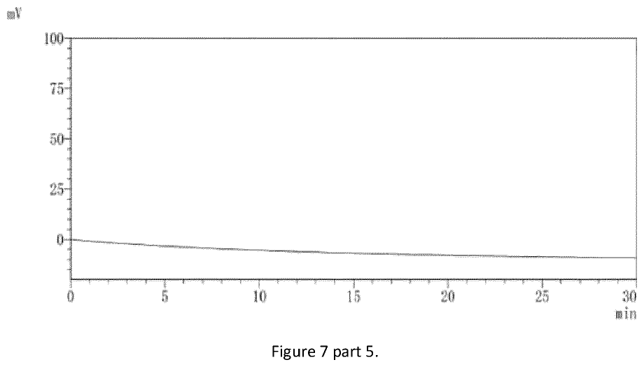

SEC-HPLC profiles obtained for the samples are also shown in FIG. 7. SEC-HPLC analyses show that all three antibody samples exhibit the best concentration of ideal heterotetrametric antibodies composed of two heavy and two light chains and very few aggregates <5%.

### Example 6—Determination of KD of Antibody/Antigen Interaction Via Surface Plasmon Resonance (SPR) Technology (Biacore 8K)

In this study, Kd of 3 humanised antibodies for its soluble antigen via Biacore 8K has been determined. L1-CAM recombinant protein (His Tag) has been used, which has been obtained from Sinobiological under catalogue number 10140-H08H (https://www.sinobiological.com/recombinant-proteins/human-l1cam-1010-h08h as assessed on Sep. 2, 2022). In the SPR experiment, antigen is immobilized on sensor chip and solutions containing antibody at various concentrations are flown over the antigen. real time measurement of kinetics parameters is performed, including their on/off rates (ka and kd). Based thereon, binding affinity constant KD is determined. Materials used are summarized in Table E8.

TABLE E8
Material information

| Product | Type | Concentration | Purity | MW (kDa) |
|---|---|---|---|---|
| L1-CAM | Ligand | 1.00 mg/ml | >90% | 143 |
| CE7-varant4- | Analyte | 2.54 mg/ml | >90% | 150 |
| AG | | | | |
| CE7-varant7- | Analyte | 3.26 mg/ml | >90% | 150 |
| AG | | | | |
| CE7- | Analyte | 1.99 mg/ml | >90% | 150 |
| varant18-AG | | | | |
| chimeric | Analyte | 2.10 mg/ml | >90% | 150 |
| cHCE7 | | | | |
| Deglycosilated | Analyte | 3.40 mg/ml | >90% | 150 |
| cHCE7 | | | | |

The following buffers were used in the experiment:
Antigen dilution buffer: NaAc pH 4.5 (20 mM NaAc pH 4.5)
Running buffer/Antibody dilution buffer: HBS-EP+ (0.01 M Hepes pH 7.4, 0.15 M NaCl, 3 mM EDTA, 0.01% Surfactant P20)
Regeneration buffer: glycine pH 1.5

CM5 sensor chip was used, which is a glass slide coated with a thin layer of gold, to which a matrix of carboxymethylated dextran is covalently attached. The gold is required for generation of the SPR response, while the dextran matrix allows covalent immobilization of biomolecules using maleimide chemistry.

### Method

Channel 1: No coupling, used as negative control.

Channel 2: Immobilization of L1-CAM (10 μg/ml) on CM5 sensor chip using maleimide EDC/NHS coupling. The response measures changes in refractive index and is related to variations in mass close to the sensor surface. Therefore, the response is proportional to the number of antibody molecules interacting with antigen.

Antibody at a defined concentration is flown over CM5 chip and response captured over time, showing the progress of the interaction and association/dissociation cycle. Then, regeneration is performed to remove all remaining bound antibody from chip, and a new concentration is tested, as described herein. After different concentrations are successively tested, the kinetics parameters and affinity are calculated using BIA-evaluation software.

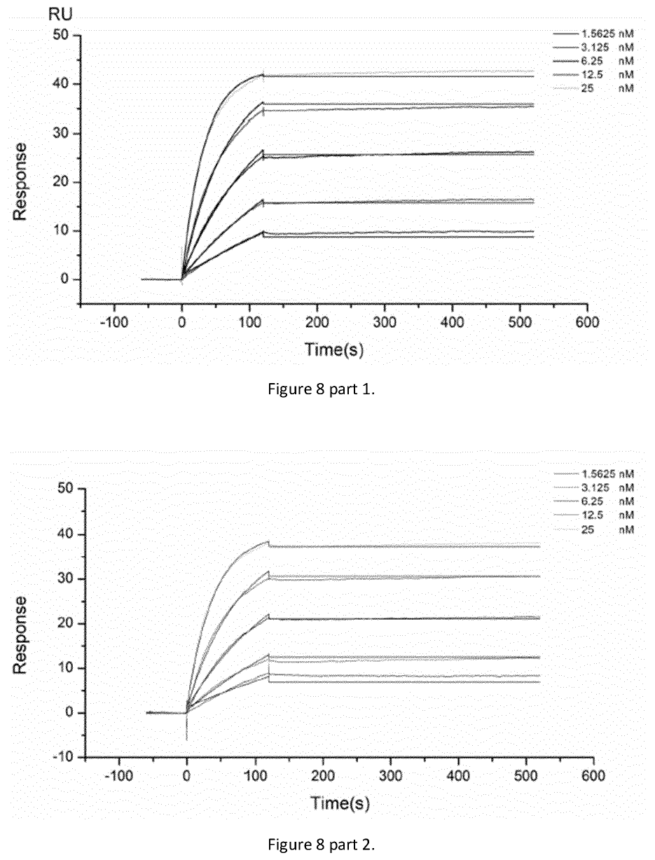

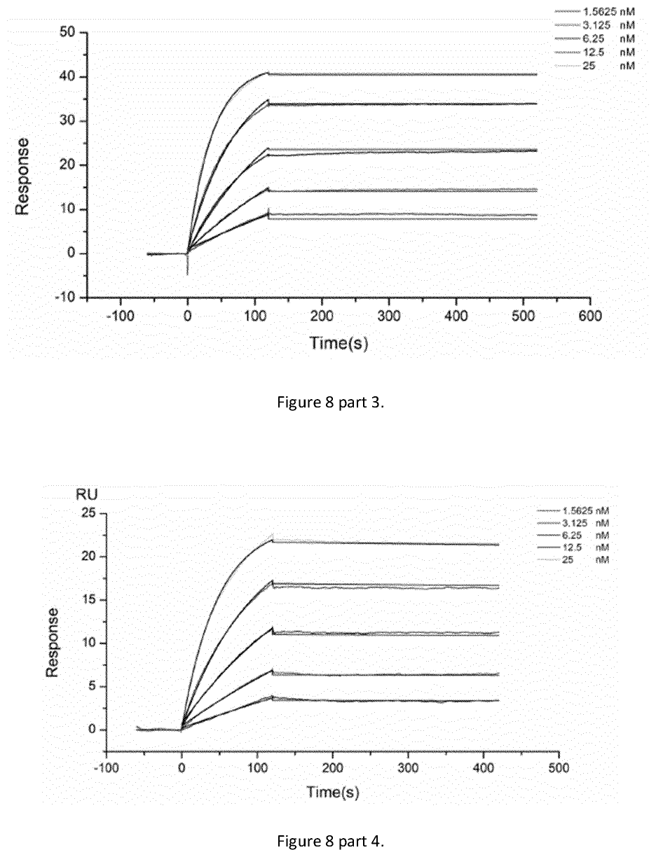

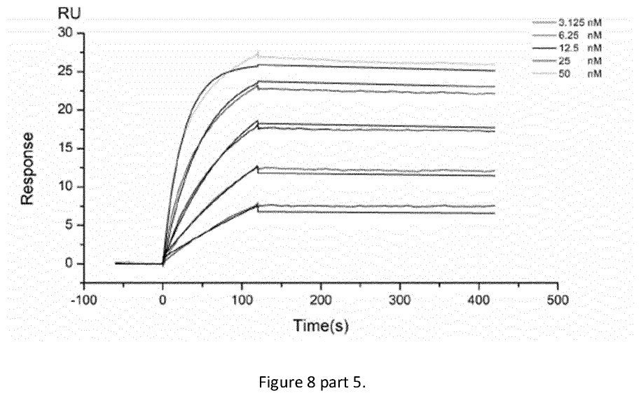

The following concentrations were tested: 1.56 nM, 3.125 nM, 6.25 nM, 32 nM, 12.5 nM and 25 nM. The sensorgrams are shown in FIG. 8, and the results are summarized in Table E9.

TABLE E9

| | Ka (1/Ms) | Kd (1/s) | KD (M) |
|---|---|---|---|
| CE7-varant4-AG | 1.23 × 10⁶ | 8.18 × 10⁻⁷ | 6.64 × 10⁻¹³ |
| CE7-varant7-AG | 1.03 × 10⁶ | 3.73 × 10⁻⁸ | 3.62 × 10⁻¹⁴ |
| CE7-varant18-AG | 1.09 × 10⁶ | 1.35 × 10⁻⁶ | 1.24 × 10⁻¹² |
| chimeric cHCE7 | 8.45 × 10⁵ | 4.39 × 10⁻⁵ | 5.19 × 10⁻¹¹ |
| Deglycosylated | 8.06 × 10⁵ | 9.59 × 10⁻⁵ | 1.2 × 10⁻¹⁰ |
| cHCE7 | | | |

The 3 humanised aglycosylated antibodies and the two control antibodies against L1-CAM have KD in 10⁻¹⁰ to 10⁻¹³ range indicative of very high affinity to the antigen for the following reasons:
Higher KD in the 10⁻¹³ range, indicative of a very strong interaction,
More favourable kinetics parameters, indicative of a fast association and no dissociation,
Sensorgram shape demonstrating a clear concentration-response relationship.

### Example 7—Determination of KD of Antibody/Antigen Interaction Via Surface Plasmon Resonance (SPR) Technology (Biacore 8K)

The KD of four monoclonal antibodies for their soluble antigen was determined by using Biacore 8K, as in Example 6.

Table E10 summarizes the material used in this study.

TABLE E10
Material information

| Product | Type | Concentration | Purity | MW (kDa) |
|---|---|---|---|---|
| L1-CAM | Ligand | 1.00 mg/ml | >90% | 143 |
| CE7-variant7 | Analyte | 0.90 mg/ml | >90% | 150 |
| CE7-variant26 | Analyte | 0.67 mg/ml | >90% | 150 |
| CE7-variant27 | Analyte | 0.94 mg/ml | >90% | 150 |
| CE7-variant28 | Analyte | 0.97 mg/ml | >90% | 150 |

It is noted that variants 26, 27 and 28 include N297A mutation and are accordingly aglycosylated. Variant 7 as used herein does not include this mutation and is expected to be glycosylated.

For the description of experimental conditions, see Example 6.

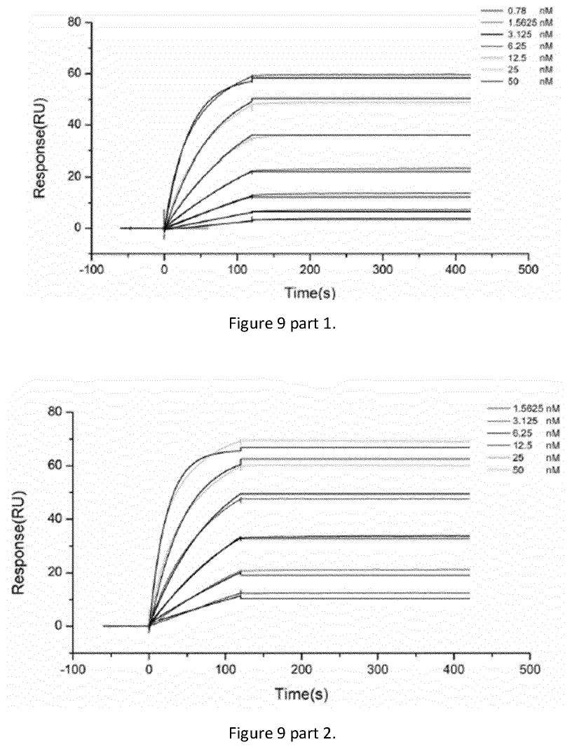

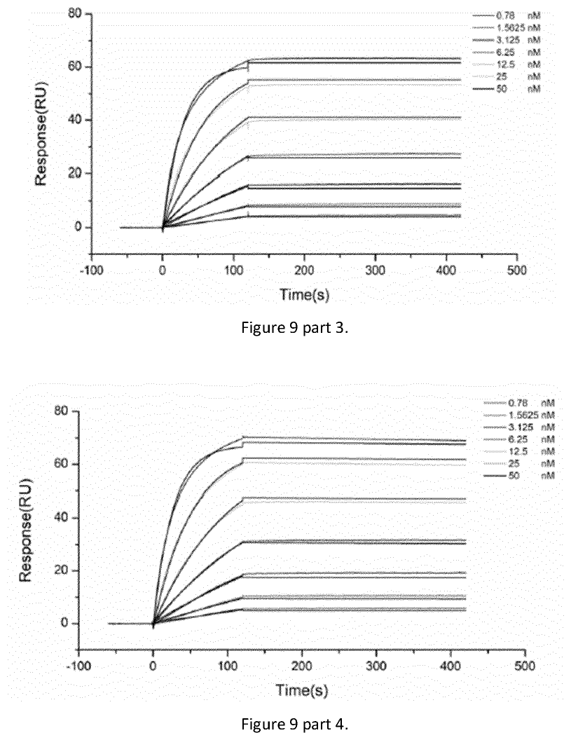

The results are summarized in FIG. 9 and Table E11.

TABLE E11
Kinetics parameters and affinity of antibody/antigen interaction

| | Ka (1/Ms) | Kd (1/s) | KD (M) |
|---|---|---|---|
| CE7-variant7 | 6.15 × 10⁵ | 5.51 × 10⁻⁷ | 8.96 × 10⁻¹³ |
| CE7-variant26 | 8.91 × 10⁵ | 1.65 × 10⁻⁷ | 1.86 × 10⁻¹³ |
| CE7-variant27 | 7.16 × 10⁵ | 1.02 × 10⁻⁷ | 1.43 × 10⁻¹³ |
| CE7-variant28 | 7.87 × 10⁵ | 3.62 × 10⁻⁵ | 4.6 × 10⁻¹¹ |

### Example 8—Production of Liability Variants 1 to 8

Starting from sequences of the Heavy chain (HC) and light chain (LC) variable regions of the additional 8 liability variants antibodies resulted from the in-silico humanization step, full-length human IgG1 Kappa antibodies harboring mutations L234A, L235A, P331 S and N297A were designed. It is to be noted that these liability variants include mutations that have been performed on the chimeric CE7 antibody framework.

The cDNA coding for the variable regions of the HC and LC were chemically synthesized with optimization for expression in CHO cells and subcloned in ProteoGenix's proprietary mammalian cells expression vectors containing backbones for human IgG1 heavy chain constant region and human kappa light chain constant region (https://www.proteogenix.science/product/xtencho-starter-kit). Sequences coding for signal peptides were added in 5′/Nter position. The sequences are illustrated below.

>CE7-VH-chimeric-WH33Y (L234A L235A + P331S N297A)
[SEQ ID NO.: 117]
GAATTCgccgccaccATGAAGCACCTGTGGTTTTTCCTGCTGCTGGTGGCCGCCCCTAGATGGGTGCT
GTCCCAGGTGCAGCTGCAGCAGCCTGGCGCCGAGCTGGTGAAGCCTGGCGCTTCCGTGAAGCTGTCCT
GTAAGGCCTCCGGCTACACCTTCACCGGCTACTACATGCACTGGGTGAAGCAGAGACCTGGCCACGGC
CTGGAGTGGATCGGCGAGATCAATCCCAGCAATGGCAGGACAAATTACAATGAGAGGTTCAAGTCCAA
GGCCACCCTGACCGTGGATAAGAGCAGCACAACAGCCTTCATGCAGCTGAGCGGCCTGACCAGCGAGG
ATTCCGCCGTGTACTTTTGTGCCAGAGATTACTACGGCACCAGCTACAACTTCGACTACTGGGGCCAG
GGCACAACACTGACAGTGAGCAGCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-VH-chimeric-WH33F (L234A L235A + P331S N297A)
[SEQ ID NO.: 118]
GAATTCgccgccaccATGAAGCACCTGTGGTTCTTCCTGCTGCTGGTGGCCGCCCCCAGGTGGGTTCT
GAGCCAGGTGCAGCTGCAGCAGCCCGGCGCTGAGCTGGTGAAGCCCGGAGCTTCCGTGAAGCTGTCCT
GCAAGGCCAGCGGCTACACCTTTACCGGCTACTTCATGCACTGGGTGAAGCAGAGACCCGGCCACGGC
CTGGAGTGGATCGGCGAAATCAACCCCAGCAACGGCAGAACCAACTACAATGAGAGATTCAAGTCCAA
GGCCACCCTGACCGTGGATAAGAGCAGCACAACAGCCTTCATGCAGCTGTCCGGCCTGACAAGCGAGG
ACAGCGCCGTGTACTTCTGTGCCAGAGATTACTACGGCACCAGCTACAATTTCGATTACTGGGGCCAG
GGCACAACCCTGACAGTGTCCTCCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-VH-chimeric-WH33L (L234A L235A + P331S N297A)
[SEQ ID NO.: 119]
GAATTCgccgccaccATGAAGCACCTGTGGTTCTTCCTGCTGCTGGTGGCCGCCCCTAGATGGGTGCT
GTCCCAGGTGCAGCTGCAGCAGCCCGGCGCTGAGCTGGTGAAGCCTGGCGCTTCCGTGAAGCTGTCCT
GTAAGGCCAGCGGCTACACCTTCACCGGCTACCTGATGCACTGGGTGAAGCAGAGACCTGGCCACGGC
CTGGAGTGGATCGGCGAGATCAATCCCAGCAATGGCAGAACAAATTACAACGAGAGGTTCAAGTCCAA
GGCCACCCTGACCGTGGACAAGTCCTCCACCACCGCCTTCATGCAGCTGTCCGGCCTGACCAGCGAGG
ACAGCGCCGTGTACTTCTGTGCCAGGGACTACTACGGCACCAGCTACAATTTCGACTACTGGGGCCAG
GGCACCACACTGACCGTGTCCAGCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-VH-chimeric-GH55A (L234A L235A + P331S N297A)
[SEQ ID NO.: 120]
GAATTCgccgccaccATGAAGCACCTGTGGTTCTTTCTGCTGCTGGTGGCCGCCCCTAGGTGGGTGCT
GTCCCAGGTGCAGCTGCAGCAGCCTGGCGCCGAGCTGGTGAAGCCCGGAGCTTCCGTGAAGCTGTCCT
GCAAGGCCAGCGGCTACACCTTCACAGGCTACTGGATGCACTGGGTGAAGCAGAGGCCCGGCCACGGC
CTGGAGTGGATCGGAGAGATCAACCCCTCCAACGCCAGGACCAATTACAACGAGAGATTCAAATCCAA
GGCCACCCTGACCGTGGACAAGAGCAGCACAACAGCCTTCATGCAGCTGAGCGGCCTGACAAGCGAGG
ATTCCGCCGTGTACTTTTGCGCCAGGGATTACTACGGCACAAGCTACAATTTTGATTACTGGGGCCAG
GGCACCACCCTGACCGTCAGCAGCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-VH-chimeric (L234A L235A + P331S N297A)
[SEQ ID NO.: 121]
GAATTCgccgccaccATGAAGCACCTGTGGTTCTTTCTGCTGCTGGTGGCCGCCCCCAGGTG
GGTTCTGAGCCAGGTGCAGCTGCAGCAGCCTGGCGCCGAGCTGGTGAAGCCTGGCGCTTCCG
TGAAGCTGAGCTGCAAGGCCTCCGGCTACACATTCACTGGCTACTGGATGCACTGGGTGAAG
CAGAGACCTGGCCACGGCCTGGAGTGGATCGGCGAGATCAATCCCAGCAATGGCAGAACCAA
CTACAACGAGAGGTTCAAGTCCAAGGCCACCCTGACCGTGGATAAGAGCTCCACCACCGCCT
TCATGCAGCTGTCCGGCCTGACCAGCGAGGACAGCGCCGTGTACTTTTGTGCCAGAGATTAC
TACGGCACCAGCTACAACTTTGATTACTGGGGCCAGGGCACAACCCTGACAGTGAGCTCCGC
TAGCACCAAGGGACCTTCTGTGTTCCCTCTGGCTCCTTCTTCTAAGTCCACTTCCGGTGGTA
CAGCAGCTCTGGGTTGTCTGGTGAAGGATTACTTCCCAGAACCAGTGACTGTGTCCTGGAAC
TCCGGAGCTCTGACTTCTGGAGTGCATACTTTCCCAGCAGTGCTGCAATCTAGCGGACTGTA
CTCTCTGTCTTCCGTGGTGACTGTGCCTTCTTCTTCCCTGGGGACTCAAACTTACATCTGCA
ACGTGAACCACAAGCCCTCCAACACCAAGGTGGACAAGAAGGTGGAGCCAAAGAGCTGCGAT
AAGACCCACACCTGTCCACCTTGTCCAGCTCCAGAAGCCGCCGGTGGGCCTTCTGTGTTTCT
GTTCCCACCTAAGCCAAAGGATACCCTGATGATCTCTAGGACCCCAGAAGTGACCTGTGTGG
TCGTCGATGTGTCTCATGAAGACCCTGAAGTGAAGTTCAACTGGTACGTGGACGGGGTGGAA
GTGCATAACGCAAAGACCAAGCCCAGGGAAGAGCAATACGCTTCCACCTACAGGGTGGTCTC
CGTCCTGACAGTCCTGCATCAGGATTGGCTGAACGGCAAGGAGTACAAGTGCAAGGTCTCCA
ATAAAGCCCTGCCTGCCAGCATCGAGAAAACCATTAGCAAAGCCAAAGGCCAGCCCAGGGAG
CCCCAGGTCTATACACTGCCCCCCAGCAGGGAGGAGATGACAAAAAATCAGGTCAGCCTGAC
ATGCCTGGTCAAAGGCTTTTATCCCAGCGACATTGCCGTCGAGTGGGAGTCCAATGGCCAGC
CCGAGAATAATTATAAAACAACACCCCCCGTCCTGGACAGCGACGGCAGCTTTTTTCTGTAT
AGCAAACTGACAGTCGATAAAAGCAGGTGGCAGCAGGGCAATGTCTTTTCCTGCAGCGTCAT
GCACGAGGCCCTGCACAATCACTATACTCAGAAAAGCCTGAGCCTGTCCCCCGGGAAATGAa
agctt
>CE7-VL-chimeric
[SEQ ID NO.: 122]
GAATTCgccgccaccATGGTGCTGCAGACCCAGGTGTTTATCTCCCTGCTGCTGTGGATCAG
CGGCGCCTACGGCGATATCCAGATGACCCAGAGCTCCAGCTCCTTTAGCGTGAGCCTGGGCG
ACCGGGTGACCATCACCTGTAAGGCTAATGAGGACATCAATAACAGGCTGGCTTGGTACCAG
CAGACCCCCGGCAATAGCCCTAGGCTGCTGATCAGCGGCGCTACCAATCTGGTGACCGGCGT
GCCTAGCAGGTTTAGCGGCTCCGGCTCCGGCAAGGATTACACCCTGACCATCACCTCCCTGC
AGGCCGAGGATTTTGCTACCTACTATTGTCAGCAGTACTGGAGCACCCCTTTTACCTTTGGC
TCCGGCACCGAGCTGGAGATCAAGcgtacgGTGGCTGCACCTTCTGTGTTCATCTTCCCTCC
ATCTGATGAGCAGCTGAAGTCTGGAACCGCATCTGTCGTCTGTCTGCTGAACAACTTTTACC
CCAGGGAGGCTAAGGTCCAATGGAAGGTGGACAACGCCCTGCAGTCTGGTAATAGCCAGGAA
AGCGTGACCGAACAGGATTCCAAGGACTCCACCTACTCCCTGTCCTCCACACTGACACTGAG
CAAAGCCGACTATGAAAAGCACAAAGTGTATGCCTGCGAGGTCACTCATCAGGGCCTGTCCA
GCCCCGTGACTAAAAGCTTTAATAGGGGGGAGTGCTGAGCGGCCGC
>CE7-VL-chimeric-NL31L
[SEQ ID NO.: 123]
GAATTCgccgccaccATGGTGCTGCAGACCCAGGTGTTCATCAGCCTGCTGCTGTGGATCAGCGGCGC
TTATGGCGACATCCAGATGACCCAGAGCTCCTCCTCCTTTTCCGTGTCCCTGGGCGATCGGGTGACCA
TCACCTGTAAGGCCAATGAGGATATCAACCTGAGGCTGGCCTGGTACCAGCAGACCCCTGGCAACAGC
CCTAGGCTGCTGATCTCCGGCGCTACCAATCTGGTGACCGGCGTGCCTAGCCGGTTTAGCGGCTCCGG
CAGCGGCAAGGACTATACCCTGACCATCACCAGCCTGCAGGCCGAGGACTTTGCTACCTATTATTGTC
AGCAGTACTGGAGCACCCCCTTTACCTTTGGCTCCGGCACCGAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-VL-chimeric-NL31A
[SEQ ID NO.: 124]
GAATTCgccgccaccATGGTGCTGCAGACCCAGGTGTTCATCTCCCTGCTGCTGTGGATCTCCGGCGC
TTATGGCGATATCCAGATGACCCAGAGCTCCAGCAGCTTCTCCGTGAGCCTGGGCGATAGGGTGACCA
TCACCTGTAAGGCCAACGAGGATATCAATGCTAGGCTGGCTTGGTATCAGCAGACCCCTGGCAATAGC
CCCCGGCTGCTGATCTCCGGCGCCACAAACCTGGTGACCGGCGTGCCTTCCAGGTTCTCCGGCAGCGG
CTCCGGCAAGGATTATACCCTGACCATCACCAGCCTGCAGGCTGAGGATTTTGCTACCTACTACTGTC
AGCAGTATTGGAGCACCCCCTTTACCTTTGGCTCCGGCACCGAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-VL-chimeric-WL92Y
[SEQ ID NO.: 125]
GAATTCgccgccaccATGGTGCTGCAGACCCAGGTGTTTATCAGCCTGCTGCTGTGGATCTCCGGCGC
TTATGGCGATATCCAGATGACCCAGAGCTCCTCCTCCTTTAGCGTGAGCCTGGGCGATCGGGTGACCA
TCACCTGTAAGGCCAACGAGGACATCAATAACAGGCTGGCTTGGTACCAGCAGACCCCTGGCAACAGC
CCTCGGCTGCTGATCAGCGGCGCCACCAATCTGGTGACCGGCGTGCCCAGCAGGTTCAGCGGCAGCGG
ATCCGGCAAGGACTACACCCTGACCATCACCAGCCTGCAGGCTGAGGACTTTGCCACCTATTACTGTC
AGCAGTACTATTCCACCCCTTTCACCTTCGGCTCCGGCACCGAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-VL-chimeric-WL92F
[SEQ ID NO.: 126]
GAATTCgccgccaccATGGTGCTGCAGACCCAGGTGTTTATCTCCCTGCTGCTGTGGATCAGCGGCGC
TTATGGCGACATCCAGATGACCCAGAGCAGCTCCAGCTTCTCCGTGTCCCTGGGCGACCGGGTGACCA
TCACCTGTAAGGCCAATGAGGATATCAACAACCGGCTGGCCTGGTATCAGCAGACCCCTGGCAACAGC
CCCAGGCTGCTGATCTCCGGCGCCACCAATCTGGTGACCGGCGTGCCCTCCCGGTTTTCCGGCAGCGG
AAGCGGCAAGGATTACACCCTGACCATCACCTCCCTGCAGGCTGAGGATTTCGCTACCTACTATTGCC
AGCAGTACTTTTCCACCCCCTTTACCTTTGGCTCCGGCACCGAGCTGGAGATCAAGcgtacgGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
The sequences for the expected proteins produced are illustrated below.
>CE7-VH-chimeric-WH33Y (L234A L235A + P331S N297A)
[SEQ ID NO.: 127]
MKHLWFFLLLVAAPRWVLSQVQLQQPGAELVKPGASVKLSCKASGYTFTGYYMHWVKQRPGHGLEWIG
EINPSNGRTNYNERFKSKATLTVDKSSTTAFMQLSGLTSEDSAVYFCARDYYGTSYNFDYWGQGTTLT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-VH-chimeric-WH33F (L234A L235A + P331S N297A)
[SEQ ID NO.: 128]
MKHLWFFLLLVAAPRWVLSQVQLQQPGAELVKPGASVKLSCKASGYTFTGYFMHWVKQRPGHGLEWIG
EINPSNGRTNYNERFKSKATLTVDKSSTTAFMQLSGLTSEDSAVYFCARDYYGTSYNFDYWGQGTTLT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKFNWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-VH-chimeric-WH33L (L234A L235A + P331S N297A)
[SEQ ID NO.: 129]
MKHLWFFLLLVAAPRWVLSQVQLQQPGAELVKPGASVKLSCKASGYTFTGYLMHWVKQRPGHGLEWIG
EINPSNGRTNYNERFKSKATLTVDKSSTTAFMQLSGLTSEDSAVYFCARDYYGTSYNFDYWGQGTTLT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-VH-chimeric-GH55A (L234A L235A + P331S N297A)
[SEQ ID NO.: 130]
MKHLWFFLLLVAAPRWVLSQVQLQQPGAELVKPGASVKLSCKASGYTFTGYWMHWVKQRPGHGLEWIG
EINPSNARTNYNERFKSKATLTVDKSSTTAFMQLSGLTSEDSAVYFCARDYYGTSYNFDYWGQGTTLT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-VH-chimeric (L234A L235A + P331S N297A)
[SEQ ID NO.: 131]
MKHLWFFLLLVAAPRWVLSQVQLQQPGAELVKPGASVKLSCKASGYTFTGYWMHWVKQRPGHGLEWIG
EINPSNGRTNYNERFKSKATLTVDKSSTTAFMQLSGLTSEDSAVYFCARDYYGTSYNFDYWGQGTTLT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-VL-chimeric
[SEQ ID NO.: 132]
MVLQTQVFISLLLWISGAYGDIQMTQSSSSFSVSLGDRVTITCKANEDINNRLAWYQQTPGNSPRLLI
SGATNLVTGVPSRESGSGSGKDYTLTITSLQAEDFATYYCQQYWSTPFTFGSGTELEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-VL-chimeric-NL31L
[SEQ ID NO.: 133]
MVLQTQVFISLLLWISGAYGDIQMTQSSSSFSVSLGDRVTITCKANEDINLRLAWYQQTPGNSPRLLI
SGATNLVTGVPSRESGSGSGKDYTLTITSLQAEDFATYYCQQYWSTPFTFGSGTELEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-VL-chimeric-NL31A
[SEQ ID NO.: 134]
MVLQTQVFISLLLWISGAYGDIQMTQSSSSFSVSLGDRVTITCKANEDINARLAWYQQTPGNSPRLLI
SGATNLVTGVPSRESGSGSGKDYTLTITSLQAEDFATYYCQQYWSTPFTFGSGTELEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-VL-chimeric-WL92Y
[SEQ ID NO.: 135]
MVLQTQVFISLLLWISGAYGDIQMTQSSSSFSVSLGDRVTITCKANEDINNRLAWYQQTPGNSPRLLI
SGATNLVTGVPSRESGSGSGKDYTLTITSLQAEDFATYYCQQYYSTPFTFGSGTELEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-VL-chimeric-WL92F
[SEQ ID NO.: 136]
MVLQTQVFISLLLWISGAYGDIQMTQSSSSFSVSLGDRVTITCKANEDINNRLAWYQQTPGNSPRLLI
SGATNLVTGVPSRESGSGSGKDYTLTITSLQAEDFATYYCQQYFSTPFTEGSGTELEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC

An endotoxin-free DNA preparation was done for the constructions obtained as described above. The eight antibodies were expressed by combining HC and LC as follows:

TABLE E12

| Antibody ID | Heavy Chain | Light Chain |
|---|---|---|
| LV1 | pTXs1-CE7-VH-chimeric-WH33Y | pTXs1-CE7-VL-chimeric |
| LV2 | pTXs1-CE7-VH-chimeric-WH33F | pTXs1-CE7-VL-chimeric |
| LV3 | pTXs1-CE7-VH-chimeric-WH33L | pTXs1-CE7-VL-chimeric |
| LV4 | pTXs1-CE7-VH-chimeric-GH55A | pTXs1-CE7-VL-chimeric |
| LV5 | pTXs1-CE7-VH-chimeric | pTXs1-CE7-VL-chimeric-NL31L |
| LV6 | pTXs1-CE7-VH-chimeric | pTXs1-CE7-VL-chimeric-NL31A |
| LV7 | pTXs1-CE7-VH-chimeric | pTXs1-CE7-VL-chimeric-WL92Y |
| LV8 | pTXs1-CE7-VH-chimeric | pTXs1-CE7-VL-chimeric-WL92F |

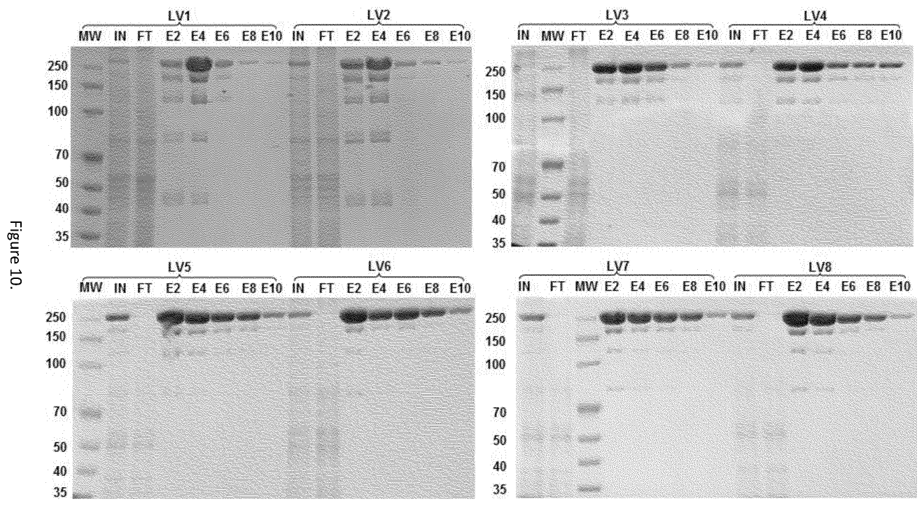

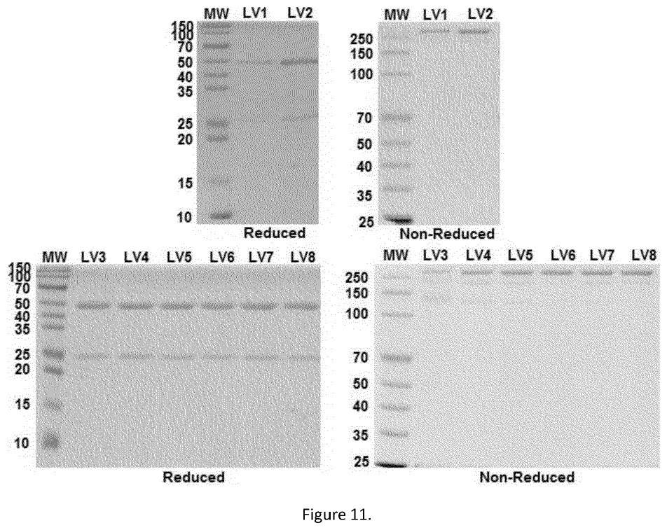

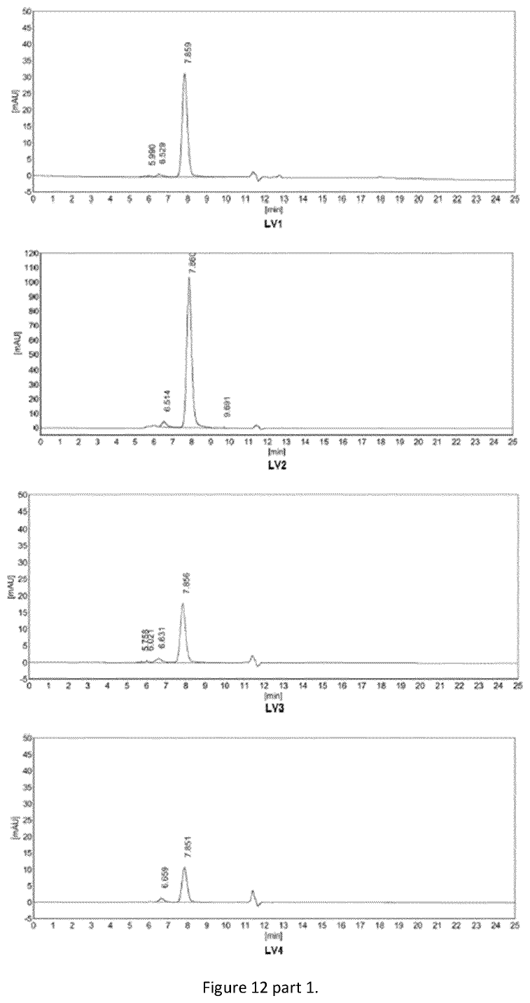

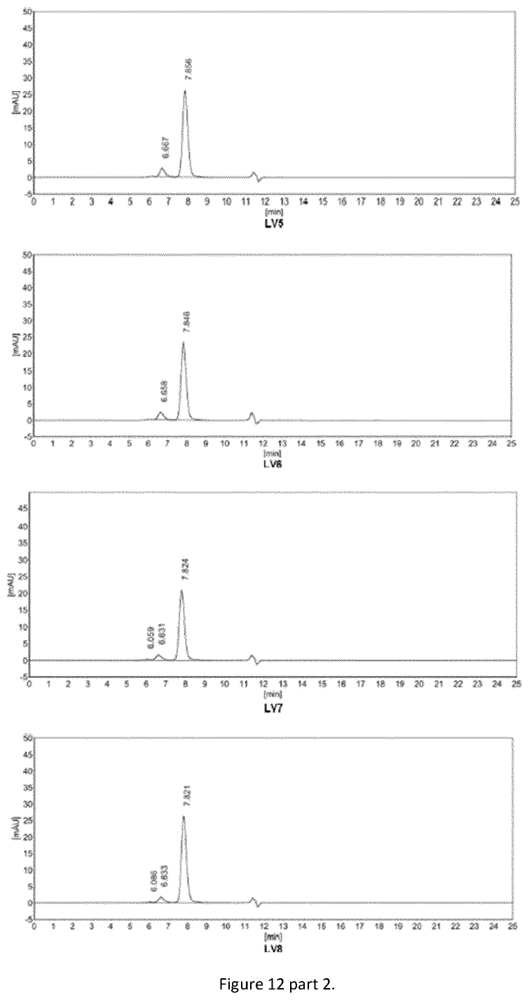

The methods as described in Example 2 or Example 3 were used. Purification profiles and final QC are illustrated in FIGS. 10 and 11. The yield and purity of obtained antibodies are summarized in Table E13.

TABLE E13
Yield and purity obtained for the rAb pilot productions

| Anti- | Concen- | Specifi- | | | |
|---|---|---|---|---|---|
| body | tration | cation | Quantity | Yield* | Purity** |
| LV1 | 0.59 mg/ml | 1.30 ml/vial | 2vials | 1.53 mg/ | >90% |
| | | | | 30 ml | |
| LV2 | 1.73 mg/ml | 0.70 ml/vial | 1 vial | 1.21 mg/ | >90% |
| | | | | 30 ml | |
| LV3 | 0.37 mg/ml | 1.40 ml/vial | 2vials | 1.04 mg/ | >90% |
| | | | | 30 ml | |
| LV4 | 0.20 mg/ml | 1.60 ml/vial | 4vials | 1.28 mg/ | >90% |
| | | | | 30 ml | |
| LV5 | 0.51 mg/ml | 1.58 ml/vial | 3vials | 2.42 mg/ | >90% |
| | | | | 30 ml | |
| LV6 | 0.54 mg/ml | 1.50 ml/vial | 3vials | 2.43 mg/ | >90% |
| | | | | 30 ml | |
| LV7 | 0.40 mg/ml | 1.50 ml/vial | 3vials | 1.80 mg/ | >90% |
| | | | | 30 ml | |
| LV8 | 0.48 mg/ml | 1.57 ml/vial | 3vials | 2.26 mg/ | >90% |
| | | | | 30 ml | |

*Obtained after purification for the 30 ml-culture test.
**Based on full length antibody observed on non-reduced PAGE analyses in FIG. 2.
Final buffer: PBS pH 7.5.

### Example 9—SEC-HPLC Analysis for Liability Variants

### Short Protocol Description

SEC-HPLC analysis was performed evaluate the purity level and to quantify the proportion of soluble aggregates for the liability antibody samples. The analyses were performed with a HPLC Waters 2695 device equipped with a Photodiode Array Detector (2996).

### Buffer Preparation

Mobile phase: 100 mM Sodium sulfate, 100 mM Phosphate salt, pH 6.7 buffer (Na₂SO₄, 14.2 g/L, Na₂HPO₄·12H₂O 15.6 g/L, NaH₂PO₄ 6.8 g/L)

Air bubbles are removed from all buffers.

### Sample Preparation

Protein preparation: Starting from one extra aliquot of each final sample store at −80° C. for one night, dilution of the protein samples to 1 mg/ml with DD water
Centrifugation at 12000 g during 10 min and collection of supernatants

### Analysis Conditions

Blank control: dilution (5×) of the sample with DD water
Column: G3000SWXL, TOSOH, 7.8×300 mm
HPLC: Waters 2695
Elution gradient: mobile phase isocratic elution
Flow rate: 0.8 ml/min
Temperature: 25° C.
Injection volume: 30 μl
Detection wavelength: 280 nm
Collect date time: 25 min
Equilibration with mobile phase 100% for 5 min
Run Samples: Injection of one blank control and then injection of samples
Data Analysis

The results are summarized in Table E14.

TABLE E14
SEC-HPLC Data

| | time | Area | Height | Purity |
|---|---|---|---|---|
| | (min) | (μV/s) | (μV/s) | (%) |
| LV1 | 5.99 | 8439.5 | 307.8 | |
| | 6.529 | 17649.7 | 743.4 | |
| | 7.859 | 596216.1 | 31290.8 | 95.81% |
| LV2 | 6.514 | 65233.3 | 3416.7 | |
| | 7.86 | 1969854 | 102525 | 96.77% |
| | 9.691 | 545.1 | 25.2 | |
| LV3 | 5.758 | 2358.6 | 176.4 | |
| | 6.021 | 5902.4 | 295 | |
| | 6.631 | 27752.4 | 1040 | |
| | 7.856 | 337212 | 17366.1 | 90.35% |
| LV4 | 6.659 | 21928.3 | 1072 | |
| | 7.851 | 198552.7 | 10394.7 | 90.05% |
| LV5 | 6.667 | 50859.3 | 2497.9 | |
| | 7.856 | 499361.6 | 25958 | 90.76% |
| LV6 | 6.658 | 43482 | 2134 | |
| | 7.846 | 439005.1 | 22974.2 | 90.99% |
| | 6.658 | 43482 | 2134 | |
| LV7 | 6.059 | 6868.6 | 275.8 | |
| | 6.631 | 36428.3 | 1539.7 | |
| | 7.824 | 398441.4 | 20722.7 | 90.20% |
| LV8 | 6.086 | 4374.4 | 199.1 | |
| | 6.633 | 37835.8 | 1647.9 | |
| | 7.821 | 499656.2 | 26061.6 | 92.21% |
| SEC 300A | 5.969 | 147470.1 | 6143.9 | |
| Protein Standard | 8.013 | 296338 | 9522.8 | |
| | 9.538 | 171051.9 | 7955 | |
| | 10.444 | 170933.6 | 12339.7 | |
| | 11.951 | 107265.3 | 5496.2 | |

When several peaks are detected, data corresponding to the peak of full antibody single molecules is indicated in bold.
When a single peak is detected, an estimate of the concentration (purity) is given.

### Example 10. Differential Scanning Fluorimetry Analysis

### Short Protocol Description

DSF analyses were performed to evaluate the thermal stability (unfolding) of the 18 humanised variants antibodies. DSF work was performed by using a Nanotemper-nanoDSF system with a standard method. The mAbs were subject to a linear thermal ramp of 1° C./min, from 40° to 90° C. Tryptophan fluorescence at 350 nm and 330 nm was collected at a rate of 10 datapoints per minute. Unfolding transition midpoints were determined automatically from the 2^{nd} derivative of the fluorescence ratio (F350/F330). The samples are summarized in Table E15.

TABLE E15
DSF Samples.

| Sample | Quantity | Storage Buffer |
|---|---|---|
| CE7-variant1 | 1.00 mg/ml, 50 ul/vial | PBS, pH 7.5 |
| CE7-variant2 | 1.00 mg/ml, 50 ul/vial | PBS, pH 7.5 |
| CE7-variant3 | 1.00 mg/ml, 50 ul/vial | PBS, pH 7.5 |
| CE7-variant4 | 1.00 mg/ml, 50 ul/vial | PBS, pH 7.5 |
| CE7-variant5 | 1.00 mg/ml, 50 ul/vial | PBS, pH 7.5 |
| CE7-variant6 | 1.00 mg/ml, 50 ul/vial | PBS, pH 7.5 |
| CE7-variant7 | 1.00 mg/ml, 50 ul/vial | PBS, pH 7.5 |
| CE7-variant8 | 1.00 mg/ml, 50 ul/vial | PBS, pH 7.5 |
| CE7-variant9 | 1.00 mg/ml, 50 ul/vial | PBS, pH 7.5 |
| CE7-variant10 | 1.00 mg/ml, 50 ul/vial | PBS, pH 7.5 |
| CE7-variant11 | 1.00 mg/ml, 50 ul/vial | PBS, pH 7.5 |
| CE7-variant12 | 1.00 mg/ml, 50 ul/vial | PBS, pH 7.5 |
| CE7-variant13 | 1.00 mg/ml, 50 ul/vial | PBS, pH 7.5 |
| CE7-variant14 | 1.00 mg/ml, 50 ul/vial | PBS, pH 7.5 |
| CE7-variant15 | 1.00 mg/ml, 50 ul/vial | PBS, pH 7.5 |
| CE7-variant16 | 1.00 mg/ml, 50 ul/vial | PBS, pH 7.5 |
| CE7-variant17 | 1.00 mg/ml, 50 ul/vial | PBS, pH 7.5 |
| CE7-variant18 | 1.00 mg/ml, 50 ul/vial | PBS, pH 7.5 |

The results of the analysis are shown in Table E16.

TABLE E16
Tm values of each antibody.

| Sample | Tm 1(° C.) | Tm 2(° C.) |
|---|---|---|
| CE7- variant1 | 67.8° C. | 78.9° C. |
| CE7- variant2 | 67.9° C. | 78.7° C. |
| CE7- variant3 | 68.0° C. | 79.9° C. |
| CE7- variant4 | 67.8° C. | 78.9° C. |
| CE7- variant5 | 67.3° C. | 81.0° C. |
| CE7- variant6 | 68.0° C. | 77.3° C. |
| CE7- variant7 | 67.9° C. | 80.6° C. |
| CE7- variant8 | 68.2° C. | 80.1° C. |
| CE7- variant9 | 68.1° C. | 81.7° C. |
| CE7-variant10 | 67.5° C. | 80.6° C. |
| CE7-variant11 | 67.9° C. | 80.6° C. |
| CE7-variant12 | 68.0° C. | 79.2° C. |
| CE7-variant13 | 67.0° C. | 74.3° C. |
| CE7-variant14 | 67.2° C. | 74.2° C. |
| CE7-variant15 | 67.0° C. | 74.0° C. |
| CE7-variant16 | 67.8° C. | 75.0° C. |
| CE7-variant17 | 67.4° C. | 73.9° C. |
| CE7-variant18 | 67.4° C. | 73.9° C. |

All antibodies showed two unfolding transitions in the plot of the fluorescence ratio (F330/F350) versus temperature. The distinct unfolding events can be attributed to the different thermal stabilities of Fab- and Fc-domains of the antibodies. DSF analyses show that variants 5, 7, 8, 9, 10 and 11 have a similar thermal stability, with a Tm1 of ˜68° C. and Tm2 of ˜80° C. Variants 13, 14, 15, 16, 17 and 18 exhibit a slight decrease of the Tm2 (˜74° C.).

### Example 11. Differential Scanning Fluorimetry Analysis

Further analysis as in Example 10 has been performed on samples summarized in Table E17.

TABLE E17
DSF samples

| | | |
|---|---|---|
| CE7-variant 4 AG | 1.00 mg/ml, 50 ul/vial | PBS, pH 7.5 |
| CE7-variant 7 AG | 1.00 mg/ml, 50 ul/vial | PBS, pH 7.5 |
| CE7-variant 18 AG | 1.00 mg/ml, 50 ul/vial | PBS, pH 7.5 |
| chimeric cHCE7 | 1.00 mg/ml, 50 ul/vial | PBS, pH 7.5 |
| Deglycosilated cHCE7 | 1.00 mg/ml, 50 ul/vial | PBS, pH 7.5 |

The results of the analysis are shown in Table E18.

TABLE E18
Tm values for tested antibodies

| Sample | Tm 1(° C.) | Tm 2(° C.) |
|---|---|---|
| CE7-variant 4 AG | 58.7° C. | 80.0° C. |
| CE7-variant 7 AG | 58.3° C. | 80.2° C. |
| CE7-variant 18 AG | 58.4° C. | 74.3° C. |
| chimeric cHCE7 | 66.0° C. | 73.8° C. |
| Deglycosilated | 60.2° C. | 73.6° C. |
| cHCE7 | | |

The DSF analysis is similar to that seen in Example 10, showing two unfolding events. Tm1 and Tm2 of the chimeric antibody are comparable to Tm1 of the non glycosylated variants. The aglycosylated chimeric antibody has similar Tm1 value as the 3 humanised variants but lower than the Tm1 of the chimeric antibody, which might correspond to the unfolding event of the Fc domain. The Tm2 values for the 3 humanised aglycosylated variants are high and very similar to the Tm2 values obtained for the same non glycosylated versions. This indicates that Tm2 might correspond to the unfolding of the Fab domains.

### Example 12. Differential Scanning Fluorimetry Analysis

Further analysis as in Example 10 has been performed on samples summarized in Table E19. It is noted that all the variants described therein, except for CE7-variant7, are aglycosylated (i.e., include mutation N297A in the heavy chain).

TABLE E19
DSF samples

| | | |
|---|---|---|
| CE7-variant7 | 1.00 mg/ml, 50 ul/vial | PBS, pH 7.5 |
| CE7-variant7-AG | 1.00 mg/ml, 50 ul/vial | PBS, pH 7.5 |
| CE7-variant19 | 1.00 mg/ml, 50 ul/vial | PBS, pH 7.5 |
| CE7-variant20 | 1.00 mg/ml, 50 ul/vial | PBS, pH 7.5 |
| CE7-variant21 | 1.00 mg/ml, 50 ul/vial | PBS, pH 7.5 |
| CE7-variant22 | 1.00 mg/ml, 50 ul/vial | PBS, pH 7.5 |
| CE7-variant23 | 1.00 mg/ml, 50 ul/vial | PBS, pH 7.5 |
| CE7-variant24 | 1.00 mg/ml, 50 ul/vial | PBS, pH 7.5 |
| CE7-variant25 | 1.00 mg/ml, 50 ul/vial | PBS, pH 7.5 |
| CE7-variant26 | 1.00 mg/ml, 50 ul/vial | PBS, pH 7.5 |
| CE7-variant27 | 1.00 mg/ml, 50 ul/vial | PBS, pH 7.5 |
| CE7-variant28 | 1.00 mg/ml, 50 ul/vial | PBS, pH 7.5 |
| LV1 | 1.00 mg/ml, 50 ul/vial | PBS, pH 7.5 |
| LV2 | 1.00 mg/ml, 50 ul/vial | PBS, pH 7.5 |
| LV3 | 1.00 mg/ml, 50 ul/vial | PBS, pH 7.5 |
| LV4 | 1.00 mg/ml, 50 ul/vial | PBS, pH 7.5 |
| LV5 | 1.00 mg/ml, 50 ul/vial | PBS, pH 7.5 |
| LV6 | 1.00 mg/ml, 50 ul/vial | PBS, pH 7.5 |
| LV7 | 1.00 mg/ml, 50 ul/vial | PBS, pH 7.5 |
| LV8 | 1.00 mg/ml, 50 ul/vial | PBS, pH 7.5 |

The results of the analysis are shown in Table E20.

TABLE E20
Tm values for tested antibodies.

| Sample | Tm 1(° C.) | Tm 2(° C.) |
|---|---|---|
| CE7- variant7 | 67.8° C. | 81.9° C. |
| CE7- variant7-AG | 57.5° C. | 81.6° C. |
| CE7- variant19 | 56.8° C. | 82.4° C. |
| CE7- variant20 | 57.0° C. | 82.4° C. |
| CE7- variant21 | 56.7° C. | 82.8° C. |
| CE7- variant22 | 57.8° C. | 81.7° C. |
| CE7- variant23 | 58.1° C. | 81.9° C. |
| CE7- variant24 | 58.0° C. | 82.0° C. |
| CE7- variant25 | 58.0° C. | 80.0° C. |
| CE7- variant26 | 57.4° C. | 80.7° C. |
| CE7- variant27 | 57.0° C. | 81.4° C. |
| CE7- variant28 | 57.7° C. | 81.0° C. |
| LV1 | 58.1° C. | 74.0° C. |
| LV2 | 58.1° C. | 73.6° C. |
| LV3 | 57.6° C. | 68.2° C. |
| LV4 | 59.8° C. | 67.4° C. |
| LV5 | 57.3° C. | 73.0° C. |
| LV6 | 57.9° C. | 71.1° C. |
| LV7 | 57.5° C. | 72.6° C. |
| LV8 | 56.5° C. | 73.5° C. |

All antibodies showed two unfolding transitions in the plot of the fluorescence ratio (F330/F350) versus temperature (FIG. 1). The distinct unfolding events can be attributed to the different thermal stabilities of Fab- and Fc-domains of the antibodies. DSF analyses show that the additional variants 19 to 28 have a similar thermal stability, with a Tm1 of ˜57° C. and Tm2 of ˜80° C., very similar to Variant7-AG. Liability variants LV1 to LV8 exhibit a slight decrease of the Tm2 (˜72° C.).

### Example 13—Affinity of Antibodies for L1-CAM—An ELISA Study

The affinity of antibodies for L1-CAM has been determined using an ELISA method, according to the following protocol:
1. Antigen coating: human L1-CAM, 2 μg/ml, 100 μl/well, 37° C., 2 h
2. Blocking: 3% BSA-PBS, 300 μl/well, 37° C., 1.5 h
3. Washing: PBST, 300 μl/well, 2 times
4. Primary Antibody (humanised variants), 100 μl/well, 37° C., 30 min
5. Washing: PBST, 300 μl/well, 3 times
6. Secondary Antibody, 100 μl/well, 37° C., 30 min
7. Washing: PBST, 300 μl/well, 3 times
8. TMB Substrate Solution: 100 μl/well, 37° C., 5-10 min
9. Stop: 2M HCl, 50 μl/well
10. Reading: OD450-OD630

TABLE E21

| humanized | |
|---|---|
| Variant | EC50(ng/ml) |
| 1 | 28.8 |
| 2 | 12.78 |
| 3 | 34.26 |
| 4 | 12 |
| 4 AG | — |
| 5 | 28.24 |
| 6 | 25.45 |
| 7 | 9.585/32.7 |
| 7 AG | 25.8 |
| 8 | 12.92 |
| 9 | 19.67 |
| 10 | 13.63 |
| 11 | 14.77 |
| 12 | 12.63 |
| 13 | 50.03 |
| 14 | 15.71 |
| 15 | 11.24 |
| 15 AG | — |
| 16 | 13.23 |
| 17 | 10.79 |
| 17 AG | — |
| 18 | 9.683 |
| 18 AG | — |
| 19 | 33.5 |
| 20 | 31.7 |
| 21 | 29.9 |
| 22 | 24 |
| 23 | 37.05 |
| 24 | 39.56 |
| 25 | 27.25 |
| 26 | 12.67 |
| 27 | 20.94 |
| 28 | 70.06 |
| LV 1 | 36.19 |
| LV 2 | 45.9 |
| LV 3 | 32.84 |
| LV 4 | 39.35 |
| LV 5 | 31.77 |
| LV 6 | 26.83 |
| LV 7 | 24.65 |
| LV 8 | 33.53 |
| cHCE7 | 6.627/23.15 |
| cHCE7 degly | 13.08 |

### Example 14—GCI Studies of Antibody Binding

In this study, binding of certain antibodies of the invention to L1-CAM was examined.

### Rationale:

This experiment should investigate binding of L1-CAM to protein A/G captured antibodies in a multi-cycle kinetics experiment, see FAQ.
The ligands will be freshly captured for each analysis cycle.
The analyte will be injected in increasing concentrations over ligand and reference surfaces.
After each analysis cycle, ligand-analyte complexes will be removed from the capture surface by injection of regeneration solution.
A DMSO solvent correction curve for solvent correction will be established, see FAQ.
Raw sensorgrams will be inspected for non-specific and ligand-specific analyte binding.
Finally, data will be double-referenced and, when applicable, fitted to an appropriate kinetic model.

TABLE E22
Details of GCI setup.

| Sensor chip | PAG | |
|---|---|---|
| Ligand | Protein A/G captured antibodies (captured to ≈40 pg/mm² at | |
| | a concentration of 200 ng/ml for each analysis cycle) | |
| Surface | 2 consecutive 30 sec injections of regeneration solution, followed by | |
| regeneration | running buffer | |
| | injection for surface equilibration | |
| Regeneration | 10 mM glycine HCl pH 1.7 | |
| solution | | |
| Analyte | L1-CAM diluted in running buffer, 1:2 serial dilution with a maximum | |
| | concentration of 250 nM | |
| Running | 10 mM HEPES pH 7.4, 150 mM NaCl, 3 mM EDTA, 0.05% Tween-20 | |
| buffer | | |
| Association | 180 | sec |
| time | | |
| Dissociation | 900 | sec |
| time | | |
| Flowrate | 25 | μl/min |
| Acquisition | 10 | Hz |
| rate | | |
| Temperature | 25° | C. |
| Blank | Regularly interspaced every 4^{th} sample | |
| injections | | |
| DMSO | 0.0-+2.0% DMSO curve (DMSO concentration compared to | |
| solvent | running buffer), | |
| correction | performed at the end of the experiment | |
| Data | Double-referencing | |
| processing | | |

| | |
|---|---|
| Data fitting | 1:1 kinetic model |
| Instrument | WAVEdelta (Creoptix) |
| Software | Creoptix ™ WAVEcontrol version 4.5.13 |

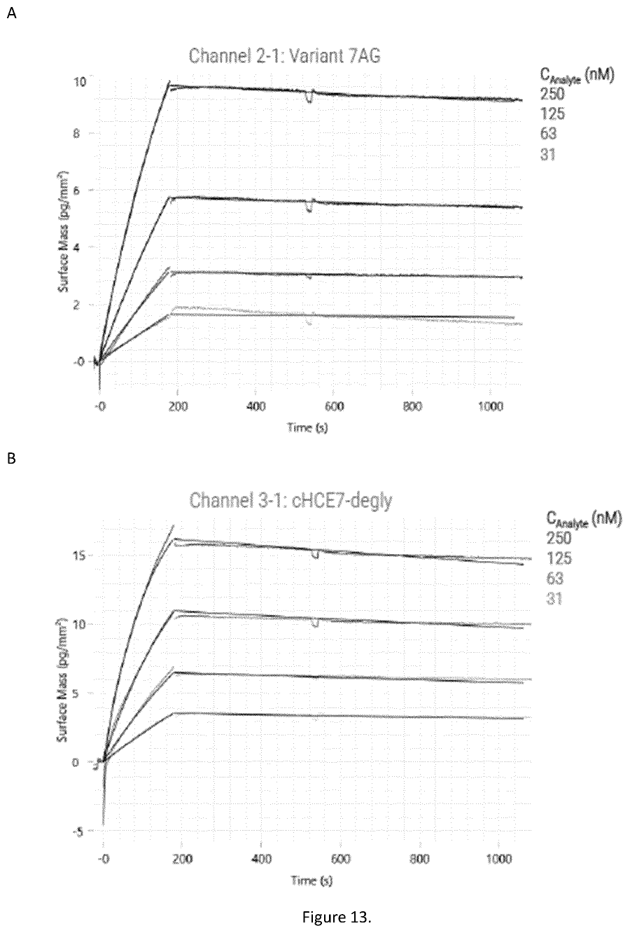

FIG. 13A shows the double referenced, solvent corrected sensorgram of the interaction between Variant 7 AG and L1-CAM, with measured responses illustrated on the graph. Dose responsive binding was detected. Fitting the data with a 1:1 interaction model, with fit curves shown in black, resulted in a calculated affinity of ≈4 nM. The ligand activity was calculated to be 28%. Lower concentrations were excluded to improve the fitting quality.

FIG. 13B shows the double referenced, solvent corrected sensorgram of the interaction between cHCE7 deglycosylated and L1-CAM, with measured responses illustrated in on the graph. Dose responsive binding was detected. Fitting the data with a 1:1 interaction model, with fit curves shown in black, resulted in a calculated affinity of ≈4 nM. The ligand activity was calculated to be 28%. Lower concentrations were excluded to improve the fitting quality.

Accordingly, this study confirms that affinity of the humanized and chimeric antibodies are comparable.

### Example 15—Functionalization of the Antibodies of the Present Invention with Azido Linker Using MTG

The humanized antibody variant (5 mg/L in PBS pH 7.5) was de-glycosylated by mixing it with N-Glycosidase (5.45 U per mg antibody) (N-Glycosidase F, 1 U/μL, REF: 11365177001, Sigma Aldrich, Germany) in a 1.5 mL reaction tube and incubated by 37° C. for 24 h under gently shaking. Completion of reaction was analyzed by LC-MS. Subsequently the buffer of de-glycosylated antibody was exchanged to Trizma buffer (50 mM pH 7.5) by using a PD-10 desalting column. De-glycosylated antibody (4 mg/ml) was hereafter incubated with a mixture of microbial transglutaminase (MTG) (2.5 U per mg antibody [Andracon™, cat. Log. T-153, Supplier Zedira, Germany], Amino-PEG₄-Azido 80 eq per antibody [Supplier Broadpharm, cat. Log. BP-21615, USA] in a 5 ml low protein binding reaction tube to modify residue Q295 in antibody heavy chain (HC) with a click-reactive azido linker. Coupling reaction was performed by 37° C. and 24 h incubation. Completion of reaction was analyzed by LC-MS (HPLC 2795 Waters, ESI-TOF LCT Premier). Coupling efficiency >90% was calculated by comparing AUC of HC (non-modified) with HC (linker modified), mass difference of linker 245 Da was utilized to identify peaks. Residual enzyme and excess of PEG linker was hereafter removed by purification by protein A affinity chromatography [Protein Sepharose 4 Fast Flow, Supplier GE Healthcare USA, cat. Log. 17.5280-20, with column Isolute® SPE cat. Log. 120.1362, Biotage, Sweden] using the manufactures protocols. In the last step the purified antibody-linker-azide conjugate was buffer exchanged (5 times) to PBS pH 7.5 using ultracentrifugation [Amicon® Ultra-15 centrifugation filter 30 kDa MWCO, cat. Log. UFC903008, Supplier Sigma Aldrich, Germany)]. Antibody linker-azido conjugate was stored at −20° C. till usage.

### Example 16—Functionalization of the Aglycosylated Antibodies of the Present Invention with Azido Linker Using MTG

The aglycosylated versions of the humanized antibodies of the invention were exposed to the same coupling protocol as presented in example 3 except that the enzymatic de-glycosylation step was skipped. Coupling efficiency with MTG was comparable to that described in Example 15.

### Example 17—Functionalization of Variant 7AG-Q295-NH-PEG₄-Azide with Metal Chelator (DBCO DOTA)

The azide-modified antibody-linker conjugate was functionalized with a metal chelator to generate a model antibody-radionuclide conjugate (ARC) comprising 2 DOTA chelators per mAb. In brief Variant 7AG-[Q295-NH-PEG₄-azide]2 (synthesized according to protocols presented in example 4) 23.2 μM in PBS pH 7.5 with addition of 1 mM ammonium acetate (pH 5.5) was incubated with a 10 times molar excess of a click reactive DBCO-DOTA (Supplier, Macrocyclics, Texas (USA), cat. Log. B-283) for 16 h at room temperature. Subsequently excess DBCO-DOTA was removed by ultracentrifugation [Vivaspin 6, 30 kDa MWCO, cat. Log. VS0621, Bio-rad, Germany].

### Example 18—Mouse Biodistribution Study with Variant 7AG-[Q295-NH-PEG₄-Click-(DOTA)]₂

The obtained huCE7-Variant 7AG-[Q295-NH-PEG₄-click-(DOTA)]₂ from example 5 was hereafter labeled with radioactive lutetium-177 and used in a mouse biodistribution study to characterize tumor uptake.

TABLE E23

| | | | | Concen- |
|---|---|---|---|---|
| | | | Amount | tration |
| | | | protein/ | mAb |
| | Activity | [μL] | dose | [mg/mL] |
| Lu-177 (as LuCl₃) | 26MBq | 0.5 | | |
| Ammonium acetate 0.5M | | 0.5 | | |
| huCE7-Variant 7-[Q295- | | 17 | 8 μg/ | 4 |
| NH-PEG₄-click-(DOTA)]₂ | | | 0.4MBq/μg | |
| PBS pH 7.5 metal free | | 17 | | |

The reaction was performed in a 1.5 mL reaction tube under gently stirring at 37° C. for one h. 1 μL sample was taken and placed in a HPLC vial with 100 μL PBS pH 7.5 and 2.5 μL 1 mM DTPA and analyzed by HPLC with gamma counter as detector to monitor labeling success.

### Cells for Tumor Model:

Her2/neu positive SKOV3ip cells were maintained in DMEM medium at 37° C. Cell media were supplemented with 10% FCS, 2 mM L-glutamine, 100 U/mL penicillin, 100 μg/mL streptomycin and 0.25 μg/mL fungizone (BioConcept, Allschwil, Switzerland). The cell line was cultivated in a humidified atmosphere containing 5% CO₂. Cells were subsequently used for injection into mice (5 mio cells per animal) to generate a Her2 & L1-CAM positive ovarian cancer tumor model.

### Mouse Tumor Model for Biodistribution:

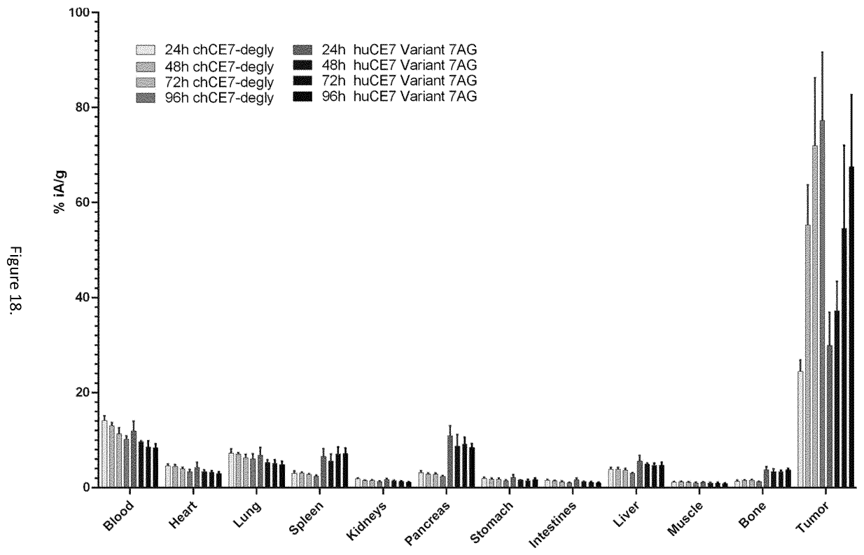

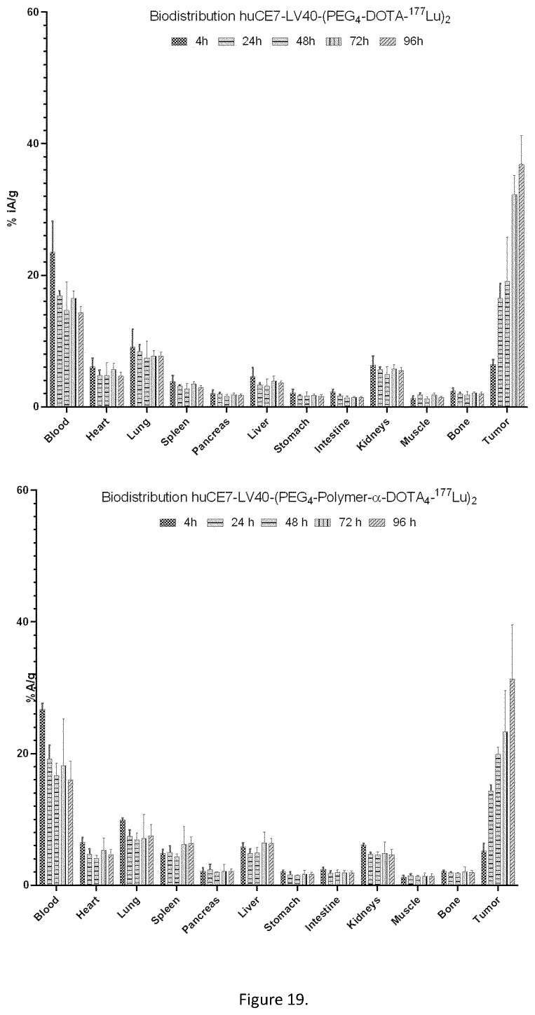

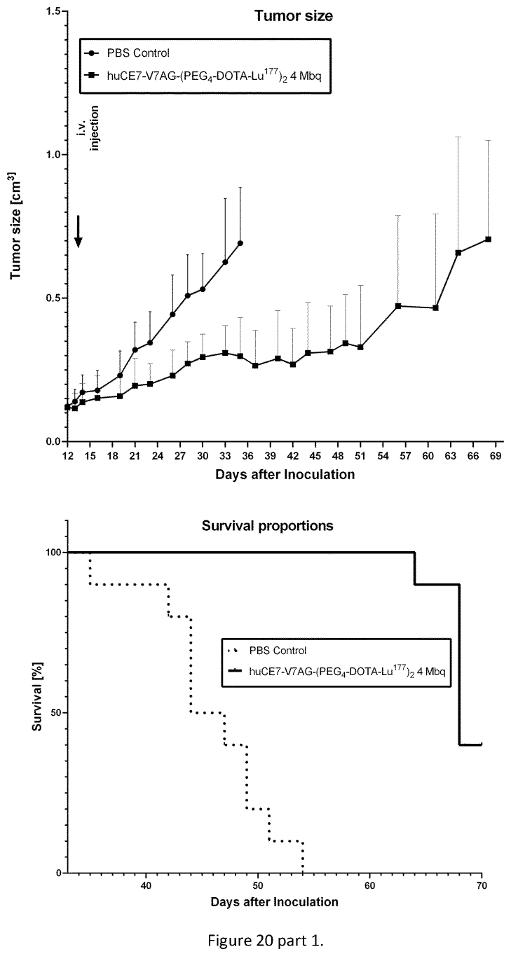

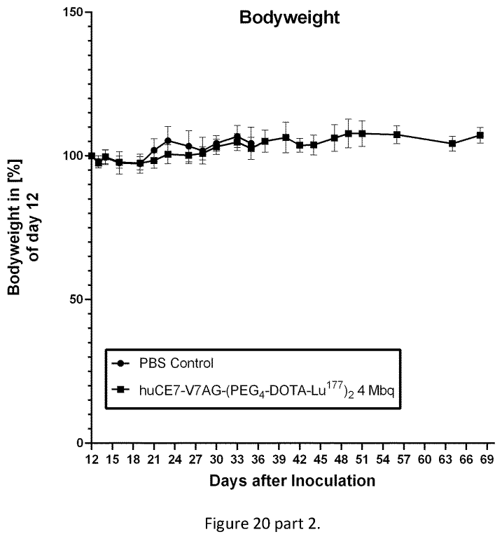

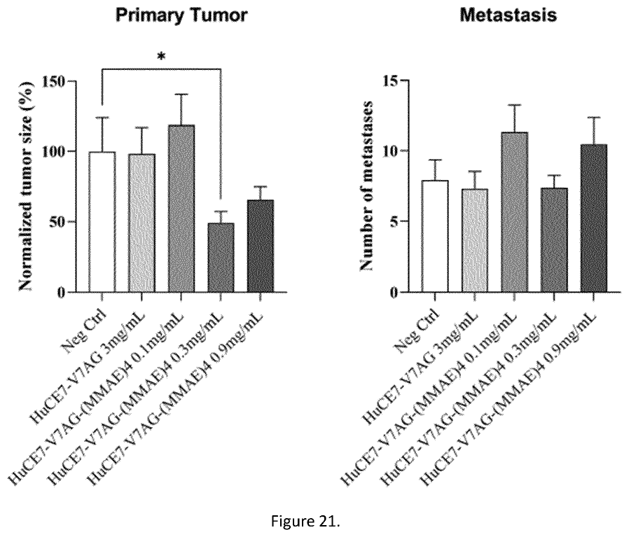

Total 32 mice bearing SKOV 3ip tumors were injected with 150 kBq huCE7 Variant 7AG-[PEG₄-DOTA (Lu-177]₂ (16 mice) in 100 μL injection solution. In this study cHCE7-[PEG₄-DOTA (Lu-177]₂ (16 mice) served as control for tumor uptake. Four mice of each group were sacrificed 24, 48, 72 and 96 h p.i. and the organs/tissues were dissected, weighted and radioactivity was measured. The % i.A./g were calculated and presented in FIG. 18.

Tumor uptake between the humanized antibody huCE7 Variant 7 AG was comparable with chimeric cHCE7 over the monitored time span of 24 to 96 h with approx. 68+/−7.7% i.A./g; 77%+/−7.2% i.A./g maximum activity per gram at 96 h indicating that humanized framework didn't interfere with tumor uptake.

Organ distribution of humanized antibody huCE7 Variant 7 AG was in the range of literature values of typical ARCs (FIG. 18).

### Example 19—Functionalization of huCE7-Variant 7AG-[Q295-NH-PEG₄-Azide with Toxin (MMAE)

The azide modified antibody-linker conjugate was functionalized with the toxin Monomethyl auristatin E (MMAE) to generate a model antibody-drug conjugate comprising 4 toxins per mAb. In brief Variant 7-[Q295-NH-PEG₄-azide]₂ synthesized according to protocols presented in example 4 was incubated with a 10 times molar excess of a click reactive DBCO-(PEG₂-vc-PAB-MMAE)₂ (Supplier, SyntaBio, USA) in PBS pH 7.5 supplemented with 10% (m/v) dimethylacetamide for 16 h at room temperature. Subsequently the ADC was purified by Sephadex G25 column into PBS pH 7.5 followed by activated carbon depletion of residual toxin linker. The resulting ADC [huCE7-V7AG-(MMAE)₄] was concentrated to approximately 3 mg/mL before a final 0.2 μm filtration.

### Analytic of DAR in ADC:

The monomer constituted 96.8% of the sample, as determined by SEC. The total concentration, as determined by SEC, was 2.8 mg/mL. Drug to antibody ration determined by MS was 3.9, and drug to antibody ratio as determined by HIC was 3.6. In total, 3.3 mg of huCE7-V7AG-(MMAE2)2 were produced.

### Example 20—Zebrafish Toxicity and Tumor Efficacy Study with huCE7-Variant 7-[Q295-NH-PEG₄-Click-Branched PEG-(MMAE)₂]₂

This study aims to evaluate huCE7-V7AG (control=non-modified mAb) and huCE7-V7AG-(MMAE)₄ antibodies' toxicity and anti-tumor effects on zebrafish embryos, primary tumor size, and metastasis formation. MAXFTN-401 cell line (L1-CAM positive TBNC), acquired from [Charles River Laboratories, MA (USA)] was used as cancer model. The anti-cancer efficacy of the antibodies was determined by the change in primary tumor size (i.e., tumor growth or reduction) and the number of tumor cells disseminated to the distal caudal venous plexus (CVP) three days after implantation, as specified below.

The study design is divided into three parts and was performed at BioReperia AB (Wahlbecksgatan 25, 582 13 Linköping)
Part 1: Toxicity evaluation of huCE7-V7AG antibody (Reduced tox)
Part 2: Toxicity evaluation of HUCE7-V7AG-(MMAE)4 antibody (Full tox evaluation)
Part 3: Evaluation of efficacy using ZTX-ONCOLEADS model (Bioreperia AB, Sweden)
   Part 1: Toxicity Evaluation of huCE7-V7AG Antibody

The reduced toxicity study for huCE7-V7AG antibody spun over 5 concentrations and vehicle control (Table E24). The antibody was administered intravenously. Every group consisted of 20 individuals of zebrafish larvae that were at 48 h post-fertilization at the time of injection and incubated at 35.5° C. during 3 days. Evaluation was performed after 72 hours of treatment and the output was evaluated at the following parameters: live/dead and LD50 concentration.

TABLE E24
Description of groups for toxicological
analysis of huCE7-V7AG antibody

| HuCE7-V7AG | | | |
|---|---|---|---|
| antibody | Embryos | | Time points |
| concentration | per | | (h) after |
| (mg/mL) | group | Administration | exposure |
| 0 (vehicle) | 20 | i.v. injection | 72 |
| 0.03 | 20 | | |
| 0.1 | 20 | | |
| 0.3 | 20 | | |
| 1 | 20 | | |
| 3 | 20 | | |

### Results:

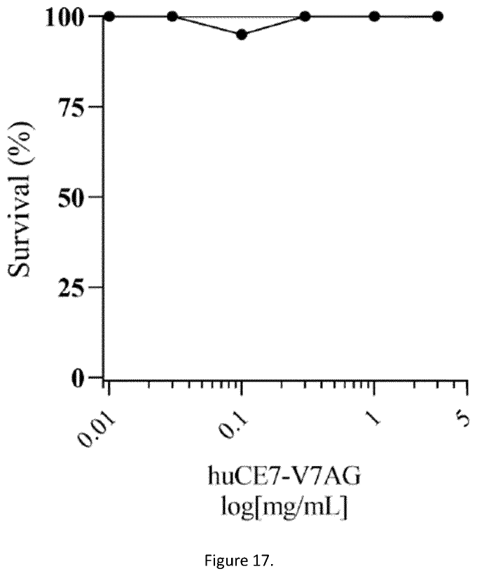

Toxicity evaluation of different concentrations of huCE7-V7AG “naked” was performed. Result showed that the antibody was very well tolerated by the embryos, with only one counted mortality (at 0.1 mg/mL concentration) out of all the experimental groups (FIG. 17). LD50 linear fit-readout therefore cannot be readily accepted as none of the experimental group had more than 50% mortality.

### Part 2: Toxicity Evaluation of HUCE7-V7AG-(MMAE)₄ Antibody

Full toxicity evaluation of HUCE7-V7AG-(MMAE)₄ antibody was decided after the tox results with huCE7-V7AG antibody. The full toxicity study for HUCE7-V7AG-(MMAE)₄ antibody spun over 5 concentrations and vehicle control (Table E25). The antibody was administered intravenously. Every group will consist of 20 individuals of zebrafish larvae that were at 48 h post-fertilization at the time of injection and incubated at 35.5° C. for 3 days. Evaluation was performed after 24, 48, and 72 hours of treatment, and the output was evaluated at the following parameters: live/dead, LT50, LD50, and non-lethal toxicities like pericardial edema, malformations, necrosis, teratogenesis, etc. Representative images of embryos were acquired at each time point.

TABLE E25
Description of groups for toxicological
analysis of HUCE7-V7AG-(MMAE)4 antibody

| HUCE7-V7AG- | | | |
|---|---|---|---|
| (MMAE)₄ antibody | | | Time points |
| concentration | Embryos | | (h) after |
| ( mg/mL) | per group | Administration | exposure |
| 0 (vehicle) | 20 | i.v. injection | 24. 48. and |
| 0.03 | 20 | | 72 |
| 0.1 | 20 | | |
| 0.3 | 20 | | |
| 1 | 20 | | |
| 3 | 20 | | |

Results: Embryo survival after exposure to HuCE7-V7Ag (MMAE)4 injected i.v.

TABLE E25.2
percentage of embryo survival

| | Percentage of Embryo survival | | | | | | | |
|---|---|---|---|---|---|---|---|---|
| | (Number of live embryos/Total number of embryos) | | | | | | | |
| Evaluation | | 0.03 | 0.1 | 0.3 | 1.0 | 2.8 | LD50 | LT50 |
| time-point | Vehicle | mg/mL | mg/mL | mg/mL | mg/mL | mg/mL | (μM) | (μM) |
| 24 h | 100 | 100 | 100 | 100 | 100 | 100 | — | — |
| 48 h | 95 | 95 | 80 | 100 | 75 | 85 | — | — |
| 72 h | 85 | 85 | 75 | 100 | 70 | 70 | — | — |

TABLE E25.3
percentage of embryos showing non-lethal toxicities after 72 hours of treatment.
Percentage of embryos showing non-lethal toxicities after 72 h of treatment

| Vehicle | 0.03 mg/mL | 0.1 mg/mL | 0.3 mg/mL | 1 mg/mL | 2.8 mg/mL |
|---|---|---|---|---|---|
| 11% | 35% | 19% | 15% | 21.4% | 85% |
| (pericardial | (pericardial | (pericardial | (pericardial | (pericardial | (pericardial |
| edema) | edema, yolk sac | edema, yolk | edema, yolk | edema, | edema, yolk |
| | edema, | sac edema, | sac edema, | malformation | sac edema, |
| | malformation of | head necrosis, | malformation | of yolk sac) | head |
| | yolk sac, small | malformation | of yolk sac, | | necrosis, |
| | eyes) | of yolk sac, | small eyes) | | malformation |
| | | small eyes) | | | of tail and |
| | | | | | yolk sac, |
| | | | | | small eyes) |

The MMAE loaded ADC showed good compatibility with the non-tumor zebrafish model. Since a 50% lethality was not reached (see Tables E25.2 and E25.3), an LT50 or LD50 concentration could not be determined; additionally, all experimental groups were within background mortality for i.v. injected embryos. Regarding non-lethal toxicities, the 2.8 mg/mL concentration showed the highest percentage of embryos with some toxicity, as expected but would still be suitable for an efficacy study. Nevertheless, the doses range for the efficacy study (Part 3) was set between 0.1 and 0.9 mg/ml.

### Part 3: Evaluation of Efficacy Using ZTX-ONCOLEADS

MAXFTN-401 cancer cells were used to evaluate the anti-tumor efficacy of huCE7-V7AG and HUCE7-V7AG-(MMAE)₄ antibodies. The study was comprised of 5 experimental groups (Table E26). Concentrations of the antibodies to test was decided based on results from toxicity evaluation. Tumor cells will be implanted subcutaneously and treatment with antibodies was administered intravenously after tumor implantation. 20 tumor-bearing embryos were included per experimental group and incubated at 35.5° C. for 3 days. The anti-tumor efficacy of antibodies was determined as the change in primary tumor size (i.e., tumor growth or reduction) and the number of tumor cells disseminated to the CVP three days after implantation.

TABLE E26
Description of experimental groups for efficacy analysis of huCE7-
V7AG and HUCE7-V7AG-(MMAE)4 antibodies using ZTX-ONCOLEADS

| | | | | | Evaluation | Evaluation |
|---|---|---|---|---|---|---|
| Tumor | | | Group | Administration | of primary | of |
| cell line | Treatment | Conc. | size | of treatment | tumor size | metastasis |
| MAXFT | Vehicle | — | 20 | i.v. | 0 and 72 h | 72 h |
| N-401 | (PBS) | | | | | |
| MAXFT | HuCE7- | 3 | 20 | i.v. | 0 and 72 h | 72 h |
| N-401 | V7AG Ab | mg/ml | | | | |
| MAXFT | HUCE7- | 0.1 | 20 | i.V. | 0 and 72 h | 72 h |
| N-401 | V7AG- | mg/ml | | | | |
| | (MMAE)₄ | | | | | |
| MAXFT | HUCE7- | 0.3 | 20 | i.v | 0 and 72 h | 72 h |
| N-401 | V7AG- | mg/ml | | | | |
| | (MMAE)₄ | | | | | |
| MAXFT | HUCE7- | 0.9 | 20 | i.v. | 0 and 72 h | 72 h |
| N-401 | V7AG- | mg/ml | | | | |
| | (MMAE)₄ | | | | | |

Detailed results and description of the study are shown in Example 28.

### Example 21—Expression and Purification of Liability Variants LV29-32

The cDNAs coding for the HC and LC sequences were chemically synthesized with optimization for expression in CHO cells and subcloned in ProteoGenix's proprietary mammalian cells' expression vector. The sequences are illustrated below:

```
>CE7-1-2-VHB-WH33Y-GH55A
  [SEQ ID NO.: 137]
GAATTCGCCGCCACCATGAAGCACCTGTGGTTCTTCCTGCTGCTGGTGGCCGCTCCTAGGTGGGTGCT
GAGCCAGGTGCAACTGGTGCAGTCCGGAGCTGAGGTGAAGAAGCCCGGAGCTTCCGTGAAGGTGAGCT
GTAAGGCCTCCGGCTACACATTCACCGGCTACTACATGCACTGGGTGAGACAGGCCCCCGGACAAGGA
CTGGAATGGATCGGAGAGATCAATCCTAGCAACGCCAGGACAAACTACAACGAGAGGTTCCAGGGCAG
GGTGACACTGACAGTGGACAAGAGCATCAGCACCGCCTACATGGAGCTGAGCAGACTGAGGTCCGATG
ACACCGCCGTGTACTACTGTGCCAGAGATTACTACGGCACCTCCTACAATTTCGACTACTGGGGCCAG
GGCACCCTGGTTACAGTGTCTTCCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-1-2-VHB-WH33F-GH55A
  [SEQ ID NO: 138]
GAATTCGCCGCCACCATGAAGCACCTGTGGTTCTTCCTGCTGCTGGTGGCCGCTCCTAGGTGGGTGCT
GAGCCAGGTGCAACTGGTGCAGTCCGGAGCTGAGGTGAAGAAGCCCGGAGCTTCCGTGAAGGTGAGCT
GTAAGGCCTCCGGCTACACATTCACCGGCTACTTCATGCACTGGGTGAGACAGGCCCCCGGACAAGGA
CTGGAATGGATCGGAGAGATCAATCCTAGCAACGCCAGGACAAACTACAACGAGAGGTTCCAGGGCAG
GGTGACACTGACAGTGGACAAGAGCATCAGCACCGCCTACATGGAGCTGAGCAGACTGAGGTCCGATG
ACACCGCCGTGTACTACTGTGCCAGAGATTACTACGGCACCTCCTACAATTTCGACTACTGGGGCCAG
GGCACCCTGGTTACAGTGTCTTCCGCTAGCACCAAGGGACCTTCTGTGTTCCCTCTGGCTCCTTCTTC
TAAGTCCACTTCCGGTGGTACAGCAGCTCTGGGTTGTCTGGTGAAGGATTACTTCCCAGAACCAGTGA
CTGTGTCCTGGAACTCCGGAGCTCTGACTTCTGGAGTGCATACTTTCCCAGCAGTGCTGCAATCTAGC
GGACTGTACTCTCTGTCTTCCGTGGTGACTGTGCCTTCTTCTTCCCTGGGGACTCAAACTTACATCTG
CAACGTGAACCACAAGCCCTCCAACACCAAGGTGGACAAGAAGGTGGAGCCAAAGAGCTGCGATAAGA
CCCACACCTGTCCACCTTGTCCAGCTCCAGAAGCCGCCGGTGGGCCTTCTGTGTTTCTGTTCCCACCT
AAGCCAAAGGATACCCTGATGATCTCTAGGACCCCAGAAGTGACCTGTGTGGTCGTCGATGTGTCTCA
TGAAGACCCTGAAGTGAAGTTCAACTGGTACGTGGACGGGGTGGAAGTGCATAACGCAAAGACCAAGC
CCAGGGAAGAGCAATACGCTTCCACCTACAGGGTGGTCTCCGTCCTGACAGTCCTGCATCAGGATTGG
CTGAACGGCAAGGAGTACAAGTGCAAGGTCTCCAATAAAGCCCTGCCTGCCAGCATCGAGAAAACCAT
TAGCAAAGCCAAAGGCCAGCCCAGGGAGCCCCAGGTCTATACACTGCCCCCCAGCAGGGAGGAGATGA
CAAAAAATCAGGTCAGCCTGACATGCCTGGTCAAAGGCTTTTATCCCAGCGACATTGCCGTCGAGTGG
GAGTCCAATGGCCAGCCCGAGAATAATTATAAAACAACACCCCCCGTCCTGGACAGCGACGGCAGCTT
TTTTCTGTATAGCAAACTGACAGTCGATAAAAGCAGGTGGCAGCAGGGCAATGTCTTTTCCTGCAGCG
TCATGCACGAGGCCCTGCACAATCACTATACTCAGAAAAGCCTGAGCCTGTCCCCCGGGAAATGAaag
ctt
>CE7-1-NL1-VLA-NL31A-WL92Y
[SEQ ID NO.: 139]
GAATTCGCCGCCACCATGGTGCTGCAGACCCAGGTGTTTATCAGCCTGCTGCTGTGGATCTCCGGCGC
TTATGGCGATATCCAGATGACCCAGTCCCCCTCCTCCCTGTCTGCTTCTGTGGGAGATAGGGTGACCA
TCACCTGTAAGGCTAATGAGGATATCAACGCTCGGCTGGCCTGGTACCAGCAGAAGCCTGGAAAGGCT
CCTAAGCTGCTGATCTCCGGCGCCACAAACCTGGTGACCGGAGTTCCTTCCCGGTTCTCCGGAAGCGG
ATCCGGAAAGGACTATACCCTGACCATCTCCAGCCTGCAGCCCGAAGATTTCGCTACCTATTACTGCC
AGCAGTATTATAGCACCCCCTTCACCTTCGGCCAGGGCACCAAACTGGAGATCAAGCGTACGGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
>CE7-1-NL1-VLA-NL31A-WL92F
  [SEQ ID NO: 140]
GAATTCGCCGCCACCATGGTGCTGCAGACCCAGGTGTTTATCAGCCTGCTGCTGTGGATCTCCGGCGC
TTATGGCGATATCCAGATGACCCAGTCCCCCTCCTCCCTGTCTGCTTCTGTGGGAGATAGGGTGACCA
TCACCTGTAAGGCTAATGAGGATATCAACGCTCGGCTGGCCTGGTACCAGCAGAAGCCTGGAAAGGCT
CCTAAGCTGCTGATCTCCGGCGCCACAAACCTGGTGACCGGAGTTCCTTCCCGGTTCTCCGGAAGCGG
ATCCGGAAAGGACTATACCCTGACCATCTCCAGCCTGCAGCCCGAAGATTTCGCTACCTATTACTGCC
AGCAGTATTTCAGCACCCCCTTCACCTTCGGCCAGGGCACCAAACTGGAGATCAAGCGTACGGTGGCT
GCACCTTCTGTGTTCATCTTCCCTCCATCTGATGAGCAGCTGAAGTCTGGAACCGCATCTGTCGTCTG
TCTGCTGAACAACTTTTACCCCAGGGAGGCTAAGGTCCAATGGAAGGTGGACAACGCCCTGCAGTCTG
GTAATAGCCAGGAAAGCGTGACCGAACAGGATTCCAAGGACTCCACCTACTCCCTGTCCTCCACACTG
ACACTGAGCAAAGCCGACTATGAAAAGCACAAAGTGTATGCCTGCGAGGTCACTCATCAGGGCCTGTC
CAGCCCCGTGACTAAAAGCTTTAATAGGGGGGAGTGCTGAGCGGCCGC
The sequences for the expected proteins produced are illustrated below.
>CE7-1-2-VHB-WH33Y-GH55A (L234A L235A + P331S + N297A)
[SEQ ID NO.: 142]
MKHLWFFLLLVAAPRWVLSQVQLVQSGAEVKKPGASVKVSCKASGYTFTGYYMHWVRQAPGQGLEWIG
EINPSNARTNYNERFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-1-2-VHB-WH33F-GH55A (L234A L235A + P331S + N297A)
  [SEQ ID NO.: 142]
MKHLWFFLLLVAAPRWVLSQVQLVQSGAEVKKPGASVKVSCKASGYTFTGYFMHWVRQAPGQGLEWIG
EINPSNARTNYNERFQGRVTLTVDKSISTAYMELSRLRSDDTAVYYCARDYYGTSYNFDYWGQGTLVT
VSSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVELFPPKPKDT
LMISRTPEVTCVVVDVSHEDPEVKENWYVDGVEVHNAKTKPREEQYASTYRVVSVLTVLHQDWLNGKE
YKCKVSNKALPASIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLSPGK
>CE7-1-NL1-VLA-NL31A-WL92Y
  [SEQ ID NO.: 143]
MVLQTQVFISLLLWISGAYGDIQMTQSPSSLSASVGDRVTITCKANEDINARLAWYQQKPGKAPKLLI
SGATNLVTGVPSRESGSGSGKDYTLTISSLQPEDFATYYCQQYYSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
>CE7-1-NL1-VLA-NL31A-WL92F
  [SEQ ID NO.: 144]
MVLQTQVFISLLLWISGAYGDIQMTQSPSSLSASVGDRVTITCKANEDINARLAWYQQKPGKAPKLLI
SGATNLVTGVPSRESGSGSGKDYTLTISSLQPEDFATYYCQQYFSTPFTFGQGTKLEIKRTVAAPSVE
IFPPSDEQLKSGTASVVCLLNNFYPREAKVQWKVDNALQSGNSQESVTEQDSKDSTYSLSSTLTLSKA
DYEKHKVYACEVTHQGLSSPVTKSENRGEC
```

### Production and Purification

### Short Protocol Description

An endotoxin-free DNA preparation was done for the constructions obtained as described hereinabove.

The four antibodies were expressed by combining HC and LC as follows:

TABLE E27

| | |
|---|---|
| Antibody ID | Co-transfection of Heavy Chain and Light chain |

| | | |
|---|---|---|
| LV29 | pTXs1-CE7-1-2-VHB- | pTXs1-CE7-1-NL1- |
| | WH33Y-GH55A | VLA-NL31A-WL92Y |
| LV30 | pTXs1-CE7-1-2-VHB- | pTXs1-CE7-1-NL1- |
| | WH33F-GH55A | VLA-NL31A-WL92F |
| LV31 | pTXs1-CE7-1-2-VHB- | pTXs1-CE7-1-NL1- |
| | WH33Y-GH55A | VLA-NL31A-WL92F |
| LV32 | pTXs1-CE7-1-2-VHB- | pTXs1-CE7-1-NL1- |
| | WH33F-GH55A | VLA |

Using the proprietary Xten transfection protocol, the plasmids were transiently co-transfected in our proprietary XtenCHO cells. Culture medium was collected when viability dropped under 50% (14 days after transfection) and purified as follows.
Clarification by 0.22 μm filtration
Equilibration, binding, and wash with PBS pH7.5
Elution by pH shift with citric acid
Neutralization with 1 M Tris-HCl pH 9.0
Analysis by PAGE and pool of the fractions of interest.
Final QC by PAGE: qualitative and quantitative by SDS-PAGE

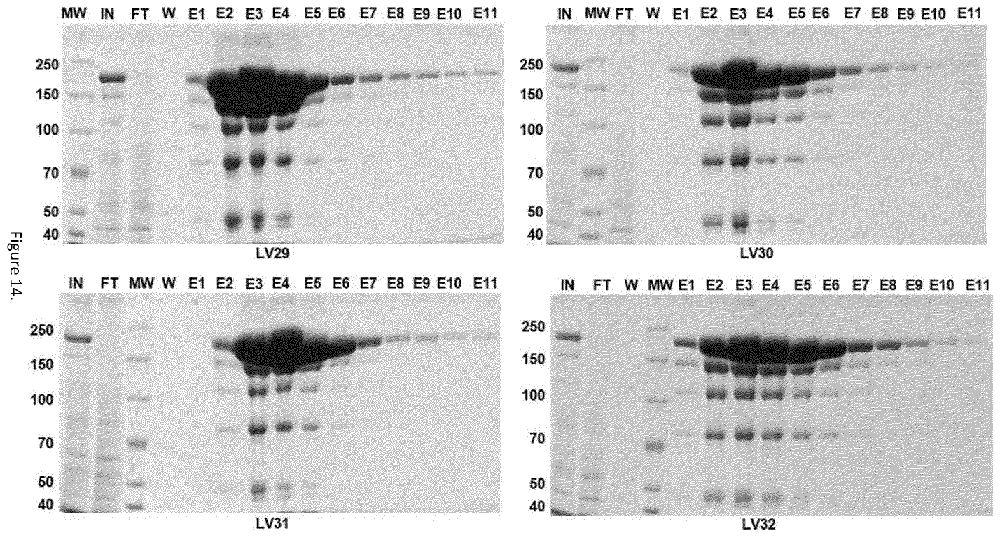

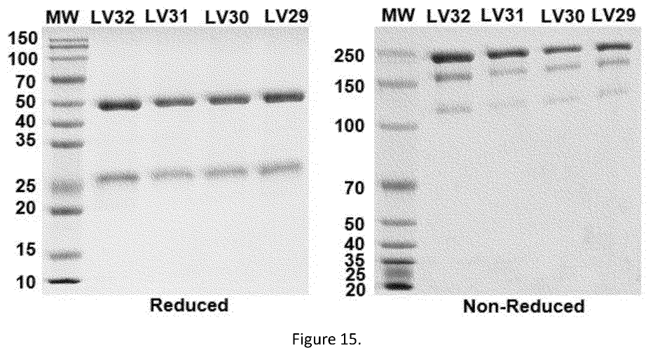

Elution fractions were pooled and buffer exchanged vs PBS, pH7.5 by dialysis method. Final samples were filtered by 0.22 um Millipore filter and assayed for endotoxin levels using a chromogenic LAL endotoxin assay kit according to IFU (Genscript Toxinsensor kit #L00350). The purification test results and QC are illustrated in FIG. 14 and FIG. 15.

The yield and purity obtained are summarized in the Table E28.

TABLE E28

| | Concen- | | Number | | Purity |
|---|---|---|---|---|---|
| Antibody | tration | Volume | of vials | Yield* | (%)** |
| LV29 | 2.44 mg/ml | 1.50 ml/vial | 2 vials | 7.32 mg/ | >90% |
| | | | | 30 ml | |
| LV30 | 2.24 mg/ml | 1.55 ml/vial | 2 vials | 6.94 mg/ | >90% |
| | | | | 30 ml | |
| LV31 | 2.36 mg/ml | 1.62 ml/vial | 2 vials | 7.65 mg/ | >90% |
| | | | | 30 ml | |
| LV32 | 1.92 mg/ml | 1.74 ml/vial | 2 vials | 6.68 mg/ | >90% |
| | | | | 30 ml | |

### Example 22—SEC-HPLC Studies of Liability Variants

### Buffer Preparation

Mobile phase: 100 mM Sodium sulfate, 100 mM Phosphate salt, pH 6.7 buffer (Na₂SO₄, 14.2 g/L, Na₂HPO₄·12H2O 15.6 g/L, NaH₂PO₄ 6.8 g/L)

Air bubbles are removed from all buffers.

Sample preparation
Protein preparation: Starting from one extra aliquot of each final sample store at −80° C. for one night, dilution of the protein samples to 1 mg/ml with DD water
Centrifugation at 12000 g during 10 min and collection of supernatants

### Analysis Conditions

Blank control: dilution (5×) of the sample with DD water
Column: G3000SWXL, TOSOH, 7.8×300 mm
HPLC: Waters 2695
Elution gradient: mobile phase isocratic elution
Flow rate: 1.0 ml/min
Temperature: 25° C.
Injection volume: 30 μl
Detection wavelength: 280 nm
Collect date time: 25 min
Equilibration with mobile phase 100% for 5 min
Run Samples: Injection of one blank control and then injection of samples
Data Analysis

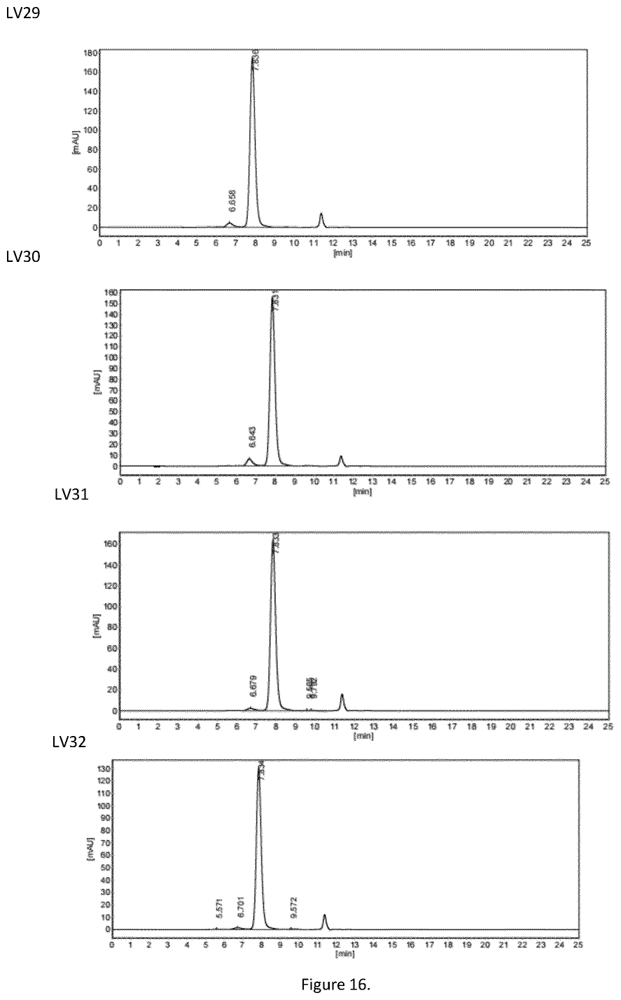

The results are shown in Table E29 and in FIG. 16.

TABLE E29
SEC-HPLC data.

| | Retention time | Area | Height | |
|---|---|---|---|---|
| Antibody | (min) | (μV/s) | (μV/s) | Purity (%) |
| LV29 | 6.658 | 106102.8 | 4256.8 | |
| | 7.836 | 3266696.4 | 174551.4 | 96.85% |
| LV30 | 6.643 | 147666.6 | 6351.1 | |
| | 7.831 | 2889088.9 | 154651.1 | 95.14% |
| LV31 | 6.679 | 66585.3 | 2292.0 | |
| | 7.833 | 3057471.8 | 163219.4 | 97.33% |
| | 9.565 | 6754.5 | 493.4 | |
| | 9.792 | 10570.5 | 588.3 | |
| LV32 | 5.571 | 8906.7 | 332.5 | |
| | 6.701 | 45735.3 | 1454.7 | |
| | 7.834 | 2446204.4 | 130462.9 | 97.3% |
| | 9.572 | 13153.4 | 472.2 | |

*Purity of full antibody single molecules detected in the sample. When several peaks are detected. data corresponding to the peak of full antibody single molecules is indicated in bold.

SEC-HPLC analyses show that all liability antibody variants exhibit very few aggregates <5%.

### Example 23—PANTA Studies of Protein Stability and Aggregation

The temperature induced aggregation of the target protein was measured via monitoring the sample turbidity change using the Prometheus PANTA back reflection optics. As protein aggregation is usually not cooperative and no well-defined process, the automatic analysis of the turbidity curves only determines the so called macro aggregation onset temperature (ON of turbidity), which is defined by the region, where a signific ant and continuous increase in the turbidity signal is first observed.

For the turbidity signal no fluorescence measurement is possible, but instead the reflection and scattering of the excitation light is measured. Thus, the turbidity detection is not as sensitive as the detection of the thermal denaturation by fluorescence, especially at low target protein concentrations. This often results in broader and less pronounced peaks in the derivative curves of the turbidity signals. Moreover, replicate measurements can often deviate in ON of turbidity by several C since protein aggregation is often influenced by the build up of local aggregation nuclei, which can differ between replicates.

For Prometheus Panta DLS the sample is filled into a thin glass capillary. Low wavelength laser light (λ=405 nm) is focused on the sample and scattered back by particles in the sample. The scatter light intensity fluctuation over time is recorded. The intensity fluctuation is analyzed with an autocorrelation function from which the diffusion coefficient is extracted by cumulant or regularization fit. By applying the diffusion coefficient to the Stokes Einstein equation, the hydrodynamic radii (rₕ) of all particles in the solution are determined. The size distribution plot shows the radius for all particle populations and the relative proportion of these species. Note that larger species scatter light more intensely (10⁶) and thus the intensity distribution is no direct representation of the amount of different size species in the sample.

Details of the method are shown in Table E30.

TABLE E30

| | |
|---|---|
| Assay type | DLS and nanoDSF |
| Target | cHCE7-degly, Variant 7AG, Variant LV-29, Variant LV-30, |
| | Variant LV-31, Variant LV-32 |
| Assay buffer | 1x PBS pH 7.4 |
| Buffer preparation | All buffers were filtered through a 200 nm syringe filter in |
| | order to remove high molecular weight particles. |
| Sample concentration | 5 μM, 2.5 μM and 1 μM were tested for the assay |
| | development step |
| | 5 μM was used for the incubation experiment |
| Sample preparation | the sample stock was centrifuged for 1 h. 4° C. at max g- |
| | force (tabletop centrifuge) to remove higher molecular |
| | weight species |
| | sample measurement was conducted with the |
| | supernatant |
| | 100 μl of each diluted samples at 5 μM was incubated for |
| | 1 week at 40° C. and measured again after incubation |
| Instrument | Prometheus Panta |
| Sensitivity | DLS: 100%; nanoDSF: 100% |
| Temperature range | DLS: 20° C.; nanoDSF: 20-95° C., heating speed 1° C./min |
| Capillary type | High sensitivity |
| Measurement mode | Duplicate measurements (DLS with ten autocorrelation |
| | functions per replicate) |
| nanoDSFanalysis | Ratio350/330 nm for protein unfolding (IP1) Cumulant ON |
| | for macroaggregation |
| | For data analysis, the current version of PR. Panta |
| | Analysis software was used (PR.Panta Analysis v1.6.3) |
| DLS analysis | Determination of the mean hydrodynamic radius (rh) and |
| | polydispersity index (PDI) from merged replicates. |
| | Depending on the particle sizing and homogeneity |
| | assessment of the sample, either a cumulant fit or a |
| | regularization fit was applied. For data analysis. the |
| | current version of the PR. Panta Analysis software |
| | (PR.Panta Analysis v1.6.3) was used. |

The results of the measurements are shown in Table E31

TABLE E31
part 1.

| | | | | Cumulant Fit | | | |
|---|---|---|---|---|---|---|---|
| Annotations | | | | Cumulant | | Cumulant | |
| Sample | target | μM | | Radius (nm) | | PDI | |
| ID | name | target | buffer | ø | σ | ø | σ |
| 1 | cHCE7- | 5 | 1x PBS | 5.64 | 0.02 | 0.04 | 0.00 |
| | degly | | pH 7.5 | | | | |
| 2 | cHCE7- | 2.5 | 1x PBS | 5.50 | 0.00 | 0.05 | 0.01 |
| | degly | | pH 7.5 | | | | |
| 3 | cHCE7- | 1 | 1x PBS | 5.42 | 0.03 | 0.09 | 0.02 |
| | degly | | pH 7.5 | | | | |
| 4 | Variant | 5 | 1x PBS | 5.83 | 0.03 | 0.11 | 0.00 |
| | 7AG | | pH 7.5 | | | | |
| 5 | Variant | 2.5 | 1x PBS | 5.72 | 0.03 | 0.09 | 0.00 |
| | 7AG | | pH 7.5 | | | | |
| 6 | Variant | 1 | 1x PBS | 5.51 | 0.02 | 0.13 | 0.00 |
| | 7AG | | pH 7.5 | | | | |
| 7 | Variant | 5 | 1x PBS | 5.77 | 0.01 | 0.03 | 0.00 |
| | LV-29 | | pH 7.5 | | | | |
| 8 | Variant | 2.5 | 1x PBS | 5.63 | 0.01 | 0.06 | 0.00 |
| | LV-29 | | pH 7.5 | | | | |
| 9 | Variant | 1 | 1x PBS | 5.51 | 0.02 | 0.10 | 0.02 |
| | LV-29 | | pH 7.5 | | | | |
| 10 | Variant | 5 | 1x PBS | 5.78 | 0.02 | 0.07 | 0.00 |
| | LV-30 | | pH 7.5 | | | | |
| 11 | Variant | 2.5 | 1x PBS | 5.80 | 0.02 | 0.07 | 0.01 |
| | LV-30 | | pH 7.5 | | | | |
| 12 | Variant | 1 | 1x PBS | 5.70 | 0.02 | 0.10 | 0.00 |
| | LV-30 | | pH 7.5 | | | | |
| 13 | Variant | 5 | 1x PBS | 5.59 | 0.02 | 0.06 | 0.00 |
| | LV-31 | | pH7.5 | | | | |
| 14 | Variant | 2.5 | 1x PBS | 5.62 | 0.00 | 0.06 | 0.01 |
| | LV-31 | | pH 7.5 | | | | |
| 15 | Variant | 1 | 1x PBS | 5.50 | 0.02 | 0.10 | 0.01 |
| | LV-31 | | pH 7.5 | | | | |
| 16 | Variant | 5 | 1x PBS | 5.68 | 0.03 | 0.07 | 0.01 |
| | LV-32 | | pH 7.5 | | | | |
| 17 | Variant | 2.5 | 1x PBS | 5.68 | 0.02 | 0.08 | 0.01 |
| | LV-32 | | pH 7.5 | | | | |
| 18 | Variant | 1 | 1x PBS | 5.53 | 0.01 | 0.12 | 0.01 |
| | LV-32 | | pH 7.5 | | | | |

TABLE E31
part 2.

| | Ratio [symbol] | | |
|---|---|---|---|
| Sample | IP #1 (° C.) | IP #2 (° C.) | ON (° C.) |

| ID | ø | σ | ø | σ | ø | σ |
|---|---|---|---|---|---|---|
| 1 | 64.37 | 0.07 | 81.43 | 0.03 | 56.38 | 0.93 |
| 2 | 64.19 | 0.05 | 81.44 | 0.09 | 57.53 | 0.23 |
| 3 | 63.95 | 0.01 | 81.41 | 0.21 | 56.70 | 1.39 |
| 4 | 57.87 | 0.03 | 79.86 | 0.02 | 51.13 | 1.39 |
| 5 | 57.80 | 0.19 | 79.76 | 0.00 | 51.13 | 0.92 |
| 6 | 57.88 | 0.09 | 79.54 | 0.01 | 52.11 | 0.93 |
| 7 | 57.66 | 0.03 | 78.92 | 0.01 | 50.80 | 0.46 |
| 8 | 57.75 | 0.07 | 78.98 | 0.04 | 49.32 | 1.62 |
| 9 | 57.95 | 0.09 | 79.05 | 0.03 | 51.12 | 0.46 |
| 10 | 57.65 | 0.02 | 78.30 | 0.04 | 50.46 | 0.46 |
| 11 | 57.63 | 0.13 | 78.40 | 0.01 | 50.13 | 0.93 |
| 12 | 57.86 | 0.16 | 78.50 | 0.03 | 50.63 | 0.23 |
| 13 | 57.73 | 0.00 | 79.01 | 0.02 | 50.13 | 0.93 |
| 14 | 57.80 | 0.05 | 79.05 | 0.00 | 50.79 | 0.46 |
| 15 | 57.78 | 0.12 | 79.05 | 0.01 | 51.12 | 0.46 |
| 16 | 57.68 | 0.02 | 78.41 | 0.02 | 49.81 | 0.46 |
| 17 | 57.83 | 0.10 | 78.43 | 0.01 | 50.79 | 0.46 |
| 18 | 57.79 | 0.07 | 78.66 | 0.00 | 49.97 | 0.69 |

TABLE E31
part 3.

| Annotations | |
|---|---|
| Turbidity [symbol] | Cumulant Radius [symbol] |
| ON (° C.) | ON (° C.) |

| Sample ID | ø | σ | ø | σ |
|---|---|---|---|---|
| 1 | 65.88 | 0.00 | 63.02 | 0.46 |
| 2 | 66.04 | 0.23 | 63.39 | 0.15 |
| 3 | 66.20 | 0.00 | 63.82 | 0.78 |
| 4 | 78.96 | 0.00 | 65.94 | 0.89 |
| 5 | 79.13 | 0.23 | 74.99 | 1.15 |
| 6 | 79.62 | 0.00 | 76.72 | 0.19 |
| 7 | 78.14 | 0.23 | 74.81 | 0.20 |
| 8 | 78.47 | 0.23 | 75.94 | 0.64 |
| 9 | 78.79 | 0.23 | 76.89 | 0.38 |
| 10 | 77.48 | 0.23 | 74.28 | 0.61 |
| 11 | 77.97 | 0.00 | 76.19 | 0.49 |
| 12 | 78.30 | 0.00 | 76.10 | 0.59 |
| 13 | 78.14 | 0.23 | 73.87 | 1.08 |
| 14 | 78.30 | 0.00 | 75.69 | 0.66 |
| 15 | 78.95 | 0.00 | 76.42 | 0.23 |
| 16 | 77.64 | 0.00 | 74.58 | 0.60 |
| 17 | 78.13 | 0.23 | 74.87 | 1.07 |
| 18 | 78.63 | 0.00 | 75.98 | 0.69 |

Tₘ: Melting temperature of a protein. This value is a thermodynamic parameter of a protein that can only strictly be determined under thermodynamic equilibrium conditions and with well-known baseline parameters for the required thermodynamic fit. However, thermal unfolding experiments are often not in equilibrium, because irreversible protein aggregation processes keep removing unfolded protein from the reaction, making the unfolding process irreversible. Heating rates faster than 1 C/min additionally decrease the equilibrium resemblance of the unfolding reaction.

ON of unfolding: Onset of thermal melting. This is the temperature at which thermal protein unfolding starts. It is determined from the first derivative curve of the thermal unfolding signal (350/330 nm ratio signal).

IP: Inflection point. Due to the constraints described above, in nanoDSF, thermal melting temperatures of proteins are determined directly from the measured data by using a smoothing algorithm (polynomal fit) and detection of inflection points in the data. Assuming an equilibrium, determined Ips correspond to the Tm values of protein.

ON of turbidity: Onset of protein macro aggregation. This is the temperature at which the protein sample starts to aggregate. It is determined from the first derivate curve of the turbidity signal. Typically, ON of Turbidity correlates with ON of unfolding. For proteins that show more than one unfolding event, aggregation may start because of the first unfolding event or of later ones.

### Summary:

High sample homogeneity was observed with DLS for all samples before and after incubation at 40° C. High thermal stability with characteristic antibody unfolding profiles was observed for the samples measured with thermal ramp DLS+nanoDSF.

### Example 24: In Silico Modeling of APRs, Design of APR Liability Variants

Herein modeling of aggregation prone regions has been performed by using the software Solubis and by using the TANGO algorithm,

For modelling the antibody wildtype (cHCE7) structures YASARA structure: Version 22.8.22 was used. Subsequently to identify suitable mutations the following software versions were used:

To calculate the effect on thermodynamic stability: FoldX: Version 3.0 Beta 6 was used.

To calculate the effect on aggregation propensity: TANGO: Version 2.2 was used.

For Solubis, which is a basically the combination of FoldX and TANGO to enable mutations in aggregation prone regions, the version was 1.0.

More information on programs can be found on https://switchlab.org/software (as assessed on Nov. 7, 2022).

In Table E32 the results of the modeling using Solubis software based on the sequence of heavy chain LV31 (as in SEQ ID NO.: 141) are presented.

TABLE E32

| Mutation | Mol | ddG | dTANGO | ComplexSum | Complex_H_L |
|---|---|---|---|---|---|
| TH30K | H | −0.81 | −145.82 | 0.00 | 0.00 |
| TH28P | H | −0.73 | −117.71 | 0.00 | 0.00 |
| TH30P | H | −0.68 | −137.54 | 0.00 | 0.00 |
| TH30R | H | −0.66 | −145.78 | 0.00 | 0.00 |
| TH28K | H | −0.47 | −128.43 | 0.00 | 0.00 |
| YH27D | H | −0.43 | −455.63 | −0.03 | −0.03 |
| TH28R | H | −0.36 | −128.43 | 0.00 | 0.00 |
| GH31K | H | 0.27 | −146.86 | 0.00 | 0.00 |

In Table E33, the results of the modeling using Solubis software based on the sequence of heavy chain LV32 (as in SEQ ID NO.: 142) are shown.

TABLE E33

| Mutation | Mol | ddG | dTANGO | ComplexSum | Complex_H_L |
|---|---|---|---|---|---|
| TH30P | H | −1.97 | −218.24 | 0.00 | 0.00 |
| TH28E | H | −1.03 | −129.40 | 0.02 | 0.02 |
| TH28K | H | −1.00 | −199.32 | −0.01 | −0.01 |
| TH30E | H | −0.86 | −157.59 | 0.00 | 0.00 |
| TH28D | H | −0.77 | −129.63 | 0.02 | 0.02 |
| TH28R | H | −0.61 | −199.33 | −0.02 | −0.02 |
| TH30D | H | −0.53 | −158.20 | 0.01 | 0.01 |
| TH30K | H | −0.45 | −229.27 | 0.00 | 0.00 |
| TH30R | H | −0.29 | −229.21 | 0.00 | 0.00 |
| GH31E | H | −0.03 | −161.37 | 0.00 | 0.00 |
| GH31P | H | 0.12 | −219.80 | 0.00 | 0.00 |
| YH27K | H | 0.26 | −185.96 | 0.00 | 0.00 |
| GH31K | H | 0.26 | −229.99 | 0.00 | 0.00 |
| HH35K | H | 0.29 | −181.97 | −0.53 | −0.53 |
| GH31R | H | 0.36 | −230.04 | 0.01 | 0.01 |

In Table E34, the results of the modeling using Solubis software based on the sequence of heavy chain chCE7variant7 (i.e., comprising a sequence obtained by concatenating C-terminus of the sequence according to SEQ ID NO.: 28 to the N-terminus of the sequence according to SEQ ID NO.: 145) are shown.

TABLE E34

| Mutation | Mol | ddG | dTANGO | ComplexSum | Complex_H_L |
|---|---|---|---|---|---|
| TH30P | H | −1.70 | −169.83 | 0.00 | 0.00 |
| TH28E | H | −1.05 | −132.06 | 0.02 | 0.02 |
| TH28D | H | −0.96 | −132.46 | 0.02 | 0.02 |
| TH28K | H | −0.95 | −158.62 | −0.01 | −0.01 |
| TH30E | H | −0.93 | −145.92 | 0.00 | 0.00 |
| TH30D | H | −0.56 | −146.03 | 0.00 | 0.00 |
| YH27E | H | −0.45 | −110.98 | 0.01 | 0.01 |
| TH28R | H | −0.45 | −158.63 | −0.02 | −0.02 |
| TH30K | H | −0.43 | −172.71 | 0.00 | 0.00 |
| TH30R | H | −0.30 | −172.69 | 0.00 | 0.00 |
| GH31E | H | −0.19 | −147.52 | 0.00 | 0.00 |
| GH31P | H | 0.00 | −169.82 | 0.00 | 0.00 |
| GH31K | H | 0.18 | −172.71 | 0.02 | 0.02 |
| HH35K | H | 0.20 | −107.61 | −0.23 | −0.23 |
| GH31R | H | 0.22 | −172.73 | 0.03 | 0.03 |
| GH31D | H | 0.36 | −146.92 | 0.00 | 0.00 |
| WH33R | H | 0.36 | −174.25 | −0.21 | −0.21 |

Table E35 presents the results of the calculations using TANGO software for different mutations in sequence of heavy chain LV31 (as in SEQ ID NO.: 141), sequence of heavy chain LV32 (as in SEQ ID NO.: 142), chCE7variant (i.e., comprising a sequence obtained by concatenating C-terminus of the sequence according to SEQ ID NO.: 28 to the N-terminus of the sequence according to SEQ ID NO.: 145).

TABLE E35

| Candidate | Mutation | Mol | ddG | dTANGO | ComplexSum | Complex_H_L |
|---|---|---|---|---|---|---|
| huCE7variant7 | G31D | H | 0.36 | −146.92 | 0.00 | 0.00 |
| huCE7variant7 | G31E | H | −0.19 | −147.52 | 0.00 | 0.00 |
| huCE7LV32 | G31E | H | −0.03 | −161.37 | 0.00 | 0.00 |
| huCE7variant7 | G31K | H | 0.18 | −172.71 | 0.02 | 0.02 |
| huCE7LV32 | G31K | H | 0.26 | −229.99 | 0.00 | 0.00 |
| huCE7LV31 | G31K | H | 0.27 | −146.86 | 0.00 | 0.00 |
| huCE7variant7 | G31P | H | 0.00 | −169.82 | 0.00 | 0.00 |
| huCE7LV32 | G31P | H | 0.12 | −219.80 | 0.00 | 0.00 |
| huCE7variant7 | G31R | H | 0.22 | −172.73 | 0.03 | 0.03 |
| huCE7LV32 | G31R | H | 0.36 | −230.04 | 0.01 | 0.01 |
| huCE7variant7 | H35K | H | 0.20 | −107.61 | −0.23 | −0.23 |
| huCE7LV32 | H35K | H | 0.29 | −181.97 | −0.53 | −0.53 |
| huCE7variant7 | T28D | H | −0.96 | −132.46 | 0.02 | 0.02 |
| huCE7LV32 | T28D | H | −0.77 | −129.63 | 0.02 | 0.02 |
| huCE7variant7 | T28E | H | −1.05 | −132.06 | 0.02 | 0.02 |
| huCE7LV32 | T28E | H | −1.03 | −129.40 | 0.02 | 0.02 |
| huCE7variant7 | T28K | H | −0.95 | −158.62 | −0.01 | −0.01 |
| huCE7LV32 | T28K | H | −1.00 | −199.32 | −0.01 | −0.01 |
| huCE7LV31 | T28K | H | −0.47 | −128.43 | 0.00 | 0.00 |
| huCE7LV31 | T28P | H | −0.73 | −117.71 | 0.00 | 0.00 |
| huCE7variant7 | T28R | H | −0.45 | −158.63 | −0.02 | −0.02 |
| huCE7LV32 | T28R | H | −0.61 | −199.33 | −0.02 | −0.02 |
| huCE7LV31 | T28R | H | −0.36 | −128.43 | 0.00 | 0.00 |
| huCE7variant7 | T30D | H | −0.56 | −146.03 | 0.00 | 0.00 |
| huCE7LV32 | T30D | H | −0.53 | −158.20 | 0.01 | 0.01 |
| huCE7variant7 | T30E | H | −0.93 | −145.92 | 0.00 | 0.00 |
| huCE7LV32 | T30E | H | −0.86 | −157.59 | 0.00 | 0.00 |
| huCE7variant7 | T30K | H | −0.43 | −172.71 | 0.00 | 0.00 |
| huCE7LV32 | T30K | H | −0.45 | −229.27 | 0.00 | 0.00 |
| huCE7LV31 | T30K | H | −0.81 | −145.82 | 0.00 | 0.00 |
| huCE7variant7 | T30P | H | −1.70 | −169.83 | 0.00 | 0.00 |
| huCE7LV32 | T30P | H | −1.97 | −218.24 | 0.00 | 0.00 |
| huCE7LV31 | T30P | H | −0.68 | −137.54 | 0.00 | 0.00 |
| huCE7variant7 | T30R | H | −0.30 | −172.69 | 0.00 | 0.00 |
| huCE7LV32 | T30R | H | −0.29 | −229.21 | 0.00 | 0.00 |
| huCE7LV31 | T30R | H | −0.66 | −145.78 | 0.00 | 0.00 |
| huCE7variant7 | W33R | H | 0.36 | −174.25 | −0.21 | −0.21 |
| huCE7LV31 | Y27D | H | −0.43 | −455.63 | −0.03 | −0.03 |
| huCE7variant7 | Y27E | H | −0.45 | −110.98 | 0.01 | 0.01 |
| huCE7LV32 | Y27K | H | 0.26 | −185.96 | 0.00 | 0.00 |

### Example 25: Production and In Vitro Characterization of huCE7 Variant LV33 to LV42

In order to implement the data for the in-silico modeling presented in Example 24 further variants were produced and characterized. huCE7 Variants LV33 to LV42 are based on a-glycosylated huCE7 Variant 7AG with the following point additional mutations in the heavy chain. The produced variants are summarized in Table E35.

TABLE E35
Liability variants of huCE7 for increase of melting
temperature, mutations in bold are based on
in-silico modeling of APRs (Example 24).

| Sample | HC (mutation) | LC (mutation) |
|---|---|---|
| LV33 | TH30P; WH33F; GH55A | NL31A; WL92Y |
| | [SEQ ID NO.: 155] | [SEQ ID NO.: 143] |
| LV34 | WH33F; AH40R; GH55A | NL31A; WL92Y |
| | [SEQ ID NO: 156] | [SEQ ID NO.: 143] |
| LV35 | WH33F; GH55A; VH68A | NL31A; WL92Y |
| | [SEQ ID NO.: 157] | [SEQ ID NO.: 143] |
| LV36 | WH33F; GH55A; LH115T | NL31A; WL92Y |
| | [SEQ ID NO.: 158] | [SEQ ID NO.: 143] |
| LV37 | WH33F; GH55A; FH241K | NL31A; WL92Y |
| | [SEQ ID NO.: 159] | [SEQ ID NO.: 143] |
| LV38 | WH33F; GH55A; LH309D | NL31A; WL92Y |
| | [SEQ ID NO.: 160] | [SEQ ID NO.: 143] |
| LV39 | WH33F; GH55A; TH307R | NL31A; WL92Y |
| | [SEQ ID NO.: 161] | [SEQ ID NO.: 143] |
| LV40 | WH33F; GH55A; TH307P | NL31A; WL92Y |
| | [SEQ ID NO.: 162] | [SEQ ID NO.: 143] |
| LV42 | TH307P | SEQ ID NO.: 97 |
| | [SEQ ID NO.: 163] | |

Sequences summarized in the Table above are also provided hereinbelow:

Heavy chain of Variant_7_AG_L234A_L235A_P331S_N297A_+ TH30P; WH33F;
GH55A, also referred to as CE7-1-2-VHB N297A (TH30P; WH33F; GH55A):
[TH30P, WH33F, GH55A]
SEQ ID NO.: 155
MKHLWFFLLL VAAPRWVLSQ VQLVQSGAEV KKPGASVKVS CKASGYTEPG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, AH4OR,
GH55A, also referred to as CE7-1-2-VHB N297A (WH33F, AH40R, GH55A):
[WH33F, AH40R, GH55A]
SEQ ID NO.: 156
MKHLWFFLLL VAAPRWVLSQ VQLVQSGAEV KKPGASVKVS CKASGYTFTG YFMHWVRQRP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
VH68A also referred to as CE7-1-2-VHB N297A (WH33F, GH55A, VH68A):
[WH33F, GH55A, VH68A]
SEQ ID NO.: 157
MKHLWFFLLL VAAPRWVLSQ VQLVQSGAEV KKPGASVKVS CKASGYTFTG YFMHWVRQAP
GQGLEWIGEI NPSNARTNYN ERFQGRATLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
LH115T also referred to as CE7-1-2-VHB N297A (WH33F, GH55A, LH115T):
[WH33F, GH55A, LH115T]
SEQ ID NO.: 158
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTTVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
FH241K also referred to as CE7-1-2-VHB N297A (WH33F, GH55A, FH241K):
[WH33F, GH55A, FH241K]
SEQ ID NO.: 159
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVKLEPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
LH309D also referred to as CE7-1-2-VHB N297A (WH33F, GH55A, LH309D):
[WH33F, GH55A, LH309D]
SEQ ID NO.: 160
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV DHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
TH307R also referred to as CE7-1-2-VHB N297A (WH33F, GH55A, TH307R):
[WH33F, GH55A, TH307R]
SEQ ID NO.: 161
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLRV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
TH307P also referred to as CE7-1-2-VHB N297A (WH33F, GH55A, TH307P):
[WH33F, GH55A, TH307P]
SEQ ID NO.: 162
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ TH307P also
referred to as CE7-1-2-VHB N297A (TH307P):
[TH307P]
SEQ ID NO.: 163
MKHLWFFLLL VAAPRWVLSQ VQLVQSGAEV KKPGASVKVS CKASGYTFTG YWMHWVRQAP
GQGLEWIGEI NPSNGRTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
The mutants discussed hereinabove are also provided in their not a-glycosylated
forms, i.e. wherein the mutation N297A is not present. The sequences are shown in
the following
Heavy chain of Variant_7_L234A_L235A_P331S_+ TH30P; WH33F; GH55A, also
referred to as CE7-1-2-VHB (TH30P; WH33F; GH55A):
[TH30P, WH33F, GH55A]
SEO ID NO.: 164
MKHLWFFLLL VAAPRWVLSQ VQLVQSGAEV KKPGASVKVS CKASGYTEPG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVFPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
MKHLWFFLLL VAAPRWVLSQ VQLVQSGAEV KKPGASVKVS CKASGYTFTG YFMHWVRQAP
GQGLEWIGEI NPSNARTNYN ERFQGRATLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, AH40R, GH55A,
also referred to as CE7-1-2-VHB (WH33F, AH40R, GH55A):
[WH33F, AH40R, GH55A]
SEQ ID NO.: 165
MKHLWFFLLL VAAPRWVLSQ VQLVQSGAEV KKPGASVKVS CKASGYTFTG YFMHWVRQRP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, VH68A also
referred to as CE7-1-2-VHB (WH33F, GH55A, VH68A):
[WH33F, GH55A, VH68A]
SEQ ID NO.: 166
MKHLWFFLLL VAAPRWVLSQ VQLVQSGAEV KKPGASVKVS CKASGYTFTG YFMHWVRQRP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, LH115T also
referred to as CE7-1-2-VHB (WH33F, GH55A, LH115T):
[WH33F, GH55A, LH115T]
SEQ ID NO.: 167
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTTVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, FH241K also
referred to as CE7-1-2-VHB (WH33F, GH55A, FH241K):
[WH33F, GH55A, FH241K]
SEQ ID NO.: 168
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVKLEPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, LH309D
also referred to as CE7-1-2-VHB (WH33F, GH55A, LH309D):
[WH33F, GH55A, LH309D]
SEQ ID NO.: 169
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV DHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, TH307R also
referred to as CE7-1-2-VHB (WH33F, GH55A, TH307R):
[WH33F, GH55A, TH307R]
SEQ ID NO.: 170
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLRV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant 7 L234A L235A P331S + WH33F, GH55A, TH307P
also referred to as CE7-1-2-VHB (WH33F, GH55A, TH307P):
[WH33F, GH55A, TH307P]
SEQ ID NO.: 171
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ TH307P also referred to
as CE7-1-2-VHB (TH307P):
[TH307P]
SEQ ID NO.: 172
MKHLWFFLLL VAAPRWVLSQ VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Further sequences, wherein the sequence part required for the
expression is removed, are provided in the following.
Heavy chain of Variant_7_AG_L234A_L235A_P331S_N297A, also referred to
as CE7-1-2-VHB (L234A L235A + P331S N297A)
SEQ ID NO.: 173
Q VQLVQSGAEV KKPGASVKVS CKASGYTFTG YWMHWVRQAP
GQGLEWIGEI NPSNGRTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A, also referred to
as CE7-1-2-VHB-WH33F-GH55A (L234A L235A + P331S N297A)
(WH33F, GH55A)
SEQ ID NO.: 174
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ TH30P; WH33F;
GH55A, also referred to as CE7-1-2-VHB N297A (TH30P; WH33F; GH55A):
[TH30P, WH33F, GH55A]
SEQ ID NO.: 175
Q VQLVQSGAEV KKPGASVKVS CKASGYTFPG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL
PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, AH40R,
GH55A, also referred to as CE7-1-2-VHB N297A (WH33F, AH40R, GH55A):
[WH33F, AH40R, GH55A]
SEQ ID NO.: 176
Q VQLVQSGAEV KKPGASVKVS CKASGYTFTG YFMHWVRQRP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
VH68A also referred to as CE7-1-2-VHB N297A (WH33F, GH55A, VH68A):
[WH33F, GH55A, VH68A]
SEQ ID NO.: 177
Q VQLVQSGAEV KKPGASVKVS CKASGYTFTG YFMHWVRQAP
GQGLEWIGEI NPSNARTNYN ERFQGRATLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
LH115T also referred to as CE7-1-2-VHB N297A (WH33F, GH55A, LH115T):
[WH33F, GH55A, LH115T]
SEQ ID NO.: 178
Q VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTTVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
FH241K also referred to as CE7-1-2-VHB N297A_(WH33F, GH55A, FH241K):
[WH33F, GH55A, FH241K]
SEQ ID NO.: 179
Q VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVKLEPPKPK DTLMISRTPE VTCVVVDVSH EDPEVKENWY
VDGVEVHNAK TKPREEQYAS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
LH309D also referred to as CE7-1-2-VHB N297A (WH33F, GH55A, LH309D):
[WH33F, GH55A, LH309D]
SEQ ID NO.: 180
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLTV DHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
TH307R also referred to as CE7-1-2-VHB N297A (WH33F, GH55A, TH307R):
[WH33F, GH55A, TH307R]
SEQ ID NO.: 181
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLRV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ WH33F, GH55A,
TH307P also referred to as CE7-1-2-VHB N297A (WH33F, GH55A, TH307P):
[WH33F, GH55A, TH307P]
SEQ ID NO.: 182
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_AG_L234A_L235A_P331S_N297A_+ TH307P also
referred to as CE7-1-2-VHB N297A (TH307P):
[TH307P]
SEQ ID NO.: 183
Q VQLVQSGAEV KKPGASVKVS CKASGYTFTG YWMHWVRQAP
GQGLEWIGEI NPSNGRTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYAS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ TH30P; WH33F; GH55A, also
referred to as CE7-1-2-VHB (TH30P; WH33F; GH55A):
[TH30P, WH33F, GH55A]
SEQ ID NO.: 184
Q VQLVQSGAEV KKPGASVKVS CKASGYTFPG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_ _+ WH33F, AH40R, GH55A,
also referred to as CE7-1-2-VHB (WH33F, AH40R, GH55A):
[WH33F, AH40R, GH55A]
SEQ ID NO.: 185
Q VQLVQSGAEV KKPGASVKVS CKASGYTFTG YFMHWVRQRP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, VH68A also
referred to as CE7-1-2-VHB (WH33F, GH55A, VH68A):
[WH33F, GH55A, VH68A]
SEQ ID NO.: 186
Q VQLVQSGAEV KKPGASVKVS CKASGYTFTG Y FMHWVRQAP
GQGLEWIGEI NPSNARTNYN ERFQGRATLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, LH115T also
referred to as CE7-1-2-VHB (WH33F, GH55A, LH115T):
[WH33F, GH55A, LH115T]
SEQ ID NO.: 187
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTTVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, FH241K also
referred to as CE7-1-2-VHB (WH33F, GH55A, FH241K):
[WH33F, GH55A, FH241K]
SEQ ID NO.: 188
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TEPAVLQSSG LYSLSSVVTV PSSSLGTQTY ICNVNHKPSN TKVDKKVEPK
SCDKTHTCPP CPAPEAAGGP SVKLEPPKPK DTLMISRTPE VTCVVVDVSH EDPEVKENWY
VDGVEVHNAK TKPREEQYNS TYRVVSVLTV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, LH309D
also referred to as CE7-1-2-VHB (WH33F, GH55A, LH309D):
[WH33F, GH55A, LH309D]
SEQ ID NO.: 189
Q VQLVQSGAEV KKPGASVKVS CKASGYTFTG YFMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLTV DHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, TH307R also
referred to as CE7-1-2-VHB (WH33F, GH55A, TH307R):
[WH33F, GH55A, TH307R]
SEQ ID NO.: 190
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLRV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ WH33F, GH55A, TH307P
also referred to as CE7-1-2-VHB (WH33F, GH55A, TH307P):
[WH33F, GH55A, TH307P]
SEQ ID NO.: 191
Q VQLVQSGAEV KKPGASVKVS CKASGYTFTG Y FMHWVRQAP
GQGLEWIGEI NPSNARTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLPV LHQDWINGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Heavy chain of Variant_7_L234A_L235A_P331S_+ TH307P also referred to
as CE7-1-2-VHB (TH307P):
[TH307P]
SEQ ID NO.: 192
Q VQLVQSGAEV KKPGASVKVS CKASGYTFTG YWMHWVRQAP
GQGLEWIGEI NPSNGRTNYN ERFQGRVTLT VDKSISTAYM ELSRLRSDDT AVYYCARDYY
GTSYNFDYWG QGTLVTVSSA STKGPSVEPL APSSKSTSGG TAALGCLVKD YFPEPVTVSW
NSGALTSGVH TFPAVLQSSG LYSLSSVVTV PSSSLGTQTY ICNVNHKPSN TKVDKKVEPK
SCDKTHTCPP CPAPEAAGGP SVELFPPKPK DTLMISRTPE VTCVVVDVSH EDPEVKENWY
VDGVEVHNAK TKPREEQYNS TYRVVSVLPV LHQDWLNGKE YKCKVSNKAL PASIEKTISK
AKGQPREPQV YTLPPSREEM TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Light chain of variant 7 CE7-1-NL1-VLA
SEQ ID NO.: 193
DIQMTQSPSS LSASVGDRVT ITCKANEDIN NRLAWYQQKP
GKAPKLLISG ATNLVTGVPS RESGSGSGKD YTLTISSLQP EDFATYYCQQ YWSTPFTFGQ
GTKLEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSEN RGEC
Light chain of variant 7 CE7-1-NL1-VLA-NL31A-WL92Y
SEQ ID NO.: 194
DIQMTQSPSS LSASVGDRVT ITCKANEDIN ARLAWYQQKP
GKAPKLLISG ATNLVTGVPS RESGSGSGKD YTLTISSLQP EDFATYYCQQ YYSTPFTFGQ
GTKLEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSEN RGEC

The antibodies were produced and purified according to protocols presented in Example 3 and analyzed by Differential scanning fluorimetry analysis according to protocol presented in Example 10. LV 32 served as a control. The results are shown in Table E36.

TABLE E36
DSF analysis of liability huCE7 variants LV33 to LV42.

| Sample | Tm 1(° C.) | Tm 2(° C.) |
|---|---|---|
| LV32 | 58.0 | 78.5 |
| (control) | | |
| LV33 | 57.8 | 76.2 |
| LV34 | 57.8 | 80.6 |
| LV35 | 57.8 | 76.3 |
| LV36 | 57.8 | 77.4 |
| LV37 | 54.2 | 79.0 |
| LV38 | 61.2 | 79.0 |
| LV39 | 58.8 | 79.0 |
| LV40 | 64.6 | 79.0 |
| LV42 | 63.9 | 81.5 |

LV38, LV40 and LV42 showed a higher melting temperature as the parent variant LV32 or variant 7 AG, indicating the positive effect of the integrated point mutations found in the in-silico modeling of Example 24 on the antibody's stability. Binding affinity, i.e. K_{D} of huCE7 variant LV38, LV40 and LV42 for the antigen target L1-CAM (CD171) were further analyzed by SRP analysis according to protocols presented in Example 6 using huCE7 variant 7 AG as control. The data is shown in Table E37.

TABLE E37
Kinetics parameters and affinity of antibody/antigen interaction

| | Ka (1/Ms) | Kd (1/s) | K_{D} (M) |
|---|---|---|---|
| huCE7 variant 7AG | 1.48 × 10⁶ | 3.53 × 10⁻⁵ | 2.38 × 10⁻¹¹ |
| (Control) | | | |
| huCE7-variant38 | 1.21 × 10⁵ | 3.23 × 10⁻⁵ | 2.68 × 10⁻¹⁰ |
| huCE7-variant40 | 7.32 × 10⁶ | 4.72 × 10⁻⁴ | 6.44 × 10⁻¹¹ |
| huCE7-variant42 | 1.92 × 10⁶ | 5.82 × 10⁻⁵ | 5.82 × 10⁻¹² |

The SPR analysis revealed that the binding for the target was preserved as K_{D} was in a comparable range to control huCE7 variant 7 AG. Different K_{D} values of control huCE7 variant 7AG as compared to Example 6 may be a result of a new batch of the antigen L1-CAM (produced by Sinobiological, China) used for preparation of the chip.

SEC-MALS, DLS & DSF of huCE7 Variant LV40 (Stability Studies)
SEC-MALS of huCE7 LV40

SEC-MALS was performed on an Agilent Infinity 1 coupled with three angle MALS detector and RI detector (Wyatt) using a flow rate of HPLC: 0.5 ml/min; 100 μl sample loop filled completely by injecting 300 μl sample; Column type: Superdex Increase 200 10/300); Sample of huCE7 LV40 at 5 mg/ml in PBS pH7.5. Measurement Chromatography data analysis and determination of the molecular mass was performed with ASTRA 8.1.2.1 software (Wyatt).

Results: The sample loop was filled with ˜111 μg sample by complete filling with 3× the sample loop Appr. 95.1% of the sample was abundant as monomeric fraction (Peak1) and 4.9% as dimeric fraction (Peak 2). A molecular mass of 143.6 kDa was observed for the monomeric antibody fraction (Peak1). A molecular mass of 302.8 kDa was observed for peak 2 which can be assigned to a dimeric antibody particle. A very weak signal of higher molecular weight species was observed in the void with a molecular mass of ˜993 kDa. Data indicate that huCE7 LV40 has a high purity and homogeneity.

DLS of huCE7 LV40:

Method: Determination of the mean hydrodynamic radius (r h) and polydispersity index (PDI) based on DLS measurements. Depending on the particle sizing and homogeneity assessment of the sample, either a cumulant fit or a regularization fit was applied. Measurement was performed on Prometheus Panta Nanotemper. For data analysis, the current version of the PR, Panta Analysis software (PR.Panta Analysis v1.6.3) was used. The results are shown in Table E38.

TABLE E38
DLS measurement, stability of huCE7 LV40

| | | | Incubation | Cumulant | | Cumulant | |
|---|---|---|---|---|---|---|---|
| | mg/ml_{—} | centri- | 40° C. | Radius (nm) | | PDI | |
| Sample | protein | fugation | 1 week | ø | σ | ø | σ |
| LV40 | 0.55 | no | no | 5.48 | 0.02 | 0.03 | 0.01 |
| LV40 | 0.55 | yes | no | 5.41 | 0.00 | 0.01 | 0.00 |
| LV40 | 0.55 | no | yes | 5.25 | 0.05 | 0.01 | 0.00 |
| LV40 | 0.55 | yes | yes | 5.20 | 0.01 | 0.00 | 0.00 |

The radius and PDI further decreased after incubation at 40 PD for 1 week, which indicates sedimentation of small amounts of higher molecular weight species. The PDI of 0.03 to 0.00 indicates highly monodisperse sample quality. The sample was robust against aggregation, indicating a high thermal stability.

Analogous further experiments were performed for LV37, LV38, LV39, and LV40, which are summarized in Table E381 below.

TABLE E38_1
DLS measurement, stability of huCE7 LV37-LV40.

| Annotations | | | | | | Cumulant Fit | | | |
|---|---|---|---|---|---|---|---|---|---|
| | | | | | incubation | Cumulant | | Cumulant | |
| Sample | target | | mg/ml_{—} | | 1 Week | Radius (nm) | | PDI | |
| ID | name | buffer | target | centrifugation | 40° C. | ø | σ | ø | σ |
| 14 | LV37 | 1xPBS | 0.55 | no | no | 5.50 | 0.04 | 0.11 | 0.03 |
| | | pH 7.5 | | | | | | | |
| 15 | LV37 | 1xPBS | 0.55 | yes | no | 5.26 | 0.00 | 0.00 | 0.00 |
| | | pH 7.5 | | | | | | | |
| 16 | LV38 | 1xPBS | 0.55 | no | no | 5.95 | 0.04 | 0.10 | 0.00 |
| | | pH 7.5 | | | | | | | |
| 17 | LV38 | 1xPBS | 0.55 | yes | no | 5.81 | 0.03 | 0.05 | 0.01 |
| | | pH 7.5 | | | | | | | |
| 18 | LV39 | 1xPBS | 0.55 | no | no | 5.58 | 0.02 | 0.10 | 0.03 |
| | | pH 7.5 | | | | | | | |
| 19 | LV39 | 1xPBS | 0.55 | yes | no | 5.34 | 0.01 | 0.01 | 0.00 |
| | | pH 7.5 | | | | | | | |
| 20 | LV40 | 1xPBS | 0.55 | no | no | 5.48 | 0.02 | 0.03 | 0.01 |
| | | pH 7.5 | | | | | | | |
| 21 | LV40 | 1xPBS | 0.55 | yes | no | 5.41 | 0.00 | 0.01 | 0.00 |
| | | pH 7.5 | | | | | | | |
| 23 | LV37 | 1xPBS | 0.55 | no | yes | 5.27 | 0.07 | 0.02 | 0.01 |
| | | pH 7.5 | | | | | | | |
| 24 | LV37 | 1xPBS | 0.55 | yes | yes | 5.24 | 0.01 | 0.01 | 0.00 |
| | | pH 7.5 | | | | | | | |
| 25 | LV38 | 1xPBS | 0.55 | no | yes | 5.31 | 0.03 | 0.02 | 0.01 |
| | | pH 7.5 | | | | | | | |
| 26 | LV38 | 1xPBS | 0.55 | yes | yes | 5.26 | 0.01 | 0.01 | 0.00 |
| | | pH 7.5 | | | | | | | |
| 27 | LV39 | 1xPBS | 0.55 | no | yes | 5.26 | 0.05 | 0.02 | 0.01 |
| | | pH 7.5 | | | | | | | |
| 28 | LV39 | 1xPBS | 0.55 | yes | yes | 5.21 | 0.01 | 0.01 | 0.00 |
| | | pH 7.5 | | | | | | | |
| 29 | LV40 | 1xPBS | 0.55 | no | yes | 5.25 | 0.05 | 0.01 | 0.00 |
| | | pH 7.5 | | | | | | | |
| 30 | LV40 | 1xPBS | 0.55 | yes | yes | 5.20 | 0.01 | 0.00 | 0.00 |
| | | pH 7.5 | | | | | | | |

TABLE E38_1
Nano-DSF measurement, stability of huCE7 LV37-LV40.

| | Ratio [symbol] | | | | | | Turbidity [symbol] | |
|---|---|---|---|---|---|---|---|---|
| Sample | ON (° C.) | | IP #1 (° C.) | | IP #2 (° C.) | | ON (° C.) | |
| ID | ø | σ | ø | σ | ø | σ | ø | σ |
| 14 | 47.81 | 0.20 | 54.04 | 0.01 | 78.04 | 0.02 | 77.33 | 0.07 |
| 15 | 47.63 | 0.07 | 54.06 | 0.02 | 78.05 | 0.02 | 77.33 | 0.20 |
| 16 | 48.43 | 0.07 | 60.95 | 0.13 | 78.10 | 0.01 | 76.91 | 0.13 |
| 17 | 47.53 | 0.00 | 60.95 | 0.01 | 78.08 | 0.03 | 76.87 | 0.07 |
| 18 | 51.27 | 0.00 | 58.42 | 0.04 | 78.23 | 0.02 | 77.28 | 0.00 |
| 19 | 51.36 | 0.00 | 58.44 | 0.02 | 78.19 | 0.07 | 77.28 | 0.13 |
| 20 | 55.33 | 0.00 | 64.17 | 0.05 | 78.05 | 0.02 | 76.50 | 0.20 |
| 21 | 55.19 | 0.20 | 64.17 | 0.04 | 78.01 | 0.01 | 76.41 | 0.20 |
| 23 | 47.45 | 0.65 | 53.94 | 0.02 | 77.88 | 0.11 | 77.12 | 0.13 |
| 24 | 47.59 | 0.07 | 54.03 | 0.06 | 77.92 | 0.08 | 77.16 | 0.07 |
| 25 | 47.53 | 0.15 | 61.00 | 0.00 | 77.95 | 0.11 | 76.66 | 0.26 |
| 26 | 47.54 | 0.01 | 61.14 | 0.08 | 77.95 | 0.04 | 76.70 | 0.07 |
| 27 | 50.77 | 0.26 | 58.33 | 0.01 | 77.98 | 0.07 | 76.93 | 0.13 |
| 28 | 50.82 | 0.07 | 58.36 | 0.01 | 78.06 | 0.02 | 77.16 | 0.07 |
| 29 | 55.05 | 0.07 | 64.22 | 0.01 | 77.91 | 0.06 | 76.33 | 0.20 |
| 30 | 55.61 | 0.07 | 64.16 | 0.02 | 78.04 | 0.01 | 76.43 | 0.07 |

Nano DSF of huCE7 LV40:

Method: Ratio 350/330 nm for protein unfolding Turbidity T ON (which is the aggregation onset temperature for macroscopic visible aggregates) for macro aggregation Measurement was performed on Prometheus Panta Nanotemper. For data analysis, the current version of PR, Panta Analysis software (PR.Panta Analysis v1.6.3) was used.

### Results:

Two unfolding transitions were observed at 64.2° C. (IP #1) and at 78.1° C. (IP #2). Protein unfolding (Ratio T ON) started at ˜55° C. Microscopic sample aggregation was observed from ˜77° C. (Turbidity T ON Scattering amplitude was high and signal/noise ratio was very good.

Stability Assessment after 1 Week Storage at 40° C.:

Two unfolding transitions were observed at 64.2° C. (IP #1) and at 78.1° C. (IP #2). Protein unfolding (Ratio T ON) started at ˜55° C. Microscopic sample aggregation was observed from ˜76° C. (Turbidity T ON). Scattering amplitude was high and signal/noise ratio was very good. No change of the thermal stability was observed before and after incubation for one week at 40° C.

### Example 26 In Vitro Characterization of huCE7 LV40

Characterization of Off-Target Effects huCE7 LV40

The objective of this study was to assess, using cell-based microarray assay techniques, the interaction of huCE7 LV40 with a panel of cell surface proteins. With this, potential off-target effects should be identified. The study was performed by Charles River laboratories (UK).

### Methodology

### Pre-Screen—Micro Array Assay

Slides were spotted with expression vectors encoding both ZsGreen1 and human L1-CAM (CD171), CD20 or EGFR, and used to reverse-transfect HEK293 cells. 2, 5 or 20 μg/mL of huCE7 LV 40, 1 μg/mL of Rituximab biosimilar or PBS only were added to the above cells/slides after fixation. Binding to target-expressing cells and un-transfected cells was assessed using an AlexaFluor 647 labelled anti-human IgG Fc (AF647 anti-hIgG Fc) detection antibody, followed by fluorescence imaging. This detection antibody has been validated previously for use in the Retrogenix Cell Microarray Technology system for detecting human IgGs and human Fc fusion proteins.

### Library Screen—Microarray Assay

For Library screening, 6105 expression vectors, encoding both ZsGreen1 and a full-length human plasma membrane protein, secreted or a cell surface-tethered human secreted protein, plus 400 human heterodimers were individually arrayed in duplicate, across cell microarray slides (‘slide-sets’). An expression vector (pIRES-hEGFR-IRES-ZsGreen1) was spotted in quadruplicate on every slide and was used to ensure that a minimal threshold of transfection efficiency had been achieved or exceeded on every slide. Human HEK293 cells were used for reverse transfection/expression. huCE7 LV 40 was added to each slide after cell fixation, giving a final concentration of 20 μg/mL. Detection of binding was performed using the same fluorescent secondary antibody as used in the pre-screen (AF647 anti-hIgG Fc). The test antibody was screened against 2 replicate slide-sets. Fluorescent images were analyzed and quantitated for transfection using ImageQuant software (GE healthcare, Version 8.2). A protein interaction is defined as a duplicate spot showing a raised signal, compared to background levels. This is achieved by visual inspection using the ImageQuant software. Interactions were classified as ‘strong, medium, weak or very weak’, depending on the intensity of the duplicate spots. The term ‘significant’ in the context of this report does not refer to statistical significance. A significant interaction is defined as a signal of weak intensity or greater.

### Confirmation Screen—Micro-Array Assay

Vectors encoding all interactions identified in the library screen, plus control vectors encoding CD20 (positive control) and EGFR (transfection and negative control), were arrayed and expressed in HEK293 cells on new slides. Confirmation screen slides and analyses were carried out as for the library screen either after cell fixation (n=2) or in the absence of fixation (n=1). Slides were treated with 20 μg/mL of huCE7 LV 40, 1 μg/mL of Rituximab biosimilar (Charles River positive control) or no test molecule (secondary only; negative control). Binding to target-expressing cells and non-transfected cells was again assessed by fluorescence imaging.

### Results:

In total, 14 library interactions (duplicate spots) were identified by analyzing fluorescence (AF647 and ZsGreen1) on ImageQuant. There were a range of intensities (signal to background) from very weak to strong. Confirmation screens: Vectors encoding all 14 interactors identified in the library screen, plus control vectors encoding CD20 and EGFR, were spotted in duplicate on new slides, and used to reverse transfect human HEK293 cells as before. All transfection efficiencies exceeded the minimum threshold. Interactions of weak intensity or above, specific to the test article were identified and are listed in Table E39 and Table E40.

After screening test antibody huCE7 LV 40 for binding against human HEK293 cells expressing 6105 human plasma membrane proteins, secreted and cell surface-tethered human secreted proteins+400 human heterodimers. huCE7 LV 40 showed a significant specific interaction with the primary target L1-CAM (CD171), both after fixation and in the absence of fixation. No additional significant specific interactions were identified, indicating high specificity for the primary target.

TABLE E39
huCE7 LV40 & controls added to fixed micro-array slides.

| | | | Interaction no | | | | | | | |
|---|---|---|---|---|---|---|---|---|---|---|
| | | | 1 | 2 | 3 | 4 | 5 | 6 | 7 | 8 |
| | | | Gene ID | | | | | | | |
| | | | | | L1CAM | | | | | |
| | | | FCGR1A | IGHG3 | (CD171) | SPNS2 | MFSD2B | CXCL12 | IGF2 | IGF1 |
| | Libary (L) or | | Unique clone number | | | | | | | |
| | Confirmation | Rep | 0044 | 2497 | 3255 | 6431 | 6466 | 4396 | 4844 | 5073 |
| Sample Id and clone | (C) screen | no. | PM | M | PM | PM | PM | PM | TS | TS |

Fixed cells results

| | | | | | | | | | | |
|---|---|---|---|---|---|---|---|---|---|---|
| huCE7 LV40 (20 μg/mL) | L | 1 | weak | med/strong | strong | weak | v. weak | v. weak | v. weak | weak |
| huCE7 LV40 (20 μg/mL) | L | 2 | v. weak | medium | strong | weak | v. weak | v. weak | v. weak | v. weak |
| huCE7 LV40 (20 μg/mL) | C | 1 | v. weak | med/strong | strong | weak | v. weak | v. weak | v. weak | weak |
| huCE7 LV40 (20 μg/mL) | C | 2 | weak | med/strong | strong | weak | v. weak | weak | v. weak | weak |
| Rituximab biosimilar | C | 1 | medium | medium | | *v. weak | *v. weak | *v. weak | *v. weak | *v. weak |
| (1 μg/mL) | | | | | | | | | | |
| Rituximab biosimilar | C | 2 | med/strong | med/strong | | *v. weak | *v. weak | *v. weak | *v. weak | v. weak |
| (1 μg/mL) | | | | | | | | | | |
| PBS (secondary only) | C | 1 | weak | med/strong | | *v. weak | *v. weak | *v. weak | *v. weak | *v. weak |
| PBS (secondary only) | C | 2 | weak | med/strong | | *v. weak | *v. weak | *v. weak | *v. weak | *v. weak |

| | | | Interaction no | | | | | | | |
|---|---|---|---|---|---|---|---|---|---|---|
| | | | 9 | 10 | 11 | 12 | 13 | 14 | 15 | 16 |
| | | | Gene ID | | | | | | | |
| | | | EDN2 | EDN1 | IGHG2 | IGHG1 | IGHG4 | PAPPA | CD20 | EGFR |
| | Libary (L) or | | Unique clone number | | | | | | | |
| | Confirmation | Rep | 5017 | 5016 | 5549 | 5548 | 5528 | 5418 | 0038 | — |
| Sample Id and clone | (C) screen | no. | TS | TS | TS | TS | TS | TS | PM | PM |

Fixed cells results

| huCE7 LV40 (20 μg/mL) | L | 1 | v. weak | v. weak | strong | strong | strong | v. weak | n/a | n/a |
|---|---|---|---|---|---|---|---|---|---|---|
| huCE7 LV40 (20 μg/mL) | L | 2 | v. weak | v. weak | strong | strong | strong | v. weak | n/a | n/a |
| huCE7 LV40 (20 μg/mL) | C | 1 | *v. weak | *v. weak | strong | strong | strong | *v. weak | | |
| huCE7 LV40 (20 μg/mL) | C | 2 | *v. weak | *v. weak | strong | strong | strong | *v. weak | | |
| Rituximab biosimilar | C | 1 | *v. weak | *v. weak | strong | strong | strong | | strong | |
| (1 μg/mL) | | | | | | | | | | |
| Rituximab biosimilar | C | 2 | *v. weak | *v. weak | strong | strong | strong | *v. weak | strong | |
| (1 μg/mL) | | | | | | | | | | |
| PBS (secondary only) | C | 1 | *v. weak | *v. weak | med/strong | strong | med/strong | | | |
| PBS (secondary only) | C | 2 | *v. weak | *v. weak | strong | strong | strong | *v. weak | | |

*interaction can be seen at enhanced contrast
PM—plasma membrane,
S—secreted,
TS—tethered secreted,
HD—heterodimer,
M—evidence of membrane

TABLE E40
huCE7 LV40 & controls added to confirmation slides in the absence of fixation (live).

| | | | Interaction no | | | | | | | |
|---|---|---|---|---|---|---|---|---|---|---|
| | | | 1 | 2 | 3 | 4 | 5 | 6 | 7 | 8 |
| | | | Gene ID | | | | | | | |
| | | | | | L1CAM | | | | | |
| | | | FCGR1A | IGHG3 | (CD171) | SPNS2 | MFSD2B | CXCL12 | IGF2 | IGF1 |
| | Libary (L) or | | Unique clone number | | | | | | | |
| | Confirmation | Rep | 0044 | 2497 | 3255 | 6431 | 6466 | 4396 | 4844 | 5073 |
| Sample Id and clone | (C) screen | no. | PM | M | PM | PM | PM | PM | TS | TS |

Live cell (no fixation) results

| | | | | | | | | | |
|---|---|---|---|---|---|---|---|---|---|
| huCE7 LV40 (20 μg/mL) | C | 1 | weak | medium | weak | v. weak | weak | v. weak | v. weak |
| Rituximab biosimilar | C | 1 | med/strong | | v. weak | *v. weak | weak | *v. weak | v. weak |
| (1 μg/mL) | | | | | | | | | |
| PBS (secondary only) | C | 1 | weak | | v. weak | v. weak | weak | v. weak | weak |

| | | | Interaction no | | | | | | | |
|---|---|---|---|---|---|---|---|---|---|---|
| | | | 9 | 10 | 11 | 12 | 13 | 14 | 15 | 16 |
| | | | Gene ID | | | | | | | |
| | | | EDN2 | EDN1 | IGHG2 | IGHG1 | IGHG4 | PAPPA | CD20 | EGFR |
| | Libary (L) or | | Unique clone number | | | | | | | |
| | Confirmation | Rep | 5017 | 5016 | 5549 | 5548 | 5528 | 5418 | 0038 | — |
| Sample Id and clone | (C) screen | no. | TS | TS | TS | TS | TS | TS | PM | PM |

Live cell (no fixation) results

| huCE7 LV40 (20 μg/mL) | C | 1 | *v. weak | *v. weak | weak/med | med/strong | med/strong | v. weak | |
|---|---|---|---|---|---|---|---|---|---|
| Rituximab biosimilar | C | 1 | | v. weak | medium | med/strong | med/strong | v. weak | med/strong |
| (1 μg/mL) | | | | | | | | | |
| PBS (secondary only) | C | 1 | | | weak/med | med/strong | med/strong | v. weak | |

*interaction can be seen at enhanced contrast
PM—plasma membrane,
S—secreted,
TS—tethered secreted,
HD—heterodimer,
M—evidence of membrane.

Characterization of Tissue Cross Reactivity huCE7 LV40

The objective of this study was to assess, using immunohistochemical techniques, the cross reactivity of huCE7 LV40 in a panel of human tissues.

### Methodology

TABLE E41
antibodies used in tissue cross reactivity study

| Identification | huCE7 LV40 (Test Article) | |
|---|---|---|
| Lot/Batch Number | 19244-022223-A01 | |
| Concentration | 5.00 | mg/mL |
| Molecular Weight | 143600 | Da |

| | |
|---|---|
| Target | L1-CAM |
| Buffer Composition | PBS, pH 7.5 |
| IgG Class | IgG1 |
| Host Species | Human |
| Purity | >97.7% (evaluated by SEC-HPLC) |

| | Ultra-LEAF Purified Human IgG1 |
|---|---|
| Identification | Isotypes (Control Article 1) |
| Lot/Batch Number | B372653 |
| Manufacturer | Biolegend |
| Product Code | 403502 |
| Concentration | 2.65 mg/mL |
| Expiry Date | 27 May 2025 |
| Storage Conditions | 2° C. to 8° C. |
| Appearance | At receipt: clear liquid |

| Identification | Mouse IgG Isotype (Control Article 2) |
|---|---|
| Lot/Batch Number | 0771891 |
| Manufacturer | ThermoFisher Scientific |
| Product Code | 31903 |
| Concentration | 5.05 mg/mL |
| Expiry Date | 11 Oct. 2023 |
| Storage | CoA: 4° C. |
| Conditions* | At Receipt: 2° C. to 8° C. |
| Appearance | At receipt: liquid |

| Identification | Purified anti-CD171 (L1) (Reference Article) |
|---|---|
| Lot/Batch Number | B382271 |
| Manufacturer | Biolegend |
| Product Code | 826701 |
| Concentration | 0.22 mg/mL |
| Expiry Date | 13 Apr. 2024 |
| Storage Conditions | 2° C. to 8° C. |
| Appearance | At receipt: clear liquid |

### Conjugation of Test Article and Control Article

To facilitate immunohistochemical detection, the Test Article and Control Article were conjugated by Labcorp (UK) with FITC using the commercial FluoroTag™ FITC Conjugation Kit (FITC1). The presence of the conjugated protein in the collected fractions was determined by measuring the absorbance at 280 nm and 495 nm. The collected fractions demonstrating the highest concentrations of FITC conjugated protein were subsequently selected and identified as huCE7 LV40-FITC and IgG1-FITC. The final protein concentrations of huCE7 LV40-FITC and IgG1-FITC were determined as 2.33 mg/mL and 2.30 mg/mL respectively.

### Test System

The test system was preparation derived from histologically normal frozen Fehler! Verweisquelle konnte nicht gefunden werden. tissues, collected from two donors of each of the tissues identified below:

TABLE E42
Used tissue samples in cross reactivity study.

| | | | |
|---|---|---|---|
| Adrenal | Eye | Oesophagus | Striated Muscle |
| Urinary Bladder | Fallopian Tube | Ovary | Testis |
| Blood Cells | Gastric Antrum | Pancreas | Thymus |
| Bone Marrow | Gastric Body | Peripheral Nerve | Thyroid |
| Breast | Heart | Pituitary | Tonsil |
| Brain - | Ileum | Placenta | Ureter |
| Cerebellum | | | |
| Brain - Cortex | Kidney | Prostate | Uterus - Cervix |
| Colon | Liver | Skin | Uterus |
| | | | Endometrium |
| Duodenum | Lung | Spinal Cord | |
| Endothelium | Lymph Node | Spleen | |

Tissues, with the exception of blood cells, were cryo-sectioned. Blood cells were prepared as smears. Details regarding preparation are documented in the study records. The panel of tissues was obtained from tissues held at Labcorp (UK).

### Staining Methods:

The immunohistochemical staining method used with the conjugated test article (huCE7 LV40-FITC) and control article (IgG1-FITC) are summarized below (staining prepared at 1.25, 0.625 and 0.3125 μg/m):
Cryo-sections are allowed to air dry for 30 to 60 minutes.
Fix in acetone for 10 minutes.
Air dry for 30 minutes.
Fix in 10% neutral buffered formalin for 30 seconds.
Wash in tap water for 10 minutes.
Assemble slides into Sequenza™ coverplates.
Wash in Dulbecco's phosphate buffered saline for 10 minutes.
Treat with DAKO REAL peroxidase-blocking solution for 10 minutes.
Wash in Dulbecco's phosphate buffered saline for 10 minutes.
Treat with milk powder blocking solution* for 30 minutes.
Wash in Dulbecco's phosphate buffered saline for 10 minutes.
Treat with antibody (diluted in milk powder antibody diluent* for 1 hour.
Wash in Dulbecco's phosphate buffered saline for 10 minutes.
Treat with rabbit anti-FITC (1:1600 dilution in milk powder antibody diluent*) for 30 minutes.
Wash in Dulbecco's phosphate buffered saline for 10 minutes.
Treat with DAKO envision anti-rabbit HRP for 30 minutes.
Wash in Dulbecco's phosphate buffered saline for 10 minutes.
Disassemble from sequenza staining system into Dulbecco's phosphate buffered saline.
Treat with DAKO liquid DAB+ substrate chromogen system for 10 minutes.
Wash in tap water for 10 minutes.
Treat with 0.5% copper sulphate for 5 minutes.
Wash in tap water for 5 minutes.
Counterstain in haematoxylin for 2 minutes.
Wash in tap water for 2 minutes.
Treat with Scott's tap water substitute for 1 minute.
Wash in tap water for 1 minute.
Dehydrate, clear and mount.
   * To be diluted using Dulbecco's Phosphate Buffered Saline

The immunohistochemical staining method used with the unconjugated reference article (anti-CD171(L1) summarized below (staining prepared at 1.25, 0.625 and 0.3125 μg/mL):
Cryo-sections are allowed to air dry for 30 to 60 minutes.
Fix in acetone for 10 minutes.
Air dry for 30 minutes.
Fix in 10% neutral buffered formalin for 30 seconds.
Wash in tap water for 10 minutes.
Assemble slides into Sequenza™ coverplates.
Wash in Dulbecco's phosphate buffered saline for 10 minutes.
Treat with DAKO REAL peroxidase-blocking solution for 10 minutes.
Wash in Dulbecco's phosphate buffered saline for 10 minutes.
Treat with protein block serum free for 30 minutes.
Wash in Dulbecco's phosphate buffered saline for 10 minutes.
Treat with antibody (0.2% bovine serum albumin*) for 1 hour.
Wash in Dulbecco's phosphate buffered saline for 10 minutes.
Treat with DAKO envision anti-mouse HRP for 30 minutes.
Wash in Dulbecco's phosphate buffered saline for 10 minutes.
Disassemble from sequenza staining system into Dulbecco's phosphate buffered saline.
Treat with DAKO liquid DAB+ substrate chromogen system for 10 minutes.
Wash in tap water for 10 minutes.
Treat with 0.5% copper sulphate for 5 minutes.
Wash in tap water for 5 minutes.
Counterstain in haematoxylin for 2 minutes.
Wash in tap water for 2 minutes.
Treat with Scott's tap water substitute for 1 minute.
Wash in water for 1 minute.
Dehydrate, clear and mount.
   *To be diluted using Dulbecco's Phosphate Buffered Saline

To facilitate immunohistochemical detection huCE7 LV40 (the “Test Article”) and Ultra-LEAF purified Human IgG1 Isotype Control (“Control Article 1”) were conjugated by Labcorp (UK) with FITC and identified as huCE7 LV40-FITC and IgG1-FITC. The respective protein concentrations were 2.33 mg/mL and 2.30 mg/mL. Purified anti-CD171 (L1) (“Reference Article”) and mouse IgG Isotype Control (“Control Article 2”) were commercially obtained, the respective protein concentrations were 0.22 mg/mL and 5.05 mg/mL.

### Summary Results:

The objective of this study was to assess, using immunohistochemical techniques, the cross reactivity of huCE7 LV40 in a panel of human tissues and compare it with reference controls. Cryo-sections from the control material and each of the human tissues for examination were prepared. The assessment of tissue viability indicated that the panel of human tissues was viable.

In the control titration positive, membranous to cytoplasmic, staining of tubular epithelium was observed in kidney-tubules (positive control material) with both LV40-FITC and anti CD171(L1). No huCE7 LV40-FITC or anti-CD171(L1) positive staining was observed in liver-hepatocytes (negative control material). Following slide evaluation, concentrations of 1.25, 0.625 and 0.3125 μg/mL were selected for use in the tissue titration.

In the tissue titration huCE7 LV40-FITC positive membranous/cytoplasmic staining was observed in peripheral nerve fibers of many tissues, neuroglial cells of the brain (cerebellum and cortex), eye and spinal cord, neuroendocrine cells of the pituitary, phaeochromocytes of the adrenal, tubular epithelium of the kidney and epithelium of the fallopian tube.

Highly comparable staining with reference anti-CD171(L1) was observed in a small tissue panel. Anti-CD171(L1) positive staining was observed in peripheral nerve fibers of the adrenal, breast, lung and ovary, pheochromocytes of the adrenal and tubular epithelium of the kidney. In contrast to huCE7 LV40 diffuse anti CD171(L1) positive staining was also observed in the extra-cellular matrix of the lymph node. This represent an important aspect of the here presented inventions as it can be postulated that a reduced uptake of huCE7 variants in healthy lymph nodes will results in a better side effect profile. The effect maybe a result of the CE7 epitope and sequence changes during humanization so that the antibody does not bind the version of L1-CAM expressed in lymph nodes.

Characterization of Fc Receptor Interaction huCE7 LV40

For radio oncology application it is favorable that the targeting mAb has no interaction with immune cells to avoid side effects. In this regard huCE7 LV40, a derivative of huCE7 Variant 7 was Fc-silenced by integration of several point mutations (N297A, L234A, L235A, P331 S). In order to verify the effect of these point mutations, surface plasmon resonance (SPR) analysis was performed with relevant receptors.

### Methodology

Effect on interaction with Fc receptor was analyzed by SPR [Instrument: Biacore T200 (C1q and FcRn) and 8K+ for (FcγR)]. Analysis temperature: 25° C., sample compartment: 12° C., Sensor chips: CM3 (C1q) and C1 (FcγR and FcRn).

### Analysis Buffer:

FcγR analyses: 10 mM HEPES pH 7.4, 150 mM NaCl, 3 mM EDTA, 0.05% Tween 20
FcRn analyses: 50 mM Na-Acetate pH 6.0, 150 mM NaCl, 3 mM EDTA, 0.05% Tween 20
C1q analyses: 10 mM HEPES pH 7.4, 300 mM NaCl, 3 mM EDTA, 0.05% Tween 20

The samples used in this study are summarized in Table E43.

TABLE E43
Samples used in FC receptor interaction study

| | | | MW | | | | | |
|---|---|---|---|---|---|---|---|---|
| Sample | Supplier | Lot | (kDa) | dissolved in | Concentration | | Storage | |
| human FcyRI/CD64 | Acro | CBV419P1-207EF1-14Y | 34.2 | H₂O (product lyophilized in | 0.1 | mg/ml | ≤−70° | C. |
| | Biosystems | | | PBS, pH 7.4, 10% Trehalose) | | | | |
| human FcyRIIIA/CD16a | Acro | BV1899-21CRF1-12S | 25.5 | H₂O (product lyophilized in | 0.1 | mg/ml | ≤−70° | C. |
| (V176) | Biosystems | | | PBS, pH 7.4) | | | | |
| human FcyRIIA/CD32a | Acro | BV1896-222QF1-13V | 24.0 | H₂0 (product lyophilized in | 0.1 | mg/ml | ≤−70° | C. |
| (H167) | Biosystems | | | PBS, pH 7.4, 10% Trehalose) | | | | |
| human FcRn / | Acro | BVG137-2132F1-13P | 45.9 | H₂0 (product lyophilized in | 0.1 | mg/ml | ≤−70° | C. |
| FCGRT&B2M | Biosystems | | | PBS, pH 7.4, 10% Trehalose) | | | | |
| human C1q | Teco | 59 | 410 | 10 mM Hepes, 300 mM NaCl | 1.12 | mg/ml | ≤−70° | C. |
| purified protein | Medical | | | pH 7.2 | | | | |
| Mabthera (Rituximab) | Medizone | N7581B14 | 150 | N/A | 10 | mg/ml | 2-8° | C. |
| LV40 | ProteoGenix | 19244-022223-A01 | 150 | PBS, pH 7.5 | 5 | mg/ml | ≤−70° | C. |

Qualitative and quantitative interaction analysis was performed with huCE7 LV40 and Rituximab (served as reference control) with reversibly immobilized human CD64, CD16a, CD32a, FcRn and C1q proteins. Concentration range was adjusted to optimal window depending on results of qualitative analysis. The study was conducted by Biaffin GmbH & Co KG (Bielefeld, Germany). The received values of binding constant (K_{D}) are presented in Table E44.

TABLE E44
Samples used in FC receptor interaction study

| | | | ratio K_{D} |
|---|---|---|---|
| | | K_{D} (M) | (K_{D} ₛₐₘₚₗₑ / K_{D control}) |
| human CD64 | Rituximab | (4.07 ± 0.02)E−11 | 389 |
| | LV40 | (1.58 ± 0.08)E−08 | |
| human CD32a | Rituximab | (3.24 ± 0.07)E−07 | — |
| (H167) | LV40 | — | |
| human CD16a | Rituximab | (5.79 ± 0.12)E−08 | — |
| (V176) | LV40 | — | |
| human FcRn | Rituximab | (3.84 ± 0.28)E−07 | 1.2 |
| | LV40 | (4.59 ± 0.33)E−07 | |
| human C1q | Rituximab | (1.29 ± 0.13)E−07 | — |
| | LV40 | — | |

### Results:

FC receptor interaction of huCE7 LV40 was not detectable for CD32a, CD16a and C1q, interaction with CD64 was reduced by a factor of 389 as compared to Rituximab control whereas the neonatal receptor binding (FcRn) was unimpaired. These results indicate that huCE7 as a low to neglectable interaction with Fc-receptors, and should not have negative influence on immune cells. Antibody-dependent cellular cytotoxicity (ADCC) and complement-dependent cytotoxicity (CDC) is therefore not expected.

Characterization of Cross Reactivity of huCE7 LV40 with Non-Human Primate L1-CAM

In order to perform a clinical translation of an antibody candidate tolerability studies in non-human primate models are required. To ensure a proper study setup the antibody must have cross reactivity for non-human primate receptor analog of L1-CAM (CD171). Therefore, huCE7 LV40 was tested for binding affinity to the non-human primate antigens from species M. fascicularis and M. mulatta using grating coupled interferometry (GCI).

### Methodology

### Antigen Immobilization

In the experiment the L1-CAM target proteins (M. fascicularis Cat. Log CPA1-1 C and M. mulatta Cat. Log CPA1-2, Supplier Sino Biological, China) were immobilized as ligand to the surface of an amine reactive sensor chip. The ligand was immobilized via amine coupling by diluting in a preconcentration buffer with a low ionic strength and a pH below the isoelectric point of the protein. Subsequently the solution was injected over the surface of an EDC NHS activated sensor chip. The positive net charge of the protein in the preconcentration buffer was used for preconcentration of the protein in the negatively charged sensor matrix, thereby driving the coupling reaction. After coupling of the ligand, unused amine reactive groups were passivated with an injection of ethanolamine. Details are presented in table E45.

TABLE E45
Experimental setup for antigen immobilization

| Sensor chip | PCP | |
|---|---|---|
| Chip conditioning | | |
| Conditioning solution | 1M NaCl, 0.1M Na-Borate, pH 9.0 | |
| Injection time | 180 | sec |
| Flowrate | 2.5 | μl/min |
| Surface activation | | |
| Activation solution | 1:1 mixture of 100 mM NHS and 400 mM EDC | |
| Injection time | 420 | sec |
| Flowrate | 3 | μl/min |
| Ligand coupling | | |
| Ligand | L1CAM proteins at 3 μg/ml in | |
| | 10 mM Sodium acetate pH 5.5 | |
| Injection time | Until target levels of ≈200 pg/mm² | |
| | are reached | |
| Flowrate | 5 | μl/min |
| Surface passivation | | |
| Passivation solution | 1M ethanolamine, pH 8.0 | |
| Injection time | 420 | sec |
| Flowrate | 3 | μl/min |
| Running buffer | 2 mM HEPES pH 7.4, 30 mM NaCl, | |
| | 0.6 mM EDTA, 0.01% Tween-20 | |
| Acquisition rate | 10 | Hz |
| Temperature: | 25° | C. |

### GCI Measurement

To investigate binding of huCE7 LV40 to amine coupled L1-CAM non-human primate proteins were analyzed in a multi cycle kinetics experiment on a [Creoptix™ WAVEdelta system using Creoptix™ WAVEcontrol software version 4.5.14]. In brief, the analyte was injected in increasing concentrations (C Analyte nM: 250, 125, 63, 31, 16, 8, 4, 2) over ligand and reference surfaces. After each analysis cycle, bound analyte was removed from the ligand by injection of regeneration solution. Raw sensor grams were inspected for nonspecific and ligand specific analyte binding. Finally, the obtained data were double referenced and fitted to an appropriate kinetic model. Detailed are presented in Table E46.

TABLE E46
Experimental setup for GCI measurement.

| Sensor chip | PCP | |
|---|---|---|
| Ligand | Amine-coupled L1CAM L1CAM M fascicularis | |
| | (≈240 pg/mm²) | |
| | Amine-coupled L1CAM M mulatta (≈190 pg/mm²) | |
| Ligand | 30 sec injections of regeneration solution, followed | |
| regeneration | by running buffer injection for surface equilibration | |
| Flowrate | 25 | μl/min |
| Regeneration | 10 mM Glycine HCl pH 1.5 | |
| solution | | |
| Analyte | LV40 diluted in running buffer, 1:2 serial | |
| | dilution with a maximum concentration of 1 μM | |
| Running buffer | 10 mM HEPES pH 7.4, 150 mM NaCl, | |
| | 3 mM EDTA, 0.05% Tween-20 | |
| Association time | 180 | sec |
| Dissociation time | 900 | sec |
| Flowrate | 40 | μl/min |
| Acquisition rate | 10 | Hz |
| Temperature | 25° | C. |

| | |
|---|---|
| Blank injections | Regularly interspaced every 4th sample |
| Data processing | Double-referencing, see FAQ |

### Results

huCE7 LV40 was titrated to the immobilized ligands in a multi cycle kinetics assay setup. Dose responsive binding of the antibody was observed. The apparent binding affinities of the interactions are in the double-digit nM range (K_{D} 44 nM for M. mulatta L1-CAM, K_{D}47 nM for M. fascicularis L1-CAM) which is suitable for later animal studies. Details are presented in table E47.

TABLE E47
Cross reactivity of huCE7 LV40 with non-human primate L1-CAM

| M. mulatta L1-CAM with huCE7 | | M. fascicularis L1-CAM with | |
|---|---|---|---|
| LV40 | | huCE7 LV40 | |
| Fit model | Bivalent | Fit model | Bivalent |
| Rₘₐₓ (pg/mm²) | 35 | Rₘₐₓ (pg/mm²) | 30 |
| kₐ₁ (M⁻¹s⁻¹) | 3.8 × 10⁵ | kₐ₁ (M⁻¹s⁻¹) | 3.6 × 10⁵ |
| k_{d1} (s⁻¹) | 1.8 × 10⁻² | k_{d1} (s⁻¹) | 1.8 × 10⁻² |
| kₐ₂ (M⁻¹s⁻¹) | 1.0 × 10⁻⁴ | kₐ₂ (M⁻¹s⁻¹) | 9.5 × 10⁵ |
| k_{d2} (s⁻¹) | 1.4 × 10⁻³ | k_{d2} (s⁻¹) | 1.5 × 10⁻³ |
| App. K_{D} (nM) | 44 | App. K_{D} (nM) | 47 |
| Sqrt Chi² (pg/mm²) | 0.487 | Sqrt Chi² (pg/mm²) | 0.393 |

### Example 27: In Vivo Characterization of huCE7 Variants

Characterization of In Vivo Biodistribution huCE7 LV40 & Polymer Modified huCE7 LV40 Radioligand in a Mouse Xenograft Model for Cis Platin Resistant Ovarian Cancer

### Methodology

### Preparation of DBCO-(Polymer-α-DOTA₄)

This hydrophilic and biocompatible copolymer was prepared by methods described in US20220409737A1 and US20210128735A1, which are herein incorporated by reference in their entirety. In brief: the random copolymer was composed obtained by RAFT polymerization using a mixture of dimethylacrylamide and acryloyl-L-lysine units monomers in an average (molar ratio: of 53 eq to 4 eq), a radical starter and a RAFT agent. After polymerization, the RAFT headgroup was converted to a reactive thiol via aminolysis and the obtained polymer was subsequently modified with DOTA chelators using amine reactive DOTA-NHS-esters as well as a click reactive DBCO headgroup via maleimide-thiol chemistry to form the resulting copolymer which can be labeled with metal ions by an interaction with which are bound by the chelator.

Preparation of antibody conjugates huCE7-LV40-(PEG₄-DOTA)₂ or polymer modified huCE7-LV40-(PEG₄-Polymer-α-DOTA₄)₂ was performed by using methods presented in Example 15 and Example 17. Radioactive labeling of both compounds was conducted with protocols as presented in Example 18.

Test system justification: The ¹⁷⁷Lu radiolabeled compounds have to be tested for in vivo behavior. Therefore, the immunocompromised mouse strain Crl:CD1-Foxn1 nu was used which was inoculated s.c. with Her2/neu; L1-CAM positive SKOVip3 cells. These cells form a tumor within 18 days.

Experimental Design: Inoculation of 5 Mio SKOVip3 cells s.c. into Crl:CD1-Foxn1 nu mice. After 18 days the ¹⁷⁷Lu-antibody conjugates which should be compared were injected i.v. A total of 40 mice bearing SKOVip3 tumors were injected with 150 kBq ¹⁷⁷Lu huCE7-LV40-(PEG₄-DOTA)₂ or huCE7-LV40-(PEG₄-Polymer-α-DOTA₄)₂ in 100 μl injection solution. For the biodistribution study the animals are sacrificed after 4, 24, 48, 72 and 96 h (4 animals per group). The organs/tissues were dissected, weighed and radioactivity was measured. The % injected activity per g organ/tissue were calculated.

### Results

Both antibody radionuclide conjugates showed a high uptake into the tumor tissue which peaked at 96 h. Uptake into other organs was low, especially in the bone tissue which represents the dose limiting organ in antibody-based radio therapies (hematotoxicity due to contact with blood cells). The pharmacokinetic profile of huCE7-LV40-(PEG₄-DOTA)₂ and the polymer modified huCE7-LV40-(PEG₄-Polymer-α-DOTA₄)₂ was comparable which indicates a high robustness of the antibodies presented in this disclosure to attachment of different linker-drug compositions.

### Conclusion

These data indicate that the in vivo targeting of L1-CAM positive tumors was successful with two different radio conjugates based on huCE7 LV40. Both test items showed an increasing tumor uptake over time and a lower uptake in the background organs resulting in favorable tumor to background ratio for therapeutic applications.

Characterization of In Vivo Efficacy of huCE7 Variant 7AG Radioligand in a Mouse Xenograft Model for Cis Platin Resistant Ovarian Cancer

In this study the in vivo proof of concept for humanized antibody variants was tested in a relevant tumor xenograft model for aggressive cis platin resistant ovarian cancer.

### Methodology

Test system justification: The ¹⁷⁷Lu radiolabeled compounds have to be tested for in-vivo behavior. Therefore, the immunocompromised mouse strain Crl:CD1-Foxn1 nu was used which was inoculated s.c. with Her2/neu; L1-CAM positive SKOVip3 cells. These cells form a tumor within 18 days.

The study used a mouse model with SKOVip3 tumor cells (cis platin resistant ovarian cancer, L1-CAM positive). 5 million cells per animal were injected, 10 mice per arm. At day 12 after inoculation the mice in the therapy arm were injected (i.v.) with a 100 μl solution of anti L1-CAM mAb huCE7-V7AG-(PEG₄-DOTA)₂ (preparation presented above in analogy to huCE7-LV40) labeled with 4 MBq ¹⁷⁷Lu, negative control arm received a 100 μl PBS pH 7.5 injection. Mice were subsequently monitored for up to 70 days (tumors size, body weight control routinely) and euthanized once the tumor size reached a certain threshold (>1 cm³) or bodyweight was reduced by 20%.

### Results

Continuous tumor growth in negative control group (by day 53 all mice had to be euthanized) was seen whereas in the treatment arm tumor size was kept under control (all mice were still alive at day 64). No change in body weight was detected which indicates high safety and tolerability of the radioligand.

### Conclusion

Given the fact that only a single injection with a low activity (4 MBq, ¹⁷⁷Lu) was used in this study the effect on overall survival is promising and shows the potential of the new humanized antibody variants in this disclosure.

### Example 28: Characterization of In-Vivo Efficacy & Tolerability of huCE7 Variant 7AG Antibody-Drug-Conjugate with MMAE in a Zebrafish Xenograft Model for Small Cell Lung Cancer

The study to evaluate a model ADC comprising the toxin Monomethyl auristatin E (MMAE) for its tolerability and efficacy in tumor xenografts was performed by BioReperia AB (Sweden).

### Methodology

### Material

The H2171 cell line was purchased from the DSMZ-German Collection of Microorganisms and Cell Cultures GmbH, Lot number ACC-544 #1. The HuCE7-V7AG antibody was manufactured by ProteoGenix SAS and provided by CIS-Pharma as a 5 mg/mL stock in PBS pH 7.5. The antibody was stored at −80° C. and used freshly thawed for the efficacy study. The huCE7-V7AG-(MMAE)₄ antibody was manufactured by Sterling Pharma Solution (UK) [using microbial Transglutaminase-based coupling chemistry for attachment of a NH₂—PEG₄-Azide linker at position Q295 and subsequent click reaction with DBCO-(PEG₂-vc-PAB-MMAE)₂] and provided directly from manufacturer as a 2.8 mg/mL stock in PBS pH 7.4. The antibody was stored at −80° C. and used freshly thawed for the efficacy study. Working concentrations of the antibodies were prepared in PBS before i.v. administration. Transgenic Tg(fli1:EGFP)y1 zebrafish embryos were used for all studies.

Toxicity Evaluation of huCE7-V7AG Antibody (Reduced Tox)

The huCE7-V7AG antibody was injected intravenously into 48 hour old zebrafish embryos. Evaluation on embryo survival and LD50 was performed after 72 hours of treatment. The results showed 95-100% embryo survival at all tested concentrations of the antibody after 72 hours of treatment (Table E48).

50% embryo mortality was not observed and LD₅₀ concentration could not be determined (ND).

TABLE E48
Evaluation of embryo survival at 72 h after i.v. injection of huCE7-V7AG antibody.

| | Percentage of Embryo survival (Number of live embryos/ | | | | | | |
|---|---|---|---|---|---|---|---|
| | Total number of embryos included in the study) | | | | | | LD50 |
| Compound | Vehicle | 0.03 mg/mL | 0.1 mg/mL | 0.3 mg/mL | 1 mg/mL | 3 mg/mL | (μM) |
| HuCE7- | 100 (20/20) | 100 (20/20) | 95 (19/100) | 100 (20/20) | 100 (20/20) | 100 (20/20) | ND |
| V7Ag | | | | | | | |

Toxicity Evaluation of huCE7-V7AG-(MMAE)4 Antibody (Full Tox)

The huCE7-V7AG-(MMAE)₄ antibody was injected intravenously into 48 hours old zebrafish embryos. Evaluation of embryo survival, non-lethal toxicities, and LD₅₀/LT₅₀ was performed after 72 hours of treatment. Table E49 shows the percentage of embryo survival after 72 hours of treatment. 70%-100% embryo survival was observed throughout the evaluation time in all tested concentrations.

TABLE E49
Percentage of embryo survival after
huCE7-V7AG-(MMAE)₄ administration
HuCE7-V7Ag (MMAE)4 treatment

| Concentration | Time | | |
|---|---|---|---|
| [mg/mL] | 24 h | 48 h | 72 h |
| 0 | 100 | 95 | 85 |
| 0.03 | 100 | 95 | 85 |
| 0.1 | 100 | 75 | 75 |
| 0.3 | 100 | 100 | 100 |
| 1 | 100 | 75 | 70 |
| 2.8 | 100 | 85 | 70 |

### Lethal Tax

LD₅₀ (Lethal dose 50) is defined as the dose at which 50% of the embryos die during the experiment. However, 50% embryo mortality was not reached at any of the concentrations tested; then, LD₅₀ could not be calculated for the huCE7-V7AG-(MMAE)₄ antibody.

### Non-Lethal Toxicity

Developmental malformations were observed throughout the evaluation time after exposure to the huCE7-V7AG-(MMAE)₄ antibody. Table E50 shows the percentage of embryos exhibiting non-lethal toxicity at each time point for every concentration tested.

TABLE E50
Non-lethal toxicity evaluation

| HuCE7-V7Ag (MMAE)4 treatment | | | | |
|---|---|---|---|---|
| | Percentage of | | | |
| | embryos showing | | | |
| | non-lethal toxicity | | | |
| Concentration | Time | | | |
| [mg/mL] | 24 h | 48 h | 72 h | Non-lethal toxicity observed |
| Vehicle | 5 | 21 | 11 | Pericardial edema |
| 0.03 | 25 | 47 | 29 | Pericardial edema, yolk sac edema, malformation of |
| | | | | head and yolk sac, small eyes |
| 0.1 | 40 | 40 | 19 | Pericardial edema, yolk sac edema, head necrosis, |
| | | | | malformation of head and yolk sac, small eyes |
| 0.3 | 30 | 30 | 15 | Pericardial edema, yolk sac edema, malformation of |
| | | | | head and yolk sac, small eyes, scoliosis |
| 1 | 40 | 60 | 21.4 | Pericardial and yolk sac edema, malformation of yolk |
| | | | | sac |
| 2.8 | 50 | 76 | 85 | Pericardial edema, yolk sac edema, head necrosis, |
| | | | | malformation of head and yolk sac, small eyes, yolk |
| | | | | sac necrosis, scoliosis |

huCE7-V7AG was well tolerated as non-toxicity were seen up to 3 mg/ml. We conclude that the huCE7-V7AG-(MMAE)₄ antibody showed average embryo mortality and more developmental defects than the huCE7-V7AG antibody, mainly due to the conjugation of the antineoplastic agent.

### Efficacy Evaluation of Antibodies on Primary Tumor and Metastasis of MAXFTN-401 Breast Cancer Cells Cell Line Using ZTX®ONCOLEADS

MAXFTN-401 cancer cells were used to evaluate the anti-tumor efficacy of huCE7-V7AG and huCE7-V7AG-(MMAE)₄ antibodies. The anti-tumor efficacy of antibodies was determined as the change in primary tumor size (i.e., tumor growth or reduction) and the number of tumor cells disseminated to the CVP three days after implantation.

Results showed that huCE7-V7AG antibody had no effect on primary tumor size or metastasis of MAXFTN-401 cells at 3 mg/mL compared to the negative control.

A possible explanation of the lower effect on the naked mAb on the tumor cells maybe the fact that the ADO was injected simultaneously with the implantation of the cells, so that the tumor had not enough time to form blood vessel and tissue before the ADO was cleared form the blood. Furthermore, MAXFTN-401 cells have a low to mid expression of the target L1-CAM on RNA level according to the supplier's database. In order to find a more suitable model a FACS analysis was performed with another cell line H2171 (small cell lung cancer)

H-2171 were purchased from the Leibniz Institute and cultured according to provider's instructions. The MDA-MB-468 cancer cell line was used as negative control and cultured according to provider's instructions. Cancer cell lines were selected based on their high L1-CAM expression according to the Charles River database.

The following procedure was performed for FACS analysis:

4 sample tubes containing 250,000 live cells per tube were prepared and incubated for 45 min at 37° C. and 5% CO₂ in culture medium. Two sample tubes were washed once in FCM buffer (PBS, 1% BSA) and then incubated for 1 h on ice with huCE7-V7AG antibody or isotype antibody at 20 μg/ml in FCM buffer. After incubation, the cells were washed once with FCM buffer and incubated on ice for 1 hour in FCM buffer with a FITC-labeled goat anti-human secondary antibody at a defined dilution (1/1500). The third sample tube was washed once in FCM buffer and incubated only with the FITC-labeled goat anti-human antibody at a defined dilution on ice for 1 hour in FCM buffer. The fourth sample tube contained only unmarked cells. Unmarked control cells were treated the same as those in tubes 1-2 and were washed with FCM buffer every time the treated samples were washed. After treatment, all sample tubes were washed twice with FCM buffer, resuspended in 200-500 μL of FCM buffer, and were immediately analyzed with Gallios flow cytometer (Beckmann Coulter). The same procedure was used for the negative control cell line

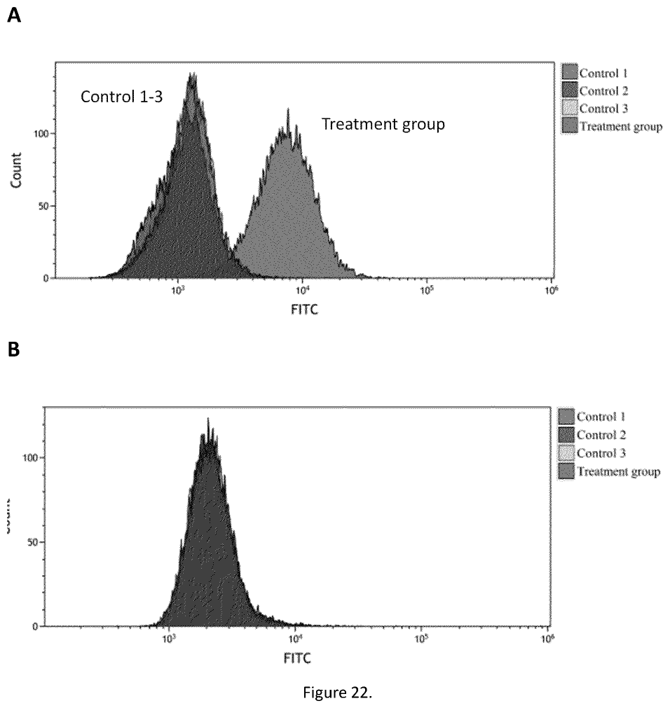

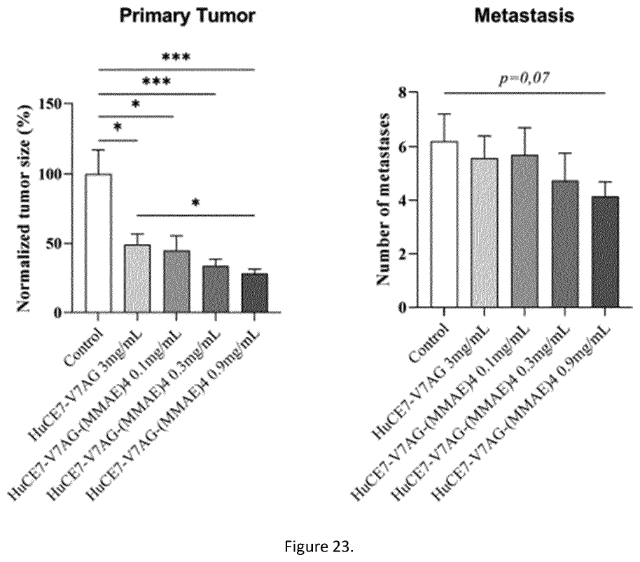

The FACS analysis indicated a strong binding of huCE7 variant 7 AG to L1-CAM antigen on H2171 cells. The binding was very specific as negative control cell line MDA-MB-468 not expression L1-CAM did not show any binding of the antibody. The results are shown in FIG. 22.

### Efficacy Evaluation of Antibodies on Primary Tumor and Metastasis of H2171 Cell Line Using ZTX®ONCOLEADS

H2171 cancer cells were used to evaluate the anti-tumor efficacy of huCE7-V7AG and huCE7-V7AG-(MMAE)₄ antibodies. The study is comprised of 5 experimental groups. Tumor cells were implanted subcutaneously and treatment with antibodies was administered intravenously at 24 h post-tumor implantation (pti) [to give enough time for the tumor to grow before ADC was applied]. 20 tumor-bearing embryos were included per experimental group and incubated at 35.5° C. for 3 days.

TABLE E51
Description of experimental groups for efficacy analysis of HuCE7-
V7AG and HuCE7-V7AG-(MMAE)4 antibodies using ZTX-ONCOLEADS.

| | | | Admin. of | Evaluation of | |
|---|---|---|---|---|---|
| | | Group | treatment at | primary tumor | Evaluation of |
| Treatment | Conc. | size | 24 h pti | size | metastasis |
| Vehicle | — | 20 | i.v. | 0 and 72 h | 72 h |
| (PBS) | | | | | |
| HuCE7-V7AG Ab | 3 mg/ml | 20 | i.v. | 0 and 72 h | 72 h |
| HuCE7-V7AG- | 0.1 mg/ml | 20 | i.v. | 0 and 72 h | 72 h |
| (MMAE)4 | | | | | |
| HuCE7-V7AG- | 0.3 mg/ml | 20 | i.v. | 0 and 72 h | 72 h |
| (MMAE)4 | | | | | |
| HuCE7-V7AG- | 0.9 mg/ml | 20 | i.v. | 0 and 72 h | 72 h |
| (MMAE)4 | | | | | |

### Summary Results

This study aimed to evaluate huCE7-V7AG and huCE7-V7AG-(MMAE)₄ antibodies' anti-tumor effect on primary tumor size, and metastasis formation of H2171 small cell lung cancer (SCLC) cells. The anti-cancer efficacy of the antibodies was determined by the change in primary tumor size (i.e., tumor growth or reduction) and the number of tumor cells disseminated to the distal caudal venous plexus (CVP) three days after implantation.

Results showed that treatment with huCE7-V7AG at 3 mg/mL and huCE7-V7AG-(MMAE)₄ at 0.1, 0.3, and 0.9 mg/mL significantly decreased primary tumor size after 2 days of treatment compared to a control group. While both huCE7-V7AG and huCE7-V7AG-(MMAE)₄ treatment antibodies displayed anti-tumor effect on the H2171 cell line, the huCE7-V7AG-(MMAE)₄ antibody treatment at 0.9 mg/mL exhibited significantly stronger anti-tumor effect than huCE7-V7AG at 3 mg/mL. Further huCE7-V7AG-(MMAE)₄ at 0.9 mg/mL decreased the number of metastasized cells to the CVP after 2 days of treatment compared to the control group. This effect was borderline significant.

### CONCLUSION

This study verified that already the naked antibody of this disclosure huCE7 Variant 7AG has the potential to inhibit tumor growth of L1-CAM positive cells and that this effect can be strengthened by addition of cytotoxic agents. The positive anti-tumor effect maybe a combination of blocking the target, especially preventing spreading of tumor cells by shielding integrin binding side in IgG like domain 6 of L1-CAM, and delivery of cytotoxic agent into the tumor cell.

1. An antibody or an antigen binding fragment thereof that specifically binds to L1-CAM (CD171), the antibody or the antigen binding fragment thereof comprising:
a variable heavy chain region comprising:
CDR-H1 characterized by a sequence selected from
a sequence according to SEQ ID NO.:1 (GYWMH),
a sequence according to SEQ ID NO.: 2 (GYYMH),
a sequence according to SEQ ID NO.: 3 (GYFMH), and
a sequence according to SEQ ID NO.: 4 (GYLMH); and
CDR-H2 characterized by a sequence selected from
a sequence according to SEQ ID NO.: 5 (EINPSNGRTNYNERFQG),
a sequence according to SEQ ID NO.: 6 (EINPSNGRTNYNEKFQG),
a sequence according to SEQ ID NO.: 7 (EINPSNGRTNYNERFKS),
a sequence according to SEQ ID NO.: 8 (EINPSNGRTNYNERLKS),
a sequence according to SEQ ID NO.: 9 (EINPSNARTNYNERFQG),
a sequence according to SEQ ID NO.: 10 (EINPSNARTNYNEKFQG)
a sequence according to SEQ ID NO.: 11 (EINPSNARTNYNERFKS) and
a sequence according to SEQ ID NO.: 12 (EINPSNARTNYNERLKS); and
CDR-H3 characterized by a sequence according to SEQ ID NO.: 13 (DYYGTSYNFDY); and
a variable light chain region comprising:
CDR-L1 characterized by a sequence selected from
a sequence according to SEQ ID NO.: 14 (RANEDINNRLA),
a sequence according to SEQ ID NO.: 15 (KANEDINNRLA),
a sequence according to SEQ ID NO.: 16 (QANEDINNRLA),
a sequence according to SEQ ID NO.: 17 (RANEDINARLA),
a sequence according to SEQ ID NO.: 18 (KANEDINARLA),
a sequence according to SEQ ID NO.: 19 (QANEDINARLA),
a sequence according to SEQ ID NO.: 20 (RANEDINLRLA),
a sequence according to SEQ ID NO.: 21 (KANEDINLRLA), and
a sequence according to SEQ ID NO.: 22 (QANEDINLRLA); and
CDR-L2 characterized by a sequence selected from
a sequence according to SEQ ID NO.: 23 (GATNLVT) and
a sequence according to SEQ ID NO.: 24 (GASNLVS); and
CDR-L3 characterized by a sequence selected from
a sequence according to SEQ ID NO.: 25 (QQYWSTPFT),
a sequence according to SEQ ID NO.: 26 (QQYYSTPFT) and
a sequence according to SEQ ID NO.: 27 (QQYFSTPFT),

2. The antibody or the antigen-binding fragment thereof of claim 1, wherein the antibody or the antigen binding fragment thereof is a monoclonal antibody, a chimeric antibody, a recombinant antibody, an antigen-binding fragment of a recombinant antibody, a single chain antibody, a humanized antibody, a bispecific antibody, a multi-specific antibody, or an antibody displayed upon the surface of a phage or displayed upon the surface of a chimeric antigen receptor (CAR) T cell.

3. The antibody or the antigen-binding fragment thereof of claim 1, wherein the antibody or the antigen binding fragment thereof is a monoclonal antibody, or wherein the antibody or the antigen binding fragment thereof is an IgG1 antibody.

4. (canceled)

5. The antibody or the antigen-binding fragment thereof of claim 1, wherein the variable heavy chain region comprises CDR-H1 characterized by a sequence according to SEQ ID NO.: 1, CDR-H2 characterized by a sequence according to SEQ ID NO.: 5, 6, 9 or 10, and/or CDR-H3 characterized by a sequence according to SEQ ID NO.: 13, and/or wherein the variable light chain region comprises CDR-L1 characterized by a sequence according to SEQ ID NO.: 14, 15, 17, 18, or 21, CDR-L2 characterized by a sequence according to SEQ ID NO.: 23, and/or CDR-L3 characterized by a sequence according to SEQ ID NO.: 25.

6. (canceled)

7. The antibody or the antigen-binding fragment thereof of claim 1, wherein the variable heavy chain region comprises CDR-H1 characterized by a sequence according to SEQ ID NO.: 1, CDR-H2 characterized by a sequence according to SEQ ID NO.: 5 or 6, and/or CDR-H3 characterized by a sequence according to SEQ ID NO.: 13, and/or wherein the variable light chain region comprises CDR-L1 characterized by a sequence according to SEQ ID NO.: 14 or 15, CDR-L2 characterized by a sequence according to SEQ ID NO.: 23, and/or CDR-L3 characterized by a sequence according to SEQ ID NO.: 25.

8-11. (canceled)

12. The antibody or the antigen-binding fragment thereof of claim 1, wherein the variable heavy chain region is characterized by a sequence at least 90% identical to a sequence selected from
a sequence according to SEQ ID NO.: 28 (QVQLVQSGAEVKKPGASVKVSCKASGYTFTGYWMHWVRQAPGQGLEWI GEINPSNGRTNYNERFQGRVTLTVDKSISTAYMELSRLRSDDTAVYYCARD YYGTSYNFDYWGQGTLVTVSS),
a sequence according to SEQ ID NO.: 29 (QVQLVQSGAEVKKPGASVKVSCKASGYTFTGYWMHWVRQAPGQGLEWI GEINPSNGRTNYNERFKSRVTLTVDKSISTAYMELSRLRSDDTAVYFCARD YYGTSYNFDYWGQGTLVTVSS),
a sequence according to SEQ ID NO.: 30 (QVQLQQWGAGLLKPSETLSLTCAAYGYTFTGYWMHWIRQPPGKGLEWIG EINPSNGRTNYNERLKSRVTLSVDKSKNQASLKLSSVTAADTAVYFCARDY YGTSYNFDYWGQGTLVTVSS),
a sequence according to SEQ ID NO.: 31 (QVQLVQSGAEVKKPGASVKVSCKASGYTFTGYWMHWVRQAPGQGLEWI GEINPSNGRTNYNEkFQGRVTLTVDKSISTAYMELSRLRSDDTAVYYCARD YYGTSYNFDYWGQGTLVTVSS), and
a sequence according to SEQ ID NO.: 32 (QVQLVQSGAEVKKPGASVKVSCKASGYTFTGYWMHWVRQAPGQGLEW mGEINPSNGRTNYNEkFQGRVTLTVDKSISTAYMELSRLRSDDTAVYYCAR DYYGTSYNFDYWGQGTLVTVSS)

13. (canceled)

14. The antibody or the antigen-binding fragment thereof of claim 1, wherein the variable light chain region is characterized by a sequence at least 90% identical to, preferably at least 95% identical to, more preferably identical to a sequence selected from
a sequence according to SEQ ID NO.: 33 (DIQMTQSPSSLSASVGDRVTITCKANEDINNRLAWYQQKPGKAPKLLISGA TNLVTGVPSRFSGSGSGKDYTLTISSLQPEDFATYYCQQYWSTPFTFGQGTK LEIK),
a sequence according to SEQ ID NO.: 34 (DIQMTQSPSSLSASVGDRVTITCKANEDINNRLAWYQQKPGKAPKLLISGA TNLVTGVPSRFSGSGSGKDYTLTISSLQPEDIATYYCQQYWSTPFTFGQGTK LEIK),
a sequence according to SEQ ID NO.: 35 (EIVMTQSPATLSVSPGERATLSCRANEDINNRLAWYQQKPGQAPRLLISGA TNLVTGIPARFSGSGSGKEFTLTISSLQSEDFAVYYCQQYWSTPFTFGQGTK LEIK),
a sequence according to SEQ ID NO.: 36 (DIQMTQSPSSLSASVGDRVTITCRANEDINNRLAWYQQKPGKAPKLLISGA TNLVTGVPSRFSGSGSGKDYTLTISSLQPEDFATYYCQQYWSTPFTFGQGTK LEIK), and
a sequence according to SEQ ID NO.: 37 (DIQMTQSPSSLSASVGDRVTITCRANEDINNRLAWYQQKPGKAPKLLISGAs NLVsGVPSRFSGSGSGKDYTLTISSLQPEDFATYYCQQYWSTPFTFGQGTKL EIK).

15. (canceled)

16. The antibody or the antigen-binding fragment thereof of claim 1,
wherein the variable heavy chain region is characterized by a sequence according to SEQ ID NO.: 28 and the variable light chain region is characterized by a sequence according to SEQ ID NO.: 33; or
wherein the variable heavy chain region is characterized by a sequence according to SEQ ID NO.: 32 and the variable light chain region is characterized by a sequence according to SEQ ID NO.: 36; or
wherein the variable heavy chain region is characterized by a sequence according to SEQ ID NO.: 14 and the variable light chain region is characterized by a sequence according to SEQ ID NO.: 37.

17. The antibody or the antigen-binding fragment thereof of claim 1,
wherein the variable heavy chain region is characterized by a sequence at least 90% identical to a sequence:
FH0-CDR-H1-FH1-CDR-H2-FH2-CDR-H3-FH3
wherein CDR-H1, CDR-H2 and CDR-H3 are as defined in claim 1, wherein:
FH0 is characterized by a sequence according to SEQ ID NO: 38 (QVQLVQSGAEVKKPGASVKVSCKASGYTFT) or a sequence according to SEQ ID NO: 39 (QVQLQQWGAGLLKPSETLSLTCAAYGYTFT),
FH1 is characterized by a sequence according to SEQ ID NO: 40 (WVRQAPGQGLEWIG) or a sequence according to SEQ ID NO.: 41 (WIRQPPGKGLEWIG)
FH2 is characterized by a sequence according to SEQ ID NO: 42 (RVTLTVDKSISTAYMELSRLRSDDTAVYFCAR) or a sequence according to SEQ ID NO.: 43 (RVTLSVDKSKNQASLKLSSVTAADTAVYFCAR), and
FH3 is characterized by a sequence according to SEQ ID NO:44 (WGQGTLVTVSS), and
wherein the variable light chain region is characterized by a sequence at least 90% identical to a sequence:
FL0-CDR-L1-FL1-CDR-L2-FL2-CDR-L3-FL3
wherein CDR-L1, CDR-L2 and CDR-L3 are as defined in claim 1, wherein:
FL0 is characterized by a sequence according to SEQ ID NO: 45 (DIQMTQSPSSLSASVGDRVTITC) or a sequence according to SEQ ID NO: 46 (EIVMTQSPATLSVSPGERATLSC),
FL1 is characterized by a sequence according to SEQ ID NO: 47 (WYQQKPGKAPKLLIS) or a sequence according to SEQ ID NO.: 48 (WYQQKPGQAPRLLIS),
FL2 is characterized by a sequence according to SEQ ID NO: 49 (GVPSRFSGSGSGKDYTLTISSLQPEDIATYYC) or a sequence according to SEQ ID NO.: 50 (GIPARFSGSGSGKEFTLTISSLQSEDFAVYYC), and
FL3 is characterized by a sequence according to SEQ ID NO:51 (FGQGTKLEIK).

18. The antibody or the antigen-binding fragment thereof of claim 1, wherein the heavy chain of the antibody or the antigen-binding fragment thereof comprises a sequence at least 90% identical to a sequence selected from sequences according to SEQ ID NO.: 110, 142, 155 to 163 and
wherein the light chain of the antibody or the antigen-binding fragment thereof comprises a sequence at least 90% identical to a sequence according to SEQ ID NO.: 97 or 143.

19. The antibody or an antigen binding fragment thereof of claim 1,
wherein the heavy chain comprises a sequence at least 90% identical to a sequence according to SEQ ID NO.: 155, and wherein the light chain comprises a sequence at least 90% identical to a sequence according to SEQ ID NO.: 143
or
wherein the heavy chain comprises a sequence at least 90% identical to a sequence according to SEQ ID NO. 110, 142, 155 to 163, and wherein the light chain comprises a sequence at least 90% identical to a sequence according to SEQ ID NO.: 97 or 143,
or
wherein the heavy chain comprises a sequence at least 90% identical to a sequence selected from sequences according to SEQ ID NO. 95 or 164 to 172, and wherein the light chain comprises a sequence at least 90% identical to a sequence according to SEQ ID NO.: 97 or 143,
or
wherein the heavy chain comprises a sequence at least 90% identical to a sequence selected from sequences according to SEQ ID NO. 173 to 183, and wherein the light chain comprises a sequence at least 90% identical to a sequence according to SEQ ID NO.: 193,
or
wherein the heavy chain comprises a sequence at least 90% identical to a sequence selected from sequences according to SEQ ID NO. 173 to 183, and
wherein the light chain comprises a sequence at least 90% identical to a sequence according to SEQ ID NO.: 194,
or
wherein the heavy chain comprises a sequence at least 90% identical to a sequence selected from sequences according to SEQ ID NO. 184 to 192, and
wherein the light chain comprises a sequence at least 90% identical to a sequence according to SEQ ID NO.: 193,
or
wherein the heavy chain comprises a sequence at least 90% identical to a sequence selected from sequences according to SEQ ID NO. 184 to 192, and
wherein the light chain comprises a sequence at least 90% identical to a sequence according to SEQ ID NO.: 194,
or
wherein the heavy chain comprises a sequence at least 90% identical to a sequence according to SEQ ID NO.: 175, and wherein the light chain comprises a sequence at least 90% identical to a sequence according to SEQ ID NO.: 194.

20-26. (canceled)

27. The antibody or the antigen-binding fragment thereof of claim 1, wherein the heavy chain further comprises at least one point mutation in Fc part that influences antibody-dependent cell-mediated cytotoxicity (ADCC), complement-dependent cytotoxicity (CDC), serum half-life and/or glycosylation status of the antibody, and/or wherein the at least one point mutation is selected from L234A, L234F, L235A, L235E, L235Q, G236A, M252Y, S254T, T256E, S267E, H268F, N297A, K322A, K322Q, S324T, P331S, and I332E, and/or wherein the antibody or the antigen-binding fragment thereof is characterized by a dissociation constant K_{D} to L1-CAM (CD171) not exceeding 10⁻¹¹ M, as measured in a Biacore-based assay.

28. (canceled)

29. (canceled)

30. A polynucleotide encoding at least one variable heavy chain sequence and/or at least one variable light chain sequence as described in claim 1.

31. A host cell comprising the polynucleotide of claim 30.

32. An immunoconjugate comprising an antibody or an antigen-binding fragment thereof of claim 1 and an active agent.

33. The immunoconjugate of claim 32, wherein the antibody or the antigen-binding fragment thereof is linked to the active agent through a linker moiety, optionally wherein said linker moiety comprises a polymer carrier, to which at least one active agent is attached, and/or
wherein the active agent is a cytotoxic agent or a prodrug thereof, or wherein the active agent is selected from maytansinoid, calicheamicin, pyrrolobenzodiazepine (PBD), nemorubicin and its derivatives, PNU-159682, anthracycline, duocarmycin, vinca alkaloid, taxane, trichothecene, CC1065, camptothecin, elinafide, Exatecan, Deruxtecan, topotecan, irinotecan, SN38, and belotecan, or wherein the active agent is a radionuclide, or wherein the active agent is a radionuclide selected from copper-67, strontium-89, yttrium-90, iodine-131, samarium-153, terbium-161, lutetium-177, astatine-211, radium-223 actinium 225, fluorine-18, scandium-43, scandium-44, copper-61, copper-64, gallium-68, zirconium-89, indium-111, iodine-123, terbium-152, and terbium-155.

34. (canceled)

35. (canceled)

36. A pharmaceutical composition comprising the immunoconjugate of claim 32 and a pharmaceutically acceptable carrier.

37-43. (canceled)

44. A pharmaceutical composition comprising the antibody or the antigen-binding fragment thereof of claim 1, and a pharmaceutically acceptable carrier.

45. A method of treatment of an L1-CAM associated cancer, the method comprising administering to an individual in need thereof of the antibody or antigen binding fragment thereof of claim 1.

46. The method of treatment of claim 45, wherein the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer, and head and neck squamous cell carcinoma.

47. A method of treatment of an L1-CAM associated cancer, the method comprising administering to an individual in need thereof of the immunoconjugate of claim 32.

48. The method of treatment of claim 47, wherein the L1-CAM (CD171) associated cancer is selected from leukemia, Ewing's sarcoma, neuroblastoma, osteosarcoma, glioblastoma multiforme, ovarian cancer, endometrial cancer, uterine cancer, triple negative breast cancer, quadruple-negative breast cancer, melanoma, clear cell renal cell cancer, pheochromacytoma and paraganglioma, mesothelioma, small cell lung cancer (SCLC), non-small cell lung cancer, NSCLC, pancreatic ductal cancer, colon cancer, pancreatic cancer, hepatocellular carcinoma, gastric cancer, cholangiocarcinoma, carcinoid, neuroendocrine tumors, gastrointestinal stromal tumor (GIST), pheochromocytoma, glioma, pancreatic neuroectodermal cancer, pancreatic adenocarcinoma, colorectal cancer, renal cell carcinoma, tumor blood vessels, chondrosarcoma, esophageal adenocarcinoma, oligodendroglioma, astrocytoma, ependymoma, pancreatic neuroendocrine carcinoma, adrenal adenoma, leiomyosarcoma, liposarcoma, granular cell tumor of the ovary, schwannoma, primitive neuroectodermal tumor (PNET), epitheliod sarcoma, esthesioneuroblastoma, medulloblastoma, capillary hemangioma, Kaposi sarcoma, rhabdomyosarcoma, submaxillary salivary gland cancer, prostate cancer, and head and neck squamous cell carcinoma.

49. A method of diagnosis of an L1-CAM associated cancer, the method comprising administering to a subject the immunoconjugate of claim 32, wherein the active agent is a moiety that enables detection and localization of said immunoconjugate, and monitoring said immunoconjugate to determine if an L1-CAM (CD171) associated cancer is detected.

50. The method of diagnosis of claim 49, wherein said active agent comprises a radionucleotide.

51. The antibody or an antigen binding fragment thereof of claim 1, wherein the heavy chain comprises a sequence at least 90% identical to a sequence according to SEQ ID NO. 110, or a sequence according to SEQ ID NO. 110 wherein the mutation N297A has been reversed; and wherein the light chain comprises a sequence at least 90% identical to a sequence according to SEQ ID NO.: 97.

52. The antibody or an antigen binding fragment thereof of claim 1, wherein the heavy chain comprises a sequence at least 90% identical to a sequence according to SEQ ID NO. 173, or a sequence according to SEQ ID NO. 173 wherein mutation N297A is further present; and wherein the light chain comprises a sequence at least 90% identical to a sequence according to SEQ ID NO.: 193.
